(19) United States
(12) Patent Application Publication
(10) Pub. No.: US 2024/0245699 A1
(43) Pub. Date: Jul. 25, 2024

(54) PYRROLE COMPOUNDS, A PROCESS FOR THEIR PREPARATION AND PHARMACEUTICAL COMPOSITIONS CONTAINING THEM

(71) Applicants: LES LABORATOIRES SERVIER, Suresnes Cedex (FR); VERNALIS (R&D) LTD, Cambridge (GB)

(72) Inventors: Arnaud LE TIRAN, Croissy sur Seine (FR); Thierry LE DIGUARHER, Saint Denis de I'Hôtel (FR); Jérôme-Benoît STARCK, Rueil-Malmaison (FR); Jean-Michel HENLIN, Suresnes (FR); Anne-Françoise GUILLOUZIC, Nanterre (FR); Guillaume DE NANTEUIL, Suresnes (FR); Olivier GENESTE, Rueil-Malmaison (FR); Imre FEJES, Budapest (HU); János TATAI, Budapest (HU); Miklós NYERGES, Leányfalu (HU); James Edward Paul DAVIDSON, Great Shelford (GB); James Brooke MURRAY, Linton (GB); I-Jen CHEN, Cambridge (GB); Didier DURAND, Chambourcy (FR)

(21) Appl. No.: 18/591,522

(22) Filed: Feb. 29, 2024

Related U.S. Application Data
(63) Continuation of application No. 17/341,751, filed on Jun. 8, 2021.
Application No. 17/341,751 is a continuation of application No. 16/865,855, filed on May 4, 2020, now Pat. No. 11,058,689.
Application No. 16/865,855 is a continuation of application No. 16/271,171, filed on Feb. 8, 2019, now Pat. No. 10,688,101.
Application No. 16/271,171 is a continuation of application No. 15/419,228, filed on Jan. 30, 2017, now Pat. No. 10,265,323.
Application No. 15/419,228 is a continuation of application No. 14/797,532, filed on Jul. 13, 2015, now Pat. No. 9,598,427.
Application No. 14/797,532 is a division of application No. 14/336,311, filed on Jul. 21, 2014, now Pat. No. 9,108,983.

(30) Foreign Application Priority Data
Jul. 23, 2013 (FR) 1357258

(51) Int. Cl. A61K 31/5383; A61K 9/20; A61K 31/4725; A61K 31/496; A61K 31/506; A61K 31/5377; A61K 45/06; C07D 401/10; C07D 401/14; C07D 405/14; C07D 471/04; C07D 491/10; C07D 491/107; C07D 498/04; C07D 498/14

(52) U.S. Cl. CPC A61K 31/5383; A61K 9/2059; A61K 31/4725; A61K 31/496; A61K 31/506; A61K 31/5377; A61K 45/06; C07D 401/10; C07D 401/14; C07D 405/14; C07D 471/04; C07D 491/10; C07D 491/107; C07D 498/04; C07D 498/14

(57) ABSTRACT

Compounds of formula (I):

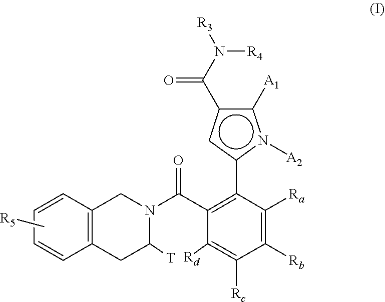

wherein A₁, A₂, Rₐ, R_{b}, R_{c}, R_{d}, R₃, R₄, R₅ and T are as defined in the description.

Medicinal products containing the same which are useful in treating pathologies involving a deficit in apoptosis, such as cancer, auto-immune diseases, and diseases of the immune system.

The present invention relates to new pyrrole compounds, to a process for their preparation and to pharmaceutical compositions containing them.

The compounds of the present invention are new and have very valuable pharmacological characteristics in the field of apoptosis and cancerology.

Apoptosis, or programmed cell death, is a physiological process that is crucial for embryonic development and maintenance of tissue homeostasis.

Apoptotic-type cell death involves morphological changes such as condensation of the nucleus, DNA fragmentation and also biochemical phenomena such as the activation of caspases which cause damage to key structural components of the cell, so inducing its disassembly and death. Regulation of the process of apoptosis is complex and involves the activation or repression of several intracellular signalling pathways (Cory S. et al., Nature Review Cancer, 2002, 2, 647-656).

Deregulation of apoptosis is involved in certain pathologies. Increased apoptosis is associated with neurodegenerative diseases such as Parkinson's disease, Alzheimer's disease and ischaemia. Conversely, deficits in the implementation of apoptosis play a significant role in the development of cancers and their chemoresistance, in auto-immune diseases, inflammatory diseases and viral infections. Accordingly, absence of apoptosis is one of the phenotypic signatures of cancer (Hanahan D. et al., Cell 2000, 100, 57-70).

The anti-apoptotic proteins of the Bcl-2 family are associated with numerous pathologies. The involvement of proteins of the Bcl-2 family is described in numerous types of cancer, such as colorectal cancer, breast cancer, small-cell lung cancer, non-small-cell lung cancer, bladder cancer, ovarian cancer, prostate cancer, chronic lymphoid leukaemia, follicular lymphoma, myeloma, etc. Overexpression of the anti-apoptotic proteins of the Bcl-2 family is involved in tumorigenesis, in resistance to chemotherapy and in the clinical prognosis of patients affected by cancer. There is, therefore, a therapeutic need for compounds that inhibit the anti-apoptotic activity of the proteins of the Bcl-2 family.

In addition to being new, the compounds of the present invention have pro-apoptotic properties making it possible to use them in pathologies involving a defect in apoptosis, such as, for example, in the treatment of cancer, auto-immune diseases and diseases of the immune system.

The present invention relates more especially to compounds of formula (I):

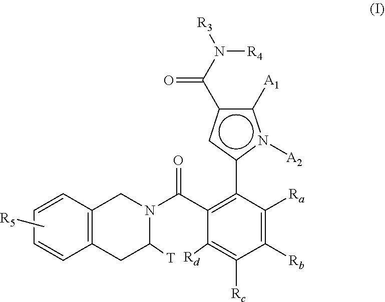

wherein:
   A₁ and A₂, each independently of the other, represent a hydrogen or halogen atom, a linear or branched (C₁-C₆)polyhaloalkyl group, a linear or branched (C₁-C₆)alkyl group or a cycloalkyl group,
   T represents a hydrogen atom, a linear or branched (C₁-C₆)alkyl group optionally substituted by from one to three halogen atoms, a group (C₁-C₄)alkyl-NR₁R₂, or a group (C₁-C₄)alkyl-OR₆,
   R₁ and R₂, each independently of the other, represent a hydrogen atom or a linear or branched (C₁-C₆)alkyl group,
   or R₁ and R₂ form with the nitrogen atom carrying them a heterocycloalkyl,
   R₃ represents a linear or branched (C₁-C₆)alkyl group, a linear or branched (C₂-C₆)alkenyl group, a linear or branched (C₂-C₆)alkynyl group, a cycloalkyl group, a (C₃-C₁₀)cycloalkyl-(C₁-C₆)alkyl group wherein the alkyl moiety is linear or branched, a heterocycloalkyl group, an aryl group or a heteroaryl group, it being understood that one or more of the carbon atoms of the preceding groups, or of their possible substituents, may be deuterated,
   R₄ represents an aryl group, a heteroaryl group, a cycloalkyl group or a linear or branched (C₁-C₆)alkyl group, it being understood that one or more of the carbon atoms of the preceding groups, or of their possible substituents, may be deuterated,
   R₅ represents a hydrogen or halogen atom, a linear or branched (C₁-C₆)alkyl group, or a linear or branched (C₁-C₆)alkoxy group,
   R₆ represents a hydrogen atom or a linear or branched (C₁-C₆)alkyl group,
   Rₐ, R_{b}, R_{c} and R_{d}, each independently of the others, represent R₇, a halogen atom, a linear or branched (C₁-C₆)alkoxy group, a hydroxy group, a linear or branched (C₁-C₆)polyhaloalkyl group, a trifluoromethoxy group, —NR₇R₇′, nitro, R₇—CO—(C₀-C₆)alkyl-, R₇—CO—NH—(C₀-C₆)alkyl-, NR₇R₇′—CO—(C₀-C₆)alkyl-, NR₇R₇′—CO—(C₀-C₆)alkyl-O—, R₇—SO₂—NH—(C₀-C₆)alkyl-, R₇—NH—CO—NH—(C₀-C₆)alkyl-, R₇—O—CO—NH—(C₀-C₆)alkyl-, a heterocycloalkyl group, or the substituents of one of the pairs (Rₐ,R_{b}), (R_{b},R_{c}) or (R_{c},R_{d}) form together with the carbon atoms carrying them a ring composed of from 5 to 7 ring members, which may contain from one to 2 hetero atoms selected from oxygen and sulphur, it also being understood that one or more carbon atoms of the ring defined hereinbefore may be deuterated or substituted by from one to 3 groups selected from halogen and linear or branched (C₁-C₆)alkyl,
   R₇ and R₇′, each independently of the other, represent a hydrogen, a linear or branched (C₁-C₆)alkyl, a linear or branched (C₂-C₆)alkenyl, a linear or branched (C₂-C₆)alkynyl, an aryl or a heteroaryl, or R₇ and R₇′, together with the nitrogen atom carrying them, form a heterocycle composed of from 5 to 7 ring members,
it being understood that:
   “aryl” means a phenyl, naphthyl, biphenyl or indenyl group,
   “heteroaryl” means any mono- or bi-cyclic group composed of from 5 to 10 ring members, having at least one aromatic moiety and containing from 1 to 4 hetero atoms selected from oxygen, sulphur and nitrogen (including quaternary nitrogens),
   “cycloalkyl” means any mono- or bi-cyclic, non-aromatic, carbocyclic group containing from 3 to 10 ring members,
   “heterocycloalkyl” means any mono- or bi-cyclic, non-aromatic, condensed or spiro group composed of from 3 to 10 ring members and containing from 1 to 3 hetero atoms selected from oxygen, sulphur, SO, SO₂ and nitrogen,
it being possible for the aryl, heteroaryl, cycloalkyl and heterocycloalkyl groups so defined and the groups alkyl, alkenyl, alkynyl and alkoxy to be substituted by from 1 to 3 groups selected from optionally substituted, linear or branched (C₁-C₆)alkyl, (C₃-C₆)spiro, optionally substituted, linear or branched (C₁-C₆)alkoxy, (C₁-C₆)alkyl-S—, hydroxy, oxo (or N-oxide where appropriate), nitro, cyano, —COOR′, —OCOR′, NR′R″, linear or branched (C₁-C₆)polyhaloalkyl, trifluoromethoxy, (C₁-C₆)alkylsulphonyl, halogen, optionally substituted aryl, heteroaryl, aryloxy, arylthio, cycloalkyl, heterocycloalkyl optionally substituted by one or more halogen atoms or alkyl groups, it being understood that R′ and R″, each independently of the other, represent a hydrogen atom or an optionally substituted, linear or branched (C₁-C₆)alkyl group,
to their enantiomers and diastereoisomers, and to addition salts thereof with a pharmaceutically acceptable acid or base.

Among the pharmaceutically acceptable acids there may be mentioned, without implying any limitation, hydrochloric acid, hydrobromic acid, sulphuric acid, phosphonic acid, acetic acid, trifluoroacetic acid, lactic acid, pyruvic acid, malonic acid, succinic acid, glutaric acid, fumaric acid, tartaric acid, maleic acid, citric acid, ascorbic acid, oxalic acid, methanesulphonic acid, camphoric acid etc.

Among the pharmaceutically acceptable bases there may be mentioned, without implying any limitation, sodium hydroxide, potassium hydroxide, triethylamine, tert-butylamine etc.

Advantageously, A₁ represents a hydrogen atom or a methyl group.

Furthermore, A₂ preferably represents a linear or branched (C₁-C₆)alkyl group optionally substituted by a group selected from halogen, hydroxy, linear or branched (C₁-C₆)alkoxy, NR′R″ and morpholine.

In another embodiment of the invention, A₂ represents a linear or branched (C₁-C₆)polyhaloalkyl group or a cyclopropyl group.

Even more preferably, A₁ and A₂ both represent a methyl group.

In a preferred embodiment of the invention, T represents a linear or branched (C₁-C₆)alkyl.

In another preferred embodiment, T represents a group alkyl(C₁-C₄)—NR₁R₂, and more particularly a group alkyl(C₁-C₄)—NR₁R₂ wherein R₁ and R₂ form with the nitrogen atom carrying them a heterocycloalkyl.

In preferred compounds of the invention, T represents a methyl, aminomethyl, (morpholin-4-yl)methyl, (4-methylpipérazin-1-yl)methyl, 2-(morpholin-4-yl)ethyl, [2-(morpholin-4-yl)ethoxy]methyl, hydroxymethyl, [2-(dimethylamino)ethoxy]methyl, hexahydropyrazino[2,1-c] [1,4]oxazin-8 (1H)-ylmethyl, 1-oxa-6-azaspiro[3.3]hept-6-yl-methyl, 3-(morpholin-4-yl)propyl or trifluoromethyl group. Even more preferably, T represents a (morpholin-4-yl)methyl, methyl or 3-(morpholin-4-yl)propyl group.

Preferably, Rₐ and R_{d} each represent a hydrogen atom and (R_{b},R_{c}), together with the carbon atoms carrying them, form a 1,3-dioxolane group or a 1,4-dioxane group; or Rₐ, R_{c} and R_{d} each represent a hydrogen atom and R_{b} represents a hydrogen or halogen atom or a methoxy group. Even more preferably, Rₐ and R_{d} each represent a hydrogen atom and (R_{b},R_{c}), together with the carbon atoms carrying them, form a 1,3-dioxolane group; or Rₐ, R_{c} and R_{d} each represent a hydrogen atom and R_{b} represents a halogen, preferably a chlorine or fluorine atom.

In another embodiment of the invention, Rₐ and R_{d} each represent a hydrogen atom, R_{b} represents a hydrogen or halogen atom and R_{c} a hydroxy or methoxy group, or: Rₐ and R_{d} each represent a hydrogen atom, R_{b} represents a hydroxy or methoxy group and R_{c} a halogen atom.

Alternatively, Rₐ, R_{b} and R_{d} advantageously each represent a hydrogen atom and R_{c} represents a group selected from R₇—CO—NH—(C₀-C₆)alkyl-, R₇—SO₂—NH—(C₀-C₆)alkyl-, R₇—NH—CO—NH—(C₀-C₆)alkyl- and R₇—O—CO—NH—(C₀-C₆)alkyl-. For those specific compounds, R₃ preferably represents a linear or branched (C₁-C₆)alkyl or a heteroaryl optionally substituted by a linear or branched (C₁-C₆)alkyl, and R₄ represents a 4-hydroxyphenyl group. Even more preferably, R₃ represents a methyl.

Preferred R₄ groups are as follows: phenyl; 4-hydroxyphenyl; 3-fluoro-4-hydroxyphenyl; 2-hydroxypyrimidine; 3-hydroxypyridine. Even more preferably, R₄ represents a 4-hydroxyphenyl group.

In preferred compounds of the invention, R₃ represents a linear or branched (C₁-C₆)alkyl group (preferably methyl), aryl or heteroaryl, all being optionally substituted. The groups aryl and heteroaryl are more especially preferred. Finally, R₃ preferably represents a group selected from phenyl, 1H-pyrazole, 1H-indole, 1H-indazole, pyridine, pyrimidine, 1H-pyrrolo[2,3-b]pyridine, 2,3-dihydro-1H-pyrrolo[2,3-b]pyridine, 1H-benzimidazole, 1H-pyrrole, 1H-pyrrolo[2,3-c]pyridine, 1H-pyrrolo[3,2-b]pyridine, 5H-pyrrolo[3,2-d]pyrimidine, thiophene, pyrazine, 1H-pyrazolo[3,4-b]pyridine, 1,2-oxazole, and 1H-pyrazolo[1,5-a]pyrimidine, those groups optionally having one or more substituents selected from halogen, linear or branched (C₁-C₆)alkyl, linear or branched (C₁-C₆)alkoxy, cyano, cyclopropyl, oxetane, tetrahydrofuran, —CO—O—CH₃, trideuteriomethyl, 2-(morpholin-4-yl)ethyl and 2-(morpholin-4-yl)ethoxy. More preferably, R₃ represents a group 1-methyl-1H-pyrazol-4-yl, pyridin-4-yl, 1-methyl-1H-pyrrolo[2,3-b]pyridin-5-yl, 5-cyano-1-methyl-1H-pyrrol-3-yl, 5-cyano-1,2-dimethyl-1H-pyrrol-3-yl, 1-methyl-2,3-dihydro-1H-pyrrolo[2,3-b]pyridin-5-yl, 5-cyano-2-methyl-1-(trideuteriomethyl)-1H-pyrrol-3-yl.

Preferred compounds according to the invention are included in the following group:
5-(5-chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide,
5-(5-chloro-2-{[(3R)-3-methyl-3,4-dihydroisoquinolin-2 (1H)-yl]carbonyl}phenyl)-N-(4-hydroxyphenyl)-1,2-dimethyl-N-(pyridin-4-yl)-1H-pyrrole-3-carboxamide,
N-(4-hydroxyphenyl)-1,2-dimethyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide,
N-(4-hydroxyphenyl)-1,2-dimethyl-5-(6-{[(3R)-3-methyl-3,4-dihydroisoquinolin-2 (1H)-yl]carbonyl}-1,3-benzodioxol-5-yl)-N-(pyridin-4-yl)-1H-pyrrole-3-carboxamide,
5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide,
5-(5-chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-b]pyridin-5-yl)-1H-pyrrole-3-carboxamide,
5-(5-chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(pyridin-4-yl)-1H-pyrrole-3-carboxamide,
5-(5-chloro-2-{[(3S)-3-(morpholin-4-ylmethyl)-3,4-dihydroisoquinolin-2 (1H)-yl]carbonyl}phenyl)-N-(5-cyano-1-methyl-1H-pyrrol-3-yl)-N-(4-hydroxyphenyl)-1,2-dimethyl-1H-pyrrole-3-carboxamide,
N-(5-cyano-1-methyl-1H-pyrrol-3-yl)-5-(5-fluoro-2-{[(3S)-3-(morpholin-4-yl-methyl)-3,4-dihydroisoquinolin-2 (1H)-yl]carbonyl}phenyl)-N-(4-hydroxyphenyl)-1,2-dimethyl-1H-pyrrole-3-carboxamide,
5-(5-chloro-2-{[(3S)-3-(morpholin-4-ylmethyl)-3,4-dihydroisoquinolin-2 (1H)-yl]-carbonyl}phenyl)-N-(5-cyano-1,2-dimethyl-1H-pyrrol-3-yl)-N-(4-hydroxyphenyl)-1,2-dimethyl-1H-pyrrole-3-carboxamide,
5-(5-chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-b]pyridin-5-yl)-1H-pyrrole-3-carboxamide,
N-(5-cyano-1,2-dimethyl-1H-pyrrol-3-yl)-5-(5-fluoro-2-{[(3S)-3-(morpholin-4-yl-methyl)-3,4-dihydroisoquinolin-2 (1H)-yl]carbonyl}phenyl)-N-(4-hydroxyphenyl)-1,2-dimethyl-1H-pyrrole-3-carboxamide,
5-(5-chloro-2-{[(3S)-3-(morpholin-4-ylmethyl)-3,4-dihydroisoquinolin-2 (1H)-yl]carbonyl}phenyl)-N-[5-cyano-2-methyl-1-(trideuteriomethyl)-1H-pyrrol-3-yl]-N-(4-hydroxyphenyl)-1,2-dimethyl-1H-pyrrole-3-carboxamide,
   their enantiomers and diastereoisomers, and addition salts thereof with a pharmaceutically acceptable acid or base.

The invention relates also to a process for the preparation of compounds of formula (I), which process is characterised in that there is used as starting material the compound of formula (II):

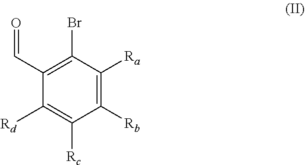

wherein Rₐ, R_{b}, R_{c} and R_{d} are as defined for formula (I),
which compound of formula (II) is subjected to a Heck reaction, in an aqueous or organic medium, in the presence of a palladium catalyst, of a base, of a phosphine and of the compound of formula (III):

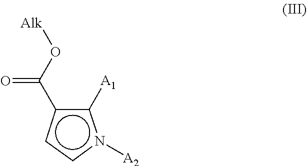

wherein the groups A₁ and A₂ are as defined for formula (I) and Alk represents a linear or branched (C₁-C₆)alkyl,
to obtain the compound of formula (IV):

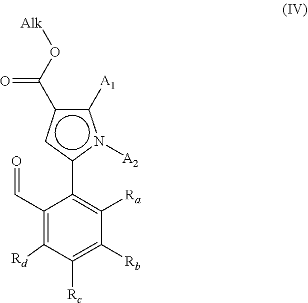

wherein A₁, A₂, Rₐ, R_{b}, R_{c} and R_{d} are as defined for formula (I) and Alk is as defined hereinbefore,
the aldehyde function of which compound of formula (IV) is oxidised to a carboxylic acid to form the compound of formula (V):

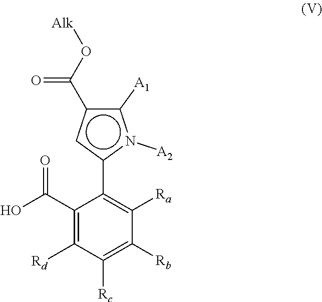

wherein A₁, A₂, Rₐ, R_{b}, R_{c} and R_{d} are as defined for formula (I) and Alk is as defined hereinbefore,
which compound of formula (V) is then subjected to peptide coupling with a compound of formula (VI):

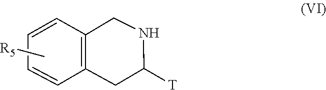

wherein T and R₅ are as defined for formula (I),
to yield the compound of formula (VII):

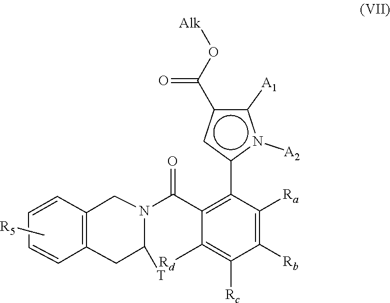

wherein A₁, A₂, Rₐ, R_{b}, R_{c}, R_{d}, T and R₅ are as defined for formula (I) and Alk is as defined hereinbefore,
the ester function of which compound of formula (VII) is hydrolysed to yield the corresponding carboxylic acid or carboxylate, which may be converted into an acid derivative such as the corresponding acyl chloride or anhydride before being coupled with an amine NHR₃R₄ wherein R₃ and R₄ have the same meanings as for formula (I), to yield the compound of formula (I),
which compound of formula (I) may be purified according to a conventional separation technique, which is converted, if desired, into its addition salts with a pharmaceutically acceptable acid or base and which is optionally separated into its isomers according to a conventional separation technique,
it being understood that, at any time considered appropriate in the course of the above-described process, certain groups (hydroxy, amino . . . ) of the reagents or intermediates of synthesis may be protected and then deprotected according to the requirements of synthesis.

More particularly, when one of the groups R₃ or R₄ of the amine NHR₃R₄ is substituted by a hydroxy function, the latter may be subjected beforehand to a protection reaction prior to any coupling with the carboxylic acid formed from the compound of formula (VII), or with a corresponding acid derivative thereof, the resulting protected compound of formula (I) subsequently undergoes a deprotection reaction and is then optionally converted into one of its addition salts with a pharmaceutically acceptable acid or base.

The present invention relates also to an alternative process for the preparation of compounds of formula (I′), which are particular cases of the compounds of formula (I) as defined hereinbefore:

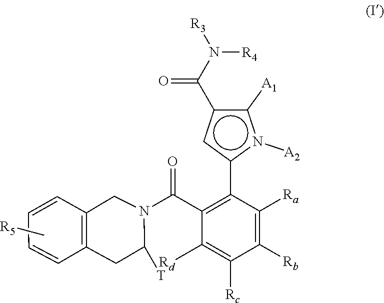

wherein:
   A₁, A₂, Rₐ, R_{d}, R₃, R₄, T and R₅ are as defined for formula (I),
   R_{b} and R_{c} are such that one represents a hydrogen and the other a group selected from R₇—CO—NH—(C₀-C₆)alkyl-, R₇—SO₂—NH—(C₀-C₆)alkyl-, R₇—NH—CO—NH—(C₀-C₆)alkyl- and R₇—O—CO—NH—(C₀-C₆)alkyl-,
which preparation process uses as starting material a compound of formula (II′):

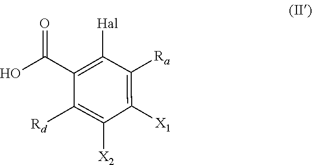

wherein:
   Rₐ and R_{d} are as defined for formula (I),
   Hal represents a halogen atom,
   X₁ and X₂ are such that one represents a (C₀-C₆)alkyl-NH₂ group while the other represents a hydrogen atom,
which compound of formula (II′) is then subjected to peptide coupling with a compound of formula (VI):

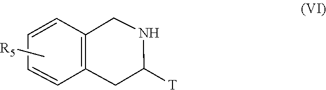

wherein T and R₅ are as defined for formula (I),
to yield the compound of formula (III′):

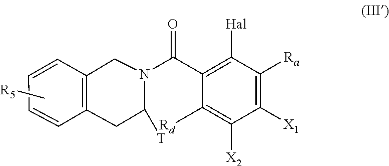

wherein:
   Rₐ, R_{d}, T and R₅ are as defined for formula (I),
   Hal represents a halogen atom,
   X₁ and X₂ are such that one represents a (C₀-C₆)alkyl-NH₂ group while the other represents a hydrogen atom,
which compound of formula (III′) is subjected to a Heck reaction, in an aqueous or organic medium, in the presence of a palladium catalyst, of a base, of a phosphine and of a compound of formula (IV′):

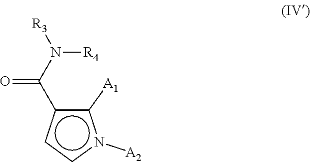

wherein A₁, A₂, R₃ and R₄ are as defined for formula (I),
to form the compound of formula (V′):

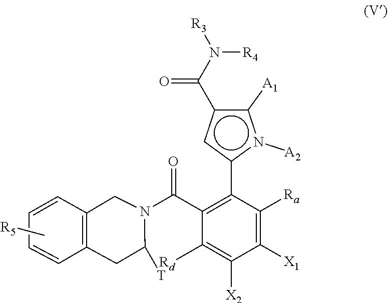

wherein:
   A₁, A₂, Rₐ, R_{d}, R₃, R₄, T and R₅ are as defined for formula (I),
   X₁ and X₂ are such that one represents a (C₀-C₆)alkyl-NH₂ group while the other represents a hydrogen atom,
which compound of formula (V′) is then subjected to an acylation or sulphonylation reaction to yield the compound of formula (I′),
which compound of formula (I′) may be purified according to a conventional separation technique, which is converted, if desired, into its addition salts with a pharmaceutically acceptable acid or base and which is optionally separated into its isomers according to a conventional separation technique,
it being understood that, at any time considered appropriate in the course of the above-described process, certain groups (hydroxy, amino . . . ) of the reagents or intermediates of synthesis may be protected and then deprotected according to the requirements of synthesis.

The compounds of formulae (II), (III), (II′), (IV′), (VI) and the amine NHR₃R₄ are either commercially available or can be obtained by the person skilled in the art using conventional chemical reactions described in the literature.

Pharmacological study of the compounds of the invention has shown that they have pro-apoptotic properties. The ability to reactivate the apoptotic process in cancerous cells is of major therapeutic interest in the treatment of cancers, auto-immune diseases and diseases of the immune system.

More especially, the compounds according to the invention will be useful in the treatment of chemo- or radio-resistant cancers, and in malignant haemopathies and small-cell lung cancer.

Among the cancer treatments envisaged there may be mentioned, without implying any limitation, cancers of the bladder, brain, breast and uterus, chronic lymphoid leukaemias, colorectal cancer, cancers of the oesophagus and liver, lymphoblastic leukaemias, non-Hodgkin lymphomas, melanomas, malignant haemopathies, myelomas, ovarian cancer, non-small-cell lung cancer, prostate cancer and small-cell lung cancer. Among non-Hodgkin lymphomas, there may be mentioned more preferably follicular lymphomas, mantle cell lymphomas, diffuse large B-cell lymphomas, small lymphocytic lymphomas and marginal zone B-cell lymphomas.

The present invention relates also to pharmaceutical compositions comprising at least one compound of formula (I) in combination with one or more pharmaceutically acceptable excipients.

Among the pharmaceutical compositions according to the invention there may be mentioned more especially those that are suitable for oral, parenteral, nasal, per- or trans-cutaneous, rectal, perlingual, ocular or respiratory administration, especially tablets or dragdes, sublingual tablets, sachets, paquets, capsules, glossettes, lozenges, suppositories, creams, ointments, dermal gels, and drinkable or injectable ampoules.

The dosage varies according to the sex, age and weight of the patient, the administration route, the nature of the therapeutic indication, or of any associated treatments, and ranges from 0.01 mg to 1 g per 24 hours in one or more administrations.

Furthermore, the present invention relates also to the association of a compound of formula (I) with an anticancer agent selected from genotoxic agents, mitotic poisons, anti-metabolites, proteasome inhibitors, kinase inhibitors and antibodies, and also to pharmaceutical compositions comprising that type of association and their use in the manufacture of medicaments for use in the treatment of cancer.

The compounds of the invention may also be used in association with radiotherapy in the treatment of cancer.

Finally, the compounds of the invention may be linked to monoclonal antibodies or fragments thereof or linked to scaffold proteins that can be related or not to monoclonal antibodies.

Antibody fragments must be understood as fragments of Fv, scFv, Fab, F(ab′)2, F(ab′), scFv-Fc type or diabodies, which generally have the same specificity of binding as the antibody from which they are descended. According to the present invention, antibody fragments of the invention can be obtained starting from antibodies by methods such as digestion by enzymes, such as pepsin or papain, and/or by cleavage of the disulfide bridges by chemical reduction. In another manner, the antibody fragments comprised in the present invention can be obtained by techniques of genetic recombination likewise well known to the person skilled in the art or else by peptide synthesis by means of, for example, automatic peptide synthesizers such as those supplied by the company Applied Biosystems, etc.

Scaffold proteins that can be related or not to monoclonal antibodies are understood to mean a protein that contains or not an immunoglobulin fold and that yields a binding capacity similar to a monoclonal antibody. The man skilled in the art knows how to select the protein scaffold. More particularly, it is known that, to be selected, such a scaffold should display several features as follow (Skerra A., J. Mol. Recogn., 13, 2000, 167-187): phylogenetically good conservation, robust architecture with a well-known three-dimensional molecular organization (such as, for example, crystallography or NMR), small size, no or only a low degree of post-translational modifications, easy to produce, express and purify. Such a protein scaffold can be, but without limitation, a structure selected from the group consisting in fibronectin and preferentially the tenth fibronectin type III domain (FNfn10), lipocalin, anticalin (Skerra A., J. Biotechnol., 2001, 74(4):257-75), the protein Z derivative from the domain B of staphylococcal protein A, thioredoxin A or any protein with a repeated domain such as an “ankyrin repeat” (Kohl et al., PNAS, 2003, vol. 100, No. 4, 1700-1705), “armadillo repeat”, “leucine-rich repeat” or “tetratricopeptide repeat”. There could also be mentioned a scaffold derivative from toxins (such as, for example, scorpion, insect, plant or mollusc toxins) or protein inhibitors of neuronal nitric oxide synthase (PIN).

The following Preparations and Examples illustrate the invention without limiting it in any way.

### Preparation 1: 4-Chloro-2-[4-(ethoxycarbonyl)-1,5-dimethyl-1H-pyrrol-2-yl]benzoic acid

Step A: Ethyl 1,2-dimethyl-1H-pyrrole-3-carboxylate

To a solution of 10 g of ethyl 2-methyl-1H-pyrrole-3-carboxylate (65.3 mmol) and 8.95 mL (130.6 mmol) of methyl iodide in 70 mL of dimethylformamide placed at 0° C. there are added, in three portions, 2.61 g (65.3 mmol) of sodium hydride (NaH) 60%. The batch is then stirred at 0° C. for 1 hour. Then, the reaction mixture is hydrolysed by the addition of 420 mL of ice-cold water. The reaction mixture is then diluted with ethyl acetate, successively washed with 0.1M aqueous hydrochloric acid (HCl) solution, saturated aqueous LiCl solution and then brine. The organic phase is then dried over MgSO₄, filtered, concentrated to dryness and purified by chromatography over silica gel (petroleum ether/AcOEt gradient).

¹H NMR: δ (400 MHz; dmso-d6; 300K): 6.65 (d, 1H pyrrole); 6.3 (1d, 1H pyrrole); 4.1 (1q, 2H, OCH₂CH₃); 3.5 (s, 3H N-pyrrole); 2.4 (s, 3H pyrrole); 1.5 (It, 3H OCH₂CH₃)

IR: ν: >C═O: 1688 cm⁻¹; ν: C—O—C: 1172 cm⁻¹

Step B: Ethyl 5-(5-chloro-2-formylphenyl)-1,2-dimethyl-1H-pyrrole-3-carboxylate

To a solution of 10.5 g of the compound obtained in Step A (62.8 mmol) in 65 mL of N,N-dimethylacetamide there are successively added 15.2 g of 2-bromo-4-chlorobenzaldehyde (69 mmol), 12.3 g of potassium acetate (125.6 mmol) and then the batch is stirred under argon for 20 minutes. There are then added 2.2 g of palladium catalyst PdCl₂(PPh₃)₂ (3.14 mmol). The reaction mixture is then heated at 130° C. overnight. The mixture is allowed to return to ambient temperature and it is then diluted with dichloromethane. Animal charcoal is added (2 g per g of product) and the batch is stirred at ambient temperature for 1 hour and then filtered. The organic phase is then washed with water, dried over MgSO₄ and concentrated to dryness. The crude product thereby obtained is purified by chromatography over silica gel (petroleum ether/AcOEt gradient). The title product is obtained in the form of a solid.

¹H NMR: δ (400 MHz; dmso-d6; 300K): 9.8 (s, 1H, formyl); 7.91-7.69-7.61 (d, 3H, aromatic Hs); 6.5 (s, 1H pyrrole); 4.2 (q, 2H, OCH₂CH₃); 3.4 (s, 3H, CH₃—N-pyrrole); 2.55 (s, 3H pyrrole); 1.28 (t, 3H, OCH₂CH₃)

Step C: 4-Chloro-2-[4-(ethoxycarbonyl)-1,5-dimethyl-1H-pyrrol-2-yl]benzoic acid

A solution is prepared containing 12.85 g of the compound obtained in Step B (42 mmol) and 35.7 mL (336 mmol) of 2-methyl-2-butene in a mixture containing 20 mL of acetone and 20 mL of tetrahydrofuran. There are added, dropwise, 200 mL of an aqueous solution containing a mixture of 13.3 g of sodium chlorite (NaClO₂) (147 mmol) and 14.5 g of sodium hydrogen phosphate (NaHPO₄) (105 mmol). The batch is then vigorously stirred at ambient temperature for 7 hours. The reaction mixture is then concentrated to remove the acetone. Ethyl acetate is added, and the organic phase is washed with water, dried over MgSO₄ and then concentrated to dryness. The residue is then taken up in a minimum of ethyl ether. The solid then obtained is filtered off, washed with ether and then dried in vacuo at 40° C. overnight. The title product is obtained in the form of a solid, which is subsequently used without being otherwise purified.

¹H NMR: δ (400 MHz; dmso-d6; 300K): 13 (m, 1H COOH); 7.85-7.6-7.41 (d, dd, wd, 3H, aromatic Hs); 6.3 (s, 1H, H pyrrole); 4.15 (q, 2H, OCH₂CH₃); 3.25 (s, 3H, CH₃—N-pyrrole); 2.5 (s, 3H, CH₃-pyrrole); 1.25 (t, 3H, OCH₂CH₃)

IR: ν: —OH: 3100-2500 cm⁻¹ acid; ν: >C═O: 1681 cm⁻¹ ester+acid

### Preparation 2: 2-[4-(Ethoxycarbonyl)-1-methyl-1H-pyrrol-2-yl]benzoic acid

The procedure is in accordance with the process of Preparation 1, replacing, on the one hand, the ethyl 2-methyl-1H-pyrrole-3-carboxylate used in Step A by ethyl 1H-pyrrole-3-carboxylate and, on the other hand, the 2-bromo-4-chlorobenzaldehyde used in Step B by 2-bromo-benzaldehyde.

### Preparation 3: 4-Chloro-2-[4-(ethoxycarbonyl)-1-methyl-1H-pyrrol-2-yl]benzoic acid

The procedure is in accordance with the process of Preparation 1, replacing the ethyl 2-methyl-1H-pyrrole-3-carboxylate used in Step A by ethyl 1H-pyrrole-3-carboxylate.

### Preparation 4: 6-[4-(Ethoxycarbonyl)-1-methyl-1H-pyrrol-2-yl]-1,3-benzodioxole-5-carboxylic acid

The procedure is in accordance with the process of Preparation 1, replacing, on the one hand, the ethyl 2-methyl-1H-pyrrole-3-carboxylate used in Step A by ethyl 1H-pyrrole-3-carboxylate and, on the other hand, the 2-bromo-4-chlorobenzaldehyde used in Step B by 6-bromo-1,3-benzodioxole-5-carbaldehyde.

IR: ν: —OH: 3500-2300 cm⁻¹ acid; ν: >C═O: 1688-1670 cm⁻¹ ester+acid

### Preparation 5: 4-Chloro-2-[4-(ethoxycarbonyl)-1-ethyl-1H-pyrrol-2-yl]benzoic acid

The procedure is in accordance with the process of Preparation 1, replacing in Step A, on the one hand, the ethyl 2-methyl-1H-pyrrole-3-carboxylate by ethyl 1H-pyrrole-3-carboxylate and, on the other hand, the methyl iodide by ethyl iodide (see the protocol described in U.S. Pat. No. 6,258,805 B1).

### Preparation 6: 4-Chloro-2-[1-cyclopropyl-4-(ethoxycarbonyl)-1H-pyrrol-2-yl]benzoic acid

The procedure is in accordance with the process of Preparation 1, replacing in Step A, on the one hand, the ethyl 2-methyl-1H-pyrrole-3-carboxylate by ethyl 1H-pyrrole-3-carboxylate and, on the other hand, the methyl iodide by cyclopropylboronic acid (see the protocol described in Bénard S. et al, Journal of Organic Chemistry 73(16), 6441-6444, 2008).

### Preparation 7: 4-Chloro-2-[4-(ethoxycarbonyl)-1-(propan-2-yl)-1H-pyrrol-2-yl]benzoic acid

The procedure is in accordance with the process of Preparation 1, replacing in Step A, on the one hand, the ethyl 2-methyl-1H-pyrrole-3-carboxylate by ethyl 1H-pyrrole-3-carboxylate and, on the other hand, the methyl iodide by isopropyl iodide (see the protocol described in Okada E. et al, Heterocycles 34(7), 1435-1441, 1992).

### Preparation 8: 4-Fluoro-2-[4-(methoxycarbonyl)-1,5-dimethyl-1H-pyrrol-2-yl]benzoic acid

The procedure is in accordance with the process of Preparation 1, replacing, in Step A, the ethyl 2-methyl-1H-pyrrole-3-carboxylate by methyl 2-methyl-1H-pyrrole-3-carboxylate and also the 2-bromo-4-chlorobenzaldehyde used in Step B by 2-bromo-4-fluorobenzaldehyde.

IR: ν: —OH: 2727-2379 cm⁻¹ acid; ν: >C═O: 1687 cm⁻¹

### Preparation 9: 6-{1-[2-(Benzyloxy)ethyl]-4-(ethoxycarbonyl)-5-methyl-1H-pyrrol-2-yl}-1,3-benzodioxole-5-carboxylic acid

Step A: Ethyl 1-[2-(benzyloxy)ethyl]-2-methyl-1H-pyrrole-3-carboxylate

The procedure is in accordance with the process of Step A of Preparation 1, replacing the methyl iodide used as alkylating agent by benzyl 2-bromoethyl ether.

¹H NMR: δ (400 MHz; dmso-d6; 300K): 7.32 (t, 2H, aromatic Hs, H meta benzyl ether); 7.3 (t, 1H, aromatic H, H para benzyl ether); 7.23 (d, 2H, aromatic Hs, H ortho benzyl ether); 6.72 (d, 1H, H-pyrrole); 6.35 (d, 1H, H-pyrrole); 4.48 (s, 2H, aliphatic Hs, O—CH₂-Ph); 4.15 (q, 2H, aliphatic Hs, O—CH₂—CH₃); 4.1 (t, 2H, aliphatic Hs, CH₂—O—CH₂-Ph); 3.7 (t, 2H, aliphatic Hs, CH₂—CH₂—O—CH₂-Ph); 2.45 (s, 3H, CH₃-pyrrole); 1.25 (t, 3H, aliphatic Hs, O—CH₂—CH₃)

IR: ν: >C═O: 1689 cm⁻¹

Step B: 6-{1-[2-(Benzyloxy)ethyl]-4-(ethoxycarbonyl)-5-methyl-1H-pyrrol-2-yl}-1,3-benzodioxole-5-carboxylic acid

The procedure is in accordance with the processes of Steps B and C of Preparation 1, replacing the 2-bromo-4-chlorobenzaldehyde by 6-bromo-1,3-benzodioxole-5-carbaldehyde.

¹H NMR: δ (400 MHz; dmso-d6; 300K): 7.35 (s, 1H, aromatic H, H benzodioxole); 7.30 (m, 3H, aromatic Hs, H benzyl ether); 7.25 (s, 1H, aromatic H, H benzodioxole); 7.10 (d, 2H, aromatic Hs, H ortho benzyl ether); 12.55 (broad s, 1H, COOH); 6.75 (s, 1H, H-pyrrole); 6.15 (broad s, 2H, aliphatic Hs, O—CH₂—O); 4.30 (s, 2H, aliphatic Hs, O—CH₂-Ph); 4.15 (q, 2H, aliphatic Hs, O—CH₂—CH₃); 3.9 (m, 2H, aliphatic Hs, CH₂—CH₂—O—CH₂-Ph); 3.40 (t, 2H, aliphatic Hs, CH₂—CH₂—O—CH₂-Ph); 2.50 (s, 3H, CH₃-pyrrole); 1.25 (t, 3H, aliphatic Hs, O—CH₂—CH₃)

IR: ν: —OH: 3200-2300 cm⁻¹ acid; ν: >C═O: 1687 cm⁻¹ acid

### Preparation 10: 2-{1-[3-(Benzyloxy)propyl]-4-(ethoxycarbonyl)-5-methyl-1H-pyrrol-2-yl}-4-fluorobenzoic acid

The procedure is in accordance with the process of Preparation 9, replacing the benzyl 2-bromoethyl ether used in Step A by benzyl 3-bromopropyl ether and also the 6-bromo-1,3-benzodioxole-5-carbaldehyde used in Step B by 2-bromo-4-fluorobenzaldehyde.

IR: ν: —OH: 3200-2305 cm⁻¹ acid; ν: >C═O: 1690 cm⁻¹ acid

### Preparation 11: 6-[4-(Ethoxycarbonyl)-1,5-dimethyl-1H-pyrrol-2-yl]-1,3-benzodioxole-5-carboxylic acid

The procedure is in accordance with the process of Preparation 1, replacing the 2-bromo-4-chlorobenzaldehyde used in Step B by 6-bromo-1,3-benzodioxole-5-carbaldehyde.

### Preparation 12: 4-Methoxy-2-[4-(methoxycarbonyl)-1,5-dimethyl-1H-pyrrol-2-yl]benzoic acid

The procedure is in accordance with the process of Preparation 1, replacing the ethyl 2-methyl-1H-pyrrole-3-carboxylate used in Step A by methyl 2-methyl-1H-pyrrole-3-carboxylate and also the 2-bromo-4-chlorobenzaldehyde used in Step B by 2-bromo-4-methoxybenzaldehyde.

IR: ν: —OH: 3000-2500 cm⁻¹ acid; ν: >C═O: 1693+1670 cm⁻¹ acid+ester

### Preparation 13: 7-[4-(Methoxycarbonyl)-1,5-dimethyl-1H-pyrrol-2-yl]-2,3-dihydro-1,4-benzodioxin-6-carboxylic acid

The procedure is in accordance with the process of Preparation 1, replacing in Step A the ethyl 2-methyl-1H-pyrrole-3-carboxylate by methyl 2-methyl-1H-pyrrole-3-carboxylate and also the 2-bromo-4-chlorobenzaldehyde used in Step B by 7-bromo-2,3-dihydro-1,4-benzodioxin-6-carbaldehyde.

IR: ν: —OH: 3100-2500 cm⁻¹ acid; ν: >C═O: 1690+1674 cm⁻¹ acid+ester

### Preparation 14: 4-Fluoro-5-methoxy-2-[4-(methoxycarbonyl)-1,5-dimethyl-1H-pyrrol-2-yl]benzoic acid

The procedure is in accordance with the process of Preparation 1, replacing in Step A the ethyl 2-methyl-1H-pyrrole-3-carboxylate by methyl 2-methyl-1H-pyrrole-3-carboxylate and also the 2-bromo-4-chlorobenzaldehyde used in Step B by 2-bromo-4-fluoro-5-methoxybenzaldehyde.

### Preparation 15: 4-Fluoro-5-hydroxy-2-[4-(methoxycarbonyl)-1,5-dimethyl-1H-pyrrol-2-yl]benzoic acid

The procedure is in accordance with the process of Preparation 1, replacing in Step A the ethyl 2-methyl-1H-pyrrole-3-carboxylate by methyl 2-methyl-1H-pyrrole-3-carboxylate and also the 2-bromo-4-chlorobenzaldehyde used in Step B by 2-bromo-4-fluoro-5-hydroxy-benzaldehyde.

### Preparation 16: 6-[4-(Ethoxycarbonyl)-1-methyl-5-trifluoromethyl-1H-pyrrol-2-yl]-1,3-benzodioxole-5-carboxylic acid

The procedure is in accordance with the process of Preparation 1, replacing in Step A the ethyl 2-methyl-1H-pyrrole-3-carboxylate by ethyl 2-(trifluoromethyl)-1H-pyrrole-3-carboxylate and also the 2-bromo-4-chlorobenzaldehyde used in Step B by 6-bromo-1,3-benzodioxole-5-carbaldehyde.

### Preparation 17: 6-[4-(Ethoxycarbonyl)-1-ethyl-5-methyl-1H-pyrrol-2-yl]-1,3-benzodioxole-5-carboxylic acid

The procedure is in accordance with the process of Preparation 11, replacing the methyl iodide by ethyl iodide (see the protocol described in U.S. Pat. No. 6,258,805 B1).

### Preparation 18: 6-[4-(Ethoxycarbonyl)-1-methyl-5-(trifluoromethyl)-1H-pyrrol-2-yl]-1,3-benzodioxole-5-carboxylic acid

Step A: Ethyl 1-methyl-2-(trifluoromethyl)pyrrole-3-carboxylate

Ethyl 4,4,4-trifluoro-3-oxo-butanoate (29 ml, 0.219 mmol) is added to methylamine (40% solution in water) (50 ml, 0.580 mmol) cooled to 10° C.; a white precipitate forms. 1,2-Dibromoethyl acetate (prepared according to Molecules, 16, 9368-9385; 2011) is added dropwise. The reactor is then sealed and heated at 70° C. for 45 minutes. The reaction mixture is cooled, then extracted with ethyl acetate, dried over sodium sulphate (Na₂SO₄) and evaporated to dryness. The crude reaction product obtained is purified by chromatography over silica gel using heptane and ethyl acetate as eluants. The expected compound is obtained in the form of crystals.

¹H NMR (400 MHz, dmso-d6) δ ppm: 7.11 (d, 1H), 6.52 (d, 1H), 4.21 (quad, 2H), 3.8 (s, 3H), 1.27 (t, 3H)

¹⁹F NMR (400 MHz, dmso-d6) δ ppm: −53.9

IR (ATR) cm¹ 3145+3128 ν —CH, 1711 ν>C═O, 1183+1117+1078 ν —CF3

Step B: 6-[4-(Ethoxycarbonyl)-1-methyl-5-(trifluoromethyl)-1H-pyrrol-2-yl]-1,3-benzo-dioxole-5-carboxylic acid

The procedure is in accordance with the protocol described in Steps B and C of Preparation 1, replacing the 2-bromo-4-chlorobenzaldehyde used in Step B by 6-bromo-1,3-benzodioxole-5-carbaldehyde.

### Preparation 19: 6-[1-(2-Benzyloxyethyl)-4-ethoxycarbonyl-5-methyl-1H-pyrrol-2-yl]-1,3-benzodioxole-5-carboxylic acid

Step A: Ethyl 1-(2-benzyloxyethyl)-2-methyl-pyrrole-3-carboxylate

Ethyl 2-methyl-1H-pyrrole-3-carboxylate (10 g, 65.3 mmol) is dissolved in 100 mL of dimethylformamide under argon cooled to 0° C., and then 2-bromoethoxymethylbenzene (28.1 g, 130.6 mmol) is added all at once. The reaction mixture is placed under stirring. There is then added thereto, at 0° C., in three portions, NaH (1.72 g, 71.83 mmol) over a period of 15 minutes. The reaction mixture is stirred for 15 minutes at 0° C., and then for 15 hours at ambient temperature. It is then poured into an ice bath and then extracted 3 times with ethyl acetate. The organic phase is washed 3 times with saturated aqueous lithium chloride solution, dried over MgSO₄, filtered and then evaporated to dryness. The residue thereby obtained is purified by chromatography over silica gel using petroleum ether and ethyl acetate as eluants. The expected compound is obtained in the form of an oil.

¹H NMR (400 MHz, dmso-d6) δ ppm: 7.32 (t, 2H), 7.3 (t, 1H), 7.23 (d, 2H), 6.72 (d, 1H), 6.35 (d, 1H), 4.48 (s, 2H), 4.15 (quad., 2H), 4.1 (t, 2H), 3.7 (t, 2H), 2.45 (s, 3H), 1.25 (t, 3H) IR (ATR) cm⁻¹: 1689 ν —C═O

Step B: Ethyl 1-(2-benzyloxyethyl)-5-(6-formyl-1,3-benzodioxol-5-yl)-2-methyl-pyrrole-3-carboxylate

The procedure is in accordance with the process of Step B of Preparation 1, replacing the 2-bromo-4-chlorobenzaldehyde by 6-bromo-1,3-benzodioxole-5-carbaldehyde.

¹H NMR (400 MHz, dmso-d6) S ppm: 9.5 (s, 1H), 7.3 (s, 1H), 7.25 (m, 3H), 7.05 (m, 2H), 7 (s, 1H), 6.4 (s, 1H), 6.2 (bs, 2H), 4.25 (s, 2H), 4.2 (quad., 2H), 4.05 (m, 2H), 3.4 (m, 2H), 2.55 (s, 3H), 1.25 (t, 3H)

Step C: 6-[1-(2-Benzyloxyethyl)-4-ethoxycarbonyl-5-methyl-1H-pyrrol-2-yl]-1,3-benzo-dioxole-5-carboxylic acid

The procedure is in accordance with the process of Step C of Preparation 1.

¹H NMR (400 MHz, dmso-d6) δ ppm: 12.55 (bs, 1H), 7.35 (s, 1H), 7.3 (m, 3H), 7.2 (m, 3H), 6.75 (s, 1H), 6.15 (s, 2H), 4.3 (s, 2H), 4.15 (quad., 2H), 3.9 (m, 2H), 3.4 (t, 2H), 2.5 (s, 3H), 1.25 (t, 3H)

IR (ATR) cm⁻¹:3200-2300 ν —OH, 1687 (+ shoulder) ν-C═O carboxylic acid+conjugated ester

### Preparation 20: 6-[4-(Ethoxycarbonyl)-1-ethyl-5-methyl-1H-pyrrol-2-yl]-1,3-benzo-dioxole-5-carboxylic acid

The procedure is in accordance with the protocol described in Preparation 11, replacing the methyl iodide by ethyl iodide.

¹H NMR (400 MHz, dmso-d6) δ ppm: 12.49 (bs, 1H), 7.33 (s, 1H), 6.89 (s, 1H), 6.17 (s, 2H), 6.13 (s, 1H), 4.15 (quad, 2H), 3.69 (quad, 2H), 2.51 (s, 3H), 1.24 (t, 3H), 1.01 (t, 3H)

### Preparation 21: 6-[4-(Ethoxycarbonyl)-1-(2-fluoroethyl)-5-methyl-1H-pyrrol-2-yl]-1,3-benzodioxole-5-carboxylic acid

The procedure is in accordance with the process of Preparation 11, replacing the methyl iodide by 1-bromo-2-fluoroethane.

¹H NMR (400 MHz, dmso-d6, 300K) δ ppm: 12.53 (bs, 1H), 7.34 (s, 1H), 6.9 (s, 1H), 6.16 (s, 1H), 6.16 (s, 2H), 4.4 (dt, 2H), 4.15 (quad, 2H), 4.01 (m, 2H), 2.51 (s, 3H), 1.24 (t, 3H)

¹⁹F NMR (400 MHz, dmso-d6, 300 K) δ ppm: −222

IR: ν: —OH: 3700-2400 cm⁻¹ acid; ν: >C═O: 1689 cm⁻¹ acid; ν: >CF: 1213 cm⁻¹

### Preparation 22: 6-{4-(Ethoxycarbonyl)-1-methyl-5-[2-(morpholin-4-yl)ethyl]-1H-pyrrol-2-yl}-1,3-benzodioxole-5-carboxylic acid

Step A: 3,3-Diethoxypropanoic acid

To a solution of 25 g of ethyl 3,3-diethoxypropionate (131 mmol) in 79 mL of methanol there are added 13.1 mL of aqueous 35% sodium hydroxide solution (452 mmol). The reaction mixture is stirred at ambient temperature for 3 hours. The reaction mixture is then concentrated to remove the methanol. After dissolving the undissolved material by adding water, aqueous 5N HCl solution is added to obtain a pH of 5. Dichloromethane is added and then the organic phase is washed with brine. After drying over MgSO₄ and concentrating to dryness, the title product is obtained in the form of an oil which is used in the next Step without being otherwise purified.

¹H NMR (400 MHz, dmso-d6) δ ppm: 12.2 (s, 1H), 4.8 (t, 1H), 3.58/3.47 (2m, 4H), 2.5 (d, 2H), 1.09 (t, 6H)

Step B: Ethyl 5,5-diethoxy-3-oxo-pentanoate

To a solution of 16 mL of 3-ethoxy-3-oxomalonic acid (135 mmol) in 40 mL of tetrahydrofuran there are added, under argon, 21.9 g of powdered magnesium (90.4 mmol).

The mixture obtained is then heated at 80° C. for 7 hours. After returning to ambient temperature, this mixture is transferred by cannula to a solution of 10 g of the compound obtained in Step A (61.7 mmol) in 64 mL of tetrahydrofuran to which there has previously been added, in portions, 11 g of carbonyldiimidazole (66 mmol). The reaction mixture is stirred for 3 days at ambient temperature. After concentration, the residue is taken up in a mixture of ethyl acetate and aqueous sodium hydrogen sulphate (NaHSO₄) solution. The mixture is stirred vigorously until no more gas is evolved. After separation of the phases, the organic phase is washed successively with water, saturated aqueous NaHCO₃ solution and finally brine. After drying over MgSO₄ and concentrating to dryness, the title product is obtained in the form of an oil which is used in the next Step without being otherwise purified.

¹H NMR (400 MHz, dmso-d6) δ ppm: 4.84 (t, 1H), 4.08 (q, 2H), 3.59 (s, 2H), 3.56/3.46 (2m, 4H), 2.8 (d, 2H), 1.18 (t, 3H), 1.09 (t, 6H)

Step C: Ethyl 2-(2,2-diethoxyethyl)-1-methyl-pyrrole-3-carboxylate

To a solution of 11.8 g of the compound obtained in Step B (50.8 mmol) in 76 mL of water there are added, dropwise at 0° C., 6.6 ml of aqueous 40% methylamine solution (76.2 mmol). The reaction mixture is stirred and gently reheated to ambient temperature over 5 hours. After returning to 0° C., 8.8 mL of aqueous 40% methylamine solution (102 mmol) and then 16.6 mL of aqueous 50% chloroacetaldehyde solution (127 mmol) are each added dropwise at a temperature of less than 10° C. The reaction mixture is stirred at ambient temperature for 16 hours and then diluted with ethyl acetate. The organic phase is washed with brine, dried over MgSO₄ and concentrated to dryness. The crude product thereby obtained is purified by chromatography over silica gel using petroleum ether and ethyl acetate as eluants. The title product is obtained in the form of an oil.

¹H NMR (400 MHz, dmso-d6) δ ppm: 6.7 (d, 1H), 6.33 (d, 1H), 4.55 (t, 1H), 4.15 (q, 2H), 3.6 (m, 2H), 3.6 (s, 3H), 3.3 (m, 2H), 3.15 (d, 2H), 1.25 (t, 3H), 1.05 (t, 6H)

Step D: Ethyl 1-methyl-2-(2-morpholinoethyl)pyrrole-3-carboxylate

To a solution of 3.8 g of the compound obtained in Step C (14.05 mmol) in 28 mL of tetrahydrofuran there are added 58 mL of aqueous 10% sulphuric acid solution. The reaction mixture is stirred at ambient temperature for 2 hours and then diluted with a mixture of ethyl acetate and water. After separation, the organic phase is washed with brine, dried over MgSO₄ and concentrated to dryness. To a solution of the residue thereby obtained in 70 mL of dichloroethane there are added a solution of 13.5 mL of morpholine (14.5 mmol) in a mixture composed of 30 mL of dichloroethane and 3.6 mL of 4N aqueous HCL solution in dioxane (14.5 mmol), and then 7.4 g of sodium triacetoxyborohydride (NaBH(OAc)₃) (35.13 mmol). The reaction mixture is stirred at ambient temperature for 3 hours and then diluted with a mixture of dichloromethane and saturated aqueous NaHCO₃ solution. After separation of the phases and extraction of the aqueous phase with dichloromethane, the organic phases are dried over MgSO₄ and concentrated to dryness. The crude product thereby obtained is purified by chromatography over silica gel using dichloromethane and methanol as eluants. The title product is isolated in the form of an oil.

¹H NMR (400 MHz, dmso-d6) δ ppm: 6.66 (d, 1H), 6.31 (d, 1H), 4.14 (q, 2H), 3.58 (s, 3H), 3.57 (m, 4H), 3.05 (m, 2H), 2.45-2.38 (m, 6H), 1.24 (t, 3H)

Step E: 6-{4-(Ethoxycarbonyl)-1-methyl-5-[2-(morpholin-4-yl)ethyl]-1H-pyrrol-2-yl}-1,3-benzodioxole-5-carboxylic acid

The procedure is in accordance with Steps B and C of Preparation, 1 replacing the 2-bromo-4-chlorobenzaldehyde used in Step B by 6-bromo-1,3-benzodioxole-5-carbaldehyde.

### Preparation 23: 6-[4-(Ethoxycarbonyl)-1-methyl-5-(morpholin-4-ylmethyl)-1H-pyrrol-2-yl]-1,3-benzodioxole-5-carboxylic acid

Step A: Ethyl 4,4-diethoxy-3-oxo-butanoate

To a solution of 40 g of ethyl diethoxyacetate (227 mmol) in 67 mL of ethyl acetate there are added under argon, in portions, 6 g of sodium (261 mmol). The reaction mixture is then stirred at ambient temperature for 48 hours. There are added 10 mL of methanol, and then the mixture is hydrolysed with 65 mL of water. The pH of the reaction mixture is adjusted to pH 6 by adding 1N aqueous HCl solution. The mixture is separated and then extracted with ethyl acetate. The organic phases are combined, washed with brine, dried over MgSO₄, filtered and concentrated to dryness. The title product is obtained in the form of an oil which is used in the next Step without being otherwise purified.

¹H NMR (400 MHz, CDCl₃) δ ppm: 3.6 (s, 2H), 1.28 (t, 9H), 3.7-3.6 (2m, 4H), 4.7 (s, 1H), 4.2 (quad, 2H)

Step B: Ethyl 2-(2,2-diethoxymethyl)-1-methyl-pyrrole-3-carboxylate

The title compound is obtained in accordance with the process described in Step C of Preparation 22, starting from the ethyl 4,4-diethoxy-3-oxo-butanoate obtained in the preceding Step.

¹H NMR (400 MHz, dmso-d6) δ ppm: 6.73 (d, 1H), 6.32 (d, 1H), 6.23 (s, 1H), 4.17 (q, 2H), 3.7 (s, 3H), 3.68/3.43 (2m, 4H), 1.26 (t, 3H), 1.13 (t, 6H)

Step C: Ethyl 1-methyl-2-(2-morpholinomethyl)pyrrole-3-carboxylate

The title compound is obtained in accordance with the process described in Step D of Preparation 22, starting from the ethyl 2-(2,2-diethoxymethyl)-1-methyl-pyrrole-3-carboxylate obtained in the preceding Step.

¹H NMR (400 MHz, dmso-d6) δ ppm: 6.74 (d, 1H), 6.32 (d, 1H), 4.15 (q, 2H), 3.8 (s, 2H), 3.65 (s, 3H), 3.5 (m, 4H), 2.32 (m, 4H), 1.22 (t, 3H)

Step D: 6-[4-(Ethoxycarbonyl)-1-methyl-5-(morpholin-4-ylmethyl)-1H-pyrrol-2-yl]-1,3-benzodioxole-5-carboxylic acid

The procedure is in accordance with the protocol described in Steps B and C of Preparation 1, replacing the 2-bromo-4-chlorobenzaldehyde used in Step B by 6-bromo-1,3-benzodioxole-5-carbaldehyde.

### Preparation 24: 6-[4-(Methoxycarbonyl)-5-(2-methoxyethyl)-1-methyl-1H-pyrrol-2-yl]-1,3-benzodioxole-5-carboxylic acid

Step A: Methyl 2-(2-methoxyethyl)-1-methyl-pyrrole-3-carboxylate

The title compound is obtained in accordance with the protocol of Step C of Preparation 22, using methyl 5-methoxy-3-oxovalerate.

¹H NMR (400 MHz, dmso-d6) δ ppm: 6.69 (wd, 1H), 6.31 (wd, 1H), 3.69 (s, 3H), 3.59 (s, 3H), 3.49 (t, 2H), 3.2 (s, 3H), 3.11 (t, 2H)

Step B: 6-[4-(Methoxycarbonyl)-5-(2-methoxyethyl)-1-methyl-1H-pyrrol-2-yl]-1,3-benzo-dioxole-5-carboxylic acid

The procedure is in accordance with the protocol described in Steps B and C of Preparation 1, replacing the 2-bromo-4-chlorobenzaldehyde used in Step B by 6-bromo-1,3-benzodioxole-5-carbaldehyde.

### Preparation 25: 2-[4-(Ethoxycarbonyl)-1,5-dimethyl-1H-pyrrol-2-yl]benzoic acid

The procedure is in accordance with the process of Preparation 1, replacing the 2-bromo-4-chlorobenzaldehyde used in Step B by 2-bromobenzaldehyde.

¹H NMR (400 MHz, dmso-d6) δ ppm: 12.81 (m, 1H), 7.84 (m, 1H), 7.59 (m, 1H), 7.51 (m, 1H), 7.35 (m, 1H), 6.23 (s, 1H), 4.16 (q, 2H), 3.24 (s, 3H), 2.5 (s, 3H), 1.25 (t, 3H)

### Preparation 26: 5-Fluoro-2-[4-(methoxycarbonyl)-1,5-dimethyl-1H-pyrrol-2-yl]benzoic acid

The procedure is in accordance with the process of Preparation 1, replacing in Step A the ethyl 2-methyl-1H-pyrrole-3-carboxylate by methyl 2-methyl-1H-pyrrole-3-carboxylate and also the 2-bromo-4-chlorobenzaldehyde used in Step B by 2-bromo-5-fluorobenzaldehyde.

¹H NMR (400 MHz, dmso-d6) δ ppm: 7.6 (dd, 1H), 7.42 (m, 2H), 6.22 (s, 1H), 4.15 (q, 2H), 3.21 (s, 3H), 2.5 (s, 3H), 1.23 (t, 3H)

¹⁹F NMR (400 MHz, dmso-d6) δ ppm: −113

### Preparation 27: 2-[4-(Ethoxycarbonyl)-1,5-dimethyl-1H-pyrrol-2-yl]-5-fluoro-4-methoxybenzoic acid

The procedure is in accordance with the process of Preparation 1, replacing in Step A the ethyl 2-methyl-1H-pyrrole-3-carboxylate by methyl 2-methyl-1H-pyrrole-3-carboxylate and also the 2-bromo-4-chlorobenzaldehyde used in Step B by 2-bromo-4-methoxy-5-fluorobenzaldehyde.

¹H NMR (400 MHz, dmso-d6) δ ppm: 9.65 (d, 1H), 7.67 (d, 1H), 7.24 (d, 1H), 6.48 (s, 1H), 4.19 (q, 2H), 3.97 (s, 3H), 3.38 (s, 3H), 2.56 (s, 3H), 1.26 (t, 3H).

### Preparation 28: 2-(4-Ethoxycarbonyl-1,5-dimethyl-1H-pyrrol-2-yl)-4-fluoro-5-methoxybenzoic acid

The procedure is in accordance with Preparation 1, replacing the 2-bromo-4-chlorobenzaldehyde used in Step B by 4-fluoro-5-methoxybenzaldehyde.

¹H NMR (400 MHz, dmso-d6) δ ppm: 12.9 (m, 1H), 7.6 (d, 1H), 7.22 (d, 1H), 6.23 (s, 1H), 4.2 (quad, 2H), 3.95 (s, 3H), 3.25 (s, 3H), 2.5 (s, 3H), 1.25 (t, 3H)

### Preparation 29: 5-Chloro-2-[4-(ethoxycarbonyl)-1,5-dimethyl-1H-pyrrol-2-yl]benzoic acid

The procedure is in accordance with the process of Preparation 1, replacing the 2-bromo-4-chlorobenzaldehyde used in Step B by 2-bromo-5-chlorobenzaldehyde.

### Preparation 30: 2-{1-[2-(Benzyloxy)ethyl]-4-(ethoxycarbonyl)-5-methyl-1H-pyrrol-2-yl}-4-chlorobenzoic acid

The procedure is in accordance with the process of Preparation 19, replacing the 6-bromo-1,3-benzodioxole-5-carbaldehyde by 2-bromo-4-chlorobenzaldehyde.

### Preparation 31: 5-Methoxy-2-[4-(methoxycarbonyl)-1,5-dimethyl-1H-pyrrol-2-yl]benzoic acid

The procedure is in accordance with the process of Preparation 1, replacing in Step A the ethyl 2-methyl-1H-pyrrole-3-carboxylate by methyl 2-methyl-1H-pyrrole-3-carboxylate and also the 2-bromo-4-chlorobenzaldehyde used in Step B by 2-bromo-5-methoxy-benzaldehyde.

¹H NMR (400 MHz, dmso-d6) δ ppm: 12.8 (bs, 1H), 7.34 (wd, 1H), 7.26 (d, 1H), 7.15 (dd, 1H), 6.19 (s, 1H), 3.84 (s, 3H), 3.69 (s, 3H), 3.22 (s, 3H), 2.5 (s, 3H)

### Preparation 32: 6-[1-(2,2-Difluoroethyl)-4-(ethoxycarbonyl)-5-methyl-1H-pyrrol-2-yl]-1,3-benzodioxole-5-carboxylic acid

The procedure is in accordance with the protocol of Preparation 19, replacing the 2-bromoethoxymethylbenzene used in Step A by 2-bromo-1,1-difluoro-ethane.

### Preparation 1′: (3R)-3-Methyl-1,2,3,4-tetrahydroisoquinoline hydrochloride

Step A: {(3S)-2-[(4-Methylphenyl)sulphonyl]-1,2,3,4-tetrahydroisoquinolin-3-yl}methyl 4-methylbenzenesulphonate

To a solution of 30.2 g of [(3S)-1,2,3,4-tetrahydroisoquinolin-3-yl]methanol (185 mmol) in 750 mL of dichloromethane there are successively added 91.71 g of tosyl chloride (481 mmol) and then, dropwise, 122.3 mL of N,N,N-triethylamine (740 mmol). The reaction mixture is then stirred at ambient temperature for 20 hours. It is then diluted with dichloromethane, washed successively with 1M HCl solution, saturated aqueous NaHCO₃ solution and then brine until neutral. The organic phase is then dried over MgSO₄, filtered and concentrated to dryness. The solid obtained is then dissolved in a minimum volume of dichloromethane and then cyclohexane is added until a precipitate is formed. This precipitate is then filtered off and washed with cyclohexane. After drying, the title product is obtained in the form of crystals.

¹H NMR: δ (400 MHz; dmso-d6; 300K): 7.75 (d, 2H, aromatic Hs, ortho 0-tosyl); 7.6 (d, 2H, aromatic Hs, ortho N-tosyl); 7.5 (d, 2H, aromatic Hs, meta O-tosyl); 7.3 (d, 2H, aromatic Hs, meta N-tosyl); 7.15-6.9 (m, 4H, aromatic Hs, tetrahydroisoquinoline); 4.4-4.15 (dd, 2H, aliphatic Hs, tetrahydroisoquinoline); 4.25 (m, 1H, aliphatic H, tetrahydroisoquinoline); 4.0-3.8 (2dd, 2H, aliphatic Hs, CH₂—O-tosyl); 2.7 (2dd, 2H, aliphatic Hs, tetrahydroisoquinoline); 2.45 (s, 3H, O—SO₂-Ph-CH₃); 2.35 (s, 3H, N—SO₂-Ph-CH₃)

IR: ν: —SO₂: 1339-1165 cm⁻¹

Step B: (3R)-3-Methyl-2-[(4-methylphenyl)sulphonyl]-1,2,3,4-tetrahydroisoquinoline

To a suspension of 8.15 g (214.8 mmol) of lithium aluminium hydride (LiAlH₄) in 800 mL of methyl tert-butyl ether (MTBE) there are added 101.2 g of the ditosyl compound obtained in Step A (214.8 mmol) dissolved in 200 mL of MTBE. The batch is then heated at 50° C. for 2 hours. It is allowed to cool and placed at 0° C., and there are then added, dropwise, 12 mL of 5N NaOH solution. The batch is stirred at ambient temperature for 45 minutes. The solid thereby obtained is then filtered off and washed with MTBE and then with dichloromethane. The filtrate is then concentrated to dryness. The title product is then obtained in the form of a solid.

¹H NMR: δ (400 MHz; dmso-d6; 300K): 7.70 (d, 2H, aromatic Hs, ortho N-tosyl); 7.38 (d, 2H, aromatic Hs, meta N-tosyl); 7.2-7.0 (m, 4H, aromatic Hs, tetrahydroisoquinoline); 4.4 (m, 2H, aliphatic Hs, tetrahydroisoquinoline); 4.3 (m, 1H, aliphatic H, tetrahydroisoquinoline); 2.85-2.51 (2dd, 2H, aliphatic Hs, tetrahydroisoquinoline); 2.35 (s, 3H, N—SO₂-Ph-CH₃); 0.90 (d, 3H, tetrahydroisoquinoline-CH₃)

IR: ν: —SO₂: 1332-1154 cm⁻¹

Step C: (3R)-3-Methyl-1,2,3,4-tetrahydroisoquinoline

To a solution of 31.15 g (103.15 mmol) of the monotosyl compound obtained in Step B in 500 mL of anhydrous methanol there are added, in portions, 3.92 g (161 mmol) of magnesium turnings. The batch is stirred in the presence of ultrasound for 96 hours. The reaction mixture is then filtered and the solid is washed several times with methanol. The filtrate is then concentrated to dryness. After purification by chromatography over silica gel using dichloromethane and ammonia-in-ethanol as eluants, the title product is obtained in the form of an oil.

¹H NMR: δ (400 MHz; dmso-d6; 300K): 7.05 (m, 4H, aromatic Hs, tetrahydroisoquinoline); 3.90 (m, 2H, aliphatic Hs, tetrahydroisoquinoline); 2.85 (m, 1H, aliphatic H, tetrahydroisoquinoline); 2.68-2.4 (2dd, 2H, aliphatic Hs, tetrahydroisoquinoline); 1.12 (d, 3H, tetrahydroisoquinoline-CH₃); 2.9-2.3 (m, broad, 1H, HN (tetrahydroisoquinoline))

IR: ν: —NH: 3248 cm⁻¹

Step D: (3R)-3-Methyl-1,2,3,4-tetrahydroisoquinoline hydrochloride

To a solution of 14.3 g (97.20 mmol) of the compound obtained in Step C in 20 mL of anhydrous ethanol there are added, dropwise, 100 mL of a 1M solution of HCl in ether. The batch is stirred at ambient temperature for 1 hour and then filtered. The crystals thereby obtained are washed with ethyl ether. After drying, the title product is obtained in the form of crystals.

¹H NMR: δ (400 MHz; dmso-d6; 300K): 9.57 (m, broad, 2H, NH₂⁺ (tetrahydroisoquinoline); 7.22 (m, 4H, aromatic Hs, tetrahydroisoquinoline); 4.27 (s, 2H, aliphatic Hs, tetrahydroisoquinoline); 3.52 (m, 1H, aliphatic H, tetrahydroisoquinoline); 3.03-2.85 (2dd, 2H, aliphatic Hs, tetrahydroisoquinoline); 1.39 (d, 3H, tetrahydroisoquinoline-CH₃)

IR: ν: —NH₂⁺: 3000-2300 cm⁻¹; ν: aromatic —CH: 766 cm⁻¹

### Preparation 2′:tert-Butyl [(3S)-1,2,3,4-tetrahydroisoquinolin-3-ylmethyl]carbamate

Step A: Benzyl (3S)-3-(hydroxymethyl)-3,4-dihydro-1H-isoquinoline-2-carboxylate

This compound is obtained using a protocol from the literature (R. B. Kawthekar et al South Africa Journal of Chemistry 63, 195, 2009) starting from 15 g of (3S)-1,2,3,4-tetrahydroisoquinolin-3-ylmethanol (91.9 mmol) in the presence of benzyl chloroformate and triethylamine in solution in dichloromethane. After purification by chromatography over silica gel using petroleum ether and ethyl acetate as eluants, the title product is obtained in the form of an oil.

¹H NMR: δ (300 MHz; DMSO-d6; 300K): 7.33 (m, 5H, aromatic Hs, O-benzyl); 7.15 (s, 4H, aromatic Hs, H tetrahydroisoquinoline); 5.13 (s, 2H, CH₂-Ph); 4.73 (d, 1H, H tetrahydroisoquinoline); 4.47 (m, H, CH₂OH); 4.36 (m, 1H, H tetrahydroisoquinoline); 4.28 (d, 1H, H tetrahydroisoquinoline); 3.39 (dd, 1H, CH₂OH); 3.23 (dd, 1H, CH₂OH); 2.93 (dd, 1H, H tetrahydroisoquinoline); 2.86 (dd, 1H, H tetrahydroisoquinoline)

IR: ν: OH: 3416 cm⁻¹; ν: <C═O 1694 cm⁻¹; ν:aromatic>C—H: 754 cm⁻¹

Step B: Benzyl (3S)-3-(azidomethyl)-3,4-dihydroisoquinoline-2 (1H)-carboxylate

This compound is obtained using a protocol from the literature (D. Pagé et al J. Med. Chem, 44, 2387, 2001) starting from 23 g of the compound obtained in Step A (77.3 mmol) in the presence of diphenylphosphoryl azide and triphenylphosphine in solution in THF. After purification by chromatography over silica gel using petroleum ether and ethyl acetate as eluants, the title product is obtained in the form of an oil.

¹H NMR: δ (400 MHz; DMSO-d6; 300K): 7.36 (m, 5H, aromatic Hs, O-benzyl); 7.19 (m, 4H, aromatic Hs, H tetrahydroisoquinoline); 5.16 (s, 2H, CH₂-Ph); 4.76 (d, 1H, H tetrahydroisoquinoline); 4.53 (m, 1H, H tetrahydroisoquinoline); 4.30 (m, 1H, H tetrahydroisoquinoline); 3.28 (m, 2H, CH₂N₃); 3.06 (dd, 1H, H tetrahydroisoquinoline); 2.78 (dd, 1H, H tetrahydroisoquinoline)

IR: ν: N₃: 2095 cm⁻¹; ν: <C═O: 1694 cm⁻¹ ν: aromatic>C—H: 754 cm⁻¹

Step C: Benzyl (3S)-3-(aminomethyl)-3,4-dihydroisoquinoline-2 (1H)-carboxylate

To a solution of 20.9 g (64.5 mmol) of the azido compound obtained in Step B in 650 mL of THF, there are successively added 25.5 g (97.2 mmol) of triphenylphosphine and 157 mL of water. The batch is refluxed for 2 hours 30 minutes. The reaction mixture is then concentrated to dryness and the residue oil is then taken up in isopropyl ether. A white precipitate appears; it is filtered off and washed with isopropyl ether. The filtrate is then concentrated to dryness and then purified by chromatography over silica gel using dichloromethane and methanol as eluants. The title product is obtained in the form of an oil.

¹H NMR: δ (400 MHz; DMSO-d6; 300K): 7.40 (m, 5H, aromatic Hs, O-benzyl); 7.20 (m, 4H, aromatic Hs, H tetrahydroisoquinoline); 5.15 (s, 2H, CH₂-Ph); 4.75-4.3 (m, 2H, H tetrahydroisoquinoline); 4.30 (d, 1H, H tetrahydroisoquinoline); 2.90 (m, 2H, CH₂NH₂); 2.45 (m, 2H, H tetrahydroisoquinoline); 1.40 (m, 2H, NH₂)

IR: ν: NH₂: 3400-3300 cm⁻¹; ν: <C═O: 1688 cm⁻¹

Step D: Benzyl (3S)-3-{[(tert-butoxycarbonyl)amino]methyl}-3,4-dihydroisoquinoline-2 (1H)-carboxylate

To a solution of 18.4 g (62.1 mmol) of the compound obtained in Step C in 630 mL of dichloromethane there are successively added 17.5 mL (124 mmol) of triethylamine and, in portions, 14.9 g (68.3 mmol) of di-tert-butyl dicarbonate. The batch is stirred at ambient temperature for 2 h. The reaction mixture is then concentrated and ethyl acetate is then added. The organic phase is successively washed with 1M HCl solution, brine, saturated aqueous NaHCO₃ solution and then brine. After drying, concentrating to dryness and purifying by chromatography over silica gel using petroleum ether and ethyl acetate as eluants, the title product is obtained in the form of an oil.

¹H NMR: δ (400 MHz; DMSO-d6; 300K): 7.35 (m, 5H, aromatic Hs, O-benzyl); 7.15 (m, 4H, aromatic Hs, H tetrahydroisoquinoline); 6.51 (m, 1H, NHBoc); 5.12 (s, 2H, CH₂-Ph); 4.76 (d, 1H, H tetrahydroisoquinoline); 4.51 (m, 1H, H tetrahydroisoquinoline); 4.36 (d, 1H, H tetrahydroisoquinoline); 2.95 (m, 3H, H tetrahydroisoquinoline+CH₂NHBoc); 2.71 (d, 1H, H tetrahydroisoquinoline); 1.34 (s, 9H, NHBoc)

IR: ν: NH: 3351 cm⁻¹; ν: <C═O: 1686 cm⁻¹

Step E: tert-Butyl [(3S)-1,2,3,4-tetrahydroisoquinolin-3-ylmethyl]carbamate

To a solution of 21 g (53 mmol) of the compound obtained in Step D in 600 mL of ethyl acetate there are added 2.1 g of palladium-on-carbon 10%. The batch is stirred at ambient temperature under a pressure of 1.3 bars of dihydrogen for 5 hours. The reaction mixture is then filtered and then concentrated to dryness. The title product is obtained in the form of a solid.

¹H NMR: δ (400 MHz; DMSO-d6; 300K): 7.15 (m, 4H, aromatic Hs, H tetrahydro-isoquinoline); 6.85 (t, 1H, NHBoc); 3.90 (m, 2H, H tetrahydroisoquinoline); 3.00 (m, 2H, CH₂NHBoc); 2.80 (m, 1H, H tetrahydroisoquinoline); 2.65 (dd, 1H, H tetrahydro-isoquinoline); 2.40 (dd, 1H, H tetrahydroisoquinoline); 1.40 (s, 9H, NHBoc)

IR: ν: NH: 3386-3205 cm⁻¹ (NH amide); ν: <C═O: 1688 cm⁻; ν: NH: 1526 cm⁻¹ (NH amine)

### Preparation 3′: (3S)-3-(4-Morpholinylmethyl)-1,2,3,4-tetrahydroisoquinoline

Step A: Benzyl (3S)-3-(4-morpholinylcarbonyl)-3,4-dihydro-2 (1H)-isoquinoline-carboxylate

To a solution of 5 g of (3S)-2-[(benzyloxy)carbonyl]-1,2,3,4-tetrahydro-3-isoquinolinecarboxylic acid (16 mmol) in 160 mL of dichloromethane there are added 1.5 mL of morpholine (17.6 mmol), then 9 mL of N,N,N-triethylamine (64 mmol), 3.3 g of 1-ethyl-3-(3′-dimethylaminopropyl)-carbodiimide (EDC) (19.2 mmol) and 2.6 g of hydroxybenzotriazole (HOBt) (19.2 mmol). The reaction mixture is stirred at ambient temperature overnight; it is then poured into ammonium chloride solution and extracted with ethyl acetate. The organic phase is then dried over MgSO₄, and then filtered and evaporated to dryness. The crude product thereby obtained is then purified by chromatography over silica gel using dichloromethane and methanol as eluants. The product is obtained in the form of a foam.

¹H NMR: δ (400 MHz; dmso-d6; 353K): 7.30 (m, 5H benzyl); 7.15 (m, 4H, aromatic Hs); 5.2-5.0 (m, 3H, 2H benzyl, 1H dihydroisoquinoline); 4.75-4.5 (2d, 2H dihydroisoquinoline); 3.55-3.3 (m, 8H morpholine); 3.15-2.9 (2dd, 2H dihydroisoquinoline)

IR: ν: >C═O: 1694; 1650 cm⁻¹

Step B: Benzyl (3S)-3-(4-morpholinylmethyl)-3,4-dihydro-2 (1H)-isoquinolinecarboxylate

To a solution of 5.3 g of the product obtained in Step A (13.9 mmol) in 278 mL of tetrahydrofuran there are added 14 mL of borane-dimethylsulphide complex (BH₃Me₂S) (27.8 mmol) at ambient temperature. The batch is heated for 4 hours at 80° C. It is allowed to return to ambient temperature and there are then added 7 mL (14 mmol) of BH₃Me₂S. The reaction mixture is again heated at 80° C. for 2 hours. The tetrahydrofuran is then evaporated off and then there is slowly added methanol and then 5.6 mL of 5N aqueous HCl solution (27.8 mmol). The mixture is stirred at ambient temperature overnight, and then at 80° C. for 1 hour. Saturated aqueous NaHCO₃ solution is then added to the reaction mixture placed at 0° C. until a pH of 8 is obtained, and extraction with ethyl acetate is then carried out. The organic phase is then dried over MgSO₄, and then filtered and evaporated to dryness. The title product is obtained in the form of an oil.

¹H NMR: δ (400 MHz; dmso-d6; 353K): 7.43-7.30 (unresolved peak, 5H benzyl); 7.19 (m, 4H, aromatic Hs); 5.16 (m, 2H, 2H benzyl); 4.79-4.29 (d, 2H dihydroisoquinoline); 4.58 (m, 1H dihydroisoquinoline); 3.50 (m, 4H morpholine); 3.02-2.80 (dd, 2H dihydroisoquinoline); 2.42-2.28 (unresolved peak, 5H, 4H morpholine, 1H morpholine); 2.15 (dd, 1H morpholine)

IR: ν: >CH: 2810 cm⁻¹; ν: >C═O: 1694 cm⁻¹; ν: >C—O—C<: 1114 cm⁻¹; ν: >CH—Ar: 751; 697 cm⁻¹

Step C: (3S)-3-(4-Morpholinylmethyl)-1,2,3,4-tetrahydroisoquinoline

To a solution of 4.9 g of the compound of Step B (13.4 mmol) in 67 mL of ethanol there is added 0.980 g of palladium dihydroxide (20% by weight) at ambient temperature. The reaction mixture is placed under 1.2 bars of hydrogen at ambient temperature for 4 hours. It is then passed through a Whatman filter and the palladium is then rinsed several times with ethanol. The filtrate is evaporated to dryness. The title product is obtained in the form of an oil.

¹H NMR: δ (400 MHz; dmso-d6; 300K): 7.12-7.0 (unresolved peak, 4H, aromatic Hs); 3.92 (s, 2H tetrahydroisoquinoline); 3.60 (t, 4H morpholine); 2.98 (m, 1H tetrahydroisoquinoline); 2.68 (dd, 1H tetrahydroisoquinoline); 2.5-2.3 (unresolved peak, 8H, 1H tetrahydroisoquinoline, 6H morpholine, 1H NH)

IR: ν: >NH: 3322 cm⁻¹; ν: >C—O—C<: 1115 cm⁻¹; ν: >CH—Ar: 742 cm⁻¹

### Preparation 4′:(3S)-3-[(4-Methyl-1-piperazinyl)methyl]-1,2,3,4-tetrahydroisoquinoline

The procedure is in accordance with the process of Preparation 3′, replacing the morpholine used in Step A by 1-methyl-piperazine.

### Preparation 5′:(3S)-3-[2-(Morpholin-4-yl)ethyl]-1,2,3,4-tetrahydroisoquinoline hydrochloride

Step A: tert-Butyl (3S)-3-(2-morpholino-2-oxo-ethyl)-3,4-dihydro-1H-isoquinoline-2-carboxylate

To a solution of 3 g (10.30 mmol) of [(3S)-2-(tert-butoxycarbonyl)-1,2,3,4-tetrahydroisoquinolin-3-yl]acetic acid in 100 mL of dichloromethane, there are added dropwise 1.10 mL (11.32 mmol) of morpholine, still dropwise 4.3 mL (30.9 mmol) of triethylamine, 2.20 g (12.40 mmol) of EDC and 1.70 g (1.68 mmol) of HOBt (hydroxybenzotriazole). The batch is stirred at ambient temperature for 15 hours. The reaction mixture is then diluted with dichloromethane, successively washed with 1M HCl solution, saturated aqueous NaHCO₃ solution and then brine until neutral. The organic phase is then dried over MgSO₄, filtered and concentrated to dryness. After purification by chromatography over silica gel using dichloromethane and methanol as eluants, the title product is obtained in the form of an oil.

¹H NMR: δ (400 MHz; dmso-d6; 300K): 7.20-7.10 (m, 4H, aromatic Hs, tetrahydroisoquinoline); 4.70 (m, 1H, aliphatic Hs, CH tetrahydroisoquinoline); 4.75-4.20 (2m, 2H, aliphatic Hs, CH₂ alpha to N tetrahydroisoquinoline); 3.60 (m, 8H, aliphatic Hs, morpholine); 3.00 and 2.70 (2dd, 2H, aliphatic H, tetrahydroisoquinoline); 2.50-2.20 (2d, 2H, aliphatic Hs, CH₂CO); 1.40 (s, 9H, ^{t}Bu)

IR: ν: C═O: 1687; 1625 cm¹

Step B: 1-(Morpholin-4-yl)-2-[(3S)-1,2,3,4-tetrahydroisoquinolin-3-yl]ethanone hydrochloride

To a solution of 2.88 g (7.18 mmol) of the compound obtained in Step A in 16 mL of dichloromethane, there are added dropwise 80 mL (80 mmol) of 1M solution of HCl in ether. The batch is stirred at ambient temperature for 15 hours, and then the suspension is filtered and the precipitate washed with ether. After drying, the title product is obtained in the form of a solid.

¹H NMR: δ (400 MHz; dmso-d6; 300K): 9.80-9.50 (m, 2H, NH₂+); 7.30-7.10 (m, 4H, aromatic Hs, tetrahydroisoquinoline); 4.30 (m, 2H, aliphatic Hs, CH₂ alpha to N tetrahydroisoquinoline); 3.80 (m, 1H, aliphatic Hs, CH tetrahydroisoquinoline); 3.70-3.40 (2m, 8H, aliphatic Hs, morpholine); 3.15 and 2.8 (m, 4H, aliphatic H, CH₂ tetrahydroisoquinoline and CH₂CO)

IR: ν: —NH₂+: 2800-1900 cm⁻¹; ν: C═O: 1620 cm⁻¹

Step C: (3S)-3-[2-(Morpholin-4-yl)ethyl]-1,2,3,4-tetrahydroisoquinoline hydrochloride

A solution of 2.2 g (7.44 mmol) of the compound obtained in Step B in 22 mL of MTBE and 5 mL of dichloromethane is prepared. After cooling in an ice bath at 0° C., there are added thereto, dropwise, 15 mL (15 mmol) of 1M LiAlH₄ solution in tetrahydrofuran. The batch is then stirred at ambient temperature for 6 hours. It is placed at 0° C., and there is then added, dropwise, 1 mL of 5N NaOH solution. The batch is stirred at ambient temperature for 45 minutes. The solid is then filtered off and washed with MTBE and then with dichloromethane and the filtrate is concentrated to dryness. The oil thereby obtained is diluted with dichloromethane and there are added, dropwise, 6.3 mL of a 1M solution of HCl in ether. The batch is stirred at ambient temperature for 1 hour and then filtered. The crystals thereby obtained are washed with ethyl ether. After drying, the title product is obtained in the form of a solid.

¹H NMR: δ (400 MHz; dmso-d6; 300K): 11.35+9.80 (2m, 2H, NH₂+); 10.00 (m, H, NH+); 7.20 (m, 4H, aromatic Hs, tetrahydroisoquinoline); 4.30 (s, 2H, aliphatic Hs, CH₂ alpha to N tetrahydroisoquinoline); 4.00+3.85 (2m, 4H, aliphatic Hs, CH₂ alpha to N morpholine); 3.70 (m, 1H, aliphatic Hs, CH tetrahydroisoquinoline); 3.55-3.30 (m, 4H, aliphatic Hs, CH alpha to O morpholine and CH₂-morpholine); 3.15 (dd, 1H, aliphatic H, CH₂ tetrahydroisoquinoline); 3.10 (m, 2H, aliphatic H, CH alpha to O morpholine); 2.90 (dd, 1H, aliphatic H, CH₂ tetrahydroisoquinoline); 2.30+2.15 (2m, 2H, aliphatic H, CH₂-tetrahydroisoquinoline)

IR: ν: NH⁺/—NH₂⁺: between 3500 and 2250 cm⁻¹; ν: C═C: weak 1593 cm⁻¹; ν: aromatic C—H: 765 cm⁻¹

### Preparation 6′: (3R)-3-[3-(Morpholin-4-yl)propyl]-1,2,3,4-tetrahydroisoquinoline

Step A: {(3S)-2-[(4-Methylphenyl)sulphonyl]-1,2,3,4-tetrahydroisoquinolin-3-yl}methyl 4-methylbenzenesulphonate

The process is the same as that of Step A of Preparation 1′.

Step B: tert-Butyl 2-({(3R)-2-[(4-methylphenyl)sulphonyl]-1,2,3,4-tetrahydroisoquinolin-3-yl}methyl)-3-(morpholin-4-yl)-3-oxopropanoate

To a suspension of 1 g of NaH (60%) (25.08 mmol) in 30 mL of MTBE there are added, dropwise, a solution of 5 g of tert-butyl 3-morpholino-3-oxopropanoate (21.81 mmol) in 20 mL of anhydrous MTBE. This suspension stirred at ambient temperature for 1 hour and then the compound obtained in Step A is added in the form of a powder. The batch is stirred at 60° C. for 30 hours. 100 mL of saturated aqueous ammonium chloride solution are added. The resulting solution is extracted with dichloromethane. The organic phase is then dried over MgSO₄, filtered and concentrated to dryness. After purification by chromatography over silica gel using dichloromethane and MeOH as eluants, the expected product is obtained in the form of an oil.

¹H NMR (500 MHz, dmso-d6) δ ppm: 7.63/7.59 (2d, 2H), 7.3/7.26 (2d, 2H), 7.13 (m, 2H), 7.09/6.97 (2t, 2H), 4.64/4.55/4.36/4.28 (2AB, 2H), 4.25/4.11 (2m, 1H), 3.81 (m, 1H), 3.73-3.48 (m, 4H), 3.57-3.32 (m, 4H), 2.51 (m, 2H), 2.32/2.31 (2s, 3H), 1.88/1.79 (2m, 2H), 1.39/1.38 (2s, 9H) IR (ATR) cm¹: ν: >C═O: 1731 (ester); ν: >C═O: 1644 (amide); ν: —SO2: 1334-1156; ν: >C—O—C<: 1115; γ: >CH—Ar: 815-746-709

Step C: 2-({(3R)-2-[(4-Methylphenyl)sulphonyl]-1,2,3,4-tetrahydroisoquinolin-3-yl}methyl)-3-(morpholin-4-yl)-3-oxopropanoic acid

To a solution of 9.5 g (17.97 mmol) of the compound obtained in Step B in 40 mL of dioxane there are added, dropwise, 20 mL of a 4M solution of HCl in dioxane. The batch is stirred at ambient temperature for 48 hours and then the solution is concentrated to dryness. After drying, the expected product is obtained in the form of an oil.

¹H NMR (400 MHz, dmso-d6) δ ppm: 12.75 (m, 1H), 7.6 (2*d, 2H), 7.3 (2*d, 2H), 7.1/6.95 (2*m, 4H), 4.7-4.2 (d, 2H), 4.25/4.12 (2*m, 1H), 3.9-3.3 (m, 9H), 2.55 (d, 2H), 2.3 (2*s, 3H), 1.8 (t, 2H)

IR (ATR) cm⁻¹: ν: —OH: 3500 to 2000; ν: >C═O: 1727 (acid); ν: >C═O: 1634 (amide); ν: —SO2:1330-1155

Step D: 3-[(3R)-2-[(4-Methylphenyl)sulphonyl]-1,2,3,4-tetrahydroisoquinolin-3-yl]-1-(morpholin-4-yl)propan-1-one

To a solution of 7.80 g (16.51 mmol) of the compound obtained in Step C in 100 mL of DMSO there are added 1.16 g (19.83 mmol) of solid sodium chloride (NaCl) and then, dropwise, 5 mL of water. The batch is stirred at 130° C. for 1 hour and then the solution is concentrated to %. The reaction mixture is then diluted with dichloromethane and washed successively with saturated aqueous lithium chloride solution and then with brine. The organic phase is then dried over MgSO₄, filtered and concentrated to dryness. After purification by chromatography over silica gel using cyclohexane and ethyl acetate as eluants, the expected product is obtained in the form of an oil.

¹H NMR (400 MHz, dmso-d6) δ ppm: 7.65 (d, 2H), 7.3 (d, 2H), 7.15/7 (2 m, 4H), 4.6 (d, 1H), 4.25 (d, 1H), 4.2 (m, 1H), 3.5 (m, 4H), 3.4 (2 m, 4H), 2.6 (2 dd, 2H), 2.35 (s, 3H), 2.3 (m, 2H), 1.5 (quad., 2H)

IR (ATR) cm⁻¹: ν: >C═O: 1639; ν: —SO2: 1331-1156; γ: >CH—Ar: 815-675

Step E: (3R)-2-[(4-Methylphenyl)sulphonyl]-3-[3-(morpholin-4-yl)propyl]-1,2,3,4-tetrahydroisoquinoline

To a solution of 6.0 g (14.0 mmol) of the compound obtained in Step D in 60 mL of MTBE and 14 mL of dichloromethane there are added 1.06 g (28 mmol) of LAH in portions over 5 minutes. The batch is stirred at ambient temperature for 15 hours. There are added, dropwise, 1.5 mL of water and stirring is carried out for 15 minutes. There are then added, dropwise, 1.5 mL of 5M sodium hydroxide solution and stirring is carried out for 15 minutes. The reaction mixture is then diluted with MTBE and dichloromethane. The suspension is then filtered and the precipitate is washed with MTBE and dichloromethane. The organic phase is then dried over MgSO₄, filtered and concentrated to dryness. After purification by chromatography over silica gel using dichloromethane and ammonia-in-ethanol as eluants, the expected product is obtained in the form of an oil.

¹H NMR (400 MHz, dmso-d6) δ ppm: 7.68 (d, 2H), 7.32 (d, 2H), 7.1 (unresolved peak, 4H), 4.65/4.23 (AB, 2H), 4.2 (m, 1H), 3.55 (t, 4H), 2.7/2.6 (ABX, 2H), 2.35 (s, 3H), 2.25 (t, 4H), 2.2 (t, 2H), 1.4/1.3 (2m, 4H).

IR (ATR) cm⁻¹: ν: —SO2: 1333-1158

Step F: (3R)-3-[3-(Morpholin-4-yl)propyl]-1,2,3,4-tetrahydroisoquinoline

To a solution of 1.50 g (3.62 mmol) of the compound obtained in Step E in 20 mL of anhydrous methanol there are added 2.0 g (82.3 mmol), in portions, of magnesium turnings. The batch is stirred in the presence of ultrasound for 96 hours. The reaction mixture is then filtered, the solid is washed several times with methanol, and the filtrate is concentrated to dryness. After purification by chromatography over silica gel using dichloromethane and ammonia-in-ethanol as eluants, the expected product is obtained in the form of an oil.

¹H NMR (400 MHz, dmso-d6) δ ppm: 7.3 (d, 2H), 7.1 (t, 2H), 7.1 (d+t, 3H), 7 (d, 2H), 3.9 (s, 2H), 3.55 (t, 4H), 2.75 (m, 1H), 2.72/2.45 (dd, 2H), 2.35 (t, 4H), 2.25 (t, 2H), 1.6 (m, 2H), 1.45 (m, 2H)

IR (ATR) cm⁻¹: ν: >NH2+/NH+: 3500-2300; ν: >C—O—C<: 1115

### High-Resolution Mass Spectrometry (ESI+−/FIA/HR):

C₁₆H₂₄N₂O Empirical formula:

[M+H]⁺ calculated: 261.1961

[M+H]⁺ measured: 261.1959

### Preparation 7′: (3S)-3-[(9aS)—Octahydropiperazino[2,1-c]morpholin-8-ylmethyl]-1,2,3,4-tetrahydroisoquinoline trihydrochloride

Step A: tert-Butyl (9aS)-4-oxo-octahydropiperazino[2,1-c]morpholine-8-carboxylate

The synthesis of this compound is known in the literature (J. Med. Chem. 2012, 55, 5887 for the opposite enantiomer).

Step B: (9aS)—Octahydropiperazino[2,1-c]morpholin-4-one hydrochloride

A 4M solution of HCl in dioxane (60 mL, 240 mmol) is added to the compound tert-butyl (9aS)-4-oxo-octahydropiperazino[2,1-c]morpholine-8-carboxylate (11.8 g, 46.0 mmol) cooled using an ice bath. The solution is then stirred at ambient temperature for 2 hours, and then at 50-60° C. for 1.5 hours. The solution is then evaporated to dryness. The residue is co-evaporated with dioxane (3×20 mL) and then dried in vacuo to obtain the expected compound in the form of a solid.

¹H NMR (400 MHz, dmso-d6) δ ppm: 2.80-2.94 (m, 2H), 2.94-3.05 (m, 1H), 3.23-3.37 (m, 2H), 3.59-3.69 (m, 1H), 3.83-3.93 (m, 1H), 3.95-4.04 (m, 1H), 4.02-4.13 (m, 2H), 4.45-4.55 (m, 1H), 9.58 (br s, 2H)

Step C: tert-Butyl (3S)-3-[(9aS)-4-oxo-octahydropiperazino[2,1-c]morpholine-8-carbonyl]-1,2,3,4-tetrahydroisoquinoline-2-carboxylate

EDC (3.90 g, 20.3 mmol) is added to a solution of the compound of Step B (3.02 g, 15.7 mmol), (3S)-2-[(tert-butoxy)carbonyl]-1,2,3,4-tetrahydroisoquinoline-3-carboxylic acid (4.6 g, 16.6 mmol), triethylamine (8.0 mL, 57.4 mmol) and HOBt (2.72 g, 20.1 mmol) in dichloromethane (150 mL). The mixture is stirred at ambient temperature for 21 hours. 1N aqueous HCl solution (105 mL) is added and the precipitate formed is filtered off using a Buchner funnel. The phases of the filtrate are separated. The aqueous phase is extracted with dichloromethane (2×10 mL). The combined organic phases are washed with 3N aqueous HCl solution (35 mL), then with aqueous 5% potassium bicarbonate solution (2×35 mL) and finally with brine (35 mL). The organic phase is dried over Na₂SO₄, filtered and then concentrated under reduced pressure. The product is purified by chromatography over silica gel using ethyl acetate and heptane as eluants to obtain the expected compound in the form of a solid.

¹H NMR (400 MHz, CDCl₃) δ ppm: 1.38-1.57 (m, 9H), 2.39-2.89 (m, 2H), 2.89-3.34 (m, 3H), 3.34-3.70 (m, 2H), 3.90-4.06 (m, 1H), 4.09-4.27 (m, 2H), 4.30-5.00 (m, 5H), 5.20-5.37 (m, 1H), 7.03-7.24 (m, 4H)

Step D: (3S)-3-[(9aS)—Octahydropiperazino[2,1-c]morpholin-8-ylmethyl]-1,2,3,4-tetrahydroisoquinoline trihydrochloride

4M HCl solution in dioxane (45 mL, 180 mmol) is added to the compound of Step C (6.6 g, 46.0 mmol) cooled in an ice bath. The suspension is then stirred at ambient temperature for 24 hours and then it is evaporated to dryness. The residue is co-evaporated with MTBE and then dried in vacuo. The solid thereby obtained is suspended in tetrahydrofuran (160 mL), and then LiAlH₄ (3.0 g, 79.1 mmol) is added. The suspension is refluxed for 6.5 hours, and it is then cooled in an ice bath. Water (3 mL) is then added over a period of 7 minutes. After 0.5 hours, 2N aqueous sodium hydroxide solution (6 mL) is added. Water (6 mL) is again added 0.25 hours later. Finally, Celite (7 g) and Na₂SO₄ (25 g) are added 0.5 hours later. The suspension is filtered over Celite and rinsed with tetrahydrofuran (2×100 mL). The filtrate is concentrated to dryness. The oil thereby obtained is dissolved in MTBE (50 mL). The resulting solution is filtered and the filtrate concentrated. The residue is dissolved in methanol (60 mL), and then 4M HCl solution in dioxane (20 mL) is added. The solution is heated to 40° C. and treated with activated charcoal (0.66 g), with stirring, for 1 hour. The suspension is filtered over Celite and rinsed with warm methanol. The filtrate is concentrated until the product starts to crystallise out. Crystallisation is allowed to continue for 16 hours at ambient temperature. The solid obtained is filtered off and rinsed with a mixture of 2-propanol/MTBE (4/6) (2×20 mL), and then with MTBE (2×20 mL). After drying, the expected compound is obtained.

¹H NMR (400 MHz, D₂O) δ ppm: 2.28-2.44 (m, 1H), 2.74-3.00 (m, 4H), 3.08-3.27 (m, 3H), 3.27-3.42 (m, 2H), 3.43-3.56 (m, 2H), 3.56-3.69 (m, 2H), 3.76-3.95 (m, 2H), 4.00-4.22 (m, 2H), 4.35-4.50 (m, 2H), 7.20-7.41 (m, 4H)

¹³C NMR (100 MHz, D₂O) δ ppm: 29.00, 44.58, 50.30, 51.08, 51.17, 52.75, 53.17, 58.08, 61.60, 64.61, 66.37, 127.14, 127.71, 128.00, 128.77, 129.55, 131.15

MS (ESI): [M+H]⁺ 288.16

### Preparation 8′: (3S)-3-(1-Oxa-6-azaspiro[3.3]hept-6-ylmethyl)-1,2,3,4-tetrahydro-isoquinoline

Step A: (3S)-3-(Iodomethyl)-2-[(4-methylphenyl)sulphonyl]-1,2,3,4-tetrahydro-isoquinoline

The compound of Step A of Preparation 6′ (4.0 g; 8.48 mmol) in acetonitrile (10 mL) is placed in a 27-ml microwave tube and then sodium iodide (1.40 g; 9.33 mmol) is added. The reaction mixture is heated for 5 hours at 100° C. using microwaves (200 W). It is then filtered and the solid is washed with dichloromethane. The filtrate is evaporated to dryness and then the residue is purified by chromatography over silica gel using heptane and ethyl acetate as eluants. The title compound is obtained in the form of an oil.

¹H NMR (400 MHz, dmso-d6) δ ppm: 7.64 (d, 2H), 7.28 (d, 2H), 7.15-7 (m, 4H), 4.5/4.3 (2d, 2H), 4.14 (m, 1H), 3.22 (m, 2H), 2.82 (m, 2H), 2.31 (s, 3H)

IR (ATR) cm⁻¹: 1897 ν−Ar, 1333+1156 ν−SO2

Step B: (3S)-2-[(4-Methylphenyl)sulphonyl]-3-(1-oxa-6-azaspiro[3.3]hept-6-ylmethyl)-1,2,3,4-tetrahydroisoquinoline

The iodinated compound (2.5 g; 5.85 mmol) obtained in the preceding Step is dissolved in acetonitrile (50 mL). 1-Oxa-6-azaspiro[3.3]heptane oxalate (1.21 g; 6.36 mmol) is added, followed by potassium carbonate (1.61 g; 11.7 mmol). The reaction mixture is heated for 15 hours at reflux. The reaction mixture is filtered and washed with acetonitrile, and then is evaporated to dryness.

The compound is purified by chromatography over silica gel using dichloromethane and ammonia-in-methanol as eluants. The title compound is obtained in the form of an oil.

¹H NMR (400 MHz, dmso-d6) δ ppm: 7.68 (d, 2H), 7.32 (d, 2H), 7.14-7 (m, 4H), 4.53/4.2 (dd, 2H), 4.34 (t, 2H), 3.95 (m, 1H), 3.5/3.4/2.98 (3m, 4H), 2.7 (t, 2H), 2.68-2.58 (m, 2H), 2.34 (s, 3H), 2.31-2.24 (m, 2H)

IR (ATR) cm⁻¹: 1333+1156 ν —SO2

Step C: (3S)-3-(1-Oxa-6-azaspiro[3.3]hept-6-ylmethyl)-1,2,3,4-tetrahydroisoquinoline

The tosylated compound of the above Step (1.3 g; 3.26 mmol) is dissolved in 10 mL of methanol. Powdered magnesium (633 mg; 26.08 mmol) is added in portions of 160 mg every 3 hours. The reaction mixture is stirred in an ultrasound bath 15 hours. It is then filtered over Celite, washed with copious amounts of methanol, and then evaporated to dryness. The compound is purified by chromatography over silica gel using dichloromethane and ammonia-in-methanol as eluants. The compound is obtained in the form of an oil.

¹H NMR (400 MHz, dmso-d6) δ ppm: 7.01 (m, 4H), 4.46 (t, 2H), 3.85 (s, 2H), 3.51/3.05 (dd, 2H), 2.73 (t, 2H), 2.61/2.4 (m, 4H), 2.4 (m, 1H), 2.4 (m, 1H)

IR (ATR) cm⁻¹: 3325 ν>NH

### Preparation 9′: (3S)-3-[(9aR)—Octahydropiperazino[2,1-c]morpholin-8-ylmethyl]-1,2,3,4-tetrahydroisoquinoline trihydrochloride

Step A: tert-Butyl (9aR)-4-oxo-octahydropiperazino[2,1-c]morpholine-8-carboxylate

Synthesis of this compound is described in the literature (J. Med. Chem. 2012, 55, 5887).

Step B: (9aR)—Octahydropiperazino[2,1-c]morpholin-4-one hydrochloride

A 4M solution of HCl in dioxane (39 mL, 154 mmol) is added to the compound of Step A (11.3 g, 44.1 mmol). The solution is then stirred at ambient temperature for 5 hours, and then a 4M solution of HCl in dioxane (12 mL, 48 mmol) is added again. The mixture is stirred for 16 hours. The solution is then evaporated to dryness to yield the expected product in the form of a solid.

¹H NMR (400 MHz, dmso-d6) δ ppm: 2.79-2.93 (m, 2H), 3.02 (td, J=13.1, 2.7 Hz, 1H), 3.22-3.34 (m, 2H), 3.59-3.67 (m, 1H), 3.86-3.96 (m, 1H), 3.96-4.01 (m, 1H), 4.05 (AB q, J=13.3 Hz, 2H), 4.48 (dd, J=14.1, 2.5 Hz, 1H), 9.71 (br. s, 1H), 9.91 (br. s, 1H)

Step C: tert-Butyl (3S)-3-[(9aR)-4-oxo-octahydropiperazino[2,1-c]morpholine-8-carbonyl]-1,2,3,4-tetrahydroisoquinoline-2-carboxylate

EDC (5.17 g, 27.0 mmol) is added to a solution of the compound of the above Step (4.0 g, 20.7 mmol), (3S)-2-[(tert-butoxy)carbonyl]-1,2,3,4-tetrahydroisoquinoline-3-carboxylic acid (6.04 g, 21.8 mmol), triethylamine (11.6 mL, 83.1 mmol) and HOBt (3.65 g, 27.0 mmol) in dichloromethane (100 mL). The mixture is stirred at ambient temperature for 16 hours. 1N aqueous HCl solution (70 mL) is added and the precipitate formed is filtered off using a Buchner funnel. The phases of the filtrate are separated. The organic phase is washed with saturated aqueous potassium carbonate solution and is then concentrated under reduced pressure. The residue is pre-absorbed onto silica gel and is purified by chromatography over silica gel using ethyl acetate and heptane as eluants to yield the expected compound in the form of a solid.

¹H NMR (400 MHz, dmso-d6) δ ppm: 1.25-1.58 (m, 9H), 2.50-2.76 (m, 2H), 2.76-3.25 (m, 3H), 3.37-3.76 (m, 2H), 3.92-4.50 (m, 5H), 4.06 (s, 2H), 4.66 (d, J=15.6 Hz, 1H), 4.76-5.28 (m, 1H), 7.05-7.31 (m, 4H)

Step D: (3S)-3-[(9aR)—Octahydropiperazino[2,1-c]morpholin-8-ylmethyl]-1,2,3,4-tetrahydroisoquinoline trihydrochloride

A 4M solution of HCl in dioxane (24.0 mL, 96.2 mmol) is added to a solution of the compound of Step C (8.00 g, 12.25 mmol) in dichloromethane (25 mL). The mixture is stirred at ambient temperature for 16 hours and is then concentrated to dryness. The crude product obtained is added to a suspension of LiAlH₄ (1.97 g, 51.91 mmol) in tetrahydrofuran (140 mL). The mixture is refluxed until the reaction (monitored by LC-MS) is complete and it is then cooled to 0° C. Water (2.5 mL) is added dropwise. After stirring for 10 minutes, aqueous 2M sodium hydroxide solution (5 mL) is added dropwise. Water (5 mL) is again added after stirring for 10 minutes. There are finally added Celite (4 g) and Na₂SO₄ (12 g) after stirring for an additional 10 minutes. The suspension is filtered over Celite and the filtrate is concentrated to dryness. The crude residue thereby obtained is dissolved in methanol (80 mL), and then a 4M solution of HCl in dioxane (16.75 mL, 67.0 mmol) is added. The mixture is stirred at ambient temperature for 3 hours, and then concentrated to dryness. The residue is dissolved in a minimum of warm methanol (70 mL), and then MTBE (3-5 mL) is added. The solution is cooled at 0° C. for 1 hour in an ice-cold water bath, and the product precipitates out. A little MTBE (2-3 mL) is again added and the mixture is allowed to stand for a further 1 hour at 0° C. The solid obtained is filtered over a Buchner funnel and dried in vacuo to yield the expected compound in the form of a solid.

¹H NMR (400 MHz, CD₃OD) δ ppm: 2.65 (t, J=11.1 Hz, 1H), 2.74-2.97 (m, 4H), 3.10 (d, J=12.5 Hz, 1H), 3.19 (dd, J=17.6, 4.8 Hz, 1H), 3.25-3.55 (m, 5H), 3.59-3.74 (m, 2H), 3.82-4.15 (m, 4H), 4.45 (AB q, J=15.9 Hz, 2H), 7.21-7.35 (m, 4H).

¹³C NMR (100 MHz, CD₃OD) δ ppm: 30.21, 45.67, 50.22, 51.99, 52.90, 53.58, 53.71, 59.25, 62.58, 65.30, 67.07, 127.76, 128.40, 129.08, 129.36, 130.16, 131.95 MS (ESI): [M+H]⁺ 288.2

### Preparation 1″: N-[4-[tert-Butyl(dimethyl)silyl]oxyphenyl]-1-methyl-pyrazol-4-amine

Step A: 4-{[tert-Butyl(dimethyl)silyl]oxy}aniline

The title compound is obtained starting from 4-aminophenol in THF in the presence of imidazole and tert-butyl(dimethyl)silyl chloride in accordance with the protocol described in the literature (S. Knaggs et al, Organic & Biomolecular Chemistry, 3(21), 4002-4010; 2005).

¹H NMR: δ (400 MHz; dmso-d6; 300K): 6.45-6.55 (dd, 4H, aromatic Hs); 4.60 (m, 2H, NH₂-Ph); 0.90 (s, 9H, Si (CH₂)₂CH(CH₃)₂); 0.10 (s, 6H, Si (CH₂)₂CH(CH₃)₂)

IR: ν: —NH₂⁺: 3300-3400 cm⁻¹

Step B: N-[4-[tert-Butyl(dimethyl)silyl]oxyphenyl]-1-methyl-pyrazol-4-amine

To a solution of 30.8 g (0.137 mol) of the compound of Step A in 525 mL of anhydrous toluene there are successively added 29.8 g of sodium tert-butylate (0.310 mol), 4.55 g of Pd₂(dba)₃ (also referred to as tris(dibenzylideneacetone)dipalladium(0)) (4.96 mmol), 4.81 g of 2-di-tert-butylphosphino-2′,4′,6′-tri-isopropyl-1,1′-biphenyl (9.91 mmol) and 12.8 mL of 4-bromo-1-methyl-1H-pyrazole (0.124 mol). The batch is degassed under argon for 30 minutes and then refluxed for 3 hours. It is allowed to cool. The reaction mixture is concentrated to dryness and then taken up in dichloromethane, filtered over Celite and then concentrated to dryness again. The residue is then purified by chromatography over silica gel using dichloromethane and ethyl acetate as eluants to provide the expected product in the form of a solid.

¹H NMR: δ (400 MHz; dmso-d6; 300K): 7.55 (s, 1H, pyrazole); 7.23 (s, 1H, pyrazole); 7.18 (broad s, 1H, NH₂-Ph); 6.64 (m, 4H, aromatic Hs); 3.77 (s, 3H, CH₃-pyrazole); 0.90 (s, 9H, Si (CH₂)₂CH(CH₃)₂); 0.12 (s, 6H, Si (CH₂)₂CH(CH₃)₂)

IR: ν —NH⁺: 3275 cm⁻¹; ν Ar and C═N: 1577 and 1502 cm⁻¹; ν —Si—C—: 1236 cm⁻¹; ν —Si—O—: 898 cm⁻¹; ν —Si—C—: 828, 774 cm⁻¹

### Preparation 2″: 4-{[tert-Butyl(dimethyl)silyl]oxy}-N-phenylaniline

To a solution of 12 g of 4-anilinophenol (64.7 mmol) in 200 mL of acetonitrile there are added, at ambient temperature, 6.7 g of imidazole (97.05 mmol) and 11.7 g of tert-butyl(dimethyl)silyl chloride (77.64 mmol). The batch is stirred at 70° C. for 4 hours. The reaction mixture is then poured into water and extracted with ether. The organic phase is then dried over MgSO₄, then filtered and evaporated to dryness. The crude product thereby obtained is then purified by chromatography over silica gel using petroleum ether and dichloromethane as eluants. The title product is obtained in the form of a powder.

¹H NMR: δ (400 MHz; dmso-d6; 300K): 7.84 (s, 1H NH); 7.17 (t, 2H aniline); 6.98 (d, 2H phenoxy); 6.94 (d, 2H aniline); 6.76 (d, 2H phenoxy); 6.72 (t, 1H aniline); 0.95 (s, 9H tert-butyl); 0.15 (s, 6H dimethyl)

IR: ν: >NH: 3403 cm⁻¹; ν:>Ar: 1597 cm⁻¹

### Preparation 3″: tert-Butyl 5-[(4-{[tert-butyl(dimethyl)silyl]oxy}phenyl)amino]-1H-indole-1-carboxylate

The procedure is in accordance with the process of Preparation 1″, replacing the 4-bromo-1-methyl-1H-pyrazole used in Step B by tert-butyl 5-bromo-1H-indole-1-carboxylate.

¹H NMR: δ (400 MHz; dmso-d6; 300K): 7.85 (d, 1H); 7.78 (s, 1H); 7.55 (d, 1H); 7.15 (d, 1H); 6.95 (m, 3H); 6.75 (d, 2H); 6.58 (d, 1H); 1.65 (s, 9H); 1.00 (s, 9H); 0.2 (s, 6H)

### Preparation 4″: N-(4-{[tert-Butyl(dimethyl)silyl]oxy}phenyl)-1-methyl-1H-indol-5-amine

The procedure is in accordance with the process of Preparation 1″, replacing the 4-bromo-1-methyl-1H-pyrazole used in Step B by 5-bromo-1-methyl-1H-indole.

### Preparation 5″: N-(4-{[tert-Butyl(dimethyl)silyl]oxy}phenyl)-1-methyl-1H-indazol-5-amine

The procedure is in accordance with the process of Preparation 1″, replacing the 4-bromo-1-methyl-1H-pyrazole used in Step B by 5-bromo-1-methyl-1H-indazole.

### Preparation 6″: N-(4-{[tert-Butyl(dimethyl)silyl]oxy}phenyl)-3-fluoro-4-methylaniline

The procedure is in accordance with the process of Preparation 1′″, replacing the 4-bromo-1-methyl-1H-pyrazole used in Step B by 4-bromo-2-fluoro-1-methylbenzene.

### Preparation 7″: N-(4-{I[tert-Butyl(dimethyl)silyl]oxy}phenyl)-3-fluoroaniline

The procedure is in accordance with the process of Preparation 1″, replacing the 4-bromo-1-methyl-1H-pyrazole used in Step B by 1-bromo-3-fluorobenzene.

### Preparation 8″: 4-Benzyloxy-N-phenyl-aniline

To a solution of 4-hydroxy-N-phenyl-aniline (30 g; 162 mmol) in acetonitrile (400 mL) there are added 58 g of Cs₂CO₃ (178 mmol) and stirring is carried out for 15 minutes at ambient temperature. Benzyl bromide (22.5 mL; 178 mmol) is then added dropwise and then the reaction mixture is refluxed for 4 hours. After filtering and rinsing with acetonitrile, the filtrate is concentrated and purified by chromatography over silica gel using petroleum ether and ethyl acetate as eluants. The title product is then obtained in the form of a colourless solid.

¹H NMR: δ (400 MHz; dmso-d6; 300K): 7.80 (m, 1H, NH); 7.45 (m, 2H, aryl); 7.40 (m, 2H, aryl); 7.30 (m, 1H, aryl); 7.15 (s, 2H, aryl); 7.05 (d, 2H, aryl); 6.9-7.0 (m, 4H, aryl); 6.70 (t, 1H, aryl); 5.05 (s, 2H, benzyl).

IR: ν: >NH: 3408 cm⁻¹

### Preparation 9″: N-(4-{[tert-Butyl(dimethyl)silyl]oxy}phenyl)pyridin-4-amine

The procedure is in accordance with the process of Preparation 1″, replacing the 4-bromo-1-methyl-1H-pyrazole used in Step B by 4-bromopyridine.

IR: ν —NH—: 3200 and 2500 cm⁻¹; ν:—Si—O—: 902 cm⁻¹; ν:—Si—C—: 820 cm⁻¹

### Preparation 10″: N-(4-{[tert-Butyl(dimethyl)silyl]oxy}phenyl)-4-fluoroaniline

The procedure is in accordance with the process of Preparation 1″, replacing the 4-bromo-1-methyl-1H-pyrazole used in Step B by 1-bromo-4-fluorobenzene.

### Preparation 11″: N-(4-{[tert-Butyl(dimethyl)silyl]oxy}phenyl)-1-methyl-1H-pyrrolo[2,3-b]pyridin-5-amine

The procedure is in accordance with the process of Preparation 1″, replacing the 4-bromo-1-methyl-1H-pyrazole used in Step B by 5-bromo-1-methyl-1H-pyrrolo[2,3-b]pyridine (obtained in accordance with a protocol from the literature: Heterocycles, 60(4), 865, 2003).

IR: ν:—NH—: 3278 cm⁻¹; ν: aromatic —C═C— moieties: 1605 cm⁻¹

### Preparation 12″: N-(4-{[tert-Butyl(dimethyl)silyl]oxy}phenyl)-2-methoxypyrimidin-5-amine

The procedure is in accordance with the process of Preparation 1″, replacing the 4-bromo-1-methyl-1H-pyrazole used in Step B by 5-bromo-2-methoxypyrimidine.

### Preparation 13″: N-(4-{[tert-Butyl(dimethyl)silyl]oxy}phenyl)-1-methyl-2,3-dihydro-1H-pyrrolo[2,3-b]pyridin-5-amine

The procedure is in accordance with the process of Preparation 1″, replacing the 4-bromo-1-methyl-1H-pyrazole used in Step B by 5-bromo-1-methyl-2,3-dihydro-1H-pyrrolo-[2,3-b]pyridine.

### Preparation 14″: N-(4-{[tert-Butyl(dimethyl)silyl]oxy}phenyl)-1-methyl-1H-benzimidazol-5-amine

The procedure is in accordance with the process of Preparation 1″, replacing the 4-bromo-1-methyl-1H-pyrazole used in Step B by 5-bromo-1-methyl-1H-benzimidazole.

### Preparation 15″: N⁴-(4-{[tert-Butyl(dimethyl)silyl]oxy}phenyl)-N²,N²-dimethyl-pyridine-2,4-diamine

The procedure is in accordance with the process of Preparation 1″, replacing the 4-bromo-1-methyl-1H-pyrazole used in Step B by 4-bromo-N,N-dimethylpyridin-2-amine.

### Preparation 16″: N-(4-{[tert-Butyl(dimethyl)silyl]oxy}phenyl)pyrazolo[1,5-a]-pyrimidin-6-amine

The procedure is in accordance with the process of Preparation 1″, replacing the 4-bromo-1-methyl-1H-pyrazole used in Step B by 6-bromopyrazolo[1,5-a]pyrimidine.

IR: ν —NH—: 3272 cm⁻¹; ν —C═N—: 1634 cm⁻¹; ν —C═C—: 1616 cm⁻¹

### Preparation 17″: N—[4-[tert-Butyl(dimethyl)silyl]oxyphenyl]-1-methyl-pyrazolo[3,4-b]pyridin-5-amine

The procedure is in accordance with the process of Preparation 1″, replacing the 4-bromo-1-methyl-1H-pyrazole used in Step B by 5-bromo-1-methyl-pyrazolo[3,4-b]pyridine (obtained in accordance with a protocol from the literature: WO 2006/052568 starting from 2-methyl-pyrazol-3-amine and 2-bromopropanedial).

### Preparation 18″: 4-({4-[(tert-Butyldimethylsilyl)oxy]phenyl}amino)-1,5-dimethyl-1H-pyrrole-2-carbonitrile

Step A: 4-Bromo-1,5-dimethyl-1H-pyrrole-2-carbonitrile

A solution of bromine (6.58 mL, 0.13 mol) in acetic acid (60 mL) is added dropwise, with the aid of a dropping funnel, to a solution of 1,5-dimethyl-1H-pyrrole-2-carbonitrile (15.0 g, 0.12 mol) in acetic acid (300 mL). The batch is stirred at ambient temperature for 24 hours. The reaction mixture is then poured into a beaker containing 300 mL of water. The solid formed is filtered off and rinsed with water. It is then dissolved in dichloromethane (300 mL) and the organic phase is washed with brine, dried over Na₂SO₄, filtered and concentrated in vacuo to yield the expected product in the form of a solid.

¹H NMR (CDCl₃) δ ppm: 2.25 (s, 3H), 3.67 (s, 3H), 6.74 (s, 1H)

Step B: 4-({4-[(tert-Butyldimethylsilyl)oxy]phenyl}amino)-1,5-dimethyl-1H-pyrrole-2-carbonitrile

A solution of the compound of the above Step (1.5 g, 7.53 mmol), 4-[(tert-butyldimethylsilyl)oxy]aniline (2.02 g, 9.04 mmol), sodium tert-butylate (1.45 g, 15.06 mmol) and 2-di-tert-butylphosphino-2′,4′,6′-triisopropylbiphenyl (0.13 g, 0.30 mmol) in toluene (20 mL) is purged with nitrogen. Tris(dibenzylideneacetone)dipalladium(0) (0.28 g, 0.30 mmol) is added, and then the reaction mixture is heated at 90° C. until the reaction is complete (monitored by TLC). Heating is stopped and the mixture is allowed to return to ambient temperature. Water (75 mL) is added and the mixture is extracted with ethyl acetate (3×75 mL). The combined organic phases are washed with brine and then concentrated. The crude product is absorbed onto silica gel and purified by chromatography over silica gel using ethyl acetate and heptane as eluants. The product thereby obtained is dissolved in heptane in the warm state and is allowed to precipitate, with stirring, at ambient temperature, and then at 0° C. The solid is filtered off and the operation is repeated on the filtrate to yield the expected compound in the form of a solid.

¹H NMR (400 MHz, CDCl₃) δ ppm: 0.15 (s, 6H), 0.97 (s, 9H), 2.13 (s, 3H), 3.66 (s, 3H), 4.68 (br. s, 1H), 6.49 (d, J=8.5 Hz, 2H), 6.64 (s, 1H), 6.66 (d, J=8.7 Hz, 2H)

¹³C NMR (100 MHz, CDCl₃) δ ppm: 4.34, 9.72, 18.30, 25.88, 32.94, 101.27, 114.37, 114.70, 116.41, 120.73, 124.52, 131.23, 141.54, 148.27

MS (ESI+): [M+H]⁺ measured: 342.3

### Preparation 19″: 4-[(4-{[tert-Butyl(dimethyl)silyl]oxy}phenyl)amino]-1-methyl-1H-pyrrole-2-carbonitrile

Step A: 1-Methyl-1H-pyrrole-2-carbonitrile

N,N-Dimethylformamide (3 mL) and 1,4-diazabicyclo[2.2.2]octane (0.49 g, 4.3 mmol) are added to a solution of pyrrole-2-carbonitrile (4 g, 43.4 mmol) in dimethyl carbonate (56 mL). The solution is stirred at 90° C. for 15 hours, and is then heated at 110° C. for 8 hours. The mixture is cooled to ambient temperature, and then ethyl acetate (80 mL) is added. The phases are separated and the organic phase is washed with water (2×80 mL) and 1N aqueous HCl solution (1×80 mL). The combined aqueous phases are extracted again with ethyl acetate (1×80 mL). The combined organic phases are washed with brine (1×80 mL), dried over MgSO₄, filtered and concentrated in vacuo to obtain the expected product in the form of a liquid.

¹H NMR (400 MHz, CDCl₃) δ ppm: 3.78 (m, 2H), 6.12-6.18 (m, 1H), 6.74-6.82 (m, 1H)

Step B: 4-Bromo-1-methyl-1H-pyrrole-2-carbonitrile

N-Bromosuccinimide (6.2 g, 34.9 mmol) is added to a solution of 1-methyl-1H-pyrrole-2-carbonitrile (3.7 g, 34.9 mmol) in N,N-dimethylformamide (150 mL). The solution is stirred for 15 hours at ambient temperature. Another amount of N-bromosuccinimide (2.0 g, 11 mmol) is added and the mixture is stirred for 3 hours. Silica (7 g) is then added and the suspension is then evaporated to dryness. The material pre-absorbed onto the silica is placed on a silica gel column and the product is purified by chromatography over silica gel using ethyl acetate and heptane as eluants to obtain the expected product in the form of a solid.

¹H NMR (400 MHz, CDCl₃) δ ppm: 3.77 (s, 3H), 6.75 (d, J=1.7 Hz, 1H), 6.80 (d, J=1.7 Hz, 1H)

Step C: 4-[(tert-Butyldimethylsilyl)oxy]phenyl}amino)-1-methyl-1H-pyrrole-2-carbonitrile

Nitrogen is bubbled through a solution of 4-bromo-1-methyl-1H-pyrrole-2-carbonitrile (2.82 g, 15.2 mmol) and 4-[(tert-butyldimethylsilyl)oxy]aniline (4.08 g, 18.3 mmol) in toluene (55 mL) for 5 minutes. Sodium tert-butylate (2.92 g, 30.4 mmol), tris(dibenzylideneacetone)dipalladium(0) (556 mg, 0.6 mmol) and 2-di-tert-butylphosphino-2′,4′,6′-triisopropylbiphenyl (255 mg, 0.6 mmol) are then added to the reaction mixture. The mixture is stirred for 1 hour at 80° C. under nitrogen. The suspension is then cooled to ambient temperature and filtered over Celite. The Celite cake is then rinsed with ethyl acetate. The filtrate is washed with water and then with brine. The organic phase is dried over MgSO₄, filtered and concentrated in vacuo. The product is purified twice by chromatography over silica gel using ethyl acetate and heptane as eluants, and then by trituration in heptane to obtain the expected product in the form of a solid.

¹H NMR (400 MHz, CDCl₃) δ ppm: 0.16 (s, 6H), 0.97 (s, 9H), 3.73 (s, 3H), 6.57 (d, J=1.9 Hz, 1H), 6.64-6.66 (m, 1H), 6.70 (s, 4H); NMR

¹³C NMR (100 MHz, CDCl₃) δ ppm: −4.48, 18.17, 25.72, 35.46, 103.01, 113.56, 113.69, 115.92, 119.55, 120.67, 129.04, 139.94, 148.85

MS (ESI+): [M+H]⁺ 328.25

### Preparation 20″: N—[4-[tert-Butyl(dimethyl)silyl]oxy-3-fluoro-phenyl]-1-methyl-1H-pyrazol-4-amine

The procedure is in accordance with the protocol of Preparation 1″, replacing the 4-aminophenol used in Step A by 2-fluoro-4-aminophenol.

¹H NMR (400 MHz, dmso-d6) δ ppm: 7.59 (bs, 1H), 7.39 (m, 1H), 7.24 (d, 1H), 6.74 (dd, 1H), 6.52 (dd, 1H), 6.42 (ddd, 1H), 3.76 (s, 3H), 0.92 (s, 9H), 0.1 (d, 6H)

### Preparation 21″: 2-({4-[(tert-Butyldimethylsilyl)oxy]phenyl}amino)pyridine-4-carbonitrile

A solution composed of 2-bromo-4-pyridinecarbonitrile (5.00 g, 36.1 mmol), 4-[(tert-butyldimethylsilyl)oxy]aniline (8.06 g, 36.1 mmol), sodium tert-butylate (4.50 g, 46.9 mmol) and 2-di-tert-butylphosphino-2′,4′,6′-triisopropylbiphenyl (0.458 g, 1.08 mmol) in toluene (50 mL) is purged with nitrogen. Tris(dibenzylideneacetone)dipalladium(0) (0.99 g, 1.08 mmol) is then added to the reaction mixture, and then the batch is heated at 50° C. for 1.5 hours. The mixture is then allowed to cool to ambient temperature. Water is added and the reaction mixture is extracted with ethyl acetate (3×20 mL). The combined organic phases are washed with brine, and then concentrated under reduced pressure. The crude product is absorbed onto silica gel and purified by chromatography over silica gel using ethyl acetate and heptane as eluants. The product obtained is dissolved in the warm state in heptane and precipitates, with stirring, at ambient temperature, and then at 0° C. After filtration, the expected compound is obtained in the form of a solid.

¹H NMR (400 MHz, CDCl₃) δ ppm: 0.22 (s, 6H), 1.00 (s, 9H), 6.61 (br. s, 1H), 6.81-6.84 (m, 2H), 6.84-6.89 (m, 2H), 7.12-7.17 (m, 2H), 8.26 (dd, J=5.1, 0.9 Hz, 1H)

¹³C NMR (100 MHz, CDCl₃) δ ppm: −4.29, 18.31, 25.78, 109.11, 114.73, 117.23, 121.17, 121.74, 124.93, 132.12, 149.79, 153.45, 158.00

MS (ESI+): [M+H]⁺ 326.19

### Preparation 22″: N—[4-[tert-Butyl(dimethyl)silyl]oxyphenyl]-1-tetrahydrofuran-3-yl-pyrazol-4-amine

4-[tert-Butyl(dimethyl)silyl]oxyaniline (0.92 g, 3.48 mmol) and 4-iodo-1-tetrahydrofuran-3-yl-pyrazole (0.78 g, 3.48 mmol) dissolved in anhydrous tetrahydrofuran (20 mL) are stirred for one hour at ambient temperature in the presence of sodium tert-butylate (1.7 mL, 2M solution in THF) and chloro(2-di-tert-butylphosphino-2′,4′,6′-triisopropyl-1,1′-biphenyl)[2-(2-aminoethyl)phenyl]palladium(II) (84 mg, 0.122 mmol). The reaction mixture is filtered over Celite and then evaporated to dryness. The residue is crystallised from a mixture of heptane/ethyl acetate, filtered and washed with heptane and then purified by chromatography over silica gel using dichloromethane and methanol as eluants to yield the expected product.

¹H NMR (500 MHz, dmso-d6, 300 K) δ ppm: 7.62 (d, 1H, pyrazole-H5), 7.29 (d, 1H, pyrazole-H3), 7.26 (s, 1H, NH), 6.68 (d, 2H, Ar—H), 6.64 (d, 2H, Ar—H), 4.93 (m, 1H, THF-3′H), 3.95 (m, 1H, THF-5′H), 3.94 (m, 1H, THF-2′H), 3.87 (m, 1H, THF-2′H), 3.80 (m, 1H, THF-5′H), 2.33 (m, 1H, THF-4′H), 2.27 (m, 1H, THF-4′H), 0.93 (s, 9H, ^{t}Bu), 0.12 (s, 6H, Me)

IR: ν C—H: 2857 cm⁻¹; ν aromatic: 1505 cm⁻¹; ν Si—C: 1249 cm⁻¹

### Preparation 23″: 6-({4-[(tert-Butyldimethylsilyl)oxy]phenyl}amino)pyridine-2-carbonitrile

4-Aminophenol (3.3 g, 30.2 mmol) is added to a solution of 6-chloropyridine-2-carbonitrile (3.5 g, 25.3 mmol) in 1-methyl-2-pyrrolidinone (70 mL). The reaction mixture is heated at 140-150° C. for 16 hours in a sealed flask. The batch is then cooled to ambient temperature.

Imidazole (3.4 g, 49.9 mmol) and tert-butyl(dimethyl)silyl chloride (7.6 g, 50.4 mmol) are subsequently added and the mixture is stirred for 16 hours at ambient temperature. The mixture is diluted with water (140 mL) and the product is extracted with AcOEt (4×50 mL). The organic phases are combined and washed with water (3×50 mL), and then brine (1×50 mL). The organic phase is then dried over MgSO₄, filtered and concentrated in vacuo. The crude product is purified by chromatography over silica gel using ethyl acetate and heptane as eluants to obtain the title product.

¹H NMR (400 MHz, CDCl₃) δ ppm: 0.20 (s, 6H), 0.99 (s, 9H), 6.74 (dd, J=7.6, 4.9 Hz, 1H), 6.81-6.88 (m, 3H), 7.36-7.42 (m, 2H), 7.74 (dd, J=7.6, 1.9 Hz, 1H), 8.34 (dd, J=4.9, 1.9 Hz, 1H)

¹³C NMR (100 MHz, CDCl₃) δ ppm: −4.38, 18.21, 25.73, 92.58, 113.50, 116.53, 120.30, 123.20, 131.97, 141.67, 152.42, 152.45, 156.51

MS (ESI): [M+H]⁺ 326.24

### Preparation 24″: 4-({4-[(tert-Butyldimethylsilyl)oxy]phenyl}amino)pyrimidine-2-carbonitrile

Step A: N—{4-[(tert-Butyldimethylsilyl)oxy]phenyl}-2-chloropyrimidin-4-amine

4-Aminophenol (8.8 g, 80.6 mmol) and triethylamine (18.6 mL, 133.4 mmol) are added to a solution of 2,4-dichloropyrimidine (10.0 g, 67.1 mmol) in ethanol (150 mL). The reaction mixture is heated at 150° C. for 14 hours in a sealed flask. The batch is then cooled to ambient temperature and the solvent is evaporated off in vacuo. Dichloromethane (200 mL) is added to the residue, and then imidazole (9.1 g, 133.7 mmol) and tert-butyl(dimethyl)silyl chloride (12.1 g, 80.3 mmol) are added. The mixture is stirred for 15 hours at ambient temperature. The reaction mixture is diluted with water (200 mL). The phases are separated and the organic phase is washed with brine (1×100 mL). It is then dried over MgSO₄, filtered and concentrated in vacuo. The residue is purified by chromatography over silica gel using ethyl acetate and heptane as eluants to obtain a solid. The latter is triturated in heptane, filtered off and rinsed with heptane to yield the expected compound in the form of a solid.

¹H NMR (400 MHz, dmso-d6) δ ppm: 0.22 (s, 6H), 0.99 (s, 9H), 6.42 (d, J=5.9 Hz, 1H), 6.81 (br. s, 1H), 6.85-6.90 (m, 1H), 7.13 (d, J=8.7 Hz, 2H), 8.07 (d, J=5.9 Hz, 2H)

Step B: 4-({4-[(tert-Butyldimethylsilyl)oxy]phenyl}amino)pyrimidine-2-carbonitrile

Anhydrous N,N-dimethylformamide (10 mL) is placed under nitrogen in a flask and then the compound of Step A (670 mg, 2.0 mmol) is added. Zinc cyanide (468 mg, 4.0 mmol) and tetrakis(triphenylphosphine)palladium(0) (404 mg, 0.3 mmol) are subsequently added. Nitrogen is bubbled through the solution for 5 minutes and then the reaction mixture is stirred at 120° C. for 2 hours under a nitrogen atmosphere. The reaction, monitored by LC-MS, is complete. The mixture is cooled to ambient temperature, and then water (15 mL) is added thereto. The product is extracted with AcOEt (3×25 mL). The organic phases are combined and washed with water (4×25 mL), and then with brine (1×25 mL). The organic phase is dried over MgSO₄, filtered and concentrated in vacuo. The residue is purified by chromatography over silica gel using ethyl acetate and heptane as eluants to obtain the expected compound in the form of a solid.

¹H NMR (400 MHz, CDCL₃) δ ppm: 0.22 (s, 6H), 0.99 (s, 9H), 6.63 (d, J=6.1 Hz, 1H), 6.86-6.92 (m, 2H), 7.03 (br. s, 1H), 7.17 (d, J=8.5 Hz, 2H), 8.22 (d, J=6.1 Hz, 1H)

¹³C NMR (100 MHz, CDCL₃) δ ppm: −4.51, 18.10, 25.55, 106.32, 115.92, 121.00, 125.22, 129.73, 144.55, 154.16, 156.07, 161.56

### Preparation 25″: N—{4-[(tert-Butyldimethylsilyl)oxy]phenyl}-1-trideuteriomethyl-1H-pyrazol-4-amine

Step A: 4-Bromo-1-trideuteriomethyl-1H-pyrazole

4-Bromo-1H-pyrazole (9.05 g, 61.6 mmol) is added in portions to a suspension of NaH (60% in oil) (2.83 g, 70.8 mmol) in tetrahydrofuran (90 mL) cooled in an ice bath. After having taken away the ice bath, the solution is stirred at ambient temperature for 0.5 hours. It is again cooled in an ice bath and iodomethane-d₃ (5.0 mL, 80.3 mmol) is added. The solution is stirred at ambient temperature for 19 hours. The suspension is then concentrated. The evaporation residue is triturated with MTBE (90 mL) and filtered. The filtrate is concentrated in vacuo to obtain the expected compound in the form of an oil.

¹H NMR (400 MHz, CDCL₃) δ ppm: 7.37 (s, 1H), 7.43 (s, 1H)

Step B: N—[4-[(tert-Butyldimethylsilyl)oxy]phenyl]-1-trideuteriomethyl-1H-pyrazol-4-amine

4-Bromo-1-trideuteriomethyl-1H-pyrazole (9.6 g, 58.5 mmol), 4-[(tert-butyldimethyl-silyl)oxy]aniline (14.4 g, 64.6 mmol) and toluene (150 mL) are added to a 500-ml three-necked flask. The solution is degassed with nitrogen for 15 minutes, and then sodium tert-butylate (11.4 g, 0.12 mol), 2-di-tert-butylphosphino-2′,4′,6′-triisopropylbiphenyl (0.77 g, 1.81 mmol) and tris(dibenzylideneacetone)dipalladium(0) (1.64 g, 1.79 mmol) are successively added. The suspension is heated at 85° C. for 1.5 hours. The reaction mixture is then cooled to ambient temperature and water (270 mL) is added. The mixture is stirred for 30 minutes. Celite (30 g) is then added and the suspension is filtered on a bed of Celite. The phases of the filtrate are separated and the aqueous phase is extracted with ethyl acetate (3×200 mL). The combined organic phases are dried over Na₂SO₄ and filtered. Silica (36 g) is added to the filtrate and the batch is evaporated to dryness. The product is purified by chromatography over silica gel using ethyl acetate and heptane as eluants. The product obtained is recrystallised from heptane (80 mL) to obtain the expected compound.

¹H NMR (400 MHz, CDCl₃) δ ppm: 0.16 (s, 6H), 0.97 (s, 9H), 4.92 (s, 1H), 6.61-6.73 (m, 4H), 7.25 (s, 1H), 7.36 (s, 1H)

¹³C NMR (100 MHz, CDCl₃) δ ppm: −4.37, 18.28, 25.86, 38.67 (sept., ¹J_{C-D}=21.0 Hz), 115.12, 120.73, 123.76, 126.52, 134.74, 141.07, 148.43 MS (ESI): [M+H]⁺ 307.08

### Preparation 26″: N—[4-[tert-Butyl(dimethyl)silyl]oxyphenyl]-1-(oxetan-3-yl)-1H-pyrazol-4-amine

### Step A: 4-Bromo-1-(oxetan-3-yl)-1H-pyrazole

4-Bromo-1H-pyrazole (1.53 g, 10.7 mmol) is dissolved in anhydrous dimethylformamide (15 mL). 3-Bromooxetane (2.0 g, 14.6 mmol) and caesium carbonate (4.7 g, 14 mmol) are successively added thereto. The reaction mixture is heated for 8 hours at 130° C. in a sealed flask. At the end of the reaction, the solvent is evaporated off in vacuo and the residue is purified by chromatography over silica gel using dichloromethane containing diethylamine and methanol as eluants to yield the expected compound.

Step B: N—[4-[tert-Butyl(dimethyl)silyl]oxyphenyl]-1-(oxetan-3-yl)-1H-pyrazol-4-amine

4-[tert-Butyl(dimethyl)silyl]oxyaniline (1.5 g, 7.2 mmol) and 4-bromo-1-(oxetan-3-yl)-pyrazole (1.6 g, 7.2 mmol) dissolved in anhydrous tetrahydrofuran (25 mL) are stirred for 3 hours at ambient temperature in the presence of sodium tert-butylate (3.7 mL, 2M solution in THF) and chloro(2-di-tert-butylphosphino-2′,4′,6′-triisopropyl-1,1′-biphenyl)[2-(2-aminoethyl)phenyl]palladium(II) (101 mg, 0.145 mmol). The reaction mixture is filtered over Celite and then evaporated to dryness. The residue is purified by chromatography over silica gel using dichloromethane containing diethylamine and ethyl acetate as eluants to yield the expected product.

¹H NMR (500 MHz, dmso-d6, 300 K) δ ppm: 7.76 (s, 1H, pyrazole-5′H), 7.40 (s, 1H, pyrazole-3′H), 7.34 (br s, 1H, NH), 6.70 (d, 2H, Ar—H), 6.65 (d, 2H, Ar—H), 5.49 (m, 1H, oxetane), 4.89 (d, 4H, oxetane), 0.93 (s, 9H, ^{t}Bu), 0.13 (s, 6H, Me)

IR: ν: C—H: 2955 cm⁻¹; aromatic: 1505 cm⁻¹; Si—C: 1237 cm⁻¹

### Preparation 27″: Mixture of N-(4-{[tert-butyl(dimethyl)silyl]oxy}phenyl)-1,5-dimethyl-1H-pyrazol-4-amine and N-(4-{[tert-butyl(dimethyl)silyl]oxy}phenyl)-1,3-dimethyl-1H-pyrazol-4-amine

Step A: Mixture of 4-bromo-1,5-dimethyl-1H-pyrazole and 4-bromo-1,3-dimethyl-1H-pyrazole

To a suspension of NaH 60% in oil (0.3 g; 7.45 mmol) in tetrahydrofuran (150 mL) there is added, at 10° C., 4-bromo-3-methyl-1H-pyrazole dissolved in 15 mL of tetrahydrofuran dropwise, over 15 minutes. After stirring for 40 minutes at ambient temperature, iodomethane (0.45 mL; 7.45 mmol) is added dropwise, and then the reaction mixture is stirred overnight. After adding water, the reaction mixture is evaporated and taken up in dichloromethane. The organic phase is separated off and dried over MgSO₄, filtered and concentrated to dryness. The residue is purified by chromatography over silica gel to yield a mixture of the title compounds (4-bromo-1,3-dimethyl-pyrazole and 4-bromo-1,5-dimethyl-pyrazole respectively in a ratio of 4:6).

### 4-bromo-1,5-dimethyl-1H-pyrazole

¹H NMR (500 MHz, dmso-d6) δ ppm: 7.41 (s, 1H), 3.76 (s, 3H), 2.22 (s, 3H)

### 4-bromo-1,3-dimethyl-1H-pyrazole

¹H NMR (500 MHz, dmso-d6) δ ppm: 7.81 (s, 1H), 3.74 (s, 3H), 2.09 (s, 3H)

Step B: Mixture of N-(4-{[tert-butyl(dimethyl)silyl]oxy}phenyl)-1,5-dimethyl-1H-pyrazol-4-amine and N-(4-{[tert-butyl(dimethyl)silyl]oxy}phenyl)-1,3-dimethyl-1H-pyrazol-4-amine

The procedure is in accordance with Step B of Preparation 1″, replacing the 4-bromo-1-methyl-1H-pyrazole by the mixture of isomers from Step A. A mixture of isomers in a ratio of 4:6 is obtained (respectively N-(4-{[tert-butyl(dimethyl)silyl]oxy}phenyl)-1,5-dimethyl-1H-pyrazol-4-amine and N-(4-{[tert-butyl(dimethyl)silyl]oxy}phenyl)-1,3-dimethyl-1H-pyrazol-4-amine).

### Preparation 28″: N—[4-[tert-Butyl(dimethyl)silyl]oxyphenyl]-1-cyclopropyl-1H-pyrazol-4-amine

Step A: 4-Bromo-1-cyclopropyl-1H-pyrazole

4-Bromo-1H-pyrazole (1.76 g, 12 mmol) is dissolved in anhydrous dimethylformamide (15 mL). Cyclopropyl bromide (2.9 mL, 36 mmol) and caesium carbonate (7.8 g, 24 mmol) are successively added thereto. The reaction mixture is heated for 15 hours at 160° C. in a sealed flask. At the end of the reaction, the solvent is evaporated off in vacuo and the residue is purified by chromatography over silica gel using heptane and dichloromethane as eluants to yield the expected compound.

Step B: N—[4-[tert-Butyl(dimethyl)silyl]oxyphenyl]-1-cyclopropyl-1H-pyrazol-4-amine

4-[tert-Butyl(dimethyl)silyl]oxyaniline (1.64 g, 7.3 mmol) and 4-bromo-1-cyclopropyl-1H-pyrazole (1.4 g, 7.3 mmol) dissolved in anhydrous tetrahydrofuran (30 mL) are stirred for 3 hours at ambient temperature in the presence of sodium tert-butylate (3.7 mL, 2M solution in THF) and chloro(2-di-tert-butylphosphino-2′,4′,6′-triisopropyl-1,1′-biphenyl)[2-(2-aminoethyl)phenyl]palladium(II) (101 mg, 0.146 mmol). The reaction mixture is filtered over Celite and then evaporated to dryness. The residue is purified by chromatography over silica gel using heptane and ethyl acetate as eluants to yield the expected product.

¹H NMR (500 MHz, dmso-d6, 300 K) δ ppm: 7.61 (s, 1H, pyrazole-5′H), 7.23 (s, 1H, pyrazole-3′H), 7.22 (br s, 1H, NH), 6.65 (d, 2H, Ar—H), 6.63 (d, 2H, Ar—H), 3.64 (m, 1H, cyclopropyl-H), 1.00-0.91 (m, 4H, cyclopropyl), 0.93 (s, 9H, ^{t}Bu), 0.12 (s, 6H, Me)

IR: ν: C—H: 2930 cm⁻¹; aromatic: 1504 cm⁻¹; Si—C: 1237 cm⁻¹

### High-Resolution Mass Spectrometry (ESI+):

C₁₈H₂₇N₃OSi Empirical formula:

[M+H]⁺ calculated: 330.2003

[M+H]⁺ measured: 330.1989

### Preparation 29″: 1,5-Dimethyl-4-(phenylamino)-1H-pyrrole-2-carbonitrile

The procedure is in accordance with the protocol of Preparation 1″, replacing the 4-{[tert-butyl(dimethyl)silyl]oxy}aniline used in Step B by aniline.

¹H NMR (400 MHz, dmso-d6) δ ppm: 7.19 (s, 1H), 7.05 (t, 2H), 6.79 (s, 1H), 6.6 (m, 3H), 3.61 (s, 3H), 2.1 (s, 3H)

### Preparation 30″: 1-Methyl-N-phenyl-1H-pyrrolo[2,3-b]pyridin-6-amine

The procedure is in accordance with the protocol of Step B of Preparation 1″ using aniline and 5-bromo-1-methyl-1H-pyrrolo[2,3-b]pyridine (obtained in accordance with a protocol from the literature: Heterocycles, 60(4), 865, 2003).

¹H NMR (400 MHz, dmso-d6) δ ppm: 8.1 (d, 1H), 7.9 (s, 1H), 7.7 (d, 1H), 7.45 (d, 1H), 7.17 (t, 2H), 6.9 (d, 2H), 6.7 (t, 1H), 6.38 (d, 1H), 3.8 (s, 3H)

### Preparation 31″: N-(4-{[tert-Butyl(dimethyl)silyl]oxy}phenyl)-1-trideuteriomethyl-1H-pyrrolo[2,3-b]pyridin-5-amine

The procedure is in accordance with the process of Preparation 1″, replacing the 4-bromo-1-methyl-1H-pyrazole used in Step B by 5-bromo-1-(trideuteriomethyl)-1H-pyrrolo[2,3-b]pyridine (obtained in accordance with a protocol from the literature Heterocycles, 60(4), 865, 2003, replacing the methyl iodide by trideuterated methyl iodide).

¹H NMR (400 MHz, dmso-d6) δ ppm: 8.05 (d, 1H), 7.6 (m+d, 2H), 7.4 (d, 1H), 6.85/6.7 (2d, 4H), 6.3 (d, 1H), 0.95 (s, 9H), 0.15 (s, 6H)

### Preparation 32″: 4-({4-[(tert-Butyldimethylsilyl)oxy]phenyl}amino)-1-trideuterio-methyl-1H-pyrrole-2-carbonitrile

Step A: 1-Trideuteriomethyl-1H-pyrrole-2-carbonitrile

NaH (60% in oil) (2.61 g, 65.2 mmol) is suspended in tetrahydrofuran (110 mL) at 0° C. 1H-Pyrrole-2-carbonitrile (5 g, 54.3 mmol) is added dropwise over 10 minutes. The reaction mixture is then warmed up to ambient temperature over 30 minutes. It is again cooled to 0° C., and then iodomethane-d₃ (10.23 g, 70.6 mmol) is added. The reaction mixture is stirred for 16 hours under nitrogen at ambient temperature. The reaction mixture is then evaporated under reduced pressure, and then ethyl acetate (200 mL) and water (200 mL) are added. The phases are separated, and the aqueous phase is extracted with ethyl acetate (2×200 mL). The combined organic phases are washed with brine (1×80 mL), dried over MgSO₄, filtered and concentrated in vacuo. The residue is purified by chromatography over silica gel using ethyl acetate and heptane as eluants. The fractions are combined and evaporated in vacuo to obtain the expected compound.

¹H NMR (400 MHz, CDCl₃) δ ppm: 6.13 (dd, J=2.7, 3.9 Hz, 1H), 6.75 (dd, J=1.5, 4.1 Hz, 1H), 6.79 (dd, J=1.7, 2.6 Hz, 1H)

Step B: 4-Bromo-1-trideuteriomethyl-1H-pyrrole-2-carbonitrile

N-Bromosuccinimide (6.68 g, 37.5 mmol) is added to a solution of the compound of the above Step (4.09 g, 37.5 mmol) in N,N-dimethylformamide (188 mL). The solution is stirred for 16 hours at ambient temperature. The residue is purified by chromatography using ethyl acetate and heptane as eluants to obtain the expected compound in the form of a solid.

¹H NMR (400 MHz, CDCl₃) δ ppm: 6.75 (d, J=1.7 Hz, 1H), 6.80 (d, J=1.7 Hz, 1H)

Step C: 4-({4-[(tert-Butyldimethylsilyl)oxy]phenyl}amino)-1-trideuteriomethyl-1H-pyrrole-2-carbonitrile

Nitrogen is bubbled through a solution composed of the compound obtained in the above Step (5.85 g, 31.1 mmol), 4-[(tert-butyldimethylsilyl)oxy]aniline (8 g, 35.8 mmol), sodium tert-butylate (3.88 g, 40.4 mmol) and 2-di-tert-butylphosphino-2′,4′,6′-triisopropylbiphenyl (662 mg, 1.56 mmol) in toluene (260 mL) for 5 minutes. Tris(dibenzylideneacetone)-dipalladium(0) (1.43 g, 1.56 mmol) is then added. The mixture is stirred for 1 hour at 70° C. under nitrogen. The suspension is then cooled to ambient temperature, diluted with ethyl acetate and filtered over Celite. The Celite cake is then rinsed with ethyl acetate. The filtrate is washed with water (3 times), and then brine (once). The organic phase is dried over Na₂SO₄, filtered and concentrated in vacuo. The product is purified twice by chromatography over silica gel using ethyl acetate and heptane as eluants, and then by reverse-phase chromatography using methanol and water as eluants to obtain the expected compound in the form of a powder.

¹H NMR (400 MHz, CDCl₃) δ ppm: 0.17 (s, 6H), 0.98 (s, 9H), 4.96 (br. s, 1H), 6.57 (d, J=1.9 Hz, 1H), 6.64 (d, J=1.8 Hz, 1H), 6.70 (br. s, 4H)

¹³C NMR (100 MHz, CDCl₃) δ ppm: −4.38, 18.26, 25.83, 34.83, 102.96, 113.69, 113.71, 115.95, 119.58, 120.75, 129.14, 140.10, 148.84 MS (ESI): [M+H]⁺ 331.09

### Preparation 33″: 4-({4-[(tert-Butyldimethylsilyl)oxy]phenyl}amino)-1-trideuterio-methyl-5-methyl-1H-pyrrole-2-carbonitrile

Step A: 5-Methyl-1H-pyrrole-2-carbonitrile

The compound is prepared in accordance with the protocol described in Heterocycles 2011, 82, 1503.

Step B: 1-Trideuteriomethyl-5-methyl-1H-pyrrole-2-carbonitrile

A solution of 5-methyl-1H-pyrrole-2-carbonitrile (0.30 g, 2.82 mmol) in N,N-dimethyl-formamide (5 mL) is cooled to 0° C. NaH (60% in oil) (0.118 g, 2.96 mmol) is added in portions, and the reaction mixture is stirred at 0° C. for 30 minutes. Iodomethane-d₃ (4.15 mL, 67.2 mmol) is added in a single portion, and the reaction mixture is stirred at ambient temperature until the reaction is complete. It is then diluted with water (30 mL) and ethyl acetate (15 mL). The phases are separated and the aqueous phase is extracted a second time with ethyl acetate (15 mL). The combined organic phases are washed with water (1×50 mL), and then brine, and dried over Na₂SO₄. After filtration and concentration under reduced pressure, the residue is purified by chromatography over silica gel using ethyl acetate and heptane as eluants to yield the expected compound in the form of a solid.

¹H NMR (400 MHz, CDCl₃) δ ppm: 2.25 (s, 3H), 5.91 (dd, J=3.9, 0.6 Hz, 1H), 6.69 (d, J=3.9 Hz, 1H)

Step C: 4-Bromo-1-trideuteriomethyl-5-methyl-1H-pyrrole-2-carbonitrile

A solution of bromine (0.133 mL, 2.60 mmol) in acetic acid (1.5 mL) is added dropwise to a solution of the compound obtained in the above Step (0.305 g, 2.48 mmol) in acetic acid (5.5 mL) previously cooled to 0° C. The reaction mixture is stirred and gradually warmed up to ambient temperature over a period of 20 hours. The reaction mixture is poured into water (50 mL) and the mixture is extracted with dichloromethane (2×50 mL). The organic phase is dried over Na₂SO₄, filtered and concentrated under reduced pressure to yield the expected compound in the form of a solid.

¹H NMR (400 MHz, CDCl₃) δ ppm: 2.24 (s, 3H), 6.73 (s, 1H)

Step D: 4-({4-[(tert-Butyldimethylsilyl)oxy]phenyl}amino)-1-trideuteriomethyl-5-methyl-1H-pyrrole-2-carbonitrile

A solution composed of the compound obtained in the above Step (7.00 g, 34.6 mmol), 4-[(tert-butyldimethylsilyl)oxy]aniline (8.90 g, 39.8 mmol), sodium tert-butylate (4.33 g, 45.0 mmol) and 2-di-tert-butylphosphino-2′,4′,6′-triisopropylbiphenyl (0.441 g, 1.04 mmol) in toluene (70 mL) is purged with nitrogen. Tris(dibenzylideneacetone)dipalladium(0) (0.951 g, 1.04 mmol) is added, and then the reaction mixture is heated at 65° C. until the reaction, monitored by TLC, is complete. Heating is stopped and the mixture is cooled to ambient temperature. Water (200 mL) is added and the mixture is extracted with ethyl acetate (3 times). The combined organic phases are washed with brine, and then concentrated. The crude product is purified by chromatography over silica gel using ethyl acetate and heptane as eluants. The product obtained is dissolved in heptane in the warm state; it is allowed to precipitate out at ambient temperature and then at 0° C. to yield the expected product in the form of crystals.

¹H NMR (400 MHz, CDCl₃) δ ppm: 0.15 (s, 6H), 0.96 (s, 9H), 2.12 (s, 3H), 4.66 (br. s, 1H), 6.46-6.51 (m, 2H), 6.64 (s, 1H), 6.64-6.69 (m, 2H)

¹³C NMR (100 MHz, CDCl₃) δ ppm: −4.37, 9.64, 18.26, 25.84, 31.62-32.87 (m), 101.14, 114.35, 114.66, 116.33, 120.68, 124.51, 131.17, 141.53, 148.18

MS (ESI): [M+H]⁺ 345.13

### Preparation 34″: 5-[(4-{[tert-Butyl(dimethyl)silyl]oxy}phenyl)amino]-1-methyl-1H-pyrazole-3-carbonitrile

Step A: 5-Amino-1-methyl-1H-pyrazole-3-carbonitrile

To a suspension of 1-methyl-5-nitro-1H-pyrazole-3-carbonitrile (2 g; 13.1 mmol) in a 5 mixture of water (14 mL) and ethanol (120 mL) there are added HCl 37% (170 μL) and iron filings (5.1 g; 91 mmol). The reaction mixture is heated for 5 hours at 50° C. After cooling to ambient temperature, the reaction mixture is filtered. The filtrate is concentrated to dryness and then purified by chromatography over silica gel using dichloromethane and ethyl acetate as eluants to yield the expected compound in the form of a solid.

¹H NMR (400 MHz, dmso-d6) δ ppm: 5.8 (s, 1H), 5.7 (m, 2H), 3.6 (s, 3H)

Step B: 5-[(4-{[tert-Butyl(dimethyl)silyl]oxy}phenyl)amino]-1-methyl-1H-pyrazole-3-carbonitrile

The procedure is in accordance with the protocol of Step B of Preparation 1″, replacing the 4-bromo-1-methyl-1H-pyrazole by (4-bromophenoxy)-(tert-butyl)dimethyl-silane and the 4-{[tert-butyl(dimethyl)silyl]oxy}aniline by the compound from Step A.

¹H NMR (400 MHz, dmso-d6) δ ppm: 8.15 (s, 1H), 6.9 (d, 2H), 6.75 (d, 2H), 6.45 (s, 1H), 3.75 (s, 3H), 0.95 (s, 9H), 0.15 (s, 6H)

### Preparation 35″: 4-[(4-{[tert-Butyl(dimethyl)silyl]oxy}phenyl)amino]-5-methyl-1-[2-(morpholin-4-yl)ethyl]-1H-pyrrole-2-carbonitrile

The procedure is in accordance with the process of Preparation 33″, replacing the iodomethane-d₃ in Step B by 2-(chloroethyl)morpholine hydrochloride.

¹H NMR (400 MHz, dmso-d6) δ ppm: 6.85 (s, 1H), 6.75 (s, 1H), 6.6 (d, 2H), 6.5 (d, 2H), 4.1 (t, 2H), 3.55 (t, 4H), 2.6 (t, 2H), 2.4 (t, 4H), 2.1 (s, 3H), 0.9 (s, 9H), 0.1 (s, 6H)

### Preparation 36″: N-(4-{[tert-Butyl(dimethyl)silyl]oxy}phenyl)-2-[2-(morpholin-4-yl)ethoxy]pyrimidin-5-amine

### Step A: 4-[2-(5-Bromopyrimidin-2-yl)oxyethyl]morpholine

NaH (1.0 g, 25.0 mmol, 60% in oil) suspended in anhydrous tetrahydrofuran is cooled at 0° C. in an ice bath under argon, and then 2-morpholinoethanol (2.7 g, 20.7 mmol) is added dropwise. The ice bath is withdrawn and the suspension is stirred for 1 hour at ambient temperature. 5-Bromo-2-chloro-pyrimidine (4.0 g, 20.7 mmol) is then added at ambient temperature and the reaction mixture is stirred for 16 hours at ambient temperature. Saturated aqueous ammonium chloride solution (10 mL) and water (10 mL) are added to the reaction mixture; the pH is adjusted to 9 by adding saturated aqueous NaHCO₃ solution. The resulting solution is extracted 3 times with ethyl acetate, the organic phase is then washed with brine, dried over MgSO₄, and then evaporated to dryness. The expected compound precipitates out by means of the addition of petroleum ether.

¹H NMR (400 MHz, dmso-d6) δ ppm: 8.75 (s, 2H), 4.4 (t, 2H), 3.55 (t, 4H), 2.7 (t, 2H), 2.45 (t, 4H)

IR (ATR) cm⁻¹ 1562 ν>C═C< and C═N, 787 ν —C—H Ar

Step B: N-(4-{[tert-Butyl(dimethyl)silyl]oxy}phenyl)-2-[2-(morpholin-4-yl)ethoxy]-pyrimidin-5-amine

The procedure is in accordance with the protocol of Step B of Preparation 1″, replacing the 4-bromo-1-methyl-1H-pyrazole by the compound from Step A.

¹H NMR (400 MHz, dmso-d6) δ ppm: 8.3 (s, 2H), 7.8 (s, 1H), 6.9/6.75 (2d, 4H), 4.35 (t, 2H), 3.55 (t, 4H), 2.7 (t, 2H), 2.45 (t, 4H), 0.95 (s, 9H), 0.15 (s, 6H)

IR (ATR) cm⁻¹ 3300 ν —NH, 1506 ν —NH, 837 ν —Si-Me, 837 and 778 ν —CH Ar

### Preparation 37″: 4-({4-[(tert-Butyldimethylsilyl)oxy]phenyl}amino)-1,3-dimethyl-1H-pyrrole-2-carbonitrile

Step A: 4-Methyl-N-(prop-2-en-1-yl)benzene-1-sulphonamide

Allylamine (10.6 mL, 0.14 mol) is added over a period of 4 minutes to a solution of tosyl chloride (25.1 g, 0.13 mol) in dichloromethane (250 mL) cooled in an ice bath. Triethylamine (24 mL, 0.18 mol) is added and then the solution is stirred at ambient temperature for 1.25 hours. Aqueous 3N HCl solution (60 mL) is added and the phases are then separated. The organic phase is washed with another amount of aqueous 3N HCl solution (60 mL) and then with 5% aqueous sodium bicarbonate solution (60 mL). The organic phase is dried over Na₂SO₄, filtered and concentrated to obtain the expected compound in the form of a solid.

¹H NMR (400 MHz, CDCl₃) δ ppm: 2.43 (s, 3H), 3.54-3.63 (m, 2H), 4.50 (1s, 1H), 5.05-5.22 (m, 2H), 5.66-5.79 (m, 1H), 7.31 (d, J=8.1 Hz, 2H), 7.76 (d, J=8.1 Hz, 2H)

Step B: 4-Methyl-N-(2-methylprop-2-en-1-yl)-N-(prop-2-en-1-yl)benzene-1-sulphonamide

3-Chloro-2-methylpropene (20 mL, 0.20 mol) is added over a period of 5 minutes to a suspension of 4-methyl-N-(prop-2-en-1-yl)benzene-1-sulphonamide (27.9 g, 0.13 mol) and potassium carbonate (28.1 g, 0.20 mol) in N,N-dimethylformamide (200 mL) cooled in an ice bath. After 20 minutes, the suspension is stirred at ambient temperature for 18 hours. The suspension is concentrated to dryness. The residue is taken up in ethyl acetate (250 mL) and water (110 mL). The aqueous phase is extracted with ethyl acetate (2×50 mL). The combined organic phases are washed successively with aqueous 3N HCl solution (50 mL), water (3×50 mL), aqueous 5% potassium bicarbonate solution (50 mL), and finally brine (50 mL). The organic phase is dried over Na₂SO₄, filtered and concentrated to obtain the expected compound in the form of an oil.

¹H NMR (400 MHz, CDCl₃) δ ppm: 1.69 (s, 3H), 2.43 (s, 3H), 3.70 (s, 2H), 3.75-3.79 (m, 2H), 4.87 (d, J=25.1 Hz, 1H), 5.04-5.09 (m, 1H), 5.09-5.12 (m, 1H), 5.45-5.59 (m, 1H), 7.29 (d, J=8.1 Hz, 2H), 7.71 (d, J=8.1 Hz, 2H)

Step C: 3-Methyl-1-(4-methylbenzenesulphonyl)-1H-pyrrole

A solution of the compound obtained in Step B (15.2 g, 57.3 mmol) in toluene (550 mL) is heated at 80° C. in the presence of (1,3-bis(2,4,6-trimethylphenyl)-2-imidazolidinylidene)-dichloro(phenylmethylene)(tricyclohexylphosphine)ruthenium (Grubbs' catalyst, 2nd generation) (150 mg, 0.18 mmol) for 1 hour. 2,3-Dichloro-5,6-dicyano-p-benzoquinone (16.1 g, 70.9 mmol) is then added in one portion, and the solution is heated at 80° C. for 24 hours. The solution is filtered over Celite, and the filtrate is concentrated in vacuo. The product is purified by chromatography over silica gel using ethyl acetate and heptane as eluants to obtain the expected compound in the form of a solid.

¹H NMR (400 MHz, CDCl₃) δ ppm: 2.02 (d, J=1.0 Hz, 3H), 2.39 (s, 3H), 6.09-6.13 (m, 1H), 6.85-6.90 (m, 1H), 7.03-7.07 (m, 1H), 7.27 (d, J=8.4 Hz, 2H), 7.72 (d, J=8.4 Hz, 2H)

Step D: 3-Methyl-1-(4-methylbenzenesulphonyl)-1H-pyrrole-2-carbonitrile

Aluminium chloride (19.4 g, 0.15 mol) is added all at once to a solution of 3-methyl-1-(4-methylbenzenesulphonyl)-1H-pyrrole (13.0 g, 55.3 mmol) in 1,2-dichloroethane (230 mL) at ambient temperature. After stirring for 20 minutes, cyanogen bromide (11.11 g, 0.10 mol) is added in portions over a period of 20 minutes. After 4.5 hours, an additional amount of cyanogen bromide (1.94 g, 18.3 mmol) is added. After stirring for 17 hours at ambient temperature, the reaction mixture is poured slowly into a mixture of dichloromethane (300 mL) and water (600 mL) cooled to 0° C. The resulting mixture is stirred for 1 hour. Subsequently the phases are separated and the aqueous phase is extracted with dichloromethane (3×150 mL). The combined organic phases are washed with water (2×150 mL) and brine (150 mL). The organic phase is dried over Na₂SO₄, filtered and concentrated in vacuo to obtain the expected compound in the form of a solid.

¹H NMR (400 MHz, CDCl₃) δ ppm: 2.18 (s, 3H), 2.43 (s, 3H), 6.17 (d, J=3.2 Hz, 1H), 7.33-7.39 (m, 3H), 7.90 (d, J=8.5 Hz, 2H)

Step E: 4-Bromo-3-methyl-1-(4-methylbenzenesulphonyl)-1H-pyrrole-2-carbonitrile

N-Bromosuccinimide (12.0 g, 67.4 mmol) is added all at once to a suspension of 3-methyl-1-(4-methylbenzenesulphonyl)-1H-pyrrole-2-carbonitrile (14.45 g, 55.6 mmol) in N,N-dimethylformamide (60 mL) at ambient temperature. The mixture is stirred at ambient temperature for 29 hours and is then cooled in an ice bath. Saturated aqueous sodium bisulphite solution (90 mL), water (90 mL) and ethyl acetate (250 mL) are then added. The phases are separated, and then the aqueous phase is extracted with ethyl acetate (2×70 mL). The combined organic phases are washed with 5% aqueous potassium bicarbonate solution (90 mL), water (3×90 mL), and then brine (3×90 mL). The organic phase is dried over Na₂SO₄, filtered and concentrated in vacuo. The crude product is purified by chromatography over silica gel using toluene and heptane as eluants to obtain the expected compound in the form of a solid.

¹H NMR (400 MHz, CDCl₃) δ ppm: 2.13 (s, 3H), 2.45 (s, 3H), 7.38 (d, J=8.4 Hz, 2H), 7.43 (s, 1H), 7.92 (d, J=8.4 Hz, 2H)

Step F: 4-Bromo-1,3-dimethyl-1H-pyrrole-2-carbonitrile

Potassium hydroxide (3.65 g, 65.1 mmol) is added all at once to a suspension of 4-bromo-3-methyl-1-(4-methylbenzenesulphonyl)-1H-pyrrole-2-carbonitrile (4.66 g, 13.7 mmol) in methanol (95 mL) cooled using an ice bath. After 15 minutes, the suspension is stirred at ambient temperature for 17 hours. The methanol is evaporated to dryness. The evaporation residue is taken up in MTBE (25 mL) and washed with water (25 mL). The aqueous phase is extracted with MTBE (2×25 mL). The combined organic phases are dried over Na₂SO₄, filtered and concentrated in vacuo. The residue obtained is dissolved in tetrahydrofuran (50 mL) and the solution is cooled using an ice bath. NaH (60% in oil) (1.02 g, 25.5 mmol) is added. After 10 minutes, iodomethane (2.4 mL, 38.6 mmol) is also added. The mixture is stirred at ambient temperature for 1.5 hours. The tetrahydrofuran is evaporated to dryness. The residue is taken up in dichloromethane and washed with water. The aqueous phase is extracted with dichloromethane. The combined organic phases are dried over Na₂SO₄, filtered and concentrated in vacuo to obtain the expected compound in the form of a solid.

¹H NMR (400 MHz, CDCl₃) δ ppm: 2.16 (s, 3H), 3.71 (s, 3H), 6.74 (s, 1H)

Step G: 4-({4-[(tert-Butyldimethylsilyl)oxy]phenyl}amino)-1,3-dimethyl-1H-pyrrole-2-carbonitrile

4-Bromo-1,3-dimethyl-1H-pyrrole-2-carbonitrile (2.03 g, 10.2 mmol) and 4-[(tert-butyldimethylsilyl)oxy]aniline (3.41 g, 15.3 mmol) are dissolved in toluene (40 mL). The solution is degassed with nitrogen for 10 minutes. Sodium tert-butylate (1.18 g, 12.2 mmol), 2-di-tert-butylphosphino-2′,4′,6′-triisopropylbiphenyl (0.17 g, 0.41 mmol) and tris(dibenzylideneacetone)dipalladium(0) (0.187 g, 0.2 mmol) are then added. The mixture is heated at 100° C. for 30 minutes and then cooled to ambient temperature. Water (50 mL) and Celite (6 g) are added. The suspension is filtered over Celite, and the filtrate is diluted with MTBE. The phases are separated and the aqueous phase is extracted with MTBE (2×50 mL). The combined organic phases are dried over Na₂SO₄ and filtered. The residue is purified by chromatography over silica gel using ethyl acetate and heptane as eluants followed by reverse-phase chromatography using methanol and water as eluants. The product obtained is lyophilised to provide the expected compound in the form of a solid.

¹H NMR (400 MHz, CDCl₃) δ ppm: 0.15 (s, 6H), 0.98 (s, 9H), 2.05 (s, 3H), 3.70 (s, 3H), 4.69 (br. s, 1H), 6.55-6.57 (m, 2H), 6.64-6.69 (m, 3H)

¹³C NMR (100 MHz, CDCl₃) δ ppm: —4.37, 9.42, 18.27, 25.84, 35.50, 102.59, 113.77, 115.13, 120.72, 121.91, 126.77, 126.94, 140.98, 148.39

### Preparation 38″: 4-[(4-{[tert-Butyl(dimethyl)silyl]oxy}phenyl)amino]-1-ethyl-5-methyl-1H-pyrrole-2-carbonitrile

The procedure is in accordance with the process of Preparation 33″, replacing the iodomethane-d₃ in Step B by iodoethane.

¹H NMR (400 MHz, dmso-d6) δ ppm: 6.85 (s, 1H), 6.75 (s, 1H), 6.6/6.5 (2d, 4H), 4 (quad, 2H), 2.1 (s, 3H), 1.3 (s, 3H), 0.9 (s, 9H), 0.1 (s, 6H)

### Preparation 39″: N—[4-[tert-Butyl(dimethyl)silyl]oxyphenyl]-2-ethoxy-pyrimidin-5-amine

Step A: 5-Bromo-2-ethoxy-pyrimidine

5-Bromo-2-chloro-pyrimidine (5.0 g, 25 mmol) is dissolved in ethanol (55 mL) and sodium ethylate (1.81 g, 26.6 mmol) is added in portions. The reaction mixture is stirred for 15 hours at ambient temperature. When the reaction is complete, the solvent is evaporated off, water (200 mL) is added, and then the reaction mixture is extracted with dichloromethane (2×100 mL). The organic phase is dried over Na₂SO₄, and then evaporated to dryness to yield 5-bromo-2-ethoxy-pyrimidine.

Step B: N—[4-[tert-Butyl(dimethyl)silyl]oxyphenyl]-2-ethoxy-pyrimidin-5-amine

4-[tert-Butyl(dimethyl)silyl]oxyaniline (1.8 g, 8 mmol) and 5-bromo-2-ethoxy-pyrimidine (1.6 g, 8 mmol) dissolved in anhydrous tetrahydrofuran (30 mL) are stirred for 1 hour at ambient temperature in the presence of sodium tert-butylate (4 mL, 2M solution in THF) and chloro(2-di-tert-butylphosphino-2′,4′,6′-triisopropyl-1,1′-biphenyl)[2-(2-aminoethyl)-phenyl]palladium(II) (111 mg, 0.16 mmol). The reaction mixture is filtered over Celite and then evaporated to dryness. The residue is triturated in heptane to obtain the expected compound after filtration.

¹H NMR (500 MHz, dmso-d6, 300 K) δ ppm: 8.31 (s, 2H, pyrimidine-H), 7.80 (s, 1H, NH), 6.73 (d, 2H, Ar—H), 6.68 (d, 2H, Ar—H), 4.26 (q, 2H, CH₂CH₃), 1.31 (t, 3H, CH₂CH₃), 0.94 (s, 9H, ^{t}Bu), 0.15 (s, 6H, Me)

IR: ν: aromatic: 1504 cm⁻¹; Si—C: 1247 cm⁻¹; C—O—C: 1057 cm⁻¹

### Preparation 40″: tert-Butyl 6-[(1-methyl-1H-pyrrolo[2,3-b]pyridin-5-yl)amino]-pyridin-3-yl carbonate

Step A: 6-Bromopyridin-3-yl tert-butyl carbonate

To a solution of 5 g of 6-bromopyridin-3-ol (28.7 mmol) and 7.53 g of di-tert-butyl dicarbonate (34.5 mmol) in 50 mL of tetrahydrofuran there is added 0.18 g of 4-dimethylaminopyridine (1.4 mmol). The mixture is stirred at ambient temperature for 6 hours and then concentrated. The residue obtained is dissolved in a mixture of ethyl ether and water. After separation of the phases, the separated-off organic phase is dried over MgSO₄ and concentrated to dryness. The title product is obtained in the form of a solid which is used in the next Step without being otherwise purified.

¹H NMR (400 MHz, dmso-d6) δ ppm: 8.4 (s, 1H), 7.71 (s, 2H), 1.5 (s, 9H)

Step B: tert-Butyl 6-[(1-methyl-1H-pyrrolo[2,3-b]pyridin-5-yl)amino]pyridin-3-yl carbonate

To a solution of 2.79 g of the compound obtained in Step A (10.2 mmol) in 15 mL of toluene and 15 mL of tetrahydrofuran there are added 1.18 g of sodium tert-butylate (12.2 mmol), and then the batch is stirred under argon for 15 minutes. There is then added 0.35 g of palladium catalyst (0.5 mmol). The reaction mixture is then stirred at ambient temperature for 16 hours and then filtered. The filtrate is concentrated and taken up in a mixture of dichloromethane and water. The organic phase is separated off and then washed with water, dried over MgSO₄, filtered and concentrated to dryness. The crude product thereby obtained is purified by chromatography over silica gel using dichloromethane and ethyl acetate as eluants. The residue is then taken up in a minimum of isopropyl ether. The solid then obtained is filtered off, washed with ether and then dried. The title product is obtained in the form of a solid, which is subsequently used without being otherwise purified.

¹H NMR (400 MHz, dmso-d6) δ ppm: 9 (s, 1H), 8.35 (wd, 1H), 8.3 (wd, 1H), 8 (wd, 1H), 7.45 (dd, 1H), 7.45 (wd, 1H), 6.8 (d, 1H), 6.4 (wd, 1H), 3.8 (s, 3H), 1.5 (s, 9H)

### Preparation 41″: 3-[4-[tert-Butyl(dimethyl)silyl]oxyanilino]benzonitrile

The procedure is in accordance with the process of Preparation 1″, replacing the 4-bromo-1-methyl-1H-pyrazole used in Step B by 3-bromobenzonitrile.

¹H NMR (400 MHz, dmso-d6) δ ppm: 8.3 (s, 1H), 7.3 (t, 1H), 7.2/7.1 (2 dd, 2H), 7.15 (t, 1H), 7.05 (d, 2H), 6.8 (d, 2H), 0.95 (s, 9H), 0.2 (s, 6H)

### Preparation 42″: 4-[4-[tert-Butyl(dimethyl)silyl]oxyanilino]thiophene-2-carbonitrile

The procedure is in accordance with the process of Preparation 1″, replacing the 4-bromo-1-methyl-1H-pyrazole used in Step B by 4-bromothiophene-2-carbonitrile.

¹H NMR (400 MHz, dmso-d6) δ ppm: 8.35 (s, 1H), 7.59 (wd, 1H), 7.08 (wd, 1H), 6.95 (d, 2H), 6.75 (d, 2H), 0.94 (s, 9H), 0.16 (s, 6H)

The amines NHR₃R₄ wherein R₃ and R₄, each independently of the other, represent an aryl or heteroaryl group are obtained in accordance with processes described in the literature (Surry D. S. et al., Chemical Science, 2011, 2, 27-50, Charles M. D. et al., Organic Letters, 2005, 7, 3965-3968). The reaction protecting the hydroxy function of the 4-anilinophenol described in Preparation 2″ can be applied to various secondary amines NHR₃R₄ (as defined hereinbefore) having one or more hydroxy functions, when they are available commercially. Alternatively, the secondary amines having at least one hydroxy substituent may be synthesised directly in a protected form, i.e. starting from reagents whose hydroxy function has been protected beforehand. Among the protecting groups, tert-butyl(dimethyl)silyloxy and benzyloxy are especially preferred.

Among the amines NHR₃R₄ having a hydroxy substituent that are used for synthesising the compounds of the invention there may be mentioned: 4-(4-toluidino)phenol, 4-(4-chloroanilino)phenol, 4-(3-fluoro-4-methylanilino)phenol, 4-[4-(trifluoromethoxy)anilino]-phenol, 4-[4-hydroxyanilino]phenol, {4-[(1-methyl-1H-indol-6-yl)amino]phenyl}methanol, 4-(2,3-dihydro-1H-indol-6-ylamino)phenol, 4-[(1-methyl-2,3-dihydro-1H-indol-6-yl)amino]phenol, 4-[(1-methyl-1H-indol-6-yl)amino]phenol, 4-[(1-methyl-1H-indol-6-yl)amino]cyclohexanol, 4-[(1-methyl-1,2,3,4-tetrahydro-6-quinolinyl)amino]phenol, 4-[(4-methyl-3,4-dihydro-2H-1,4-benzoxazin-7-yl)amino]phenol, 4-[4-(diethylamino)anilino]-phenol, 4-(2,3-dihydro-1H-inden-5-ylamino)phenol, 4-[(1-methyl-1H-indazol-5-yl)amino]-phenol, 4-[(1‘-methyl-1’,2′-dihydrospiro[cyclopropane-1,3′-indol]-5′-yl)amino]phenol, 4-[(1,3,3-trimethyl-2,3-dihydro-1H-indol-5-yl)amino]phenol, 4-[4-methoxy-3-(trifluoro-methyl)anilino]phenol, 4-[4-(methylsulphanyl)-3-(trifluoromethyl)anilino]phenol, 2-fluoro-4-[(1-methyl-1H-indol-5-yl)amino]phenol, 4-[(1-ethyl-1H-indol-5-yl)amino]phenol, 4-[(1-ethyl-2,3-dihydro-1H-indol-5-yl)amino]phenol, 4-[(1-isopropyl-2,3-dihydro-1H-indol-5-yl)amino]phenol, 4-(butylamino)phenol, 3-[(1-methyl-1H-indol-5-yl)amino]-1-propanol, 4-[(1-methyl-1H-indol-5-yl)amino]-1-butanol, 4-[(3-fluoro-4-methylphenyl)amino]phenol, 4-[(3-chloro-4-methylphenyl)amino]phenol, 4-[(4-fluorophenyl)amino]phenol, 4-[(1-methyl-1H-pyrrolo[2,3-b]pyridin-5-yl)amino]phenol, 4-[(4-fluorophenyl)amino]phenol, 4-[(2-fluorophenyl)amino]phenol, 4-[(3-fluorophenyl)amino]phenol, 4-[(2,4-difluorophenyl)amino]phenol, 4-[(3,4-difluorophenyl)amino]phenol, 3-[(4-hydroxy-phenyl)amino]benzonitrile, 4-[(3-methoxyphenyl)amino]phenol, 4-[(3,5-difluorophenyl)-amino]phenol, 4-[(3-methylphenyl)amino]phenol, 4-[(4-hydroxyphenyl)amino]benzo-nitrile, 4-[(3-chlorophenyl)amino]phenol, 4-(pyrimidin-2-ylamino)phenol, 4-[(cyclobutyl-methyl)amino]phenol, 2-[(4-hydroxyphenyl)amino]benzonitrile, 4-{[(1-methyl-1H-pyrazol-4-yl)methyl]amino}phenol, 4-[(cyclopropylmethyl)amino]phenol, 4-{[(1-methyl-1H-pyrazol-3-yl)methyl]amino}phenol, 4-(but-2-yn-1-ylamino)phenol, 4-(pyrazin-2-yl-amino)phenol, 4-(pyridin-2-ylamino)phenol, 4-(pyridazin-3-ylamino)phenol, 4-(pyrimidin-5-ylamino)phenol, 4-(pyridin-3-ylamino)phenol, 4-[(3,5-difluoro-4-methoxyphenyl)-amino]phenol, 4-(pyridin-4-ylamino)phenol, 4-[(3-fluoro-4-methoxyphenyl)amino]phenol, 2-(phenylamino)pyrimidin-5-ol, 5-[(4-hydroxyphenyl)amino]-2-methoxybenzonitrile, 4-{[3-(trifluoromethyl)phenyl]amino}phenol and 4-(methylamino)phenol.

The hydroxy function(s) of the secondary amines listed above is (are) protected beforehand by a suitable protecting group prior to any coupling to an acid derivative of the compound of formula (VII) as defined in the preceding general process.

### Preparation I: tert-Butyl [2-(3-iodo-4-{[(3R)-3-methyl-3,4-dihydroisoquinolin-2 (1H)-yl]carbonyl}phenyl)ethyl]carbamate

Step A: 4-[2-(tert-Butoxycarbonylamino)ethyl]benzoic acid

tert-Butyl dicarbonate (1.64 g; 7.5 mmol) is dissolved in 1,4-dioxane (5 mL) and the solution is added to a well-stirred solution of 4-(2-aminoethyl)benzoic acid (1.0 g; 5 mmol) in 1M aqueous sodium hydroxide solution (25 mL) and 1,4-dioxane (10 mL), and the mixture is then stirred at ambient temperature for 3 hours. The 1,4-dioxane is removed by evaporation in vacuo, and then ethyl acetate (200 mL) is added to the residue. The pH of the aqueous phase is adjusted to 1.5 with 2M aqueous HCl solution. The organic phase is separated off and the aqueous phase is then extracted with ethyl acetate (100 mL). The combined organic extracts are washed with water, dried over Na₂SO₄ and evaporated to a volume of 10 mL. Heptane is added to the residue and then the solid precipitate is filtered off to yield the title compound.

¹H NMR (500 MHz, dmso-d6, 300K): 12.81 (br s, 1H, COOH), 7.85 (m, 2H, Ar-2′ and 6′H), 7.31 (m, 2H, Ar-3′ and 5′H), 6.91 and 6.51 (2×br s, 1H, NH), 3.16 (m, 2H, CH₂), 2.75 (t, 2H, CH₂), 1.35 and 1.31 (2×br s, 9H, Bu^{t}); ¹³C NMR (125 MHz, dmso-d6, 300 K): 167.8 (q), 156.0 (CH), 145.3 (q), 129.8 (2× CH), 129.3 (2×CH), 129.1 (q), 78.0 (q), 41.5 (CH₂), 35.9 (CH₂), 28.7 (CH₃)

Step B: 4-[2-(tert-Butoxycarbonylamino)ethyl]-2-iodo-benzoic acid

The compound of the above Step (0.88 g; 3.31 mmol), iodobenzene diacetate (1.07 g; 3.31 mmol), tetrabutylammonium iodide (1.22 g; 3.31 mmol), iodine (0.84 g; 3.31 mmol) and diacetoxypalladium (0.04 g; 0.165 mmol) are dissolved in 1,2-dichloroethane (8 mL), and the mixture is then heated in a microwave reactor at 80° C. for 90 minutes. The cooled mixture is partitioned between saturated aqueous Na₂CO₃ solution (30 mL) and the original solvent. The organic phase is separated off and the aqueous residue is extracted with diethyl ether (2×15 mL). The pH of the resulting aqueous solution is adjusted to 2 with 2M aqueous HCl solution and the product is then extracted with ethyl acetate (2×75 mL). The organic phase is dried over Na₂SO₄ and evaporated. The crude product is purified by chromatography over silica gel to yield the mixture of regioisomers, which are separated by preparative HPLC using water-TFA and acetonitrile as eluants. The pH of the appropriate combined fractions is adjusted to 5 with NaHCO₃, and then the acetonitrile is evaporated off under reduced pressure. The precipitate is recovered by filtration and then dried to yield the title compound.

¹H NMR (500 MHz, dmso-d6, 300K): 13.16 (broad s, 1H, COOH), 7.81 (s, 1H, Ar-3′H), 7.66 (d, 1H, Ar-6′H), 7.29 (d, 1H, Ar-5′H), 6.89 (t, 1H, NH), 3.14 (t, 1H, CH₂), 2.69 (t, 1H, CH₂), 1.35 (s, 9H, Boc)

Step C: tert-Butyl [2-(3-iodo-4-{[(3R)-3-methyl-3,4-dihydroisoquinolin-2 (1H)-yl]-carbonyl}phenyl)ethyl]carbamate

The compound of the above Step (0.714 g; 1.82 mmol), TBTU (0.73 g; 2.28 mmol), N-ethyl-N-isopropyl-propan-2-amine (0.94 mL; 5.46 mmol) and N,N-dimethylpyridin-4-amine (22 mg) are stirred in dichloromethane (20 mL) for 5 minutes, and then (3R)-3-methyl-1,2,3,4-tetrahydroisoquinoline (0.282 g; 1.92 mmol) (cf. Preparation 1′) is added. The mixture is stirred for three more hours. The reaction mixture is diluted with dichloromethane (150 mL) and then washed with water (25 mL), dried over Na₂SO₄ and evaporated. The crude product is purified by chromatography over silica gel using dichloromethane and methanol as eluants to yield the title compound.

### High-Resolution Mass Spectrometry (ESI+):

C₂₄H₂₉IN₂O₃ Empirical formula:
[M+H]⁺ calculated: 521.1303
[M+H]⁺ measured: 521.1282

IR: ν: C—H: 2931 cm⁻¹; >C═O: 1706, 1627 cm⁻¹; amide: 1505 cm⁻¹; C—O—C: 1248, 1166 cm

### Preparation II: tert-Butyl (4-Iodo-3-{[(3R)-3-methyl-3,4-dihydroisoquinolin-2 (1H)-yl]carbonyl}benzyl)carbamate

Step A: 2-Iodo-5-methyl-benzoic acid

3-Methylbenzoic acid (6.12 g; 45 mmol), iodobenzene diacetate (14.49 g; 45 mmol), tetrabutylammonium iodide (16.62 g, 45 mmol), iodine (11.43 g; 45 mmol) and diacetoxypalladium (0.5 g; 2.2 mmol) are dissolved in 1,2-dichloroethane, and then the mixture is heated in a sealed tube at 85° C. for 1 hour and 45 minutes. The cooled solution is purified by chromatography over silica gel using dichloromethane and methanol as eluants to yield the title compound.

Step B: Methyl 2-iodo-5-methyl-benzoate

The compound of the above Step (6.25 g, 23.9 mmol) is dissolved in anhydrous methanol (100 mL), and then SOCl₂ (3.6 mL, 49 mmol) is added dropwise to the well-stirred solution.

The solution is refluxed for 18 hours and then evaporated to a volume of 25 mL and then poured into crushed ice (100 g). The resulting mixture is extracted with diethyl ether (2×75 mL). The organic phase is washed with saturated aqueous NaHCO₃ solution and then brine, dried over Na₂SO₄ and evaporated to dryness to yield the title compound.

Step C: Methyl 5-(bromomethyl)-2-iodo-benzoate

The compound of the above Step (5.7 g; 20.7 mmol), N-bromo-succinimide (3.67 g; 20.7 mmol) and dibenzoyl peroxide (0.24 g; 1 mmol) are dissolved in carbon tetrachloride (40 mL) and then refluxed for 5 hours. New portions of N-bromosuccinimide (1.0 g; 5.6 mmol) and dibenzoyl peroxide (0.05 g; 0.2 mmol) are then added and heating is continued for 4 more hours. The reaction mixture is cooled to ambient temperature. The insoluble parts are removed by filtration, and the concentrated filtrate is then purified by chromatography over silica gel using heptane and ethyl acetate as eluants to yield the title compound.

Step D: Methyl 5-(aminomethyl)-2-iodo-benzoate

The compound of the above Step (0.53 g; 1.5 mmol) is dissolved in 7M methanolic ammonia (20 mL) and then stirred at ambient temperature for 1 hour. The solution is evaporated to dryness to yield the title compound, which was used in the next Step without purification.

Step E: 5-[(tert-Butoxycarbonylamino)methyl]-2-iodo-benzoic acid

Methyl 5-(aminomethyl)-2-iodo-benzoate hydrobromide (0.56 g; 1.5 mmol) was dissolved in pyridine (10 mL), then di-tert-butyl dicarbonate (0.80 g; 3.6 mmol) is added and the mixture is stirred at ambient temperature for 30 minutes and then evaporated to dryness. The residue is redissolved in methanol (10 mL), and then 2M aqueous NaOH solution (3 mL) and 2 mL of water are added. The resulting solution is stirred for 2 hours at 50° C., then diluted with water (20 mL), and the methanol is evaporated off under reduced pressure. The pH of the resulting aqueous solution is adjusted to 3 with 2M aqueous HCl solution; the product is then extracted with ethyl acetate. The organic phase is washed with brine, and then dried over Na₂SO₄ and evaporated to dryness. The crude product is triturated with DCM. The solid formed is recovered by filtration to yield the title compound.

¹H NMR (500 MHz, dmso-d6, 300 K): 13.24 (broad s, 1H, COOH), 7.91 (d, 1H, Ar-3′H), 7.58 (d, 1H, Ar-6′H), 7.47 (t, 1H, NH), 7.09 (d, 1H, (Ar-5′H), 4.09 (d, 2H, CH₂), 1.38 (s, 9H, Boc); ¹³C NMR (125 MHz, dmso-d6, 300K): 168.6 (q), 156.3 (q), 141.1 (q), 140.9 (CH), 137.1 (q), 131.6 (CH), 129.1 (CH), 92.2 (q), 78.5 (q), 28.7 (CH₃)

Step F: tert-Butyl (4-iodo-3-{[(3R)-3-methyl-3,4-dihydroisoquinolin-2 (1H)-yl]carbonyl}-benzyl)carbamate

The compound of the above Step (1.62 g; 4.3 mmol), TBTU (2.76 g; 8.59 mmol), N-ethyl-N-isopropyl-propan-2-amine (1.48 mL; 8.59 mmol) and N,N-dimethylpyridin-4-amine (10 mg) are stirred in DCM (50 mL) for 5 minutes; (3R)-3-methyl-1,2,3,4-tetrahydroisoquinoline (0.76 g; 5.15 mmol) (cf. Preparation 1′) is then added and the mixture is stirred for 15 minutes more. The insoluble parts are removed by filtration, and then the concentrated filtrate is purified by chromatography over silica gel using dichloromethane and methanol as eluants to yield the title compound.

### Preparation III: tert-Butyl (3-bromo-4-{[(3R)-3-methyl-3,4-dihydroisoquinolin-2 (1H)-yl]carbonyl}benzyl)carbamate

Step A: Methyl 4-(aminomethyl)-2-bromo-benzoate

A solution of methyl 2-bromo-4-(bromomethyl)benzoate (4.57 g; 14.84 mmol) in 50 mL of methanol is added dropwise to a well-stirred solution of ammonia in methanol (7M; 315 mL) at ambient temperature, and the solution is then stirred for 3 hours. All the solvents are evaporated off and the residue is purified by chromatography over silica gel using dichloromethane-methanol as eluants to yield the title compound.

Step B: 2-Bromo-4-[(tert-butoxycarbonylamino)methyl]benzoic acid

The compound of the above Step (4.04 g; 16.6 mmol) is dissolved in pyridine (55 mL) and then di-tert-butyl dicarbonate (5.43 g; 24.9 mmol) is added and the mixture is stirred at ambient temperature for 16 hours; it is then evaporated to dryness. The residue is redissolved in methanol (100 mL), and then 1M aqueous NaOH solution (54 mL) is added. The resulting solution is stirred for 1.5 hours at 50° C. and is then allowed to cool to ambient temperature. The pH of the solution is adjusted to 7 using 2M aqueous HCl solution; the methanol is then evaporated off under reduced pressure. The resulting aqueous solution is diluted with water (50 mL), and then the pH is adjusted to 3 using 2M aqueous HCl solution. The product is extracted with dichloromethane (2×100 mL). The organic phase is washed with brine, then dried over Na₂SO₄ and evaporated to dryness to yield the title compound which is used in the next Step without purification.

IR: ν: N—H: 3359 cm⁻¹; C—H: 2983 cm⁻¹; >C═O: 1685, 1603 cm⁻¹; amide: 1519 cm⁻¹; C—O—C: 1248, 1162, 1057 cm⁻¹

Step C: tert-Butyl (3-bromo-4-{[(3R)-3-methyl-3,4-dihydroisoquinolin-2 (1H)-yl]-carbonyl}benzyl)carbamate

The compound of the above Step (3.7 g; 11.2 mmol), TBTU (7.19 g; 22.4 mmol), N-ethyl-N-isopropyl-propan-2-amine (3.86 mL; 22.4 mmol) and N,N-dimethylpyridin-4-amine (48 mg) are stirred in dichloromethane (100 mL) for 5 minutes, and then (3R)-3-methyl-1,2,3,4-tetrahydroisoquinoline (1.98 g, 13.44 mmol) (cf. Preparation 1′) is added, and the mixture is stirred for 1 hour more. The insoluble parts are removed by filtration, and then the concentrated filtrate is purified by chromatography over silica gel using dichloromethane and methanol as eluants to yield the title compound.

### High-Resolution Mass Spectrometry (ESI+):

C₂₃H₂₇BrN₂O₃ Empirical formula:

[M+H]⁺ calculated: 459.1285

[M+H]⁺ measured: 459.1282

¹H NMR (500 MHz, dmso-d6, 300K, presence of amide rotamers): 7.66-6.87 (m, 7H, aromatic), 5.34 and 4.12 (d, 2H, CH₂-isoquinoline), 5.02 and 4.97 and 3.87 and 3.83 (m, 1H, CH₂-isoquinoline), 4.17 (d, 2H, CH₂-benzyl), 3.18-2.48 (m, 1H, CH₂-isoquinoline), 1.16 and 1.14 and 1.10 and 0.98 (d, 3H, isoquinoline CH₃), 1.31 and 1.41 (s, 9H, Boc).

### EXAMPLE 1

5-(5-chloro-2-{[(3R)-3-methyl-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide
Step A: Ethyl 5-(5-chloro-2-{[(3R)-3-methyl-3,4-dihydroisoquinolin-2 (1H)-yl]carbonyl}phenyl)-1,2-dimethyl-1H-pyrrole-3-carboxylate

To a solution of 1.4 g of the compound obtained in Preparation 1 (4.35 mmol) in 50 mL of dichloromethane there are successively added 0.7 g of the compound obtained in Preparation 1′ (4.79 mmol), 0.7 g of HOBT (5.22 mmol), 0.81 g of EDC (5.22 mmol) and 1.6 mL of triethylamine (21.7 mmol). The batch is then stirred overnight at ambient temperature. The reaction mixture is then diluted with dichloromethane and washed 3 times with saturated aqueous NaHCO₃ solution. The organic phase is then dried over MgSO₄, filtered, concentrated to dryness, and then purified by chromatography over silica gel using dichloromethane and methanol as eluants.

¹H NMR: δ (500 MHz; dmso-d6; 300K): 7.6-7.3 (m, 3H, aromatic Hs, 4-chlorophenyl); 7.2-6.85 (m, 4H, aromatic Hs, tetrahydroisoquinoline); 6.45-6.3 (m, 1H, H pyrrole); 5.0-4.8-3.8 (m, 1H, aliphatic H, tetrahydroisoquinoline); 5.3-3.75 (dd, 2H, aliphatic Hs tetrahydroisoquinoline); 4.2-4.0 (m, 2H, OCH₂CH₃); 3.25 (s, 3H, CH₃—N-pyrrole); 3.0-2.2 (m, 2H, aliphatic Hs, tetrahydroisoquinoline); 2.5 (s, 3H, CH₃-pyrrole); 1.25 (t, 3H, OCH₂CH₃); 1.05 (d, 3H, tetrahydroisoquinoline-CH₃)

IR: ν: >C═O: 1693 cm⁻¹ ester; ν: >C═O: 1625 cm⁻¹ amide

Step B: 5-(5-Chloro-2-{[(3R)-3-methyl-3,4-dihydroisoquinolin-2 (1H)-yl]carbonyl}-phenyl)-1,2-dimethyl-1H-pyrrole-3-carboxylic acid

To a solution of 1.7 g of the compound obtained in Step A (3.77 mmol) in 5 mL of dioxane there is added 0.317 g of LiOH (7.5 mmol) dissolved in 5 mL of water. The batch is heated in a microwave apparatus for 4 hours at 100° C. (power 140 W). The reaction mixture is then filtered and concentrated to dryness. The residue thereby obtained is taken up in dichloromethane (50 mL) and then saturated aqueous NH₄Cl solution is added thereto. The organic phase is washed with water and then with brine, dried over MgSO₄, filtered and concentrated to dryness. The title product is obtained in the form of a solid and is used directly in the next Step.

¹H NMR: δ (500 MHz; dmso-d6; 300K): 11.05 (broad s, 1H, COOH), 7.5-7.2 (m, 3H, aromatic Hs, 4-chlorophenyl); 7.2-6.9 (m, 4H, aromatic Hs, tetrahydroisoquinoline); 6.45-6.2 (m, 1H, aliphatic H, H pyrrole); 5.3-3.75 (dd, 2H, aliphatic Hs, tetrahydroisoquinoline); 5.0-4.8-3.8 (m, 1H, aliphatic H, tetrahydroisoquinoline); 3.5-3.2 (s, 3H, CH₃—N-pyrrole); 3.0-2.1 (aliphatic Hs, CH₃-tetraisoquinoline); 2.5-2.4-1.98 (m, 3H, CH₃-pyrrole); 1.05-0.52 (m, 3H, aliphatic Hs, CH₃-tetraisoquinoline)

IR: ν: —OH: 3500-2000 cm⁻¹ carboxylic acid; ν: >C═O: 1699+1658 cm⁻¹ carboxylic acid; ν: >C═N—: 1625 cm⁻¹ amide

Step C: N-(4-{[tert-Butyl(dimethyl)silyl]oxy}phenyl)-5-(5-chloro-2-{[(3R)-3-methyl-3,4-dihydroisoquinolin-2 (1H)-yl]carbonyl}phenyl)-1,2-dimethyl-N-(1-methyl-1H-pyrazol-4-yl)-1H-pyrrole-3-carboxamide

To a solution of 0.65 g of the compound obtained in Step B (1.54 mmol) in 15 mL of dichloroethane there is added, dropwise, 0.244 mL of 1-chloro-N,N,2-trimethyl-prop-1-en-1-amine. The reaction mixture is stirred at ambient temperature for 1 hour, and there are then added 0.56 g of the compound of Preparation 1″ (1.84 mmol), 10 mL of dichloroethane and 0.376 g of 4-dimethylaminopyridine (DMAP) (3.07 mmol). The batch is stirred at 110° C. overnight. The reaction mixture is concentrated, dissolved in dichloromethane and washed with saturated aqueous NaHCO₃ solution. The title product is obtained in the form of an oil and is used directly in the next Step.

Step D: 5-(5-Chloro-2-{[(3R)-3-methyl-3,4-dihydroisoquinolin-2 (1H)-yl]carbonyl}-phenyl)-N-(4-hydroxyphenyl)-1,2-dimethyl-N-(1-methyl-1H-pyrazol-4-yl)-1H-pyrrole-3-carboxamide

To a solution of 0.8 g of the compound obtained in Step C (1.03 mmol) in 2 mL of methanol, there are added 1.55 mL of a 1M solution of potassium hydroxide in methanol (1.55 mmol).

The batch is stirred at ambient temperature for 30 minutes. The reaction mixture is then diluted with dichloromethane and successively washed with 1M aqueous HCl solution, saturated aqueous NaHCO₃ solution and then brine until neutral. The organic phase is then dried over MgSO₄, filtered and evaporated. The crude product thereby obtained is purified by chromatography over silica gel using dichloromethane and methanol as eluants. The solid thereby obtained is dissolved in a mixture of water/acetonitrile until dissolution is complete, filtered and then lyophilised.

### Elemental Microanalysis: (%, Theoretical:Measured)

% C=68.74:68.25; % H=5.43:5.69; % N=11.79:11.66; % Cl=5.97:5.95

### High-Resolution Mass Spectrometry (ESI+):

C₃₄H₃₂ClN₅O₃ Empirical formula:

[M+H]⁺ calculated: 594.2266

[M+H]⁺ measured: 594.2289

### EXAMPLE 2

5-(2-{[(3S)-3-(Aminomethyl)-3,4-dihydroisoquinolin-2 (1H)-yl]carbonyl}phenyl)-1-methyl-N,N-diphenyl-1H-pyrrole-3-carboxamide
Step A: tert-Butyl {[(3S)-2-{2-[4-(diphenylcarbamoyl)-1-methyl-1H-pyrrol-2-yl]benzoyl}-1,2,3,4-tetrahydroisoquinolin-3-yl]methyl}carbamate

The title compound is obtained in accordance with the process of Example 1 using the acid of Preparation 2 and tert-butyl [(3S)-1,2,3,4-tetrahydroisoquinolin-3-ylmethyl]carbamate (see Preparation 2′) in Step A, and N-phenylaniline in Step C.

Step B: 5-(2-{[(3S)-3-(Aminomethyl)-3,4-dihydroisoquinolin-2 (1H)-yl]carbonyl} phenyl)-1-methyl-N,N-diphenyl-1H-pyrrole-3-carboxamide

A solution of the NH-Boc compound of Step A in dichloromethane is placed at 0° C. 10 molar equivalents of trifluoroacetic acid are added thereto dropwise. The batch is stirred at ambient temperature for 4 hours until the starting material has disappeared completely. The reaction mixture is then concentrated to dryness, taken up again and co-evaporated twice with toluene, then taken up in a mixture of acetonitrile/H₂O and finally lyophilised. The title product is then obtained after a neutralisation step.

### Elemental Microanalysis: (%, Theoretical:Measured)

% C=77.75:77.27; % H=5.97:5.73; % N=10.36:10.44

### EXAMPLE 3

5-(5-Chloro-2-{[(3S)-3-[(4-methylpiperazin-1-yl)methyl]-3,4-dihydro-isoquinolin-2 (1H)-yl]carbonyl}phenyl)-N-(4-hydroxyphenyl)-N-(1H-indol-5-yl)-1-methyl-1H-pyrrole-3-carboxamide
Step A: tert-Butyl 5-[(4-{[tert-butyl(dimethyl)silyl]oxy}phenyl){[5-(5-chloro-2-{[(3S)-3-[(4-methylpiperazin-1-yl)methyl]-3,4-dihydroisoquinolin-2(H)-yl]carbonyl}phenyl)-1-methyl-1H-pyrrol-3-yl]carbonyl}amino]-1H-indole-1-carboxylate

The title compound is obtained in accordance with the process of Example 1 using the acid of Preparation 3 and (3S)-3-[(4-methyl-1-piperazinyl)methyl]-1,2,3,4-tetrahydro-isoquinoline (see Preparation 4′) in Step A, and the compound of Preparation 3″ in Step C.

Step B: 5-(5-Chloro-2-{[(3S)-3-[(4-methylpiperazin-1-yl)methyl]-3,4-dihydroisoquinolin-2 (1H)-yl]carbonyl}phenyl)-N-(4-hydroxyphenyl)-N-(1H-indol-5-yl)-1-methyl-1H-pyrrole-3-carboxamide

To a solution of 455 mg (0.49 mmol) of the product obtained in Step A in 5 mL of methanol there are added 135 mg (2.5 mmol) of KOH. After stirring for 3 hours at ambient temperature, the reaction mixture is concentrated, treated with saturated aqueous NaHCO₃ solution and extracted with methylene chloride. The organic phase is then dried over MgSO₄, filtered and evaporated to dryness. The crude product thereby obtained is then purified by chromatography over silica gel using dichloromethane and methanol as eluants to yield the expected product in the form of a foam.

### Elemental Microanalysis: (%, Theoretical:Measured)

% C=70.72:68.54; % H=5.79:5.37; % N=11.78:11.41; % Cl=4.97:4.79

### High-Resolution Mass Spectrometry (ESI+):

C₄₂H₄₁ClN₆O₃ Empirical formula:

[M+H]⁺ calculated: 713.3007

[M+H]⁺ measured: 713.2973

### EXAMPLE 4

N-(4-Hydroxyphenyl)-N-(1H-indol-5-yl)-1-methyl-5-(6-{[(3S)-3-[(4-methylpiperazin-1-yl)methyl]-3,4-dihydroisoquinolin-2 (1H)-yl]carbonyl}-1,3-benzodioxol-5-yl)-1H-pyrrole-3-carboxamide

The title compound is obtained in accordance with the process of Example 3 using the compound of Preparation 4 in Step A.

### Elemental Microanalysis: (%, Theoretical:Measured)

% C=71.45:69.32; % H=5.86:5.22; % N=11.63:11.08

### High-Resolution Mass Spectrometry (ESI+):

C₄₃H₄₂N₆O₅ Empirical formula:

[M+H]⁺ calculated: 723.3295

[M+H]⁺ measured: 723.3262

Unless otherwise mentioned, the compounds of the following Examples are synthesised in accordance with the process of Example 1 using, in Step A: (i) the appropriate acid obtained according to one of Preparations 1 to 32 and (ii) the appropriate tetrahydroisoquinoline compound obtained according to one of Preparations 1′ to 9′ and, in Step C: (iii) the suitable NHR₃R₄ amine (a non-exhaustive list is proposed in Preparations 1″ to 42″. The compounds thereby obtained are optionally subjected to a step of conversion into salt form in the presence of HCl in ether. After filtration and lyophilisation in a mixture of acetonitrile/water, the hydrochloride of the expected compound is obtained.

### EXAMPLE 5

5-(5-Chloro-2-{[(3S)-3-[(4-methylpiperazin-1-yl)methyl]-3,4-dihydroisoquinolin-2 (1H)-yl]carbonyl}phenyl)-N-(4-hydroxyphenyl)-1-methyl-N-(1-methyl-1H-indol-5-yl)-1H-pyrrole-3-carboxamide hydrochloride

### Elemental Microanalysis: (%, Theoretical:Measured)

% C=64.54:64.9; % H=5.67:5.49; % N=10.5:10.4; % Cl=13.29:12.52; % Cl—=8.86:7.39

### EXAMPLE 6

5-(5-Chloro-2-{[(3S)-3-[(4-methylpiperazin-1-yl)methyl]-3,4-dihydroisoquinolin-2 (1H)-yl]carbonyl}phenyl)-1-ethyl-N-(4-hydroxyphenyl)-N-(1-methyl-1H-indol-5-yl)-1H-pyrrole-3-carboxamide

### High-Resolution Mass Spectrometry (ESI+):

C₄₄H₄₅ClN₆O₃ Empirical formula:

[M+H]⁺ calculated: 741.3320

[M+H]⁺ measured: 741.3326

### EXAMPLE 7

5-(5-Chloro-2-{[(3S)-3-[(4-METHYLPIPERAZIN-1-yl)METHYL]-3,4-dihydroisoquinolin-2 (1H)-yl]carbonyl}phenyl)-1-cyclopropyl-N-(4-hydroxyphenyl)-N-(1-methyl-1H-indol-5-yl)-1H-pyrrole-3-carboxamide

### High-Resolution Mass Spectrometry (ESI+):

C₄₅H₄₅ClN₆O₃ Empirical formula:

[M+H]⁺ calculated: 753.3320

[M+H]⁺ measured: 753.3306

### EXAMPLE 8

5-(5-Chloro-2-{[(3S)-3-[(4-methylpiperazin-1-yl)methyl]-3,4-dihydroisoquinolin-2 (1H)-yl]carbonyl}phenyl)-N-(4-hydroxyphenyl)-N-(1-methyl-1H-indol-5-yl)-1-(propan-2-yl)-1H-pyrrole-3-carboxamide

### High-Resolution Mass Spectrometry (ESI+):

C₄₅H₄₇ClN₆O₃ Empirical formula:

[M+H]⁺ calculated: 755.3476

[M+H]⁺ measured: 755.3466

### EXAMPLE 9

N-(4-Hydroxyphenyl)-1-methyl-N-(1-methyl-H-indol-5-yl)-5-(6-{[(3S)-3-[(4-methylpiperazin-1-yl)methyl]-3,4-dihydroisoquinolin-2 (1H)-yl]carbonyl}-1,3-benzodioxol-5-yl)-1H-pyrrole-3-carboxamide hydrochloride

### Elemental Microanalysis: (%, Theoretical:Measured)

% C=65.26:65.8; % H=5.73:5.47; % N=10.38:10.48; % Cl=8.76:8.46; % Cl—=8.76:8.02

### EXAMPLE 10

5-(5-Fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-phenyl-1H-pyrrole-3-carboxamide hydrochloride

### Elemental Microanalysis: (%, Theoretical:Measured)

% C=69.11:68.56; % H=5.8:5.31; % N=8.06:8.13; % Cl—=5.1:4.68

### EXAMPLE 11

5-(5-Fluoro-2-{[(3S)-3-(morpholin-4-ylmethyl)-3,4-dihydroisoquinolin-2 (1H)-yl]carbonyl}phenyl)-N-(4-hydroxyphenyl)-1-(3-hydroxypropyl)-2-methyl-N-phenyl-1H-pyrrole-3-carboxamide hydrochloride
Step A: 1-[3-(Benzyloxy)propyl]-5-(5-fluoro-2-{[(3S)-3-(morpholin-4-ylmethyl)-3,4-dihydroisoquinolin-2(H)-yl]carbonyl}phenyl)-N-(4-hydroxyphenyl)-2-methyl-N-phenyl-1H-pyrrole-3-carboxamide

The title compound is obtained in accordance with the process of Example 1 using the acid of Preparation 10 and the compound of Preparation 3′ in Step A, and the compound of Preparation 2″ in Step C.

Step B: 5-(5-Fluoro-2-{[(3S)-3-(morpholin-4-ylmethyl)-3,4-dihydroisoquinolin-2 (1H)-yl]carbonyl}phenyl)-N-(4-hydroxyphenyl)-1-(3-hydroxypropyl)-2-methyl-N-phenyl-1H-pyrrole-3-carboxamide hydrochloride

The compound of Step A is subjected to a deprotection reaction in accordance with a protocol analogous to that described in Step B of Example 23.

### Elemental Microanalysis: (%, Theoretical:Measured)

% C=68.24:67.96; % H=6:5.79; % N=7.58:7.61; % Cl—=4.8:4.75

### High-Resolution Mass Spectrometry (ESI+):

C₄₂H₄₃FN₄O₅ Empirical formula:

[M+H]⁺ calculated: 703.3296

[M+H]⁺ measured: 703.33294

### EXAMPLE 12

5-(5-Fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-indazol-5-yl)-1H-pyrrole-3-carboxamide hydrochloride

### Elemental Microanalysis: (%, Theoretical:Measured)

% C=67.33:67.39; % H=5.65:5.18; % N=11.22:11.52; % Cl—=4.73:3.82

### EXAMPLE 13

N-(3-Fluoro-4-methylphenyl)-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Theoretical:Measured)

% C=66.97:66.72; % H=5.62:5.21; % N=7.44:7.59; % Cl—=4.71:4.54

### EXAMPLE 14

N-(4-Hydroxyphenyl)-1,2-dimethyl-N-(1-methyl-1H-indazol-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Theoretical:Measured)

% C=66.62:66.59; % H=5.59:4.67; % N=10.84:10.85; % Cl—=4.57:4.24

### EXAMPLE 15

N-(4-Hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-N-phenyl-1H-pyrrole-3-carboxamide hydrochloride

### Elemental Microanalysis: (%, Theoretical:Measured)

% C=68.28:67.07; % H=5.73:5.09; % N=7.77:7.75; % Cl—=4.92:5.71

### High-Resolution Mass Spectrometry (ESI+):

C₄₁H₄₀N₄O₆ Empirical formula:

[M+H]⁺ calculated: 685.3026

[M+H]⁺ measured: 685.3033

### EXAMPLE 16

N-(3-Fluorophenyl)-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Theoretical:Measured)

% C=66.62:66.11; % H=5.45:5.2; % N=7.58:7.89; % Cl—=4.8:5.59

### EXAMPLE 17

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-phenyl-1H-pyrrole-3-carboxamide hydrochloride

### Elemental Microanalysis: (%, Theoretical:Measured)

% C=67.51:66.91; % H=5.66:5.05; % N=7.87:7.88; % Cl—=4.98:5.75

### EXAMPLE 18

1-(2-Hydroxyethyl)-N-(4-hydroxyphenyl)-2-methyl-5-(6-{[(3S)-3-[2-(morpholin-4-yl)ethyl]-3,4-dihydroisoquinolin-2 (1H)-yl]carbonyl}-1,3-benzodioxol-5-yl)-N-phenyl-1H-pyrrole-3-carboxamide hydrochloride
Step A: 1-[2-(Benzyloxy)ethyl]-N-[4-(benzyloxy)phenyl]-2-methyl-5-(6-{[(3S)-3-[2-(morpholin-4-yl)ethyl]-3,4-dihydroisoquinolin-2 (1H)-yl]carbonyl}-1,3-benzodioxol-5-yl)-N-phenyl-1H-pyrrole-3-carboxamide

The title compound is obtained in accordance with the process of Example 1 using the acid of Preparation 9 and (3S)-3-[2-(morpholin-4-yl)ethyl]-1,2,3,4-tetrahydroisoquinoline (see Preparation 5′) in Step A, and 4-(benzyloxy)-N-phenylaniline (see Preparation 8″) in Step C.

Step B: 1-(2-Hydroxyethyl)-N-(4-hydroxyphenyl)-2-methyl-5-(6-{[(3S)-3-[2-(morpholin-4-yl)ethyl]-3,4-dihydroisoquinolin-2 (1H)-yl]carbonyl}-1,3-benzodioxol-5-yl)-N-phenyl-1H-pyrrole-3-carboxamide hydrochloride

The compound of Step A is subjected to a deprotection reaction in accordance with a protocol analogous to that described in Step B of Example 23.

### High-RESOLUTION Mass Spectrometry (ESI+):

C₃₈H₄₀N₆O₅ Empirical formula:

[M+H]⁺ calculated: 729.3283

[M+H]⁺ measured: 729.3282

### EXAMPLE 19

N-(4-Hydroxyphenyl)-5-(5-methoxy-2-{[(3S)-3-(morpholin-4-ylmethyl)-3,4-dihydroisoquinolin-2 (1H)-yl]carbonyl}phenyl)-1,2-dimethyl-N-phenyl-1H-pyrrole-3-carboxamide hydrochloride

### Elemental Microanalysis: (%, Theoretical:Measured)

% C=69.63:69.45; % H=6.13:5.94; % N=7.92:7.79; % Cl—=5.01:4.58

### EXAMPLE 20

N-(4-Hydroxyphenyl)-5-(5-methoxy-2-{[(3R)-3-methyl-3,4-dihydroisoquinolin-2 (1H)-yl]carbonyl}phenyl)-1,2-dimethyl-N-(pyridin-4-yl)-1H-pyrrole-3-carboxamide hydrochloride

### Elemental Microanalysis: (%, Theoretical:Measured)

% C=69.39:69.66; % H=5.66:5.46; % N=8.99:8.92; % Cl—=5.69:5.29

### High-Resolution Mass Spectrometry (ESI+):

C₃₆H₃₅N₄O₄ Empirical formula:

[M+H]⁺ calculated: 587.2653

[M+H]⁺ measured: 587.2649

### EXAMPLE 21

N-(4-Hydroxyphenyl)-1,2-dimethyl-N-(1-methyl-1H-pyrazol-4-yl)-5-(7-{[(3S)-3-(morpholin-4-ylmethyl)-3,4-dihydroisoquinolin-2 (1H)-yl]carbonyl}-2,3-dihydro-1,4-benzodioxin-6-yl)-1H-pyrrole-3-carboxamide hydrochloride

### Elemental Microanalysis: (%, Theoretical:Measured)

% C=64.99:64.67; % H=5.86:5.67; % N=11.37:11.27; % Cl—=4.8:4.71

### High-Resolution Mass Spectrometry (ESI+):

C₄₀H₄₃N₆O₆ Empirical formula:

[M+H]⁺ calculated: 703.3236

[M+H]⁺ measured: 703.3239

### EXAMPLE 22

N-(4-Hydroxyphenyl)-1,2-dimethyl-5-(7-{[(3S)-3-(morpholin-4-ylmethyl)-3,4-dihydroisoquinolin-2 (1H)-yl]carbonyl}-2,3-dihydro-1,4-benzodioxin-6-yl)-N-phenyl-1H-pyrrole-3-carboxamide hydrochloride

### Elemental Microanalysis: (%, Theoretical:Measured)

% C=68.61:67.97; % H=5.89:5.59; % N=7.62:7.55; % Cl—=4.82:4.27

### EXAMPLE 23

1-(2-Hydroxyethyl)-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide
Step A: 1-[2-(Benzyloxy)ethyl]-N-(4-{[tert-butyl(dimethyl)silyl]oxy}phenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide

The title compound is obtained in accordance with the process of Example 1 using the acid of Preparation 9 and the compound of Preparation 1″ in Step C.

Step B: N-(4-{[tert-Butyl(dimethyl)silyl]oxy}phenyl)-1-(2-hydroxyethyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide

To a solution of 6.86 g (8.19 mmol) of the compound of Step A in 70 mL of anhydrous methanol there are added 1.37 g of Pd/C 10%. The batch is degassed for 0.5 hours and then stirred at ambient temperature under dihydrogen pressure (1.5 bar) for 12 hours. The reaction mixture is then filtered and then concentrated to dryness. The expected product is obtained in the form of a solid.

¹H NMR: δ (500 MHz; dmso-d6; 300K): 7.7-7.4 (s, 1H, H pyrazole); 7.1-6.8 (s, 1H, H pyrazole); 7.3-6.9 (m, 4H, aromatic Hs, H tetrahydroisoquinoline); 7.0-6.7 (m, 2H, aromatic Hs); 6.9-6.4 (m, 2H, aromatic Hs); 6.8-6.4 (m, 2H, aromatic Hs); 6.1 (m, 2H, OCH₂O); 5.2, 5.0, 4.7 (4s, H, H-pyrrole (presence of conformational isomers); 5.15 (d, 1H, aliphatic H, H tetrahydroisoquinoline); 4.9 (m, 1H, aliphatic H, H tetrahydroisoquinoline); 4.9-4.8 (m, 1H, CH₂OH); 4.7 (m, 1H, aliphatic H, H tetrahydroisoquinoline); 3.9 (d, 1H, aliphatic H, H tetrahydroisoquinoline); 3.85 (m, 1H, aliphatic H, H tetrahydroisoquinoline); 3.7 (m, 2H, aliphatic Hs, CH₂CH₂OH); 3.75 (2s, 3H,); 3.5-3.2 (m, 2H, aliphatic Hs, CH₂CH₂OH); 3.0-2.4 (m, 2H, aliphatic H, H tetrahydroisoquinoline); 2.4-2.3 (4s, 3H, CH₃-pyrrole); 1.5, 0.95, 0.75 (4 d, 3H, CH₃-THIQ); 0.85 (broad s, 9H, Si(CH₃)₂(CH(CH₃)₂); 0.15 (m, 6H, Si(CH₃)₂(CH(CH₃)₂)

IR: ν: —OH: 3346 cm⁻¹ carboxylic acid; ν: >C═O: 1621 cm⁻¹

Step C: 1-(2-Hydroxyethyl)-N-(4-hydroxyphenyl)-2-methyl-5-(6-{[(3R)-3-methyl-3,4-dihydroisoquinolin-2(H)-yl]carbonyl}-1,3-benzodioxol-5-yl)-N-(1-methyl-1H-pyrazol-4-yl)-1H-pyrrole-3-carboxamide

The phenol function of the compound of Step B is deprotected following the process of Step D of Example 1.

### Elemental Microanalysis: (%, Theoretical:Measured)

% C=68.23:68.12; % H=5.57:5.29; % N=11.05:10.79

### High-Resolution Mass Spectrometry (ESI+):

C₃₆H₃₆N₅O₆ Empirical formula:

[M+H]⁺ calculated: 634.2662

[M+H]⁺ measured: 634.2660

### EXAMPLE 24

N-(4-Hydroxyphenyl)-5-(5-methoxy-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrazol-4-yl)-1H-pyrrole-3-carboxamide hydrochloride

### Elemental Microanalysis: (%, Theoretical:Measured)

% C=65.86:64.47; % H=6.09:5.88; % N=11.82:11.45; % Cl—=4.98:6.7

### High-Resolution Mass Spectrometry (ESI+):

C₃₉H₄₂N₆O₅ Empirical formula:

[M+H]⁺ calculated: 675.3289

[M+H]⁺ measured: 675.3287

### EXAMPLE 25

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide hydrochloride

### Elemental Microanalysis: (%, Theoretical:Measured)

% C=63.77:62.83; % H=5.63:5.83; % N=11.74:11.29; % Cl—=4.95:5.42

### EXAMPLE 26

N-(4-Hydroxyphenyl)-2-methyl-1-[2-(methylamino)ethyl]-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide hydrochloride

The procedure is in accordance with Steps A, B and C of the process of Example 30 replacing the sodium azide used in Step B by methylamine. After purification over silica gel (CH₂Cl₂/MeOH/NH₃ gradient), the compound obtained, 0.3 g (0.46 mmol), is dissolved in 5 mL of anhydrous methanol and there is added, dropwise, 0.464 mL (0.47 mmol) of 1M HCl solution in ether. The batch is stirred for 0.5 hours at ambient temperature. The precipitate thereby obtained is filtered off and dried and then dissolved in a mixture of CH₃CN/H₂O before being lyophilised at low temperature. The expected product is obtained in the form of a solid.

### Elemental Microanalysis: (%, Theoretical:Measured)

% C=65.05:64.64; % H=5.75:5.43; % N=12.3:12.3; % Cl—=5.19:5.39

### High-Resolution Mass Spectrometry (ESI+):

C₃₇H₃₈N₆O₅ Empirical formula:

[M+H]⁺ calculated: 647.2903

[M+H]⁺ measured: 647.2922

### EXAMPLE 27

1-[2-(Dimethylamino)ethyl]-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide hydrochloride

The procedure is in accordance with the process of Example 26, replacing the methylamine in Step B by dimethylamine.

### Elemental Microanalysis: (%, Theoretical:Measured)

% C=65.46:65.07; % H=5.93:5.87; % N=12.05:12.06; % Cl—=5.08:5.55

### High-Resolution Mass Spectrometry (ESI+):

C₃₈H₄₀N₆O₅ Empirical formula:

[M+H]⁺ calculated: 661.3060

[M+H]⁺ measured: 661.3045

### EXAMPLE 28

5-(5-Chloro-2-{[(3R)-3-methyl-3,4-dihydroisoquinolin-2 (1H)-yl]carbonyl}phenyl)-N-(4-hydroxyphenyl)-1,2-dimethyl-N-(pyridin-4-yl)-1H-pyrrole-3-carboxamide hydrochloride

### Elemental Microanalysis: (%, Theoretical:Measured)

% C=66.99:66.88; % H=5.14:5.28; % N=8.93:8.87; % Cl—=5.65:4.98

### High-Resolution Mass Spectrometry (ESI+):

C₃₅H₃₂ClN₄O₃ Empirical formula:

[M+H]⁺ calculated: 591.2157

[M+H]⁺ measured: 591.2178

### EXAMPLE 29

5-(5-Chloro-2-{[(3S)-3-{[2-(morpholin-4-yl)ethoxy]methyl}-3,4-dihydroisoquinolin-2 (1H)-yl]carbonyl}phenyl)-N-(4-hydroxyphenyl)-1,2-dimethyl-N-phenyl-1H-pyrrole-3-carboxamide hydrochloride
Step A: Methyl 5-(5-chloro-2-{[(3S)-3-(hydroxymethyl)-3,4-dihydroisoquinolin-2 (1H)-yl]carbonyl}phenyl)-1,2-dimethyl-1H-pyrrole-3-carboxylate

To a solution of 4.9 g of the compound of Preparation 1 in a mixture of 40 mL of dimethylformamide and 40 mL of tetrahydrofuran there are added 2.73 g (1.1 equivalents) of (3S)-3-hydroxymethyl-1,2,3,4-tetrahydroisoquinoline, 7.95 mL of diisopropylethylamine and 9.84 g (1.7 equivalents) of HATU. After stirring for 2 hours at ambient temperature, the reaction mixture is dried, taken up in ethyl acetate and then washed with 10% aqueous citric acid solution and water. The combined aqueous phases are extracted with ethyl acetate. The organic phases thereby obtained are combined, dried over Na₂SO₄, filtered and concentrated under reduced pressure. The crude product obtained is purified by chromatography over silica gel using dichloromethane and ethanol as eluants to yield the title compound.

¹H NMR: δ (500 MHz; dmso-d6; 300K): 7.6-7.4 (m, 3H, Cl-Ph); 7.2-6.9 (m, 4H, Ar(THIQ); 6.5-6.15 (broad 4s, 1H, pyrrole); 4.9-3.8 (6 d, 2H, NCH₂ THIQ); 4.85-3.6 (m, 1H, NCH THIQ); 4.2-4.0 (m, 2H, OCH₂ ester), 3.45-3.2 (3s, 3H, N—CH₃); 3.35-3.0 (3m, 2H, HOCH₂); 3.0-2.0 (m, 2H, THIQ); 2.5-2.1 (m, 3H, CH₃ pyrrole); 1.25-1.1 (m, 3H, CH₃ ester)

IR: ν —OH: 3388 cm¹, ν>C═O: 1693 cm⁻¹ (conjugated ester), ν>C═O: 1620 cm⁻¹ (amide)

Step B: Methyl 5-(5-chloro-2-{[(3S)-3-{[2-(morpholin-4-yl)ethoxy]methyl}-3,4-dihydroisoquinolin-2 (1H)-yl]carbonyl}phenyl)-1,2-dimethyl-1H-pyrrole-3-carboxylate

A solution of 1.7 g (3.2 mmol) of the compound obtained in the above Step in 20 mL of dimethylformamide is added, dropwise at 0° C., to a suspension of 0.280 g of NaH 60% in oil (2.2 equivalents) in 10 mL of dimethylformamide. After stirring for 5 minutes at ambient temperature, a suspension of 0.68 g of 4-(2-bromoethyl)morpholine hydrobromide (1.1 equivalents) is added dropwise. The reaction mixture is stirred at ambient temperature for 45 minutes. There are then added, in two steps over 20 hours, 2.2 equivalents of 4-(2-bromoethyl)morpholine hydrobromide, and then 3 equivalents of NaH 60% in oil. The reaction mixture is then poured into a mixture of 10% aqueous ammonium chloride and ethyl acetate. The resulting organic phase is successively washed with water, and then with saturated aqueous LiCl solution and brine. It is then dried over Na₂SO₄, filtered and concentrated under reduced pressure. The oil obtained is purified by chromatography over silica gel using dichloromethane and ethanol as eluants to provide the expected product.

¹H NMR: δ (500 MHz; dmso-d6; 300K): 7.6-7.3 (m, 3H, Cl-Ph); 7.2-6.85 (m, 4H, Ar(THIQ); 6.5-6.15 (broad s, 1H, pyrrole); 5.25-3.8 (m, 2H, NCH₂ THIQ); 5.05-3.75 (m, 1H, NCH THIQ); 4.2-4.0 (m, 2H, OCH₂ ester), 3.5-3.25 (m, 3H, N—CH₃); 3.6-2.75 (m, 8H, OCH₂ morpholinoethoxymethyl); 2.95-2.05 (m, 2H, THIQ); 2.55-2.15 (m, 6H, NCH₂ morpholinoethoxymethyl); 2.55-2.05 (m, 3H, CH₃ pyrrole), 1.25-1.1 (m, 3H, CH₃ ester)

IR: ν>C═O: 1694 cm⁻¹ (conjugated ester), ν>C═O: 1629 cm⁻¹ (amide)

Step C: 5-(5-Chloro-2-{[(3S)-3-{[2-(morpholin-4-yl)ethoxy]methyl}-3,4-dihydro-isoquinolin-2 (1H)-yl]carbonyl}phenyl)-1,2-dimethyl-1H-pyrrole-3-carboxylic acid

To a solution of 1.46 g of the compound of the above Step in 5 mL of dioxane there are added 5 mL of 1M aqueous LiOH solution. The batch is heated in a microwave apparatus (100 W) at 100° C. for 2 hours. The reaction mixture is poured into water and then extracted with ethyl ether. The ethereal phase is extracted again with 10 mL of water. The aqueous phases are acidified to pH 5-6 by adding saturated aqueous ammonium chloride solution and then extracted twice with dichloromethane. The dichloromethane phase is dried over Na₂SO₄, filtered and concentrated to dryness. The title product is obtained in the form of a meringue.

¹H NMR: δ (500 MHz; dmso-d6; 300K): 11.4 (broad s, 1H, CO₂H), 7.6-7.3 (m, 3H, Cl-Ph); 7.2-6.8 (m, 4H, Ar(THIQ); 6.5-6.2 (broad 4s, 1H, pyrrole); 5.25-3.75 (m, 2H, NCH₂ THIQ); 5.05-3.7 (3m, 1H, NCH THIQ); 3.5-3.2 (3s, 3H, N—CH₃); 3.6-2.7 (m, 8H, OCH₂ morpholinoethoxymethyl); 2.5-2.2 (m, 6H, NCH₂ morpholinoethoxymethyl); 3.0-2.0 (m, 2H, THIQ), 2.5-2.0 (3s, 3H, CH₃ pyrrole)

IR: ν —OH: 3300-2200 cm¹, ν>C═O: 1697-1662 cm⁻¹ (double band, carboxylic acid), ν>C═O: 1628 cm⁻¹ (amide)

Step D: N-(4-{[tert-Butyl(dimethyl)silyl]oxy}phenyl)-5-(5-chloro-2-{[(3S)-3-{[2-(morpholin-4-yl)ethoxy]methyl}-3,4-dihydroisoquinolin-2(H)-yl]carbonyl}phenyl)-1,2-dimethyl-N-phenyl-1H-pyrrole-3-carboxamide

The acid obtained in Step C (1.48 g) is suspended in 15 mL of 1,2-dichloroethane. 1.1 equivalents of 1-chloro-N,N,2-trimethylpropenylamine are added thereto. After stirring for 1 hour at ambient temperature, there are added 15 mL of toluene and 1.05 equivalents of the compound of Preparation 2″. The reaction mixture is heated for 14 hours at 110° C. and then dried. The crude product thereby obtained is purified by chromatography over silica gel using dichloromethane and ethanol as eluants to yield the expected product.

¹H NMR: δ (500 MHz; dmso-d6; 300K): 7.55-7.25 (m, 3H, Cl-Ph); 7.25-6.6 (m, 9H, Ar(THIQ)+phenyl); 6.8-6.5 (m, 2H, phenoxy); 7.0-6.6 (m, 2H, phenoxy); 5.7-5.05 (broad 4 s, 1H, pyrrole); 5.2-3.6 (8d, 2H, THIQ); 5.05-3.6 (4m, 1H, NCH THIQ), 3.6-2.9 (m, 8H, aliphatic morpholinoethoxymethyl); 3.4-3.2 (broad 3s, 3H, N—CH₃); 3.0-2.1 (m, 2H, THIQ), 2.35 (m, 2H, NCH₂ morpholinoethoxymethyl); 2.4-2.2 (m, 4H, NCH₂ morpholine); 2.4-2.15 (broad 3s, 3H, CH₃ pyrrole); 0.8 (broad 2s, 9H, SiC(CH₃)₃); 0.1 (4s, 6H, SiCH₃)

IR: ν>C═O: 1635-1595 cm⁻¹ (double band), ν Si—O: 1117 cm⁻¹, ν Si—C: 837 cm⁻¹

Step E: 5-(5-Chloro-2-{[(3S)-3-{[2-(morpholin-4-yl)ethoxy]methyl}-3,4-dihydroisoquinolin-2 (1H)-yl]carbonyl}phenyl)-N-(4-hydroxyphenyl)-1,2-dimethyl-N-phenyl-1H-pyrrole-3-carboxamide hydrochloride

To a solution of 0.7 g of the compound of Step D in 5 mL of methanol there is added 0.92 mL of a 1M solution of potassium hydroxide in methanol. After stirring for 1 hour 10 minutes at ambient temperature, saturated aqueous sodium bicarbonate solution is added. The product which precipitates is extracted with ethyl acetate and then washed with water and brine. The resulting aqueous phases are extracted again with ethyl acetate. The organic phases thereby obtained are dried over Na₂SO₄, filtered and concentrated under reduced pressure. The crude product obtained is purified by chromatography over silica gel using dichloromethane and ammonia-in-ethanol as eluants to provide 0.536 g of the title product, in the form of the base. The latter is dissolved in acetonitrile and converted into salt form using 1M aqueous HCl solution. After a lyophilisation step, the expected product is obtained in the form of a solid.

### Elemental Microanalysis: (%, Measured(Theoretical))

% C=66.4 (66.75);% H=5.98 (5.87);% N=7.38 (7.41);% Cl=9.42 (9.38);% Cl⁻=4.57 (4.69)

### EXAMPLE 30

1-(2-Aminoethyl)-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide hydrochloride
Step A: 2-{3-[(4-{[tert-Butyl(dimethyl)silyl]oxy}phenyl)(1-methyl-1H-pyrazol-4-yl)carbamoyl]-2-methyl-5-(6-{[(3R)-3-methyl-3,4-dihydroisoquinolin-2 (1H)-yl]carbonyl}-1,3-benzodioxol-5-yl)-1H-pyrrol-1-yl}ethyl methanesulphonate

A solution of 4.65 g of the compound obtained in Step B of Example 23 (6.22 mmol) in 100 mL of anhydrous THF is placed at 0° C. There are successively added thereto 3.5 mL (12.44 mmol) of triethylamine and then, dropwise, 0.722 mL of methanesulphonic chloride (9.33 mmol) dissolved in 20 mL of THF. The batch is then stirred at ambient temperature for 2 hours. The reaction mixture is hydrolysed by addition of saturated aqueous NaHCO₃ solution and then extracted twice with ethyl acetate. The organic phases are combined, dried over MgSO₄ and concentrated to dryness. The expected compound is obtained in the form of a glassy solid.

¹H NMR: δ (500 MHz; dmso-d6; 300K): 7.7-7.45 (s, 1H, H pyrazole); 7.1-6.9 (s, 1H, H pyrazole); 7.2-6.4 (m, 8H, aromatic Hs); 6.95-6.65 (m, 2H, aromatic Hs); 6.1 (m, 2H, OCH₂O); 5.2, 4.6 (4s, H, H-pyrrole (presence of conformational isomers); 4.9, 4.6, 3.9, 3.8 (m, 1H), 5.05-3.75 (m, 1H, aliphatic H, H tetrahydroisoquinoline); 4.3-4.05 (m, 2H, aliphatic Hs, CH₂CH₂OSO₂CH₃); 4.2-3.95 (m, 2H, aliphatic Hs, CH₂CH₂OSO₂CH₃); 3.75-2.7 (2s, 3H, CH₃-pyrazole); 3.0 (several s, 3H, CH₂OSO₂CH₃); 3.05-2.5 (several m, 2H, aliphatic Hs, H tetrahydroisoquinoline); 2.45-2.3 (several s, 3H, CH₃-pyrrole); 1.05, 0.75 (several d, 3H, CH₃-tetraisoquinoline); 0.9 (several s, 9H, Si(CH₃)₂(CH(CH₃)₂); 0.1 (m, 6H, Si(CH₃)₂(CH(CH₃)₂)

IR: ν: >C═O: 1626 cm¹, ν: —SO₂: 1349 and 1172 cm⁻¹

Step B: 1-(2-Azidoethyl)-N-(4-{[tert-butyl(dimethyl)silyl]oxy}phenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide

To a solution of 2 g (2.42 mmol) of compound of Step A in 20 mL of anhydrous DMF there is added 0.472 g (7.26 mmol) of sodium azide. The batch is stirred at 65° C. for 4 hours 30 minutes. The reaction mixture is poured into saturated aqueous NaHCO₃ solution and then extracted twice with ethyl acetate. The organic phases are combined and washed with saturated aqueous LiCl solution, dried over MgSO₄ and concentrated to dryness. After purification over silica gel using dichloromethane and ammonia-in-methanol as eluants, the expected product is obtained in the form of a foam.

¹H NMR: δ (500 MHz; dmso-d6; 300K): 7.65-7.45 (several s, 1H, H pyrazole); 7.1-6.9 (several s, 1H, H pyrazole); 7.3-6.4 (several m, 6H, aromatic Hs); 7.1-6.9 (several s, 1H, aromatic Hs); 6.8, 6.5 (2m, 2H, aromatic Hs); 6.1 (several s, 2H, OCH₂O); 5.25-4.65 (several s, 1H, H-pyrrole (presence of conformational isomers); 4.9, 4.6, 3.8 (several m, 1H); 5.05-3.7 (several m, 4H, aliphatic Hs, H-THIQ+CH₂CH₂N₃); 3.7 (several s, 3H, H CH₃-pyrazole); 3.5-3.25 (m, 2H, CH₂CH₂N₃); 3.1-2.4 (several m, 3H, CH₃-pyrrole); 2.45-2.3 (several s, 3H, CH₃-pyrrole); 1.0-0.75 (several d, 3H, CH₃-THIQ); 0.9 (several s, 9H, Si(CH₃)₂(CH(CH₃)₂); 0.1 (several s, 6H, Si(CH₃)₂(CH(CH₃)₂)

IR: ν:—N═N═N: 2100 cm¹, ν: >C═O: 1630 cm¹, ν: —Si—O—: 1035 cm⁻¹

Step C: 1-(2-Azidoethyl)-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide

To a solution of 1.12 g of the compound obtained in Step B (14.49 mmol) in 5 mL of methanol there are added, dropwise, 7.24 mL of 1M methanolic potassium hydroxide solution (72.45 mmol). The batch is stirred at ambient temperature for 3 hours. The reaction mixture is then concentrated to dryness, taken up in dichloromethane, hydrolysed with saturated aqueous NaHCO₃ solution and then extracted twice with dichloromethane. The organic phases are then combined, washed with water, dried over MgSO₄ and concentrated to dryness. The expected product is obtained in the form of a foam and used directly in the next Step.

¹H NMR: δ (500 MHz; dmso-d6; 300K): 9.60 ((s, 1H, CH₂CH₂OH); 7.65-7.45 (several s, 1H, H pyrazole); 7.1-6.9 (several s, 1H, H pyrazole); 7.3-6.4 (several m, 6H, aromatic Hs); 7.1-6.9 (several s, 1H, aromatic Hs); 6.8, 6.5 (2m, 2H, aromatic Hs); 6.15 (several s, 2H, OCH₂O); 5.35-4.8 (several s, 1H, H-pyrrole (presence of conformational isomers); 4.9, 4.6, 3.8 (several m, 1H, aliphatic H, H tetrahydroisoquinoline); 3.7 (several s, 3H, H CH₃-pyrazole); 3.5-3.25 (m, 2H, CH₂CH₂N₃); 3.1-2.4 (several m, 3H, CH₃-pyrrole); 2.45-2.3 (several s, 3H, H THIQ); 2.45-2.3 (several s, 3H, CH₃-pyrrole); 1.0-0.75 (several d, 3H, CH₃-THIQ)

IR: ν:—OH: 3171 cm¹, ν:—N═N═N: 2100 cm⁻¹, ν: >C═O: 1617 cm⁻¹

Step D: 1-(2-Aminoethyl)-N-(4-hydroxyphenyl)-2-methyl-5-(6-{[(3R)-3-methyl-3,4-dihydroisoquinolin-2(H)-yl]carbonyl}-1,3-benzodioxol-5-yl)-N-(1-methyl-1H-pyrazol-4-yl)-1H-pyrrole-3-carboxamide hydrochloride

To a solution of 1.27 g of the compound obtained in the above Step (1.93 mmol) in 15 mL of anhydrous ethanol there is added 0.154 g of Pd/C 10%. The batch is degassed for 0.5 hours. It is then stirred for 12 hours at ambient temperature under hydrogen pressure (1.5 bar). The reaction mixture is then filtered and then concentrated to dryness. After purification by chromatography over silica gel (CH₂Cl₂/MeOH/NH₃ gradient), an oily residue is obtained. The residue is dissolved in 10 mL of anhydrous ethanol and then converted into salt form by adding two molar equivalents of a 1N solution of HCl in ethanol. The product is then dried before being dissolved in a minimum of a mixture of water and acetonitrile. After a lyophilisation step at low temperature, the expected product is obtained in the form of a solid.

### Elemental Microanalysis: (%, Theoretical:Measured)

% C=64.62:63.84; % H=5.57:5.55; % N=12.56:12.36; % Cl—=5.3:5.56

### High-Resolution Mass Spectrometry (ESI+):

C₃₆H₃₆N₆O₅ Empirical formula:

[M+H]⁺ calculated: 633.2820

[M+H]⁺ measured: 633.2808

### EXAMPLE 31

N-(4-Hydroxyphenyl)-5-(5-methoxy-2-{[(3R)-3-methyl-3,4-dihydroisoquinolin-2 (1H)-yl]carbonyl}phenyl)-1,2-dimethyl-N-(1-methyl-1H-pyrazol-4-yl)-1H-pyrrole-3-carboxamide

### Elemental Microanalysis: (%, Theoretical:Measured)

% C=71.29:70.17; % H=5.98:5.98; % N=11.88:11.49

### High-Resolution Mass Spectrometry (ESI+):

C₃₅H₃₆N₅O₄ Empirical formula:

[M+H]⁺ calculated: 590.2762

[M+H]⁺ measured: 590.2778

### EXAMPLE 3

Phenyl (4-{4-[(4-hydroxyphenyl)(methyl)carbamoyl]-1,5-dimethyl-1H-pyrrol-2-yl}-3-{[(3R)-3-methyl-3,4-dihydroisoquinolin-2 (1H)-yl]carbonyl}benzyl)-carbamate
Step A: N-(4-Benzyloxyphenyl)-N,1,2-trimethyl-pyrrole-3-carboxamide

The process is analogous to that described in Step A of Example 45.

Step B: tert-Butyl [4-(4-{[4-(benzyloxy)phenyl](methyl)carbamoyl}-1,5-dimethyl-1H-pyrrol-2-yl)-3-{[(3R)-3-methyl-3,4-dihydroisoquinolin-2 (1H)-yl]carbonyl}benzyl]-carbamate

The compound of Preparation II (1.5 g; 2.96 mmol) and the compound of Step A (1.16 g; 3.44 mmol) are dissolved in dimethylacetamide (25 mL) and then nitrogen is bubbled through the solution for 5 minutes. Potassium acetate (0.586 g; 5.92 mmol) and bis(triphenylphosphine)palladium(II) dichloride (0.2 g; 0.295 mmol) are added to the mixture which is then heated to 100° C. and, after stirring for 20 minutes, water (10 μL) is added. Stirring at 100° C. under a nitrogen atmosphere is continued for 2 hours more. The reaction mixture is allowed to cool to ambient temperature and is then evaporated to a volume of 15 mL. The resulting mixture is filtered over a short Celite column and then purified by preparative HPLC using water-TFA and acetonitrile as eluants. The pH of the appropriate fractions is adjusted to 7 with NaHCO₃, and then the acetonitrile is evaporated off under reduced pressure. The aqueous residue is extracted with DCM. The organic phase is washed with brine, dried over Na₂SO₄ and evaporated to dryness to yield the title compound.

### High-Resolution Mass Spectrometry (ESI+):

C₄₀H₄₁N₅O₄ Empirical formula:

[M+H]⁺ calculated: 713.3705

[M+H]⁺ measured: 713.3709

Step C: 5-[4-(Aminomethyl)-2-{[(3R)-3-methyl-3,4-dihydroisoquinolin-2(H)-yl]-carbonyl}phenyl]-N-[4-(benzyloxy)phenyl]-N,1,2-trimethyl-1H-pyrrole-3-carboxamide hydrochloride

The compound of the above Step (1.18 g, 1.66 mmol) is dissolved/suspended in 4M HCl in dioxane (20 mL). The heterogeneous mixture is stirred for 30 minutes at ambient temperature and then evaporated to dryness to yield 1.19 g of the title compound which is used in the next Step without purification.

Step D: Phenyl (4-{4-[(4-hydroxyphenyl)(methyl)carbamoyl]-1,5-dimethyl-1H-pyrrol-2-yl}-3-{[(3R)-3-methyl-3,4-dihydroisoquinolin-2 (1H)-yl]carbonyl}benzyl)carbamate

The title compound is obtained starting from the compound of the above Step in accordance with the protocol of Step D of Example 45.

### Elemental Microanalysis: (%, Measured(Theoretical))

% C=72.83 (72.88);% H=6.01 (5.96);% N=8.02 (8.72)

### High-Resolution Mass Spectrometry (ESI+):

C₃₉H₃₈N₄O₅ Empirical formula:

[M+H]⁺ calculated: 643.2922

[M+H]⁺ measured: 643.2916

### EXAMPLE 33

5-(5-Chloro-2-{[(3S)-3-(morpholin-4-ylmethyl)-3,4-dihydroisoquinolin-2 (1H)-yl]carbonyl}phenyl)-N-(4-fluorophenyl)-N-(4-hydroxyphenyl)-1,2-dimethyl-1H-pyrrole-3-carboxamide hydrochloride

### Elemental Microanalysis: (%, Measured(Theoretical))

% C=65.75 (65.84);% H=5.38 (5.39);% N=7.62 (7.68);% Cl=9.77 (9.72);% Cl—=4.84 (4.86)

### EXAMPLE 34

5-(5-Chloro-2-{[(3S)-3-(morpholin-4-ylmethyl)-3,4-dihydroisoquinolin-2 (1H)-yl]carbonyl}phenyl)-N-(3-fluorophenyl)-N-(4-hydroxyphenyl)-1,2-dimethyl-1H-pyrrole-3-carboxamide hydrochloride

### Elemental Microanalysis: (%, Measured(Theoretical))

% C=65.61 (65.84);% H=5.09 (5.39);% N=7.76 (7.68);% Cl—=4.83 (4.86)

### EXAMPLE 35

N-(4-Hydroxyphenyl)-N,1,2-trimethyl-5-(2-{[(3R)-3-methyl-3,4-dihydroisoquinolin-2 (1H)-yl]carbonyl}-4-{[(phenoxyacetyl)amino]methyl}phenyl)-1H-pyrrole-3-carboxamide

The title compound is obtained in accordance with the process of Example 32 using 2-phenoxyacetyl chloride as acylating agent in Step D.

### Elemental Microanalysis: (%, Measured(Theoretical))

% C=72.53 (73.15);% H=5.76 (6.14);% N=8.31 (8.53)

### High-Resolution Mass Spectrometry (ESI+):

C₄₀H₄₀N₄O₅ Empirical formula:

[M+H]⁺ calculated: 657.3079

[M+H]⁺ measured: 657.3061

### EXAMPLE 36

5-(4-{[(Ethylcarbamoyl)amino]methyl}-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 32 using ethyl isocyanate as acylating agent in Step D.

### Elemental Microanalysis: (%, Measured(Theoretical))

% C=70.88 (70.8);% H=6.02 (6.62);% N=11.17 (11.8)

### High-Resolution Mass Spectrometry (ESI+):

C₃₅H₃₉N₅O₄ Empirical formula:

[M+H]⁺ calculated: 594.3100

[M+H]⁺ measured: 594.3083

### EXAMPLE 37

5-(4-{[(Benzylcarbamoyl)amino]methyl}-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 32 using benzyl isocyanate as acylating agent in Step D.

### Elemental Microanalysis: (%, Measured(Theoretical))

% C=73.21 (73.26);% H=5.98 (6.3);% N=10.23 (10.68)

### High-Resolution Mass Spectrometry (ESI+):

C₄₀H₄₁N₅O₄ Empirical formula:

[M+H]⁺ calculated: 656.3239

[M+H]⁺ measured: 656.3256

### EXAMPLE 38

5-(5-Chloro-2-{[(3S)-3-(hydroxymethyl)-3,4-dihydroisoquinolin-2 (1H)-yl]carbonyl}phenyl)-N-(4-hydroxyphenyl)-1,2-dimethyl-N-phenyl-1H-pyrrole-3-carboxamide

### Elemental Microanalysis: (%, Measured(Theoretical))

% C=70.97 (71.34);% H=5.2 (5.32);% N=6.96 (6.93)

### EXAMPLE 39

N-(4-Hydroxyphenyl)-1,2-dimethyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide

### Elemental Microanalysis: (%, Measured(Theoretical))

% C=69.81 (69.64);% H=5.6 (5.51);% N=11.55 (11.6)

### High-Resolution Mass Spectrometry (ESI+):

C₃₅H₃₄N₅O₅ Empirical formula:

[M+H]⁺ calculated: 604.2554

[M+H]⁺ measured: 604.2565

### EXAMPLE 40

Phenyl (3-{4-[(4-hydroxyphenyl)(methyl)carbamoyl]-1,5-dimethyl-1H-pyrrol-2-yl}-4-{[(3R)-3-methyl-3,4-dihydroisoquinolin-2 (1H)-yl]carbonyl}benzyl)-carbamate
Step A: N-(4-Benzyloxyphenyl)-N,1,2-trimethyl-pyrrole-3-carboxamide

The process is analogous to that described in Step A of Example 45.

Step B: 5-[5-(Aminomethyl)-2-{[(3R)-3-methyl-3,4-dihydroisoquinolin-2(H)-yl]-carbonyl}phenyl]-N-[4-(benzyloxy)phenyl]-N,1,2-trimethyl-1H-pyrrole-3-carboxamide hydrochloride

The compound of Preparation III (1.84 g; 3.99 mmol) and the compound of Step A (1.60 g; 4.48 mmol) are dissolved in dimethylacetamide (20 mL) and then nitrogen is bubbled through the solution for 5 minutes. Potassium acetate (0.78 g; 7.48 mmol) and bis(triphenylphosphine)palladium(II) dichloride (0.28 g; 0.4 mmol) are added to the mixture which is then heated to 105° C. and, after stirring for 10 minutes, water (11 μL) is added. Stirring at 105° C. under a nitrogen atmosphere is continued for 6 hours more. The reaction mixture is allowed to cool to ambient temperature, the resulting mixture is filtered over a short Celite column and is then purified by preparative HPLC using water-TFA and acetonitrile as eluants. The pH of the appropriate fractions is adjusted to 7 with NaHCO₃ and then the acetonitrile is evaporated off under reduced pressure. The solid precipitate is removed by filtration and then dried (5 mbar, 45° C., 16 hours) to form tert-butyl [3-(4-{[4-(benzyloxy)phenyl](methyl)carbamoyl}-1,5-dimethyl-1H-pyrrol-2-yl)-4-{[(3R)-3-methyl-3,4-dihydroisoquinolin-2 (1H)-yl]carbonyl}benzyl]carbamate which is dissolved/suspended in 4M HCl in dioxane (35 mL). The heterogeneous mixture is stirred for 30 minutes at ambient temperature and then evaporated to dryness to yield the title compound which is used in the next Step without purification.

### High-Resolution Mass Spectrometry (ESI+):

C₃₉H₄₀N₄O₃ Empirical formula:

[M+H]⁺ calculated: 613.3180

[M+H]⁺ measured: 613.3194

IR: ν: N—H+: 2854 cm⁻¹; >C═O: 1621 cm⁻¹; C—O—C: 1234, 1120, 1011 cm⁻¹

Step C: Phenyl (3-{4-[(4-hydroxyphenyl)(methyl)carbamoyl]-1,5-dimethyl-1H-pyrrol-2-yl}-4-{[(3R)-3-methyl-3,4-dihydroisoquinolin-2 (1H)-yl]carbonyl}benzyl)carbamate

The title compound is obtained starting from the compound of the above Step in accordance with the process of Step D of Example 45.

Elemental microanalysis: (%, measured(theoretical))

% C=73.2 (72.88);% H=5.83 (5.96);% N=8.13 (8.72)

### High-Resolution Mass Spectrometry (ESI+):

C₃₉H₃₈N₄O₅ Empirical formula:

[M+H]⁺ calculated: 643.2922

[M+H]⁺ measured: 643.2902

### EXAMPLE 41

N-(3-Fluorophenyl)-N-(4-hydroxyphenyl)-1,2-dimethyl-5-(6-{[(3R)-3-Methyl-3,4-dihydroisoquinolin-2 (1H)-yl]carbonyl}-1,3-benzodioxol-5-yl)-1H-pyrrole-3-carboxamide

### Elemental Microanalysis: (%, Measured(Theoretical))

% C=71.44 (71.95);% H=4.95 (5.22);% N=6.8 (6.8)

### High-Resolution Mass Spectrometry (ESI+):

C₃₇H₃₃FN₃O₅ Empirical formula:

[M+H]⁺ calculated: 618.2399

[M+H]⁺ measured: 618.2392

### EXAMPLE 42

N-(4-Hydroxyphenyl)-1,2-dimethyl-5-(6-{[(3R)-3-methyl-3,4-dihydroisoquinolin-2 (1H)-yl]carbonyl}-1,3-benzodioxol-5-yl)-N-(1-methyl-1H-pyrrolo[2,3-B]pyridin-5-yl)-1H-pyrrole-3-carboxamide

### Elemental Microanalysis: (%, Measured(Theoretical))

% C=71.32 (71.65);% H=5.17 (5.4);% N=10.67 (10.71)

### High-Resolution Mass Spectrometry (ESI+):

C₃₉H₃₆N₅O₅ Empirical formula:

[M+H]⁺ calculated: 654.2711

[M+H]⁺ measured: 654.2710

### EXAMPLE 43

N-(4-Hydroxyphenyl)-1,2-dimethyl-5-(6-{[(3R)-3-methyl-3,4-dihydroisoquinolin-2 (1H)-yl]carbonyl}-1,3-benzodioxol-5-yl)-N-phenyl-1H-pyrrole-3-carboxamide

### Elemental Microanalysis: (%, Measured(Theoretical))

% C=73.9 (74.11);% H=5.16 (5.55);% N=6.81 (7.01)

### High-Resolution Mass Spectrometry (ESI+):

C₃₇H₃₄N₃O₅ Empirical formula:

[M+H]⁺ calculated: 600.2493

[M+H]⁺ measured: 600.2495

### EXAMPLE 44

N-(4-Hydroxyphenyl)-1,2-dimethyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Measured(Theoretical))

% C=66.41 (66.62);% H=5.08 (5.59);% N=10.85 (10.84);% Cl—=4.68 (4.57)

### High-Resolution Mass Spectrometry (ESI+):

C₄₃H₄₂N₆O₆ Empirical formula:

[M+H]⁺ calculated: 739.3239

[M+H]⁺ measured: 739.3246

### EXAMPLE 45

Phenyl [2-(3-{4-[(4-hydroxyphenyl)(methyl)carbamoyl]-1,5-dimethyl-1H-pyrrol-2-yl}-4-{[(3R)-3-methyl-3,4-dihydroisoquinolin-2 (1H)-yl]carbonyl}-phenyl)ethyl]carbamate
Step A: N-(4-Benzyloxyphenyl)-N,1,2-trimethyl-pyrrole-3-carboxamide

To a suspension of 1,2-dimethylpyrrole-3-carboxylic acid (2.085 g; 15 mmol) in 1,2-dichloroethane (40 mL) there is added 1-chloro-N,N,2-trimethyl-prop-1-en-1-amine (2.37 mL; 17.9 mmol), and the solution formed is stirred for 1 hour. To the resulting solution there is added, dropwise, an ice-cold solution of 4-benzyloxy-N-methyl-aniline (3.73 g; 15 mmol) and N-ethyl-N-isopropyl-propan-2-amine (7.75 mL; 45 mmol) in 1,2-dichloroethane (40 mL). The reaction mixture is stirred at ambient temperature for 1 hour; it is then diluted with dichloromethane (250 mL) and washed with water (2×30 mL), dried over Na₂SO₄ and evaporated. The crude product is triturated with diethyl ether and the solid formed is filtered off to yield the title compound.

IR: ν: >C═O: 1616 cm⁻¹; amide: 1508 cm⁻¹; C—O—C: 1230, 1172, 1009 cm⁻¹

Step B: tert-Butyl {2-[3-(4-{[4-(benzyloxy)phenyl](methyl)carbamoyl}-1,5-dimethyl-1H-pyrrol-2-yl)-4-{[(3R)-3-methyl-3,4-dihydroisoquinolin-2 (1H)-yl]carbonyl}phenyl]ethyl}-carbamate

The compound of Preparation I (0.869 g; 1.67 mmol) and the compound of Step A (0.558 g; 1.67 mmol) are dissolved in dimethylacetamide (8 mL) and then nitrogen is bubbled through the solution for 5 minutes. Potassium acetate (0.33 g; 3.34 mmol) and bis(triphenylphosphine)palladium(II) dichloride (0.117 g; 0.167 mmol) are added to the mixture which is then heated to 140° C. and, after stirring for 10 minutes, water (80 μL) is added. Stirring at 140° C. under a nitrogen atmosphere is continued for 16 hours more. The reaction mixture is allowed to cool to ambient temperature and then evaporated. The residue is partitioned between dichloromethane (100 mL) and water (20 mL). The organic phase is washed with water (20 mL), dried over Na₂SO₄ and evaporated. The residue was purified by preparative HPLC using water-TFA and acetonitrile as eluants. The pH of the appropriate fractions is adjusted to 12 using aqueous NaOH solution and then the acetonitrile is evaporated off under reduced pressure. The aqueous residue is extracted with dichloromethane (2×100 mL). The organic phase is dried over Na₂SO₄ and evaporated to dryness to yield the title compound.

### High-Resolution Mass Spectrometry (ESI+):

C₄₅H₅₀N₄O₅ Empirical formula:
[M+H]⁺ calculated: 727.3861
[M+H]⁺ measured: 727.3852

IR: ν: >C═O: 1705, 1618 cm⁻¹; amide: 1509 cm⁻¹; C—O—C: 1242, 1166, 1012 cm⁻¹

Step C: 5-[5-(2-Aminoethyl)-2-{[(3R)-3-methyl-3,4-dihydroisoquinolin-2(H)-yl]-carbonyl}phenyl]-N-[4-(benzyloxy)phenyl]-N,1,2-trimethyl-1H-pyrrole-3-carboxamide hydrochloride

The compound of Step B (0.35 g; 0.48 mmol) was dissolved/suspended in 4M HCl in dioxane (3 mL). The heterogeneous mixture is stirred for 30 minutes at ambient temperature and then evaporated to dryness to yield the title compound which is used in next Step without being otherwise purified.

### High-Resolution Mass Spectrometry (ESI+):

C₄₀H₄₂N₄O₃ Empirical formula:

[M+H]⁺ calculated: 627.3337

[M+H]⁺ measured: 627.3309

IR: ν: C—H: 2931 cm⁻¹; >C═O: 1608 cm⁻¹; amide: 1508 cm⁻¹; C—O—C: 1233, 1012 cm⁻¹

Step D: Phenyl 2-(3-{4-[(4-hydroxyphenyl)(methyl)carbamoyl]-1,5-dimethyl-1H-pyrrol-2-yl}-4-{[(3R)-3-methyl-3,4-dihydroisoquinolin-2 (1H)-yl]carbonyl}phenyl)ethyl]-carbamate

The compound of Step C (0.10 g; 0.15 mmol) is dissolved in dichloromethane. Phenyl chloroformate (0.027 g; 0.17 mmol) and diisopropylethylamine (0.097 g, 0.75 mmol) are added thereto. The reaction mixture is stirred at ambient temperature for 30 minutes. After dilution with dichloromethane (100 mL), the organic phase is washed with water (20 mL), evaporated and concentrated. The residue is then dissolved in ethanol and a Pd/C catalyst is added (10 mg). The reaction mixture is hydrogenated at atmospheric pressure at ambient temperature. When the reaction is complete, the catalyst is removed by filtration, the filtrate is concentrated and the crude product is purified by chromatography over silica gel using dichloromethane and methanol as eluants, to yield the title product.

### Elemental Microanalysis: (%, Measured(Theoretical))

% C=72.36 (73.15);% H=6.15 (6.14);% N=8.14 (8.53)

### High-Resolution Mass Spectrometry (ESI+):

C₄₀H₄₀N₄O₅ Empirical formula:

[M+H]⁺ calculated: 657.3077

[M+H]⁺ measured: 657.3062

### EXAMPLE 46

N-(4-Hydroxyphenyl)-N,1,2-trimethyl-5-(2-{[(3R)-3-methyl-3,4-dihydroisoquinolin-2 (1H)-yl]carbonyl}-5-{2-[(phenoxyacetyl)amino]ethyl}phenyl)-1H-pyrrole-3-carboxamide

The title compound is obtained in accordance with the process of Example 45 using 2-phenoxyacetyl chloride as acylating agent in Step D.

### Elemental Microanalysis: (%, Measured(Theoretical))

% C=72.74 (73.41);% H=6.38 (6.31);% N=7.65 (8.35)

### High-Resolution Mass Spectrometry (ESI+):

C₄₁H₄₂N₄O₅ Empirical formula:

[M+H]⁺ calculated: 671.3235

[M+H]⁺ measured: 671.3226

### EXAMPLE 47

N-(4-Hydroxyphenyl)-1,2-dimethyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Measured(Theoretical))

% C=64.25 (64.59);% H=5.4 (5.7);% N=11.41 (11.59);% Cl—=4.93 (4.89)

### EXAMPLE 48

N-(4-Hydroxyphenyl)-N-(2-methoxypyrimidin-5-yl)-1,2-dimethyl-5-(6-{[(3R)-3-methyl-3,4-dihydroisoquinolin-2 (1H)-yl]carbonyl}-1,3-benzodioxol-5-yl)-1H-pyrrole-3-carboxamide

### Elemental Microanalysis: (%, Measured(Theoretical))

% C=68.07 (68.45);% H=5 (5.27);% N=10.74 (11.09)

### EXAMPLE 49

N-(4-Hydroxyphenyl)-1,2-dimethyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide dihydrochloride
Step A: N-(4-{[tert-Butyl(dimethyl)silyl]oxy}phenyl)-1,2-dimethyl-N-(1-methyl-1H-pyrrolo[2,3-b]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

The title compound is obtained in accordance with the process of Example 1 using the acid of Preparation 11 and (3S)-3-(4-morpholinylmethyl)-1,2,3,4-tetrahydroisoquinoline (see Preparation 3′) in Step A, and the compound of Preparation 11″ in Step C.

Step B: N-(4-Hydroxyphenyl)-1,2-dimethyl-N-(1-methyl-2,3-dihydro-1H-pyrrolo[2,3-b]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide dihydrochloride

To a solution of 0.21 g (0.2 mmol) of the compound of Step A in 3 mL of acetic acid there are added 85 mg of sodium cyanoborohydride (5 equivalents). The reaction mixture is stirred for 3 hours at ambient temperature and then overnight at 50° C. There are then added 2.6 equivalents of sodium cyanoborohydride and the reaction mixture is heated at 50° C. for 3 hours. The operation is repeated for a second time (addition of 2.6 equivalents of sodium cyanoborohydride and then heating at 50° C. for 3 hours). After coevaporation of the acetic acid in the presence of toluene, the residue is taken up in 3 mL of methanol. The pH of the solution is adjusted to 12 using 1M methanolic potassium hydroxide solution. After stirring overnight at ambient temperature, the reaction mixture is poured into saturated aqueous sodium bicarbonate solution and extracted with dichloromethane. The resulting organic phase is washed with brine, dried over Na₂SO₄, filtered and concentrated under reduced pressure. The product obtained is purified by chromatography over silica gel (dichloromethane/ethanol/ammonia 94/6/0.6) and then over a Lichroprep RP18 column (water/acetonitrile/trifluoroacetic acid). After evaporation of the acetonitrile, the product is neutralised with sodium bicarbonate. It is then extracted with ethyl acetate. The organic phase is dried over Na₂SO₄, filtered and concentrated under reduced pressure. The residue obtained is dissolved in acetonitrile and converted into salt form with 1M aqueous HCl solution. After lyophilisation, the title product is obtained in the form of a solid.

### High-Resolution Mass Spectrometry (ESI+):

C₄₃H₄₄N₆O₆ Empirical formula:

[M+H]⁺ calculated: 741.3395

[M+H]⁺ measured: 741.3400

### EXAMPLE 50

N-(4-Hydroxyphenyl)-N-(2-methoxypyrimidin-5-yl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### High-Resolution Mass Spectrometry (ESI+):

C₄₀H₄₀N₆O₇ Empirical formula:
[M+H]⁺ calculated: 717.3031
[M+H]⁺ measured: 717.3031

### EXAMPLE 51

5-(5-{[(Benzylcarbamoyl)amino]methyl}-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 40 using benzyl isocyanate as acylating agent in Step C.

### Elemental Microanalysis: (%, Measured(Theoretical))

% C=73.39 (73.26);% H=6.16 (6.3);% N=10.08 (10.68)

### High-Resolution Mass Spectrometry (ESI+):

C₄₀H₄₁N₅O₄ Empirical formula:

[M+H]⁺ calculated: 656.3239

[M+H]⁺ measured: 656.3226

### EXAMPLE 52

5-(5-{[(Ethylcarbamoyl)amino]methyl}-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 40 using ethyl isocyanate as acylating agent in Step C.

### Elemental Microanalysis: (%, Measured(Theoretical))

% C=71.4 (70.8);% H=6.41 (6.62);% N=11.24 (11.8)

### High-Resolution Mass Spectrometry (ESI+):

C₃₅H₃₉N₅O₄ Empirical formula:

[M+H]⁺ calculated: 594.3082

[M+H]⁺ measured: 594.3069

### EXAMPLE 53

N-(4-Hydroxyphenyl)-N,1,2-trimethyl-5-(2-{[(3R)-3-methyl-3,4-dihydroisoquinolin-2 (1H)-yl]carbonyl}-5-{[(phenoxyacetyl)amino]methyl}phenyl)-1H-pyrrole-3-carboxamide

The title compound is obtained in accordance with the process of Example 40 using 2-phenoxyacetyl chloride as acylating agent in Step C.

### Elemental Microanalysis: (%, Measured(Theoretical))

% C=72.32 (73.15);% H=6.21 (6.14);% N=7.84 (8.53)

### High-Resolution Mass Spectrometry (ESI+):

C₄₀H₄₀N₄O₅ Empirical formula:

[M+H]⁺ calculated: 657.3079

[M+H]⁺ measured: 657.3105

### EXAMPLE 54

N-(4-Hydroxyphenyl)-1,2-dimethyl-5-(2-{[(3R)-3-methyl-3,4-dihydroisoquinolin-2 (1H)-yl]carbonyl}-5-{[(phenoxyacetyl)amino]methyl}phenyl)-N-(1-methyl-1H-pyrrolo[2,3-B]pyridin-5-yl)-1H-pyrrole-3-carboxamide

### Elemental Microanalysis: (%, Measured(Theoretical))

% C=73.87 (73.04);% H=5.47 (5.74);% N=10.26 (10.87)

### EXAMPLE 55

5-(5-[2-({[2-(4-Fluorophenyl)ethyl]sulphonyl}amino)ethyl]-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 45 using 2-(4-fluorophenyl)ethanesulphonyl chloride as acylating agent in Step D.

### Elemental Microanalysis: (%, Measured(Theoretical))

% C=67.42 (68.12);% H=5.76 (6);% N=7.27 (7.75); S=3.68 (4.44)

### High-Resolution Mass Spectrometry (ESI+):

C₄₁H₄₃FN₄O₅S Empirical formula:

[M+H]⁺ calculated: 723.3018

[M+H]⁺ measured: 723.3011

### EXAMPLE 56

5-(5-{2-[(Benzylcarbamoyl)amino]ethyl}-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 45 using benzyl isocyanate as acylating agent in Step D.

### Elemental Microanalysis: (%, Measured(Theoretical))

% C=71.14 (73.52);% H=6.47 (6.47);% N=9.42 (10.46)

### High-Resolution Mass Spectrometry (ESI+):

C₄₁H₄₃N₅O₄ Empirical formula:

[M+H]⁺ calculated: 670.3395

[M+H]⁺ measured: 670.3390

### EXAMPLE 57

N-(4-Hydroxyphenyl)-N,1,2-trimethyl-5-(2-{[(3R)-3-methyl-3,4-dihydroisoquinolin-2 (1H)-yl]carbonyl}-5-{2-[(phenylacetyl)amino]ethyl}phenyl)-1H-pyrrole-3-carboxamide

The title compound is obtained in accordance with the process of Example 45 using phenylacetyl chloride as acylating agent in Step D.

### Elemental Microanalysis: (%, Measured(Theoretical))

% C=75.46 (75.21);% H=6.11 (6.46);% N=8.31 (8.56)

### High-Resolution Mass Spectrometry (ESI+):

C₄₁H₄₂N₄O₄ Empirical formula:

[M+H]⁺ calculated: 655.3286

[M+H]⁺ measured: 655.3285

### EXAMPLE 58

N-(4-Hydroxyphenyl)-N,1,2-trimethyl-5-(2-{[(3R)-3-methyl-3,4-dihydroisoquinolin-2 (1H)-yl]carbonyl}-5-{2-[(phenylcarbamoyl)amino]ethyl}phenyl)-1H-pyrrole-3-carboxamide

The title compound is obtained in accordance with the process of Example 45 using phenyl isocyanate as acylating agent in Step D.

Elemental microanalysis: (%, measured(theoretical))

% C=71.25 (73.26);% H=6.12 (6.3);% N=9.61 (10.68)

### High-Resolution Mass Spectrometry (ESI+):

C₄₀H₄₁N₅O₄ Empirical formula:

[M+H]⁺ calculated: 656.3239

[M+H]⁺ measured: 656.3226

### EXAMPLE 59

5-(4-[({[2-(4-Fluorophenyl)ethyl]sulphonyl}amino)methyl]-2-{[(3R)-3-methyl-3,4-dihydroisoquinolin-2 (1H)-yl]carbonyl}phenyl)-N-(4-hydroxyphenyl)-N,1,2-trimethyl-1H-pyrrole-3-carboxamide

The title compound is obtained in accordance with the process of Example 32 using 2-(4-fluorophenyl)ethanesulphonyl chloride as sulphonylation agent in Step D.

### Elemental Microanalysis: (%, Measured(Theoretical))

% H=5.19 (5.83);% N=7.28 (7.9); S=4.45 (4.52);% C=66.17 (67.78)

### High-Resolution Mass Spectrometry (ESI+):

C₄₀H₄₁FN₄O₅S Empirical formula:

[M+H]⁺ calculated: 709.2866

[M+H]⁺ measured: 709.2866

### EXAMPLE 60

5-(5-Chloro-2-{[(3S)-3-{[2-(dimethylamino)ethoxy]methyl}-3,4-dihydroisoquinolin-2 (1H)-yl]carbonyl}phenyl)-N-(4-hydroxy phenyl)-1,2-dimethyl-N-phenyl-1H-pyrrole-3-carboxamide hydrochloride

The process of Example 29 is used, on the one hand replacing the 4-(2-bromoethyl)morpholine hydrobromide used in Step B by 2-chloroethyldimethylamine hydrochloride and on the other hand adding a catalytic amount of tetrabutylammonium iodide.

### High-Resolution Mass Spectrometry (ESI+):

C₄₀H₄₁ClN₄O₄ Empirical formula:

[M+H]⁺ calculated: 677.2890

[M+H]⁺ measured: 677.2887

### EXAMPLE 61

N-(4-Hydroxyphenyl)-1,2-dimethyl-5-(6-{[(3R)-3-methyl-3,4-dihydroisoquinolin-2 (1H)-yl]carbonyl}-1,3-benzodioxol-5-yl)-N-(pyridin-4-yl)-1H-pyrrole-3-carboxamide hydrochloride

### High-Resolution Mass Spectrometry (ESI+):

C₃₆H₃₂N₄O₅ Empirical formula:

[M+H]⁺ calculated: 601.2445

[M+H]⁺ measured: 601.2424

### EXAMPLE 62

5-(5-Fluoro-4-methoxy-2-{[(3R)-3-methyl-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### Elemental Microanalysis: (%, Measured(Theoretical))

% C=68.99 (69.18);% H=5.89 (5.64);% N=11.35 (11.52)

### High-Resolution Mass Spectrometry (ESI+):

C₃₅H₃₄FN₅O₄ Empirical formula:

[M+H]⁺ calculated: 608.2668

[M+H]⁺ measured: 608.2640

### EXAMPLE 63

5-(5-Chloro-2-{[(3R)-3-methyl-3,4-dihydroisoquinolin-2 (1H)-yl]-carbonyl}phenyl)-N-(4-hydroxyphenyl)-1,2-dimethyl-N-(1-methyl-1H-pyrrolo[2,3-B]-pyridin-5-yl)-1H-pyrrole-3-carboxamide

### Elemental Microanalysis: (%, Measured(Theoretical))

% C=70.48 (70.85);% H=5.67 (5.32);% N=11.2 (10.87)

### High-Resolution Mass Spectrometry (ESI+):

C₃₈H₃₄ClN₅O₃ Empirical formula:

[M+H]⁺ calculated: 666.2242

[M+H]⁺ measured: 666.2235

### EXAMPLE 64

N,1,2-Trimethyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### High-Resolution Mass Spectrometry (ESI+):

C₃₈H₄₀N₆O₅ Empirical formula:

[M+H]⁺ calculated: 661.3133

[M+H]⁺ measured: 661.3125

### EXAMPLE 65

1,2-Dimethyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-N-phenyl-1H-pyrrole-3-carboxamide hydrochloride

### High-Resolution Mass Spectrometry (ESI+):

C₄₃H₄₂N₆O₅ Empirical formula:

[M+H]⁺ calculated: 723.3289

[M+H]⁺ measured: 723.3287

### EXAMPLE 66

4-Methylphenyl (4-{4-[(4-hydroxyphenyl)(methyl)carbamoyl]-1,5-dimethyl-1H-pyrrol-2-yl}-3-{[(3R)-3-methyl-3,4-dihydroisoquinolin-2 (1H)-yl]-carbonyl}benzyl)carbamate

The title compound is obtained in accordance with the process of Example 32 using 4-methylphenyl chloroformate as acylating agent in Step D.

### High-Resolution Mass Spectrometry (ESI+):

C₄₀H₄₀N₄O₅ Empirical formula:

[M+H]⁺ calculated: 657.3079

[M+H]⁺ measured: 657.3076

### EXAMPLE 67

5-(5-Fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide hydrochloride

### Elemental Microanalysis: (%, Measured(Theoretical))

% C=65.69 (65.28);% H=5.38 (5.77);% N=11.18 (12.02);% Cl—=5.61 (5.07)

### EXAMPLE 68

5-(5-Fluoro-4-hydroxy-2-{[(3R)-3-methyl-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

A 1N solution of tribromoborane (1.3 equivalents) in dichloromethane is poured rapidly, at ambient temperature, into a solution of the compound of Example 62 in dichloromethane. This operation is repeated twice in order to complete the reaction in 5 hours. The reaction mixture is poured into anhydrous ethanol at 5° C. After stirring for 10 minutes, saturated aqueous NaHCO₃ solution is added. The mixture is extracted with dichloromethane, dried over Na₂SO₄ and concentrated to dryness. The crude product obtained is purified by preparative HPLC using water, TFA and acetonitrile as eluants. The pH of the appropriate fractions is adjusted to 12 with saturated aqueous NaHCO₃ solution, and then the acetonitrile is evaporated off under reduced pressure. The aqueous residue is extracted with dichloromethane. The organic phase is dried over Na₂SO₄ and evaporated to dryness to yield the title compound.

### High-Resolution Mass Spectrometry (ESI+):

C₃₄H₃₂FN₅O₄ Empirical formula:

[M+H]⁺ calculated: 594.2511

[M+H]⁺ measured: 594.2517

### EXAMPLE 69

N-(4-Hydroxyphenyl)-1,2-dimethyl-5-(6-{[(3S)-3-[(4-methylpiperazin-1-yl)methyl]-3,4-dihydroisoquinolin-2 (1H)-yl]carbonyl}-1,3-benzodioxol-5-yl)-N-(1-methyl-1H-pyrrolo[2,3-B]pyridin-5-yl)-1H-pyrrole-3-carboxamide hydrochloride

### High-Resolution Mass Spectrometry (ESI+):

C₄₄H₄₅N₇O₅ Empirical formula:

[M+H]⁺ calculated: 752.3555

[M+H]⁺ measured: 752.3552

### EXAMPLE 70

N-(4-Hydroxyphenyl)-1,2-dimethyl-5-(6-{[(3S)-3-[(4-methylpiperazin-1-yl)methyl]-3,4-dihydroisoquinolin-2 (1H)-yl]carbonyl}-1,3-benzodioxol-5-yl)-N-(pyridin-4-yl)-1H-pyrrole-3-carboxamide hydrochloride

### High-Resolution Mass Spectrometry (ESI+):

C₄₁H₄₂N₆O₅ Empirical formula:

[M+H]⁺ calculated: 699.3289

[M+H]⁺ measured: 699.3293

### EXAMPLE 71

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide hydrochloride

### High-Resolution Mass Spectrometry (ESI+):

C₄₂H₄₁ClN₆O₄ Empirical formula:

[M+H]⁺ calculated: 723.3289

[M+H]⁺ measured: 723.3287

### EXAMPLE 7

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-Methyl-1H-benzimidazol-5-yl)-1H-pyrrole-3-carboxamide

### Elemental Microanalysis: (%, Measured(Theoretical))

% C=68.90 (69.17);% H=5.32 (5.67);% N=11.40 (11.52)

### EXAMPLE 73

N-(4-Hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-N-(pyridin-4-yl)-1H-pyrrole-3-carboxamide dihydrochloride

### High-Resolution Mass Spectrometry (ESI+):

C₄₀H₃₉N₅O₆ Empirical formula:

[M+H]⁺ calculated: 686.2973

[M+H]⁺ measured: 686.2971

### EXAMPLE 74

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(pyridin-4-yl)-1H-pyrrole-3-carboxamide hydrochloride

### High-Resolution Mass Spectrometry (ESI+):

C₃₉H₃₈ClN₅O₄ Empirical formula:

[M+H]⁺ calculated: 676.2685

[M+H]⁺ measured: 676.2684

### EXAMPLE 75

N-(4-Hydroxyphenyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-2-(trifluoromethyl)-1H-pyrrole-3-carboxamide hydrochloride

### Elemental Microanalysis: (%, Theoretical:Measured)

% C=60.12:59.77; % H=4.92:4.76; % N=10.79:10.39% Cl—=4.55:5.17

### High-Resolution Mass Spectrometry (ESI+):

C₃₉H₃₇F₃N₆O₆ Empirical formula:

[M+H]⁺ calculated: 743.2799

[M+H]⁺ measured: 742.2802

### EXAMPLE 76

2-(Difluoromethyl)-N-(4-hydroxyphenyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 77

N-(4-Hydroxyphenyl)-2-(methoxymethyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 78

2-(2,2-Difluoroethyl)-N-(4-hydroxyphenyl)-1-methyl-N-(1-Methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 79

N-(4-Hydroxyphenyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-2-(2,2,2-trifluoroethyl)-1H-pyrrole-3-carboxamide

### EXAMPLE 80

N-(4-Hydroxyphenyl)-2-(2-methoxyethyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 81

N-(4-Hydroxyphenyl)-1-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-2-[2-(morpholin-4-yl)ethyl]-1H-pyrrole-3-carboxamide hydrochloride

The procedure is in accordance with the general protocol of Example 1 using the compound of Preparation 22 in Step A. The product obtained is finally dissolved in acetonitrile and converted into salt form using 0.1M aqueous HCl solution. After a lyophilisation step, the expected compound is obtained in the form of a solid.

### High-Resolution Mass Spectrometry (ESI+):

C₄₀H₄₂N₆O₆ Empirical formula:

[M+H]⁺ calculated: 703.3239

[M+H]⁺ measured: 703.3236

### EXAMPLE 82

N-(4-Hydroxyphenyl)-1-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-2-(morpholin-4-ylmethyl)-1H-pyrrole-3-carboxamide

The procedure is in accordance with the general protocol of Example 1 using the compound of Preparation 23 in Step A.

### High-Resolution Mass Spectrometry (ESI+):

C₃₉H₄₀N₆O₆ Empirical formula:

[M+H]⁺ calculated: 689.3082

[M+H]⁺ measured: 689.3085

### EXAMPLE 83

N-(4-Hydroxyphenyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-2-(trifluoromethyl)-1H-pyrrole-3-carboxamide

### EXAMPLE 84

2-(Difluoromethyl)-N-(4-hydroxyphenyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 85

N-(4-Hydroxyphenyl)-2-(methoxymethyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 86

2-(2,2-Difluoroethyl)-N-(4-hydroxyphenyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 87

N-(4-Hydroxyphenyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-2-(2,2,2-trifluoroethyl)-1H-pyrrole-3-carboxamide

### EXAMPLE 88

N-(4-HYDROXYPHENYL)-2-(2-methoxyethyl)-1-methyl-N-(1-Methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

The procedure is in accordance with the general protocol of Example 1 using the appropriate Preparations. The product obtained is finally dissolved in acetonitrile and converted into salt form using 0.1M aqueous HCl solution. After a lyophilisation step, the expected compound is obtained in the form of a solid.

### Elemental Microanalysis: (%, Theoretical:Measured)

% C=65.97:66.15; % H=5.78:5.46; % N=10.26:10.24; % Cl—=4.33:4.76

### High-Resolution Mass Spectrometry (ESI/FIA/HR and MS/MS):

C₄₅H₄₆N₆O₇ Empirical formula:

[M+H]⁺ calculated: 783.3501

[M+H]⁺ measured: 783.3502

### EXAMPLE 89

N-(4-Hydroxyphenyl)-1-methyl-N-(1-methyl-1H-pyrrolo[2,3-B]pyridin-5-yl)-2-[2-(morpholin-4-yl)ethyl]-5-(6-{[(3R)-3-methyl-3,4-dihydroisoquinolin-2 (1H)-yl]carbonyl}-1,3-benzodioxol-5-yl)-1H-pyrrole-3-carboxamide

### EXAMPLE 90

N-(4-Hydroxyphenyl)-1-methyl-N-(1-methyl-1H-pyrrolo[2,3-B]pyridin-5-yl)-2-(morpholin-4-ylmethyl)-5-(6-{[(3R)-3-methyl-3,4-dihydroisoquinolin-2 (1H)-yl]carbonyl}-1,3-benzodioxol-5-yl)-1H-pyrrole-3-carboxamide

### EXAMPLE 91

N-(4-Hydroxyphenyl)-2-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 92

1-ETHYL-N-(4-Hydroxyphenyl)-2-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

The procedure is in accordance with the protocol described in Example 1, using the compounds from Preparations 20 and 3′ in Step A and the compound from Preparation 1″ in

Step C. The product obtained is finally dissolved in acetonitrile and converted into salt form using 0.1M aqueous HCl solution. After a lyophilisation step, the expected compound is obtained in the form of a solid.

### Elemental Microanalysis: (%, Theoretical:Measured)

% C=64.99:65.61; % H=5.86:5.39; % N=11.37:11.43; % Cl=4.80:4.42
[M+H]⁺ measured: 703.3236

### EXAMPLE 93

N-(4-Hydroxyphenyl)-1-(2-methoxyethyl)-2-Methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Theoretical:Measured)

% C=64.01:64.10; % H=5.90:5.63; % N=10.92:10.88; % C—=4.61:4.70.

### High-Resolution Mass Spectrometry (ESI+):

C₄₁H₄₄N₆O₇ Empirical formula:

[M+H]⁺ calculated: 733.3344

[M+H]⁺ measured: 733.3345

### EXAMPLE 94

1-(2-Fluoroethyl)-N-(4-hydroxyphenyl)-2-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

The procedure is in accordance with the protocol described in Steps A-D of Example 1, using the compounds from Preparations 21 and 3′ in Step A and the compound from Preparation 1″ in Step C. The product obtained is finally dissolved in acetonitrile and converted into salt form using 0.1M aqueous HCl solution. After a lyophilisation step, the expected compound is obtained in the form of a solid.

### Elemental Microanalysis: (%, Theoretical:Measured)

% C=63.44:63.25; % H=5.59:5.09; % N=11.10:11.02; % Cl=4.68:4.74

### High-Resolution Mass Spectrometry (ESI+):

C₄₀H₄₁FN₆O₆ Empirical formula:

[M+H]⁺ calculated: 721.3144

[M+H]⁺ measured: 721.3147

### EXAMPLE 95

1-(2,2-Difluoroethyl)-N-(4-hydroxyphenyl)-2-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

The title compound is obtained in accordance with the process of Example 1 using the acid of Preparation 32 and the compound of Preparation 3′ in Step A, and the compound of Preparation 1″ in Step C. The compound obtained is converted into salt form and lyophilised as described in the general process to obtain the title compound.

### Elemental Microanalysis: (%, Theoretical:Measured)

% C=61.97:61.89; % H=5.33:5.04; % N=10.84:10.85; % Cl⁻=4.57:4.55

### High-Resolution Mass Spectrometry (ESI+/HR, ESI−/LR):

C₄₀H₄₀F₂N₆O₆ Empirical formula:

[M+H]⁺ calculated: 739.3050

[M+H]⁺ measured: 739.3052

### EXAMPLE 96

N-(4-Hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1-[2-(morpholin-4-yl)ethyl]-1H-pyrrole-3-carboxamide hydrochloride
Step A: N-[4-[tert-Butyl(dimethyl)silyl]oxyphenyl]-2-methyl-5-[6-[(3R)-3-methyl-3,4-dihydro-1H-isoquinoline-2-carbonyl]-1,3-benzodioxol-5-yl]-N-(1-methyl-1H-pyrazol-4-yl)-1-(2-morpholinoethyl)pyrrole-3-carboxamide

The compound obtained in Step E of Example 99 is dissolved in 6 mL of tetrahydrofuran. Sodium iodide (100 mg, 0.67 mmol) is added, followed by morpholine (0.21 mL, 2.53 mmol) dropwise. The reaction mixture is heated in a sealed flask at 90° C. for 72 hours. After cooling, the solution is evaporated to dryness and the residue is purified by chromatography over silica gel using dichloromethane and methanol as eluants. The expected product is obtained in the form of a foam.

¹H NMR (500 MHz, dmso-d6) δ ppm: 7.7-7.45 (4 bs, 1H), 7.2-6.9 (m, 4H), 7.1-6.4 (m, 2H), 7.1-6.9 (4 bs, 1H), 7-6.8 (4 m, 2H), 6.75/6.48 (d+bs, 2H), 6.1 (4 bs, 2H), 5.25-4.7 (4 bs, 1H), 5.2-3.8 (m, 2H), 4.9/4.65/3.8 (3 m, 1H), 3.75 (4 s, 3H), 3.5 (bs, 4H), 3-2 (m, 10H), 2.41/2.3 (2 bs, 3H), 1.02/0.95/0.78 (3 bd, 3H), 0.88 (m, 9H), 0.1 (m, 6H)

IR (ATR) cm⁻¹ 1626 δ>C═O amides

Step B: N-(4-Hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1-[2-(morpholin-4-yl)ethyl]-1H-pyrrole-3-carboxamide hydrochloride

The procedure is in accordance with the protocol described in Step D of Example 1. The product obtained is finally subjected to a step of conversion into salt form in the presence of HCl in ether. After filtration and lyophilisation in a mixture of acetonitrile/water, the expected compound is obtained.

### High-Resolution Mass Spectrometry (ESI+):

C₄₀H₄₂N₆O₆ Empirical formula:

[M+H]⁺ calculated: 703.3239

[M+H]⁺ measured: 703.3238

### EXAMPLE 97

N-(4-Hydroxyphenyl)-2-methyl-5-(6-{[(3R)-3-methyl-3,4-dihydroisoquinolin-2 (1H)-yl]carbonyl}-1,3-benzodioxol-5-yl)-1-[2-(morpholin-4-yl)ethyl]-N-phenyl-1H-pyrrole-3-carboxamide hydrochloride

The procedure is in accordance with the protocol described in Example 96 replacing the compound of Preparation 1″ by that of Preparation 2″.

### Elemental Microanalysis: (%, Theoretical:Measured)

% C=68.61:68.12; % H=5.89:5.23; % N=7.62:7.54; % Cl—=4.82:4.66

### High-Resolution Mass Spectrometry (ESI/FIA/HR and MS/MS):

C₄₂H₄₂N₄O₆ Empirical formula:

[M+H]⁺ calculated: 699.3177

[M+H]⁺ measured: 699.3173

### EXAMPLE 98

1-[2-(Dimethylamino)ethyl]-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-phenyl-1H-pyrrole-3-carboxamide hydrochloride

The procedure is in accordance with the protocol described in Example 27 replacing the compound of Preparation 1″ by that of Preparation 2″. The product obtained is finally subjected to a step of conversion into salt form in the presence of 1M HCl in ether. After filtration and lyophilisation in a mixture of acetonitrile/water, the expected compound is obtained.

### Elemental Microanalysis: (%, Theoretical:Measured)

% C=69.30:69.20; % H=5.96:5.48; % N=8.08:8.08; % Cl—=5.11:5.03

### High-Resolution Mass Spectrometry (ESI/FIA/HR and MS/MS):

C₄₀H₄₀N₄O₅ Empirical formula:

[M+H]⁺ calculated: 657.3071

[M+H]⁺ measured: 657.3066

### EXAMPLE 99

N-(4-Hydroxyphenyl)-1-{2-[(2-methoxyethyl)(methyl)amino]ethyl}-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide hydrochloride
Step A: Ethyl 1-(2-benzyloxyethyl)-2-methyl-5-[6-[(3R)-3-methyl-3,4-dihydro-1H-isoquinoline-2-carbonyl]-1,3-benzodioxol-5-yl]pyrrole-3-carboxylate

The procedure is in accordance with the protocol described in Step A of Example 1 replacing the compound of Preparation 1 by that of Preparation 19.

¹H NMR (500 MHz, dmso-d6) δ ppm: 7.35-6.95 (m, 9H), 7-6.8 (several s, 2H), 6.35-5.85 (several s, 1H), 6.15 (several s, 2H), 5.15-3.5 (several m, 4H), 4.9/4.7/3.95 (several m, 1H), 4.4 (m, 2H), 4.2-3.95 (m, 2H), 3.55 (m, 2H), 3.1-2.35 (several m, 2H), 2.5-2.2 (several s, 3H), 1.25-1.1 (several m, 3H), 1.05-0.7 (several d, 3H)

Step B: 1-(2-Benzyloxyethyl)-2-methyl-5-[6-[(3R)-3-methyl-3,4-dihydro-1H-isoquinoline-2-carbonyl]-1,3-benzodioxol-5-yl]pyrrole-3-carboxylic acid

The procedure is in accordance with the protocol described in Step B of Example 1.

IR (ATR) cm⁻¹: 3000-2500 ν —OH, 1675-1625 ν —C═O carboxylic acid+amide

Step C: 1-(2-Benzyloxyethyl)-N-[4-[tert-butyl(dimethyl)silyl]oxyphenyl]-2-methyl-5-[6-[(3R)-3-methyl-3,4-dihydro-1H-isoquinoline-2-carbonyl]-1,3-benzodioxol-5-yl]-N-(1-methyl-1H-pyrazol-4-yl)pyrrole-3-carboxamide

The procedure is in accordance with the protocol described in Step C of Example 1.

¹H NMR (500 MHz, dmso-d6) δ ppm: 7.7-7.5 (several s, 1H), 7.35-6.5 (several m, 11H), 7.1-6.9 (several s, 1H), 6.95-6.5 (several s, 2H), 6.8/6.5 (m, 2H), 6.05 (several s, 2H), 5.25-4.7 (several s, 1H), 5.1-3.6 (several m, 4H), 4.85/4.6/3.75 (several m, 1H), 4.3 (m, 2H), 3.7 (2×s, 3H), 3.4 (m, 2H), 3.05-2.4 (several m, 2H), 2.4-2.25 (several s, 3H), 1-0.75 (several d, 3H), 0.9 (several s, 9H), 0.1 (several s, 6H)

IR (ATR) cm⁻¹: 1629 ν>C═O amides

Step D: N—[4-[tert-Butyl(dimethyl)silyl]oxyphenyl]-1-(2-hydroxyethyl)-2-methyl-5-[6-[(3R)-3-methyl-3,4-dihydro-1H-isoquinoline-2-carbonyl]-1,3-benzodioxol-5-yl]-N-(1-methyl-1H-pyrazol-4-yl)pyrrole-3-carboxamide

In a hydrogenating reactor, the compound of the above Step is dissolved in 30 mL of methanol. The solution is degassed by bubbling argon in, and palladium-on-carbon 10% (550 mg) is added. The resulting suspension is stirred under a pressure of 1 bar of hydrogen for 15 hours, and then passed through a Whatman® filter. The catalyst is rinsed with methanol and the filtrate is evaporated in vacuo. The residue thereby obtained is purified by chromatography over silica gel using dichloromethane and methanol as eluants. The expected product is obtained in the form of an oil.

¹H NMR (500 MHz, pyridine-d5) δ ppm: 7.7-7.4 (4 s, 1H), 7.3-6.9 (m, 4H), 7.1-6.8 (4 s, 1H), 7-6.7 (m, 2H), 6.9-6.4 (m, 2H), 6.8-6.4 (4 m, 2H), 6.1 (m, 2H), 5.2/5/4.7 (4 s, 1H), 5.15-3.9 (8 d, 2H), 4.9-4.8 (m, 1H), 4.9/4.7/3.85 (3 m, 1H), 3.9-3.7 (m, 2H), 3.75 (2 s, 3H), 3.5-3.2 (m, 2H), 3-2.4 (m, 2H), 2.4-2.3 (4 s, 3H), 1.05/0.95/0.75 (4 d, 3H), 0.85 (bs, 9H), 0.15-0 (m, 6H)

IR (ATR) cm⁻¹:3346 ν —OH primary alcohol, 1621ν —C═O amides

Step E: 2-[3-[[4-[tert-Butyl(dimethyl)silyl]oxyphenyl]-(1-methyl-1H-pyrazol-4-yl)-carbamoyl]-2-methyl-5-[6-[(3R)-3-methyl-3,4-dihydro-1H-isoquinoline-2-carbonyl]-1,3-benzodioxol-5-yl]pyrrol-1-yl]ethyl methanesulphonate

The compound of the above Step (2.37 g, 3.17 mmol) is dissolved in 30 mL of tetrahydrofuran. The reaction mixture is cooled to 0° C. and there are successively added triethylamine (1.8 mL, 13.9 mmol) and, dropwise, methanesulphonyl chloride (0.40 mL, 5.17 mmol). The reaction mixture is stirred for 2 hours at 0° C. The reaction mixture is then poured into saturated aqueous sodium hydrogen carbonate solution and extracted 3 times with ethyl acetate. The organic phase is washed 3 times with brine, dried over MgSO₄, filtered and then evaporated to dryness. The evaporation residue is used without purification in the next Step.

¹H NMR (500 MHz, dmso-d6) δ ppm: 7.7-7.45 (several s, 1H), 7.25-6.4 (m, 8H), 7.1-6.9 (several s, 1H), 6.95-6.65 (several s, 2H), 6.1 (several s, 2H), 5.2-4.6 (several s, 1H), 5.05-3.75 (several d, 2H), 4.9/4.6/3.9/3.8 (several m, 1H), 4.3-4.05 (m, 2H), 4.2-3.95 (m, 2H), 3.75/3.7 (2 s, 3H), 3.05-2.5 (several m, 2H), 3 (several s, 3H), 2.45-2.3 (several s, 3H), 1.05-0.75 (several d, 3H), 0.9 (several s, 9H), 0.1 (several s, 6H)

IR (ATR) cm⁻¹: 1626 ν —C═O, 1349 ν —SO₂, 1249 δ —CH3, 1172 ν —SO₂

Step F: N—[4-[tert-Butyl(dimethyl)silyl]oxyphenyl]-1-[2-[2-methoxyethyl(methyl)amino]-ethyl]-2-methyl-5-[6-[(3R)-3-methyl-3,4-dihydro-1H-isoquinoline-2-carbonyl]-1,3-benzodioxol-5-yl]-N-(1-methyl-1H-pyrazol-4-yl)pyrrole-3-carboxamide

The compound of the above Step (1.33 mg, 1.61 mmol) is dissolved in 6 mL of tetrahydrofuran. Sodium iodide (100 mg, 0.67 mmol) is added, followed by dimethylamine (0.172 g, 1.932 mmol) dropwise. The reaction mixture is heated in a sealed flask at 60° C. for 36 hours. After cooling, it is evaporated to dryness and purified by chromatography over silica gel using dichloromethane and ammonia-in-methanol as eluants. The expected product is obtained in the form of a foam.

¹H NMR (500 MHz, dmso-d6) δ ppm 7.7-7.45 (4 bs, 1H), 7.2-6.9 (m, 4H), 7.1-6.9 (4 bs, 1H), 7-6.7 (4 m, 2H), 7/6.45 (2 m, 2H), 6.8/6.45 (2 m, 2H), 6.1 (m, 2H), 5.22/5.05/4.7 (4 bs, 1H), 5.2-3.8 (m, 2H), 4.91/4.65/3.9 (3 m, 1H), 3.75 (2 bs, 3H), 3.7 (m, 2H), 3.2 (bs, 3H), 3-2 (m, 2H), 2.7-2 (ml, 6H), 2.7-2 (m, 3H), 2.45/2.35 (2 bs, 3H), 1.05/0.95/0.8 (3 bd, 3H), 0.9 (m, 9H), 0.1 (m, 6H)

IR (ATR) cm⁻¹ 1628 (shoulder) δ—C═O amides

Step G: N-(4-hydroxyphenyl)-1-[2-[2-methoxyethyl(methyl)amino]ethyl]-2-methyl-5-[6-[(3R)-3-methyl-3,4-dihydro-1H-isoquinoline-2-carbonyl]-1,3-benzodioxol-5-yl]-N-(1-methyl-1H-pyrazol-4-yl)pyrrole-3-carboxamide hydrochloride

The procedure is in accordance with the protocol described in Step D of Example 1. The product obtained is finally subjected to a step of conversion into salt form in the presence of 1M HCl in ether. After filtration and lyophilisation in a mixture of acetonitrile/water, the expected compound is obtained.

IR (ATR) cm⁻¹ 2000 to 3500 ν —NH⁺/OH, 1615 ν>C═O amides, 1237-1161 δ >C—O—C<, 745 ν>CH—Ar

### High-Resolution Mass Spectrometry (ESI+):

C₄₀H₄₄N₆O₆ Empirical formula:

[M+H]⁺ calculated: 705.3395

[M+H]⁺ measured: 705.3391

### EXAMPLE 100

5-(5-Fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide hydrochloride

The procedure is in accordance with the general protocol of Example 1 using the appropriate Preparations. The product obtained is finally dissolved in acetonitrile and converted into salt form using 0.1M aqueous HCl solution. After a lyophilisation step, the expected compound is obtained in the form of a solid.

### Elemental Microanalysis: (%, Theoretical:Measured)

% C=64.23:64.31; % H=5.80:5.43; % N=11.52:11.46; % Cl—=4.86:4.95

### High-Resolution Mass Spectrometry (ESI+):

C₃₉H₄₁F N₆O₅ Empirical formula:

[M+H]⁺ calculated: 693.3195

[M+H]⁺ measured: 693.3194

### EXAMPLE 101

5-(4-Fluoro-5-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide hydrochloride

The title compound is obtained in accordance with the process of Example 1 using the acid of Preparation 27 and the compound of Preparation 3′ in Step A, and the compound of Preparation 1″ in Step C. The compound obtained is converted into salt form and lyophilised as described in the general process to obtain the title compound.

### Elemental Microanalysis: (%, Theoretical:Measured)

% C=64.23:63.12; % H=5.80:5.20; % N=11.52:11.38. % Cl⁻=4.86:5.03

### High-Resolution Mass Spectrometry (ESI+/HR, ESI−/LR):

C₃₉H₄₁F N₆O₅ Empirical formula:

[M+H]⁺ calculated: 693.3195

[M+H]⁺ measured: 693.3195

### EXAMPLE 102

5-(5-Chloro-2-{[(3S)-3-[(4-methylpiperazin-1-yl)methyl]-3,4-dihydro-isoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide trihydrochloride

### High-Resolution Mass Spectrometry (ESI+):

C₄₃H₄₄Cl N₇O₃ Empirical formula:

[M+H]⁺ calculated: 742.3267

[M+H]⁺ measured: 742.3268

### EXAMPLE 103

5-(5-Chloro-2-{[(3S)-3-[(4-methylpiperazin-1-yl)methyl]-3,4-dihydroisoquinolin-2 (1H)-yl]carbonyl}phenyl)-1,2-dimethyl-N-(1-methyl-1H-pyrrolo[2,3-B]pyridin-5-yl)-N-phenyl-1H-pyrrole-3-carboxamide

### EXAMPLE 104

N-(4-Hydroxyphenyl)-1,2-dimethyl-N-(1-methyl-1H-pyrazolo[3,4-B]-pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 105

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 106

5-(5-Chloro-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrrolo[2,3-B]pyridin-5-yl)-N-phenyl-1H-pyrrole-3-carboxamide

The procedure is in accordance with the general protocol of Example 1 using the appropriate Preparations, it being understood that Step D is not carried out. The expected product is obtained in free base form.

### Elemental Microanalysis: (%, Theoretical:Measured)

% C=70.72:69.77; % H=5.79:5.96; % N=11.78:11.43

### High-Resolution Mass Spectrometry (ESI+)

C₄₂H₄₁Cl N₆O₃ Empirical formula:

[M+H]⁺ calculated: 713.3001

[M+H]⁺ measured: 713.2998

### EXAMPLE 107

5-(5-Chloro-2-{[(3S)-3-(morpholin-4-ylmethyl)-3,4-dihydroisoquinolin-2 (1H)-yl]carbonyl}phenyl)-N-(4-fluorophenyl)-1,2-dimethyl-N-(1-methyl-1H-pyrrolo[2,3-B]pyridin-5-yl)-1H-pyrrole-3-carboxamide

### EXAMPLE 108

5-(5-Chloro-2-{[(3S)-3-(morpholin-4-ylmethyl)-3,4-dihydroisoquinolin-2 (1H)-yl]carbonyl}phenyl)-N-(5-cyano-1-methyl-1H-pyrrol-3-yl)-N-(4-hydroxyphenyl)-1,2-dimethyl-1H-pyrrole-3-carboxamide hydrochloride

The title compound is obtained in accordance with the process of Example 1 using the acid of Preparation 1 and (3S)-3-(4-morpholinylmethyl)-1,2,3,4-tetrahydroisoquinoline (see Preparation 3′) in Step A, and the compound of Preparation 19″ in Step C. The product obtained is finally subjected to a step of conversion into salt form in the presence of HCl in ether. After filtration and lyophilisation in a mixture of acetonitrile/water, the expected compound is obtained.

### Elemental Microanalysis: (%, Theoretical:Measured)

% C=64.95:65.09; % H=5.45:5.20; % N=11.36:11.26; % Cl—=4.79:4.62

### High-Resolution Mass Spectrometry (ESI/+):

C₄₀H₃₉Cl N₆O₄ Empirical formula:

[M+H]⁺ calculated: 703.2794

[M+H]⁺ measured: 703.2789

### EXAMPLE 109

5-(5-Chloro-2-{[(3S)-3-(morpholin-4-ylmethyl)-3,4-dihydroisoquinolin-2 (1H)-yl]carbonyl}phenyl)-N-(6-cyanopyridin-2-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 23″ in Step C. The compound obtained is converted into salt form and lyophilised as described in the general process to obtain the title compound.

### High-Resolution Mass Spectrometry (ESI+−/FIA/HR, ESI−/FIA):

C₄₀H₃₇Cl N₆O₄ Empirical formula:

[M+H]⁺ calculated: 701.2638

[M+H]⁺ measured: 701.2639

### EXAMPLE 110

5-(5-Chloro-2-{[(3S)-3-(morpholin-4-ylmethyl)-3,4-dihydroisoquinolin-2 (1H)-yl]carbonyl}phenyl)-N-(4-cyanopyridin-2-yl)-N-(4-hydroxyphenyl)-1,2-dimethyl-1H-pyrrole-3-carboxamide hydrochloride

Elemental Microanalysis: (%, Theoretical:Measured)

% C=65.13:65.72; % H=5.19:4.76; % N=11.39:12.04; % Cl⁻=4.81:4.45

### High-Resolution Mass Spectrometry (ESI+−/FIA/HR, ESI−/FIA):

C₄₀H₃₇Cl N₆O₄ Empirical formula:

[M+H]⁺ calculated: 701.2638

[M+H]⁺ measured: 701.2643

### EXAMPLE 111

5-(5-Chloro-2-{[(3S)-3-(morpholin-4-ylmethyl)-3,4-dihydroisoquinolin-2 (1H)-yl]carbonyl}phenyl)-N-(4-cyanopyrimidin-2-yl)-N-(4-hydroxyphenyl)-1,2-dimethyl-1H-pyrrole-3-carboxamide

### EXAMPLE 112

5-(5-Chloro-2-{[(3S)-3-(morpholin-4-ylmethyl)-3,4-dihydroisoquinolin-2 (1H)-yl]carbonyl}phenyl)-N-[2-(dimethylamino)pyridin-4-yl]-N-(4-hydroxyphenyl)-1,2-dimethyl-1H-pyrrole-3-carboxamide

### EXAMPLE 113

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-benzimidazol-5-yl)-1H-pyrrole-3-carboxamide

### EXAMPLE 114

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(pyrazolo[1,5-A]pyrimidin-6-yl)-1H-pyrrole-3-carboxamide

### EXAMPLE 115

5-(5-Chloro-2-{[(3S)-3-[(9aS)-hexahydropyrazino[2,1-C][1,4]oxazin-8 (1H)-ylmethyl]-3,4-DIHYDROISOQUINOLIN-2 (1H)-yl]carbonyl}PHENYL)-N-(4-HYDROXYPHENYL)-1,2-dimethyl-N-(1-methyl-1H-pyrazol-4-yl)-1H-pyrrole-3-carboxamide dihydrochloride

The title compound is obtained in accordance with the process of Example 1 using the acid of Preparation 1 and the compound of Preparation 7′ in Step A, and the compound of Preparation 1″ in Step C. The compound obtained is converted into salt form and lyophilised as described in the general process to obtain the title compound.

### Elemental Microanalysis: (%, Theoretical:Measured)

% C=61.01:60.17; % H=5.74:5.09; % N=12.15:12.02; % Cl⁻=8.78:9.81

### High-Resolution Mass Spectrometry (ESI+−/FIA/HR, ESI−/FIA):

C₄₁H₄₄Cl N₇O₄ Empirical formula:

[M+H]⁺ calculated: 734.3216

[M+H]⁺ measured: 734.3220

### EXAMPLE 116

5-(5-Chloro-2-{[(3S)-3-[(9aR)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide dihydrochloride

The title compound is obtained in accordance with the process of Example 1 using the acid of Preparation 1 and the compound of Preparation 9′ in Step A, and the compound of Preparation 1″ in Step C. The compound obtained is converted into salt form and lyophilised as described in the general process to obtain the title compound.

### Elemental Microanalysis: (%, Theoretical:Measured)

% C=61.01:61.90; % H=5.74:5.65; % N=12.15:12.14; % Cl=13.15:11.51

### High-Resolution Mass Spectrometry (ESI/FIA/HR and MS/MS):

C₄₁H₄₄Cl N₇O₄ Empirical formula:

[M+H]⁺ calculated: 734.3216

[M+H]⁺ measured: 734.3218

### EXAMPLE 117

5-(5-Chloro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 118

5-(5-Chloro-2-{[(3S)-3-(difluoromethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 119

5-(5-Chloro-2-{[(3S)-3-(trifluoromethyl)-3,4-DIHYDROISOQUINOLIN-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 120

5-(5-Chloro-2-{[(3S)-3-[(3-cyanoazetidin-1-yl)methyl]-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 121

5-(5-Chloro-2-{[(3S)-3-(morpholin-4-ylmethyl)-3,4-dihydroisoquinolin-2 (1H)-yl]carbonyl}phenyl)-N-(1-ethyl-1H-pyrazol-4-yl)-N-(4-hydroxyphenyl)-1,2-dimethyl-1H-pyrrole-3-carboxamide

### EXAMPLE 122

5-(5-Chloro-2-{[(3S)-3-(morpholin-4-ylmethyl)-3,4-dihydroisoquinolin-2 (1H)-yl]carbonyl}phenyl)-N-(1-cyclopropyl-1H-pyrazol-4-yl)-N-(4-hydroxyphenyl)-1,2-dimethyl-1H-pyrrole-3-carboxamide

The title compound is obtained in accordance with the process of Example 1 using the acid of Preparation 1 and the compound of Preparation 3′ in Step A, and the compound of Preparation 28″ in Step C.

### Elemental Microanalysis: (%, Theoretical:Measured)

% C=68.12:67.94; % H=5.86:5.77; % N=11.92:11.65

### High-Resolution Mass Spectrometry (ESI+/HR, ESI−/LR):

C₄₀H₄₁Cl N₆O₄ Empirical formula:

[M+H]⁺ calculated: 705.2951

[M+H]⁺ measured: 705.2952

### EXAMPLE 123

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(trifluoromethyl)-1H-pyrazol-4-yl]-1H-pyrrole-3-carboxamide

### EXAMPLE 124

5-(5-Chloro-2-{[(3S)-3-(morpholin-4-ylmethyl)-3,4-dihydroisoquinolin-2 (1H)-yl]carbonyl}phenyl)-N-[1-(difluoromethyl)-1H-pyrazol-4-yl]-N-(4-hydroxyphenyl)-1,2-dimethyl-1H-pyrrole-3-carboxamide

### EXAMPLE 125

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-N-[1-(2-methoxyethyl)-1H-pyrazol-4-yl]-1,2-dimethyl-1H-pyrrole-3-carboxamide

### EXAMPLE 126

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(oxetan-3-yl)-1H-pyrazol-4-yl]-1H-pyrrole-3-carboxamide

The title compound is obtained in accordance with the process of Example 1 using the acid of Preparation 1 and the compound of Preparation 3′ in Step A, and the compound of Preparation 26″ in Step C.

### High-Resolution Mass Spectrometry (ESI+/HR, ESI−/LR):

C₄₀H₄₁Cl N₆O₅ Empirical formula:

[M+H]⁺ calculated: 721.2900

[M+H]⁺ measured: 721.2902

### EXAMPLE 127

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(tetrahydrofuran-3-yl)-1H-pyrazol-4-yl]-1H-pyrrole-3-carboxamide hydrochloride

The title compound is obtained in accordance with the process of Example 1 using the acid of Preparation 1 and the compound of Preparation 3′ in Step A, and the compound of Preparation 22″ in Step C. The compound obtained is converted into salt form and lyophilised as described in the general process to obtain the title compound.

### Elemental Microanalysis: (%, Theoretical:Measured)

% C=63.81:63.63; % H=5.75:5.74; % N=10.89:10.71; % Cl⁻=4.59:4.52

### High-Resolution Mass Spectrometry (ESI+/HR, ESI−/LR):

C₄₁H₄₃Cl N₆O₅ Empirical formula:

[M+H]⁺ calculated: 735.3056

[M+H]⁺ measured: 735.3061

### EXAMPLE 128

5-(5-Chloro-2-{[(3S)-3-(morpholin-4-ylmethyl)-3,4-dihydroisoquinolin-2 (1H)-yl]carbonyl}phenyl)-N-[1-(2-hydroxy-2-methylpropyl)-1H-pyrazol-4-yl]-N-(4-hydroxyphenyl)-1,2-dimethyl-1H-pyrrole-3-carboxamide

### EXAMPLE 129

5-(5-Chloro-2-{[(3S)-3-(morpholin-4-ylmethyl)-3,4-dihydroisoquinolin-2 (1H)-yl]carbonyl}phenyl)-1,2-dimethyl-N-(1-methyl-2,3-dihydro-1H-pyrrolo[2,3-B]-pyridin-5-yl)-N-phenyl-1H-pyrrole-3-carboxamide

The title compound is obtained in accordance with the process described in Step B of Example 49 using Example 106 as starting material, it being understood that the product is not subjected to the step of conversion into salt form.

### Elemental Microanalysis: (%, Theoretical:Measured)

% C=70.53:70.51; % H=6.06:5.81; % N=11.75:11.71

### High-Resolution Mass Spectrometry (ESI+−/FIA):

C₄₂H₄₃Cl N₆O₃ Empirical formula:

[M+H]⁺ calculated: 715.3158

[M+H]⁺ measured: 715.3159

### EXAMPLE 130

5-(5-Chloro-2-{[(3S)-3-(morpholin-4-ylmethyl)-3,4-dihydroisoquinolin-2 (1H)-yl]carbonyl}phenyl)-N-(4-fluorophenyl)-1,2-dimethyl-N-(1-methyl-2,3-dihydro-1H-pyrrolo[2,3-B]pyridin-5-yl)-1H-pyrrole-3-carboxamide

### EXAMPLE 131

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-N,1,2-trimethyl-1H-pyrrole-3-carboxamide

### EXAMPLE 132

5-(5-Chloro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-phenyl-1H-pyrrole-3-carboxamide

### EXAMPLE 133

5-(5-Chloro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-(1-methyl-1H-pyrrolo[2,3-B]pyridin-5-yl)-1H-pyrrole-3-carboxamide

### EXAMPLE 134

5-(5-Chloro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-(1-methyl-2,3-dihydro-1H-pyrrolo[2,3-B]pyridin-5-yl)-1H-pyrrole-3-carboxamide

### EXAMPLE 135

5-(5-Chloro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-(1-methyl-1H-pyrazol-4-yl)-1H-pyrrole-3-carboxamide

### EXAMPLE 136

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1H-pyrrole-3-carboxamide

### EXAMPLE 137

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1H-pyrrole-3-carboxamide

### EXAMPLE 138

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 139

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-1H-pyrrole-3-carboxamide

### EXAMPLE 140

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-1H-pyrrole-3-carboxamide

### EXAMPLE 141

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-1H-pyrrole-3-carboxamide

### EXAMPLE 142

5-(5-Chloro-2-{[(3S)-3-(morpholin-4-ylmethyl)-3,4-dihydroisoquinolin-2 (1H)-yl]carbonyl}phenyl)-1-ethyl-N-(4-hydroxyphenyl)-2-methyl-N-(1-Methyl-1H-pyrazol-4-yl)-1H-pyrrole-3-carboxamide

### EXAMPLE 143

5-(5-Chloro-2-{[(3S)-3-(morpholin-4-ylmethyl)-3,4-dihydroisoquinolin-2 (1H)-yl]carbonyl}phenyl)-1-ethyl-N-(4-hydroxyphenyl)-2-methyl-N-(1-methyl-1H-pyrrolo[2,3-B]pyridin-5-yl)-1H-pyrrole-3-carboxamide

### EXAMPLE 144

5-(5-Chloro-2-{[(3S)-3-(morpholin-4-ylmethyl)-3,4-dihydroisoquinolin-2 (1H)-yl]carbonyl}phenyl)-1-ethyl-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 145

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-(2-methoxyethyl)-2-methyl-N-(1-methyl-1H-pyrazol-4-yl)-1H-pyrrole-3-carboxamide

### EXAMPLE 146

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-(2-methoxyethyl)-2-methyl-N-(1-methyl-1H-pyrrolo[2,3-B]pyridin-5-yl)-1H-pyrrole-3-carboxamide

### EXAMPLE 147

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-(2-methoxyethyl)-2-methyl-N-(1-methyl-2,3-dihydro-1H-pyrrolo[2,3-B]pyridin-5-yl)-1H-pyrrole-3-carboxamide

### EXAMPLE 148

5-(5-Chloro-2-{[(3S)-3-(morpholin-4-ylmethyl)-3,4-dihydroisoquinolin-2 (1H)-yl]carbonyl}phenyl)-1-(2-fluoroethyl)-N-(4-hydroxyphenyl)-2-Methyl-N-(1-methyl-1H-pyrazol-4-yl)-1H-pyrrole-3-carboxamide

### EXAMPLE 149

5-(5-Chloro-2-{[(3S)-3-(morpholin-4-ylmethyl)-3,4-dihydroisoquinolin-2 (1H)-yl]carbonyl}phenyl)-1-(2-fluoroethyl)-N-(4-hydroxyphenyl)-2-methyl-N-(1-methyl-1H-pyrrolo[2,3-B]pyridin-5-yl)-1H-pyrrole-3-carboxamide

### EXAMPLE 150

5-(5-Chloro-2-{[(3S)-3-(morpholin-4-ylmethyl)-3,4-dihydroisoquinolin-2 (1H)-yl]carbonyl}phenyl)-1-(2-fluoroethyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 151

5-(5-Chloro-2-{[(3S)-3-(morpholin-4-ylmethyl)-3,4-dihydroisoquinolin-2 (1H)-yl]carbonyl}phenyl)-1-(2,2-difluoroethyl)-N-(4-hydroxyphenyl)-2-methyl-N-(1-methyl-1H-pyrazol-4-yl)-1H-pyrrole-3-carboxamide

### EXAMPLE 152

5-(5-Chloro-2-{[(3S)-3-(morpholin-4-ylmethyl)-3,4-dihydroisoquinolin-2 (1H)-yl]carbonyl}phenyl)-1-(2,2-difluoroethyl)-N-(4-hydroxyphenyl)-2-methyl-N-(1-methyl-1H-pyrrolo[2,3-B]pyridin-5-yl)-1H-pyrrole-3-carboxamide

### EXAMPLE 153

5-(5-Chloro-2-{[(3S)-3-(morpholin-4-ylmethyl)-3,4-dihydroisoquinolin-2 (1H)-yl]carbonyl}phenyl)-1-(2,2-difluoroethyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 154

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1-(2,2,2-trifluoroethyl)-1H-pyrrole-3-carboxamide

### EXAMPLE 155

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1-(2,2,2-trifluoroethyl)-1H-pyrrole-3-carboxamide

### EXAMPLE 156

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1-(2,2,2-trifluoroethyl)-1H-pyrrole-3-carboxamide

### EXAMPLE 157

5-(5-Chloro-2-{[(3R)-3-methyl-3,4-dihydroisoquinolin-2 (1H)-yl]carbonyl}phenyl)-N-(4-hydroxyphenyl)-2-methyl-N-(1-methyl-1H-pyrazol-4-yl)-1-[2-(morpholin-4-yl)ethyl]-1H-pyrrole-3-carboxamide

### EXAMPLE 158

5-(5-Chloro-2-{[(3R)-3-methyl-3,4-dihydroisoquinolin-2 (1H)-yl]carbonyl}phenyl)-N-(4-hydroxyphenyl)-2-methyl-N-(1-Methyl-1H-pyrrolo[2,3-B]pyridin-5-yl)-1-[2-(morpholin-4-yl)ethyl]-1H-pyrrole-3-carboxamide

### EXAMPLE 159

5-(5-Chloro-2-{[(3R)-3-methyl-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1-[2-(morpholin-4-yl)ethyl]-1H-pyrrole-3-carboxamide

### EXAMPLE 160

5-(5-Chloro-2-{[(3R)-3-methyl-3,4-dihydroisoquinolin-2 (1H)-yl]carbonyl}phenyl)-N-(4-hydroxyphenyl)-2-methyl-1-[2-(morpholin-4-yl)ethyl]-N-phenyl-1H-pyrrole-3-carboxamide

### EXAMPLE 161

5-(5-Chloro-2-{[(3R)-3-methyl-3,4-dihydroisoquinolin-2 (1H)-yl]carbonyl}phenyl)-1-[2-(dimethylamino)ethyl]-N-(4-hydroxyphenyl)-2-methyl-N-(1-methyl-1H-PYRAZOL-4-yl)-1H-pyrrole-3-carboxamide

### EXAMPLE 162

5-(5-Chloro-2-{[(3R)-3-methyl-3,4-dihydroisoquinolin-2 (1H)-yl]carbonyl}phenyl)-1-[2-(dimethylamino)ethyl]-N-(4-hydroxyphenyl)-2-methyl-N-phenyl-1H-pyrrole-3-carboxamide

### EXAMPLE 163

5-(5-Chloro-2-{[(3R)-3-methyl-3,4-dihydroisoquinolin-2 (1H)-yl]carbonyl}phenyl)-1-[2-(dimethylamino)ethyl]-N-(4-hydroxyphenyl)-2-methyl-N-(1-methyl-1H-pyrrolo[2,3-B]pyridin-5-yl)-1H-pyrrole-3-carboxamide

### EXAMPLE 164

5-(5-Chloro-2-{[(3R)-3-methyl-3,4-dihydroisoquinolin-2 (1H)-yl]carbonyl}phenyl)-1-[2-(dimethylamino)ethyl]-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 165

5-(5-Chloro-2-{[(3R)-3-methyl-3,4-dihydroisoquinolin-2 (1H)-yl]-carbonyl}phenyl)-N-(4-hydroxyphenyl)-1-{2-[(2-methoxyethyl)(methyl)amino]ethyl}-2-methyl-N-(1-methyl-1H-pyrazol-4-yl)-1H-pyrrole-3-carboxamide

### EXAMPLE 166

5-(5-Chloro-2-{[(3R)-3-methyl-3,4-dihydroisoquinolin-2 (1H)-yl]-carbonyl}phenyl)-N-(4-hydroxyphenyl)-1-{2-[(2-methoxyethyl)(methyl)amino]ethyl}-2-methyl-N-(1-methyl-1H-pyrrolo[2,3-B]pyridin-5-yl)-1H-pyrrole-3-carboxamide

### EXAMPLE 167

5-(5-Chloro-2-{[(3R)-3-methyl-3,4-dihydroisoquinolin-2 (1H)-yl]-carbonyl}phenyl)-N-(4-hydroxyphenyl)-1-{2-[(2-methoxyethyl)(methyl)amino]ethyl}-2-methyl-N-(1-methyl-2,3-dihydro-1H-pyrrolo[2,3-B]pyridin-5-yl)-1H-pyrrole-3-carboxamide

### EXAMPLE 168

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-2-(trifluoromethyl)-1H-pyrrole-3-carboxamide

### EXAMPLE 169

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-(trifluoromethyl)-1H-pyrrole-3-carboxamide

### EXAMPLE 170

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-(trifluoromethyl)-1H-pyrrole-3-carboxamide

### EXAMPLE 171

5-(5-Chloro-2-{[(3S)-3-(morpholin-4-ylmethyl)-3,4-dihydroisoquinolin-2 (1H)-yl]carbonyl}phenyl)-2-(difluoromethyl)-N-(4-hydroxyphenyl)-1-methyl-N-(1-methyl-1H-pyrazol-4-yl)-1H-pyrrole-3-carboxamide

### EXAMPLE 172

5-(5-Chloro-2-{[(3S)-3-(morpholin-4-ylmethyl)-3,4-dihydroisoquinolin-2 (1H)-yl]carbonyl}phenyl)-2-(difluoromethyl)-N-(4-hydroxyphenyl)-1-methyl-N-(1-methyl-1H-pyrrolo[2,3-B]pyridin-5-yl)-1H-pyrrole-3-carboxamide

### EXAMPLE 173

5-(5-Chloro-2-{[(3S)-3-(morpholin-4-ylmethyl)-3,4-dihydroisoquinolin-2 (1H)-yl]carbonyl}phenyl)-2-(difluoromethyl)-N-(4-hydroxyphenyl)-1-methyl-N-(1-methyl-2,3-dihydro-1H-pyrrolo[2,3-B]pyridin-5-yl)-1H-pyrrole-3-carboxamide

### EXAMPLE 174

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-(methoxymethyl)-1-methyl-N-(1-methyl-1H-pyrazol-4-yl)-1H-pyrrole-3-carboxamide

### EXAMPLE 175

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-(methoxymethyl)-1-methyl-N-(1-methyl-1H-pyrrolo[2,3-B]pyridin-5-yl)-1H-pyrrole-3-carboxamide

### EXAMPLE 176

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-(methoxymethyl)-1-methyl-N-(1-methyl-2,3-dihydro-1H-pyrrolo[2,3-B]pyridin-5-yl)-1H-pyrrole-3-carboxamide

### EXAMPLE 177

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-2-(2,2,2-trifluoroethyl)-1H-pyrrole-3-carboxamide

### EXAMPLE 178

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-(2,2,2-trifluoroethyl)-1H-pyrrole-3-carboxamide

### EXAMPLE 179

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-(2,2,2-trifluoroethyl)-1H-pyrrole-3-carboxamide

### EXAMPLE 180

5-(5-Chloro-2-{[(3S)-3-(morpholin-4-ylmethyl)-3,4-dihydroisoquinolin-2 (1H)-yl]carbonyl}phenyl)-2-(2,2-difluoroethyl)-N-(4-hydroxyphenyl)-1-methyl-N-(1-methyl-1H-pyrazol-4-yl)-1H-pyrrole-3-carboxamide

### EXAMPLE 181

5-(5-Chloro-2-{[(3S)-3-(morpholin-4-ylmethyl)-3,4-dihydroisoquinolin-2 (1H)-yl]carbonyl}phenyl)-2-(2,2-difluoroethyl)-N-(4-hydroxyphenyl)-1-methyl-N-(1-methyl-1H-pyrrolo[2,3-B]pyridin-5-yl)-1H-pyrrole-3-carboxamide

### EXAMPLE 182

5-(5-Chloro-2-{[(3S)-3-(morpholin-4-ylmethyl)-3,4-dihydroisoquinolin-2 (1H)-yl]carbonyl}phenyl)-2-(2,2-difluoroethyl)-N-(4-hydroxyphenyl)-1-methyl-N-(1-methyl-2,3-dihydro-1H-pyrrolo[2,3-B]pyridin-5-yl)-1H-pyrrole-3-carboxamide

### EXAMPLE 183

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-(2-methoxyethyl)-1-methyl-N-(1-methyl-1H-pyrazol-4-yl)-1H-pyrrole-3-carboxamide

### EXAMPLE 184

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-(2-methoxyethyl)-1-methyl-N-(1-methyl-1H-pyrrolo[2,3-B]pyridin-5-yl)-1H-pyrrole-3-carboxamide

### EXAMPLE 185

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-2-(2-methoxyethyl)-1-methyl-N-(1-methyl-2,3-dihydro-1H-pyrrolo[2,3-B]pyridin-5-yl)-1H-pyrrole-3-carboxamide

### EXAMPLE 186

5-(5-Chloro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrazol-4-yl)-2-(morpholin-4-ylmethyl)-1H-pyrrole-3-carboxamide

### EXAMPLE 187

5-(5-Chloro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-(morpholin-4-ylmethyl)-1H-pyrrole-3-carboxamide

### EXAMPLE 188

5-(5-Chloro-2-{[(3R)-3-methyl-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-(morpholin-4-ylmethyl)-1H-pyrrole-3-carboxamide

### EXAMPLE 189

5-(5-Chloro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrazol-4-yl)-2-[2-(morpholin-4-yl)ethyl]-1H-pyrrole-3-carboxamide

### EXAMPLE 190

5-(5-Chloro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-[2-(morpholin-4-yl)ethyl]-1H-pyrrole-3-carboxamide

### EXAMPLE 191

5-(5-Chloro-2-{[(3R)-3-methyl-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-[2-(morpholin-4-yl)ethyl]-1H-pyrrole-3-carboxamide

### EXAMPLE 192

5-(5-Chloro-2-{[(3S)-3-(trifluoromethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 193

5-(5-Chloro-2-{[(3S)-3-(trifluoromethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 194

5-(5-Chloro-2-{[(3S)-3-(difluoromethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 195

5-(5-Chloro-2-{[(3S)-3-(difluoromethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 196

5-(5-Chloro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 197

5-(5-Chloro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 198

5-(5-Chloro-2-{[(3S)-3-[(9AS)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 199

5-(5-Chloro-2-{[(3S)-3-[(9AS)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 200

5-(5-Chloro-2-{[(3S)-3-[(9AR)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 201

5-(5-Chloro-2-{[(3S)-3-[(9AR)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 202

5-(2-{[(3S)-3-(aminomethyl)-3,4-dihydroisoquinolin-2 (1H)-yl]carbonyl}-5-chlorophenyl)-N-(4-hydroxyphenyl)-1,2-dimethyl-N-(1-methyl-1H-pyrazol-4-yl)-1H-pyrrole-3-carboxamide

### EXAMPLE 203

5-(2-{[(3S)-3-(aminomethyl)-3,4-dihydroisoquinolin-2 (1H)-yl]carbonyl}-5-chlorophenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 204

5-(2-{[(3S)-3-(aminomethyl)-3,4-dihydroisoquinolin-2 (1H)-yl]carbonyl}-5-chlorophenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 205

5-(5-Chloro-2-{[(3S)-3-[(3-cyanoazetidin-1-yl)methyl]-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 206

5-(5-Chloro-2-{[(3S)-3-[(3-cyanoazetidin-1-yl)methyl]-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 207

5-(5-Chloro-2-{[(3S)-3-(1-oxa-6-azaspiro[3.3]hept-6-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

The title compound is obtained in accordance with the process of Example 1 using the acid of Preparation 1 and the compound of Preparation 8′ in Step A, and the compound of Preparation 1″ in Step C.

### High-Resolution Mass Spectrometry (ESI+−/FIA/HR, ESI−/FIA):

C₃₉H₃₉Cl N₆O₄ Empirical formula:

[M+H]⁺ calculated: 691.2794

[M+H]⁺ measured: 691.2796

### EXAMPLE 208

5-(5-Chloro-2-{[(3S)-3-(1-oxa-6-azaspiro[3.3]hept-6-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 209

5-(5-fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide hydrochloride

The procedure is in accordance with the general protocol of Example 1 using the appropriate Preparations. The product obtained is finally dissolved in acetonitrile and converted into salt form using 0.1M aqueous HCl solution. After a lyophilisation step, the expected compound is obtained in the form of a solid.

### Elemental Microanalysis: (%, Theoretical:Measured)

% C=66.27:66.84; % H=5.69:5.15; % N=10.78:10.71; % Cl—=4.55:4.46

### High-Resolution Mass Spectrometry (ESI+−/FIA):

C₄₃H₄₃F N₆O₅ Empirical formula:

[M+H]⁺ calculated: 743.3352

[M+H]⁺ measured: 743.3353

### EXAMPLE 210

5-(5-fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide hydrochloride
Step A: 5-(5-Fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-b]pyridin-5-yl)-1H-pyrrole-3-carboxamide

The title compound is obtained in accordance with the process of Example 1 using the compound of Preparation 14 and (3S)-3-(4-morpholinylmethyl)-1,2,3,4-tetrahydroisoquinoline (see Preparation 3′) in Step A, and the compound of Preparation 11″ in Step C.

Step B: 5-(5-Chloro-2-{[(3S)-3-(morpholin-4-ylmethyl)-3,4-dihydroisoquinolin-2 (1H)-yl]carbonyl}phenyl)-1,2-dimethyl-N-(1-methyl-2,3-dihydro-1H-pyrrolo[2,3-b]pyridin-5-yl)-N-phenyl-1H-pyrrole-3-carboxamide hydrochloride

The title compound is obtained in accordance with the process described in Step B of Example 49 starting from compound of the above Step, it being understood that the product is finally subjected to a step of conversion into salt form in the presence of 1M HCl in ether. After filtration and lyophilisation in a mixture of acetonitrile/water, the expected product is obtained.

### Elemental Microanalysis: (%, Theoretical:Measured)

% C=60.12:59.77; % H=4.92:4.76; % N=10.79:10.39; % Cl—=4.55:5.17

### High-Resolution Mass Spectrometry (ESI/FIA/HR and MS/MS):

C₄₀H₄₀N₄O₅ Empirical formula:

[M+H]⁺ calculated: 657.3071

[M+H]⁺ measured: 657.3066

### EXAMPLE 211

5-(4-fluoro-5-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 212

5-(4-fluoro-5-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 213

N-(1-ethyl-1H-pyrazol-4-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide

### EXAMPLE 214

N-(1-cyclopropyl-1H-pyrazol-4-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the compound of Preparation 28″ in Step C. The compound obtained is converted into salt form and lyophilised as described in the general process to obtain the title compound.

### Elemental Microanalysis: (%, Theoretical:Measured)

% C=66.24:66.53; % H=5.84:5.39; % N=10.59:10.92% Cl⁻=4.89:5.68

### EXAMPLE 215

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(trifluoromethyl)-1H-pyrazol-4-yl]-1H-pyrrole-3-carboxamide

### EXAMPLE 216

N-[1-(difluoromethyl)-1H-pyrazol-4-yl]-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide

### EXAMPLE 217

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-N-[1-(2-methoxyethyl)-1H-pyrazol-4-yl]-1,2-dimethyl-1H-pyrrole-3-carboxamide

### EXAMPLE 218

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(OXETAN-3-yl)-1H-pyrazol-4-yl]-1H-pyrrole-3-carboxamide

### EXAMPLE 219

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(tetrahydrofuran-3-yl)-1H-pyrazol-4-yl]-1H-pyrrole-3-carboxamide

### EXAMPLE 220

5-(5-fluoro-2-{[(3S)-3-(morpholin-4-ylmethyl)-3,4-dihydroisoquinolin-2 (1H)-yl]carbonyl}phenyl)-N-[1-(2-hydroxy-2-methylpropyl)-1H-pyrazol-4-yl]-N-(4-hydroxyphenyl)-1,2-dimethyl-1H-pyrrole-3-carboxamide

### EXAMPLE 221

5-(5-fluoro-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrrolo[2,3-B]pyridin-5-yl)-N-phenyl-1H-pyrrole-3-carboxamide

### EXAMPLE 222

5-(5-fluoro-2-{[(3S)-3-(morpholin-4-ylmethyl)-3,4-dihydroisoquinolin-2 (1H)-yl]carbonyl}phenyl)-1,2-dimethyl-N-(1-methyl-2,3-dihydro-1H-pyrrolo[2,3-B]pyridin-5-yl)-N-phenyl-1H-pyrrole-3-carboxamide

### EXAMPLE 223

5-(5-fluoro-2-{[(3S)-3-(morpholin-4-ylmethyl)-3,4-dihydroisoquinolin-2 (1H)-yl]carbonyl}phenyl)-N-(4-fluorophenyl)-1,2-dimethyl-N-(1-methyl-1H-pyrrolo[2,3-B]pyridin-5-yl)-1H-pyrrole-3-carboxamide

### EXAMPLE 224

5-(5-fluoro-2-{[(3S)-3-(morpholin-4-ylmethyl)-3,4-dihydroisoquinolin-2 (1H)-yl]carbonyl}phenyl)-N-(4-fluorophenyl)-1,2-dimethyl-N-(1-methyl-2,3-dihydro-1H-pyrrolo[2,3-B]pyridin-5-yl)-1H-pyrrole-3-carboxamide

### EXAMPLE 225

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-N,1,2-trimethyl-1H-pyrrole-3-carboxamide

### EXAMPLE 226

5-(5-fluoro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-phenyl-1H-pyrrole-3-carboxamide

### EXAMPLE 227

5-(5-fluoro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-(1-methyl-1H-pyrrolo[2,3-B]pyridin-5-yl)-1H-pyrrole-3-carboxamide

### EXAMPLE 228

5-(5-fluoro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-(1-methyl-2,3-dihydro-1H-pyrrolo[2,3-B]pyridin-5-yl)-1H-pyrrole-3-carboxamide

### EXAMPLE 229

5-(5-fluoro-2-{[(3S)-3-(morpholin-4-ylmethyl)-3,4-dihydroisoquinolin-2 (1H)-yl]carbonyl}phenyl)-N-(2-hydroxypyrimidin-5-yl)-1,2-dimethyl-N-(1-methyl-1H-pyrazol-4-yl)-1H-pyrrole-3-carboxamide

### EXAMPLE 230

N-(5-cyano-1-methyl-1H-PYRROL-3-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the compound of Preparation 19″ in Step C. The compound obtained is converted into salt form and lyophilised as described in the general process to obtain the title compound.

### Elemental Microanalysis: (%, Theoretical:Measured)

% C=66.43:67.09; % H=5.57:5.21; % N=11.62:11.48% Cl⁻=4.90:4.75

### High-Resolution Mass Spectrometry (ESI+):

C₄₀H₃₉FN₆O₄ Empirical formula:

[M+H]⁺ calculated: 687.3097

[M+H]⁺ measured: 687.3073

### EXAMPLE 231

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1H-pyrrole-3-carboxamide

### EXAMPLE 232

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1H-pyrrole-3-carboxamide

### EXAMPLE 233

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 234

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-1H-pyrrole-3-carboxamide

### EXAMPLE 235

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-1H-pyrrole-3-carboxamide

### EXAMPLE 236

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-1H-pyrrole-3-carboxamide

### EXAMPLE 237

1-ethyl-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1H-pyrrole-3-carboxamide

### EXAMPLE 238

1-ethyl-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1H-pyrrole-3-carboxamide

### EXAMPLE 239

1-ethyl-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 240

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-(2-methoxyethyl)-2-methyl-N-(1-methyl-1H-pyrazol-4-yl)-1H-pyrrole-3-carboxamide

### EXAMPLE 241

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-(2-methoxyethyl)-2-methyl-N-(1-methyl-1H-pyrrolo[2,3-B]pyridin-5-yl)-1H-pyrrole-3-carboxamide

### EXAMPLE 242

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-(2-methoxyethyl)-2-methyl-N-(1-methyl-2,3-dihydro-1H-pyrrolo[2,3-B]pyridin-5-yl)-1H-pyrrole-3-carboxamide

### EXAMPLE 243

1-(2-fluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1H-pyrrole-3-carboxamide

### EXAMPLE 244

1-(2-fluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1H-pyrrole-3-carboxamide

### EXAMPLE 245

1-(2-fluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 246

1-(2,2-difluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1H-pyrrole-3-carboxamide

### EXAMPLE 247

1-(2,2-difluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1H-pyrrole-3-carboxamide

### EXAMPLE 248

1-(2,2-difluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 249

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrazol-4-yl)-1-(2,2,2-trifluoroethyl)-1H-pyrrole-3-carboxamide

### EXAMPLE 250

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1-(2,2,2-trifluoroethyl)-1H-pyrrole-3-carboxamide

### EXAMPLE 251

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1-(2,2,2-trifluoroethyl)-1H-pyrrole-3-carboxamide

### EXAMPLE 252

5-(5-fluoro-2-{[(3R)-3-methyl-3,4-dihydroisoquinolin-2 (1H)-yl]carbonyl}phenyl)-N-(4-hydroxyphenyl)-2-methyl-N-(1-methyl-1H-pyrazol-4-yl)-1-[2-(morpholin-4-yl)ethyl]-1H-pyrrole-3-carboxamide

### EXAMPLE 253

5-(5-fluoro-2-{[(3R)-3-methyl-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1-[2-(morpholin-4-yl)ethyl]-1H-pyrrole-3-carboxamide

### EXAMPLE 254

5-(5-fluoro-2-{[(3R)-3-methyl-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1-[2-(morpholin-4-yl)ethyl]-1H-pyrrole-3-carboxamide

### EXAMPLE 255

5-(5-fluoro-2-{[(3R)-3-methyl-3,4-dihydroisoquinolin-2 (1H)-yl]carbonyl}phenyl)-N-(4-hydroxyphenyl)-2-methyl-1-[2-(morpholin-4-yl)ethyl]-N-phenyl-1H-pyrrole-3-carboxamide

### EXAMPLE 256

1-[2-(dimethylamino)ethyl]-5-(5-fluoro-2-{[(3R)-3-methyl-3,4-dihydroisoquinolin-2 (1H)-yl]carbonyl}phenyl)-N-(4-hydroxyphenyl)-2-methyl-N-(1-methyl-1H-pyrazol-4-yl)-1H-pyrrole-3-carboxamide

### EXAMPLE 257

1-[2-(dimethylamino)ethyl]-5-(5-fluoro-2-{[(3R)-3-methyl-3,4-dihydroisoquinolin-2 (1H)-yl]carbonyl}phenyl)-N-(4-hydroxyphenyl)-2-methyl-N-phenyl-1H-pyrrole-3-carboxamide

### EXAMPLE 258

1-[2-(dimethylamino)ethyl]-5-(5-fluoro-2-{[(3R)-3-methyl-3,4-dihydroisoquinolin-2 (1H)-yl]carbonyl}phenyl)-N-(4-hydroxyphenyl)-2-methyl-N-(1-methyl-1H-pyrrolo[2,3-B]pyridin-5-yl)-1H-pyrrole-3-carboxamide

### EXAMPLE 259

1-[2-(dimethylamino)ethyl]-5-(5-fluoro-2-{[(3R)-3-methyl-3,4-dihydroisoquinolin-2 (1H)-yl]carbonyl}phenyl)-N-(4-hydroxyphenyl)-2-methyl-N-(1-methyl-2,3-dihydro-1H-pyrrolo[2,3-B]pyridin-5-yl)-1H-pyrrole-3-carboxamide

### EXAMPLE 260

5-(5-fluoro-2-{[(3R)-3-methyl-3,4-dihydroisoquinolin-2 (1H)-yl]-carbonyl}phenyl)-N-(4-hydroxyphenyl)-1-{2-[(2-methoxyethyl)(methyl)amino]ethyl}-2-methyl-N-(1-methyl-1H-pyrazol-4-yl)-1H-pyrrole-3-carboxamide

### EXAMPLE 261

5-(5-fluoro-2-{[(3R)-3-methyl-3,4-dihydroisoquinolin-2 (1H)-yl]-carbonyl}phenyl)-N-(4-hydroxyphenyl)-1-{2-[(2-methoxyethyl)(methyl)amino]ethyl}-2-methyl-N-(1-methyl-1H-pyrrolo[2,3-B]pyridin-5-yl)-1H-pyrrole-3-carboxamide

### EXAMPLE 262

5-(5-fluoro-2-{[(3R)-3-methyl-3,4-dihydroisoquinolin-2 (1H)-yl]-carbonyl}phenyl)-N-(4-hydroxyphenyl)-1-{2-[(2-methoxyethyl)(methyl)amino]ethyl}-2-methyl-N-(1-methyl-2,3-dihydro-1H-pyrrolo[2,3-B]pyridin-5-yl)-1H-pyrrole-3-carboxamide

### EXAMPLE 263

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-2-(trifluoromethyl)-1H-pyrrole-3-carboxamide

### EXAMPLE 264

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-(trifluoromethyl)-1H-pyrrole-3-carboxamide

### EXAMPLE 265

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-(trifluoromethyl)-1H-pyrrole-3-carboxamide

### EXAMPLE 266

2-(difluoromethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-1H-pyrrole-3-carboxamide

### EXAMPLE 267

2-(difluoromethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-1H-pyrrole-3-carboxamide

### EXAMPLE 268

2-(difluoromethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-1H-pyrrole-3-carboxamide

### EXAMPLE 269

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-(methoxymethyl)-1-methyl-N-(1-methyl-1H-pyrazol-4-yl)-1H-pyrrole-3-carboxamide

### EXAMPLE 270

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-(methoxymethyl)-1-methyl-N-(1-methyl-1H-pyrrolo[2,3-B]pyridin-5-yl)-1H-pyrrole-3-carboxamide

### EXAMPLE 271

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-(methoxymethyl)-1-methyl-N-(1-methyl-2,3-dihydro-1H-pyrrolo[2,3-B]pyridin-5-yl)-1H-pyrrole-3-carboxamide

### EXAMPLE 272

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-2-(2,2,2-trifluoroethyl)-1H-pyrrole-3-carboxamide

### EXAMPLE 273

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-(2,2,2-trifluoroethyl)-1H-pyrrole-3-carboxamide

### EXAMPLE 274

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-(2,2,2-trifluoroethyl)-1H-pyrrole-3-carboxamide

### EXAMPLE 275

2-(2,2-difluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrazol-4-yl)-1H-pyrrole-3-carboxamide

### EXAMPLE 276

2-(2,2-difluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-1H-pyrrole-3-carboxamide

### EXAMPLE 277

2-(2,2-difluoroethyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-1H-pyrrole-3-carboxamide

### EXAMPLE 278

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-(2-methoxyethyl)-1-methyl-N-(1-methyl-1H-pyrazol-4-yl)-1H-pyrrole-3-carboxamide

### EXAMPLE 279

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-(2-methoxyethyl)-1-methyl-N-(1-methyl-1H-pyrrolo[2,3-B]pyridin-5-yl)-1H-pyrrole-3-carboxamide

### EXAMPLE 280

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-2-(2-methoxyethyl)-1-methyl-N-(1-methyl-2,3-dihydro-1H-pyrrolo[2,3-B]pyridin-5-yl)-1H-pyrrole-3-carboxamide

### EXAMPLE 281

5-(5-fluoro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrazol-4-yl)-2-(morpholin-4-ylmethyl)-1H-pyrrole-3-carboxamide

### EXAMPLE 282

5-(5-fluoro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-(morpholin-4-ylmethyl)-1H-pyrrole-3-carboxamide

### EXAMPLE 283

5-(5-fluoro-2-{[(3R)-3-methyl-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-(morpholin-4-ylmethyl)-1H-pyrrole-3-carboxamide

### EXAMPLE 284

5-(5-fluoro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrazol-4-yl)-2-[2-(morpholin-4-yl)ethyl]-1H-pyrrole-3-carboxamide

### EXAMPLE 285

5-(5-fluoro-2-{[(3R)-3-methyl-3,4-dihydroisoquinolin-2 (1H)-yl]carbonyl}phenyl)-N-(4-hydroxyphenyl)-1-methyl-N-(1-methyl-1H-pyrrolo[2,3-B]pyridin-5-yl)-2-[2-(morpholin-4-yl)ethyl]-1H-pyrrole-3-carboxamide

### EXAMPLE 286

5-(5-fluoro-2-{[(3R)-3-methyl-3,4-dihydroisoquinolin-2 (1H)-yl]carbonyl}phenyl)-N-(4-hydroxyphenyl)-1-methyl-N-(1-methyl-2,3-dihydro-1H-pyrrolo[2,3-B]pyridin-5-yl)-2-[2-(morpholin-4-yl)ethyl]-1H-pyrrole-3-carboxamide

### EXAMPLE 287

5-(5-fluoro-2-{[(3S)-3-(trifluoromethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 288

5-(5-fluoro-2-{[(3S)-3-(trifluoromethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 289

5-(5-fluoro-2-{[(3S)-3-(trifluoromethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 290

5-(2-{[(3S)-3-(difluoromethyl)-3,4-dihydroisoquinolin-2 (1H)-yl]carbonyl}-5-fluorophenyl)-N-(4-hydroxyphenyl)-1,2-dimethyl-N-(1-methyl-1H-pyrazol-4-yl)-1H-pyrrole-3-carboxamide

### EXAMPLE 291

5-(2-{[(3S)-3-(difluoromethyl)-3,4-dihydroisoquinolin-2 (1H)-yl]carbonyl}-5-fluorophenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 292

5-(2-{[(3S)-3-(difluoromethyl)-3,4-dihydroisoquinolin-2 (1H)-yl]carbonyl}-5-fluorophenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 293

5-(5-fluoro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 294

5-(5-fluoro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 295

5-(5-fluoro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 296

5-(5-fluoro-2-{[(3S)-3-(fluoromethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 297

5-(5-fluoro-2-{[(3S)-3-[(9AS)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 298

5-(5-fluoro-2-{[(3S)-3-[(9AS)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 299

5-(5-fluoro-2-{[(3S)-3-[(9AR)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 300

5-(5-fluoro-2-{[(3S)-3-[(9AR)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 301

5-(5-fluoro-2-{[(3S)-3-[(9AR)-hexahydropyrazino[2,1-C][1,4]oxazin-8 (1H)-ylmethyl]-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 302

5-(2-{[(3S)-3-(aminomethyl)-3,4-dihydroisoquinolin-2 (1H)-yl]carbonyl}-5-fluorophenyl)-N-(4-hydroxyphenyl)-1,2-dimethyl-N-(1-methyl-1H-pyrazol-4-yl)-1H-pyrrole-3-carboxamide

### EXAMPLE 303

5-(2-{[(3S)-3-(aminomethyl)-3,4-dihydroisoquinolin-2 (1H)-yl]carbonyl}-5-fluorophenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 304

5-(2-{[(3S)-3-(aminomethyl)-3,4-dihydroisoquinolin-2 (1H)-yl]carbonyl}-5-fluorophenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 305

5-(2-{[(3S)-3-[(3-cyanoazetidin-1-yl)methyl]-3,4-dihydroisoquinolin-2 (1H)-yl]carbonyl}-5-fluorophenyl)-N-(4-hydroxyphenyl)-1,2-dimethyl-N-(1-methyl-1H-pyrazol-4-yl)-1H-pyrrole-3-carboxamide

### EXAMPLE 306

5-(2-{[(3S)-3-[(3-cyanoazetidin-1-yl)methyl]-3,4-dihydroisoquinolin-2 (1H)-yl]carbonyl}-5-fluorophenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 307

5-(2-{[(3S)-3-[(3-cyanoazetidin-1-yl)methyl]-3,4-dihydroisoquinolin-2 (1H)-yl]carbonyl}-5-fluorophenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 308

5-(5-fluoro-2-{[(3S)-3-(1-oxa-6-azaspiro[3.3]hept-6-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide

### EXAMPLE 309

5-(5-fluoro-2-{[(3S)-3-(1-oxa-6-azaspiro[3.3]hept-6-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 310

N-(1-ethyl-1H-pyrazol-4-yl)-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 311

N-(1-cyclopropyl-1H-pyrazol-4-yl)-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 312

N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-N-[1-(trifluoromethyl)-1H-pyrazol-4-yl]-1H-pyrrole-3-carboxamide

### EXAMPLE 313

N-[1-(difluoromethyl)-1H-pyrazol-4-yl]-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 314

N-(4-hydroxyphenyl)-N-[1-(2-methoxyethyl)-1H-pyrazol-4-yl]-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 315

N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-N-[1-(OXETAN-3-yl)-1H-pyrazol-4-yl]-1H-pyrrole-3-carboxamide

### EXAMPLE 316

N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-N-[1-(tetrahydrofuran-3-yl)-1H-pyrazol-4-yl]-1H-pyrrole-3-carboxamide

### EXAMPLE 317

N-[1-(2-hydroxy-2-methylpropyl)-1H-pyrazol-4-yl]-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 318

1,2-dimethyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-N-phenyl-1H-pyrrole-3-carboxamide

### EXAMPLE 319

1,2-dimethyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-N-phenyl-1H-pyrrole-3-carboxamide

### EXAMPLE 320

N-(4-fluorophenyl)-1,2-dimethyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 321

N-(4-fluorophenyl)-1,2-dimethyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 322

N-(4-hydroxyphenyl)-N,1,2-trimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 323

N-(2-hydroxypyrimidin-5-yl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-N-phenyl-1H-pyrrole-3-carboxamide

### EXAMPLE 324

N-(2-hydroxypyrimidin-5-yl)-1,2-dimethyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 325

N-(2-hydroxypyrimidin-5-yl)-1,2-dimethyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 326

N-(2-hydroxypyrimidin-5-yl)-1,2-dimethyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 327

N-(5-cyano-1-methyl-1H-PYRROL-3-yl)-N-(4-hydroxyphenyl)-1,2-dimethyl-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 328

N-(4-hydroxyphenyl)-2-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 329

N-(4-hydroxyphenyl)-2-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 330

N-(4-hydroxyphenyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 331

N-(4-hydroxyphenyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 332

N-(4-hydroxyphenyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 333

1-ethyl-N-(4-hydroxyphenyl)-2-methyl-N-(1-methyl-1H-pyrrolo[2,3-B]-pyridin-5-yl)-5-(6-{[(3S)-3-(morpholin-4-ylmethyl)-3,4-dihydroisoquinolin-2 (1H)-yl]-carbonyl}-1,3-benzodioxol-5-yl)-1H-pyrrole-3-carboxamide

### EXAMPLE 334

1-ethyl-N-(4-hydroxyphenyl)-2-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 335

N-(4-hydroxyphenyl)-1-(2-methoxyethyl)-2-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 336

N-(4-hydroxyphenyl)-1-(2-methoxyethyl)-2-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 337

1-(2-fluoroethyl)-N-(4-hydroxyphenyl)-2-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 338

1-(2-fluoroethyl)-N-(4-hydroxyphenyl)-2-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 339

1-(2,2-difluoroethyl)-N-(4-hydroxyphenyl)-2-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 340

1-(2,2-difluoroethyl)-N-(4-hydroxyphenyl)-2-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 341

N-(4-hydroxyphenyl)-2-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1-(2,2,2-trifluoroethyl)-1H-pyrrole-3-carboxamide

### EXAMPLE 342

N-(4-hydroxyphenyl)-2-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1-(2,2,2-trifluoroethyl)-1H-pyrrole-3-carboxamide

### EXAMPLE 343

N-(4-hydroxyphenyl)-2-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1-(2,2,2-trifluoroethyl)-1H-pyrrole-3-carboxamide

### EXAMPLE 344

N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrrolo[2,3-B]pyridin-5-yl)-1-[2-(morpholin-4-yl)ethyl]-1H-pyrrole-3-carboxamide

### EXAMPLE 345

N-(4-hydroxyphenyl)-2-methyl-5-(6-{[(3R)-3-methyl-3,4-DIHYDROISO-QUINOLIN-2 (1H)-yl]carbonyl}-1,3-benzodioxol-5-yl)-N-(1-methyl-2,3-dihydro-1H-pyrrolo[2,3-B]pyridin-5-yl)-1-[2-(morpholin-4-yl)ethyl]-1H-pyrrole-3-carboxamide

### EXAMPLE 346

1-[2-(dimethylamino)ethyl]-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrazol-4-yl)-1H-pyrrole-3-carboxamide

### EXAMPLE 347

1-[2-(dimethylamino)ethyl]-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-1H-pyrrolo[2,3-B]pyridin-5-yl)-1H-pyrrole-3-carboxamide

### EXAMPLE 348

1-[2-(dimethylamino)ethyl]-N-(4-hydroxyphenyl)-2-methyl-5-(6-{[(3R)-3-methyl-3,4-dihydroisoquinolin-2 (1H)-yl]carbonyl}-1,3-benzodioxol-5-yl)-N-(1-methyl-2,3-dihydro-1H-pyrrolo[2,3-B]pyridin-5-yl)-1H-pyrrole-3-carboxamide

### EXAMPLE 349

N-(4-hydroxyphenyl)-1-{2-[(2-methoxyethyl)(methyl)amino]ethyl}-2-methyl-5-(6-{[(3R)-3-methyl-3,4-dihydroisoquinolin-2 (1H)-yl]carbonyl}-1,3-benzodioxol-5-yl)-N-(1-methyl-1H-pyrrolo[2,3-B]pyridin-5-yl)-1H-pyrrole-3-carboxamide

### EXAMPLE 350

N-(4-hydroxyphenyl)-1-{2-[(2-methoxyethyl)(methyl)amino]ethyl}-2-methyl-5-(6-{[(3R)-3-methyl-3,4-dihydroisoquinolin-2 (1H)-yl]carbonyl}-1,3-benzodioxol-5-yl)-N-(1-methyl-2,3-dihydro-1H-pyrrolo[2,3-B]pyridin-5-yl)-1H-pyrrole-3-carboxamide

### EXAMPLE 351

N-(4-hydroxyphenyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-2-(trifluoromethyl)-1H-pyrrole-3-carboxamide

### EXAMPLE 352

2-(difluoromethyl)-N-(4-hydroxyphenyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 353

N-(4-hydroxyphenyl)-2-(methoxymethyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 354

N-(4-hydroxyphenyl)-1-methyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-2-(2,2,2-trifluoroethyl)-1H-pyrrole-3-carboxamide

### EXAMPLE 355

N-(4-hydroxyphenyl)-1-methyl-N-(1-methyl-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-2-(2,2,2-trifluoroethyl)-1H-pyrrole-3-carboxamide

### EXAMPLE 356

N-(4-hydroxyphenyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-2-(2,2,2-trifluoroethyl)-1H-pyrrole-3-carboxamide

### EXAMPLE 357

2-(2,2-difluoroethyl)-N-(4-hydroxyphenyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide

### EXAMPLE 358

N-(4-hydroxyphenyl)-2-(2-methoxyethyl)-1-methyl-N-(1-methyl-2,3-dihydro-1H-pyrrolo[2,3-B]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

The title compound is obtained in accordance with the process described in Step B of Example 49 using Example 88 as starting material, it being understood that the product is finally dissolved in acetonitrile and converted into salt form in 0.1M aqueous HCl solution. After a lyophilisation step, the expected product is obtained in the form of a solid.

### Elemental Microanalysis: (%, Theoretical:Measured)

% C=65.80:64.71; % H=6.01:5.74; % N=10.23:10.03; % Cl—=4.32:6.47

### High-Resolution Mass Spectrometry (ESI/+):

C₄₅H₄₈N₆O₇ Empirical formula:

[M+H]⁺ calculated: 785.3657

[M+H]⁺ measured: 785.3658

### EXAMPLE 359

N-(4-hydroxyphenyl)-1-methyl-5-(6-{[(3R)-3-methyl-3,4-dihydroisoquinolin-2 (1H)-yl]carbonyl}-1,3-benzodioxol-5-yl)-N-(1-methyl-2,3-dihydro-1H-pyrrolo[2,3-B]pyridin-5-yl)-2-(morpholin-4-ylmethyl)-1H-pyrrole-3-carboxamide

### EXAMPLE 360

N-(4-hydroxyphenyl)-1-methyl-5-(6-{[(3R)-3-methyl-3,4-dihydroisoquinolin-2 (1H)-yl]carbonyl}-1,3-benzodioxol-5-yl)-N-(1-methyl-2,3-dihydro-1H-pyrrolo[2,3-B]pyridin-5-yl)-2-[2-(morpholin-4-yl)ethyl]-1H-pyrrole-3-carboxamide

### EXAMPLE 361

N-(4-hydroxyphenyl)-1,2-dimethyl-N-(1-methyl-1H-pyrazol-4-yl)-5-(6-{[(3S)-3-(trifluoromethyl)-3,4-dihydroisoquinolin-2 (1H)-yl]carbonyl}-1,3-benzodioxol-5-yl)-1H-pyrrole-3-carboxamide

### EXAMPLE 362

N-(4-hydroxyphenyl)-1,2-dimethyl-N-(1-methyl-1H-pyrrolo[2,3-B]pyridin-5-yl)-5-(6-{[(3S)-3-(trifluoromethyl)-3,4-dihydroisoquinolin-2 (1H)-yl]carbonyl}-1,3-benzodioxol-5-yl)-1H-pyrrole-3-carboxamide

### EXAMPLE 363

N-(4-hydroxyphenyl)-1,2-dimethyl-N-(1-methyl-2,3-dihydro-1H-pyrrolo[2,3-B]pyridin-5-yl)-5-(6-{[(3S)-3-(trifluoromethyl)-3,4-dihydroisoquinolin-2 (1H)-yl]carbonyl}-1,3-benzodioxol-5-yl)-1H-pyrrole-3-carboxamide

### EXAMPLE 364

5-(6-{[(3S)-3-(difluoromethyl)-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrazol-4-yl)-1H-pyrrole-3-carboxamide

### EXAMPLE 365

5-(6-{[(3S)-3-(difluoromethyl)-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 366

5-(6-{[(3S)-3-(difluoromethyl)-3,4-dihydroisoquinolin-2 (1H)-yl]carbonyl}-1,3-benzodioxol-5-yl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 367

5-(6-{[(3S)-3-(fluoromethyl)-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrazol-4-yl)-1H-pyrrole-3-carboxamide

### EXAMPLE 368

5-(6-{[(3S)-3-(fluoromethyl)-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 369

5-(6-{[(3S)-3-(fluoromethyl)-3,4-dihydroisoquinolin-2 (1H)-yl]carbonyl}-1,3-benzodioxol-5-yl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 370

5-(6-{[(3S)-3-[(9AS)-hexahydropyrazino[2,1-C][1,4]oxazin-8 (1H)-ylmethyl]-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrazol-4-yl)-1H-pyrrole-3-carboxamide

### EXAMPLE 371

5-(6-{[(3S)-3-[(9AS)-hexahydropyrazino[2,1-C][1,4]oxazin-8 (1H)-ylmethyl]-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 372

5-(6-{[(3S)-3-[(9AS)-hexahydropyrazino[2,1-C][1,4]oxazin-8 (1H)-ylmethyl]-3,4-dihydroisoquinolin-2 (1H)-yl]carbonyl}-1,3-benzodioxol-5-yl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 373

5-(6-{[(3S)-3-[(9AR)-hexahydropyrazino[2,1-C][1,4]oxazin-8 (1H)-ylmethyl]-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrazol-4-yl)-1H-pyrrole-3-carboxamide

### EXAMPLE 374

5-(6-{[(3S)-3-[(9AR)-hexahydropyrazino[2,1-C][1,4]oxazin-8 (1H)-ylmethyl]-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 375

5-(6-{[(3S)-3-[(9AR)-hexahydropyrazino[2,1-C][1,4]oxazin-8 (1H)-ylmethyl]-3,4-dihydroisoquinolin-2 (1H)-yl]carbonyl}-1,3-benzodioxol-5-yl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 376

5-(6-{[(3S)-3-(aminomethyl)-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrazol-4-yl)-1H-pyrrole-3-carboxamide

### EXAMPLE 377

5-(6-{[(3S)-3-(aminomethyl)-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 378

5-(6-{[(3S)-3-(aminomethyl)-3,4-dihydroisoquinolin-2 (1H)-yl]carbonyl}-1,3-benzodioxol-5-yl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 379

5-(6-{[(3S)-3-[(3-cyanoazetidin-1-yl)methyl]-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrazol-4-yl)-1H-pyrrole-3-carboxamide

### EXAMPLE 380

5-(6-{[(3S)-3-[(3-cyanoazetidin-1-yl)methyl]-3,4-dihydroisoquinolin-2 (1H)-yl]carbonyl}-1,3-benzodioxol-5-yl)-N-(4-hydroxyphenyl)-1,2-dimethyl-N-(1-methyl-1H-pyrrolo[2,3-B]pyridin-5-yl)-1H-pyrrole-3-carboxamide

### EXAMPLE 381

5-(6-{[(3S)-3-[(3-cyanoazetidin-1-yl)methyl]-3,4-dihydroisoquinolin-2 (1H)-yl]carbonyl}-1,3-benzodioxol-5-yl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

### EXAMPLE 382

N-(4-hydroxyphenyl)-1,2-dimethyl-N-(1-methyl-1H-pyrazol-4-yl)-5-(6-{[(3S)-3-(1-oxa-6-azaspiro[3.3]hept-6-ylmethyl)-3,4-dihydroisoquinolin-2 (1H)-yl]carbonyl}-1,3-benzodioxol-5-yl)-1H-pyrrole-3-carboxamide

### EXAMPLE 383

N-(4-hydroxyphenyl)-1,2-dimethyl-N-(1-methyl-1H-pyrrolo[2,3-B]pyridin-5-yl)-5-(6-{[(3S)-3-(1-oxa-6-azaspiro[3.3]hept-6-ylmethyl)-3,4-dihydroisoquinolin-2 (1H)-yl]carbonyl}-1,3-benzodioxol-5-yl)-1H-pyrrole-3-carboxamide

### EXAMPLE 384

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(pyridin-4-yl)-1H-pyrrole-3-carboxamide hydrochloride

### Elemental Microanalysis: (%, Theoretical:Measured)

% C=67.28:67.73; % H=5.65:5.30; % N=10.06:9.41% Cl—=5.09:5.79

### EXAMPLE 385

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide hydrochloride

### High-Resolution Mass Spectrometry (ESI+):

C₄₁H₄₁Cl N₆O₄ Empirical formula:

[M+H]⁺ calculated: 713.3253

[M+H]⁺ measured: 713.3272

### EXAMPLE 386

5-(5-Chloro-2-{[(3S)-3-(morpholin-4-ylmethyl)-3,4-dihydroisoquinolin-2 (1H)-yl]carbonyl}phenyl)-N-(5-cyano-1,2-dimethyl-1H-PYRROL-3-yl)-N-(4-hydroxyphenyl)-1,2-dimethyl-1H-pyrrole-3-carboxamide hydrochloride
Step A: N-[4-[tert-Butyl(dimethyl)silyl]oxyphenyl]-5-[5-chloro-2-[(3S)-3-(morpholino-methyl)-3,4-dihydro-1H-isoquinoline-2-carbonyl]phenyl]-N-(5-cyano-1,2-dimethyl-pyrrol-3-yl)-1,2-dimethyl-pyrrole-3-carboxamide

The title compound is obtained in accordance with the process of Example 1 using the acid of Preparation 1 and the compound of Preparation 3′ in Step A, and the compound of Preparation 18″ in Step C.

IR: ν —CN—: 2210 cm⁻¹; ν —C═O—: 1631 cm⁻¹

Step B: 5-(5-Chloro-2-{[(3S)-3-(morpholin-4-ylmethyl)-3,4-dihydroisoquinolin-2 (1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide hydrochloride

The compound of Step A is deprotected in accordance with the protocol described in Step D of Example 1. The product thereby obtained is finally subjected to a step of conversion into salt form in the presence of HCl in ether. After filtration and lyophilisation in a mixture of acetonitrile/water, the expected product is obtained.

¹H NMR (500 MHz, dmso-d6) δ ppm: 11.2 (bs, 1H), 9.39 (bs, 1H), 7.83 (d, 1H), 7.54 (d, 1H), 7.33 (s, 1H), 7.14 (m, 2H), 7 (m, 2H), 6.8 (d, 2H), 6.62 (d, 2H), 6.57 (bs, 1H), 5.26 (s, 1H), 5.26 (m, 1H), 4.64/4.03 (AB, 2H), 4.01/3.92 (2m, 4H), 3.75/3.43/3.15/3.02 (4m, 4H), 3.59 (s, 3H), 3.3/3.15 (2m, 2H), 2.97 (s, 3H), 2.69/2.52 (dd+d, 2H), 2.06 (s, 3H), 1.91 (s, 3H)

### Elemental Microanalysis: (%, Theoretical:Measured)

% C=65.34:65.50; % H=5.62:5.15; % N=11.15:10.84% Cl—=4.70:4.44

### High-Resolution Mass Spectrometry (ESI+):

C₄₁H₄₁Cl N₆O₄ Empirical formula:

[M+H]⁺ calculated: 717.2952

[M+H]⁺ measured: 717.2951

### EXAMPLE 387

N-(4-hydroxyphenyl)-1,2-dimethyl-N-(1-methyl-1H-pyrrolo[2,3-C]pyridin-5-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

### Elemental Microanalysis: (%, Theoretical:Measured)

% C=66.62:66.36; % H=5.59:5.62; % N=10.84:10.72% Cl—=4.57:4.55

### High-Resolution Mass Spectrometry (ESI+):

C₄₃H₄₂N₆O₆ Empirical formula:

[M+H]⁺ calculated: 739.3239

[M+H]⁺ measured: 739.3241

### EXAMPLE 388

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide dihydrochloride

The title compound is obtained in accordance with the process described in Step B of Example 49 using Example 71 as starting material, it being understood that the product is finally subjected to a step of conversion into salt form in the presence of 1M HCl in ether. After filtration and lyophilisation in a mixture of acetonitrile/water, the expected compound is obtained.

### Elemental Microanalysis: (%, Theoretical:Measured)

% C=62.73:62.96; % H=5.64:4.95; % N=10.45:10.32; % Cl=13.23:12.91

### High-Resolution Mass Spectrometry (ESI+):

C₄₂H₄₃Cl N₆O₄ Empirical formula:

[M+H]⁺ calculated: 731.3107

[M+H]⁺ measured: 731.3111

### EXAMPLE 389

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[2,3-C]pyridin-5-yl)-1H-pyrrole-3-carboxamide hydrochloride

The procedure is in accordance with the general protocol of Example 1 using the appropriate Preparations. The product obtained is finally dissolved in acetonitrile and converted into salt form using 0.1M aqueous HCl solution. After a lyophilisation step, the expected compound is obtained in the form of a solid.

### Elemental Microanalysis: (%, Theoretical:Measured)

% C=65.88:66.28; % H=5.53:5.15; % N=10.98:10.95; % Cl—=4.63:4.47

### High-Resolution Mass Spectrometry (ESI/FIA/HR and MS/MS):

C₄₂H₄₁Cl N₆O₄ Empirical formula:

[M+H]⁺ calculated: 729.2951

[M+H]⁺ measured: 729.2954

### EXAMPLE 390

5-(5-Chloro-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrrolo[2,3-B]pyridin-5-yl)-N-phenyl-1H-pyrrole-3-carboxamide

The procedure is in accordance with the general protocol of Example 1 using the appropriate Preparations, it being understood that Step D is not carried out. The expected product is obtained in free base form.

### Elemental Microanalysis: (%, Theoretical:Measured)

% C=70.72:69.77; % H=5.79:5.96; % N=11.78:11.43

### High-Resolution Mass Spectrometry (ESI+):

C₄₂H₄₁Cl N₆O₃ Empirical formula:

[M+H]⁺ calculated: 713.3001

[M+H]⁺ measured: 713.2998

### EXAMPLE 391

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrrolo[3,2-B]pyridin-5-yl)-1H-pyrrole-3-carboxamide hydrochloride

### Elemental Microanalysis: (%, Theoretical:Measured)

% C=65.88:65.69; % H=5.33:4.87; % N=10.98:10.86; % Cl—=4.63:4.51

### High-Resolution Mass Spectrometry (ESI+−/FIA):

C₄₂H₄₁Cl N₆O₄ Empirical formula:

[M+H]⁺ calculated: 729.2951

[M+H]⁺ measured: 729.2953

### EXAMPLE 392

5-(5-Chloro-2-{[(3R)-3-[3-(morpholin-4-yl)propyl]-3,4-dihydroisoquinolin-2 (1H)-yl]carbonyl}phenyl)-N-(4-hydroxyphenyl)-1,2-dimethyl-N-phenyl-1H-pyrrole-3-carboxamide hydrochloride

### Elemental Microanalysis: (%, Theoretical:Measured)

% C=68.19:68.33; % H=6.00:5.49; % N=10.78:10.71; % Cl—=4.55:4.46; % Cl=9.58:9.78

### High-Resolution Mass Spectrometry (ESI+−/FIA):

C₄₂H₄₃Cl N₄O₄ Empirical formula:

[M+H]⁺ calculated: 703.3046

[M+H]⁺ measured: 703.3042

### EXAMPLE 393

5-(5-Chloro-2-{[(3R)-3-[3-(morpholin-4-yl)propyl]-3,4-dihydroisoquinolin-2 (1H)-yl]carbonyl}phenyl)-N-(4-hydroxyphenyl)-1,2-dimethyl-N-(1-methyl-1H-pyrrolo[2,3-B]pyridin-5-yl)-1H-pyrrole-3-carboxamide hydrochloride

### High-Resolution Mass Spectrometry (ESI+−/FIA):

C₄₄H₄₅Cl N₆O₄ Empirical formula:

[M+H]⁺ calculated: 757.3264

[M+H]⁺ measured: 757.3263

### EXAMPLE 394

5-(5-Chloro-2-{[(3R)-3-[3-(morpholin-4-yl)propyl]-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide

The title compound is obtained in accordance with the process described in Step B of Example 49 using Example 393 as starting material, it being understood that the product is not subjected to the step of conversion into salt form.

### Elemental Microanalysis: (%, Theoretical:Measured)

% C=69.60:69.56; % H=6.21:6.24; % N=11.07:11.08

### High-Resolution Mass Spectrometry (ESI+−/FIA):

C₄₄H₄₇Cl N₆O₄ Empirical formula:

[M+H]⁺ calculated: 759.3420

[M+H]⁺ measured: 759.3422

### EXAMPLE 395

5-(5-Chloro-2-{[(3S)-3-(morpholin-4-ylmethyl)-3,4-dihydroisoquinolin-2 (1H)-yl]carbonyl}phenyl)-N-(3-cyano-4-METHOXYPHENYL)-N-(4-hydroxyphenyl)-1,2-dimethyl-1H-pyrrole-3-carboxamide hydrochloride

The procedure is in accordance with the general protocol of Example 1 using the appropriate Preparations. The product obtained is finally dissolved in acetonitrile and converted into salt form using 0.1M aqueous HCl solution. After a lyophilisation step, the expected compound is obtained in the form of a solid.

### High-Resolution Mass Spectrometry (ESI/FIA/HR and MS/MS):

C₄₂H₄₀Cl N₅O₅ Empirical formula:

[M+H]⁺ calculated: 730.2791

[M+H]⁺ measured: 730.2790

### EXAMPLE 396

N-(3-fluoro-4-hydroxyphenyl)-1,2-dimethyl-N-(1-methyl-1H-pyrazol-4-yl)-5-(6-{[(3S)-3-(morpholin-4-ylmethyl)-3,4-dihydroisoquinolin-2 (1H)-yl]carbonyl}-1,3-benzodioxol-5-yl)-1H-pyrrole-3-carboxamide hydrochloride

The title compound is obtained in accordance with the process of Example 1 using the acid of Preparation 11 and (3S)-3-(4-morpholinylmethyl)-1,2,3,4-tetrahydroisoquinoline (see Preparation 3′) in Step A, and the compound of Preparation 20″ in Step C. The product obtained is finally subjected to a step of conversion into salt form in the presence of 1M HCl in ether. After filtration and lyophilisation in a mixture of acetonitrile/water, the expected compound is obtained.

### Elemental Microanalysis: (%, Theoretical:Measured)

% C=65.79:65.43; % H=5.39:5.19; % N=11.31:11.21; % Cl—=4.77:4.34

### High-Resolution Mass Spectrometry (ESI/FIA/HR and MS/MS):

C₃₉H₃₉F N₆O₆ Empirical formula:

[M+H]⁺ calculated: 707.2988

[M+H]⁺ measured: 707.2988

### EXAMPLE 397

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-N-(2-methoxypyrimidin-5-yl)-1,2-dimethyl-1H-pyrrole-3-carboxamide

### High Resolution Mass Spectrometry (ESI+):

C₃₉H₃₉Cl N₆O₅ Empirical formula:

[M+H]⁺ calculated: 707.2743

[M+H]⁺ measured: 707.2746

### EXAMPLE 398

5-(5-Chloro-2-{[(3S)-3-(morpholin-4-ylmethyl)-3,4-dihydroisoquinolin-2 (1H)-yl]carbonyl}phenyl)-N-(3-cyanophenyl)-N-(4-hydroxyphenyl)-1,2-dimethyl-1H-pyrrole-3-carboxamide hydrochloride

### Elemental Microanalysis: (%, Theoretical:Measured)

% C=66.85:66.75; % H=5.34:5.42; % N=9.51:9.73; % Cl=9.62:9.67; % Cl⁻=4.81:4.71

### High-Resolution Mass Spectrometry (ESI+−/FIA/HR, ESI−/FIA):

C₄₁H₃₈Cl N₅O₄ Empirical formula:

[M+H]⁺ calculated: 700.2685

[M+H]⁺ measured: 700.2686

### EXAMPLE 399

5-(5-Chloro-2-{[(3S)-3-(morpholin-4-ylmethyl)-3,4-dihydroisoquinolin-2 (1H)-yl]carbonyl}phenyl)-N-(3-fluoro-4-hydroxyphenyl)-1,2-dimethyl-N-(1-methyl-1H-pyrazol-4-yl)-1H-pyrrole-3-carboxamide hydrochloride

The title compound is obtained in accordance with the process of Example 1 using the acid of Preparation 1 and (3S)-3-(4-morpholinylmethyl)-1,2,3,4-tetrahydroisoquinoline (see Preparation 3′) in Step A, and the compound of Preparation 20″ in Step C. The product obtained is finally subjected to a step of conversion into salt form in the presence of 1M HCl in ether. After filtration and lyophilisation in a mixture of acetonitrile/water, the expected compound is obtained.

### Elemental Microanalysis: (%, Theoretical:Measured)

% C=62.21:61.32; % H=5.36:5.18; % N=11.46:11.14; % Cl=9.66:10.16; % Cl⁻=4.83:5.23

### High-Resolution Mass Spectrometry (ESI+−/FIA/HR, ESI−/FIA):

C₃₈H₃₈Cl F N₆O₄ Empirical formula:

[M+H]⁺ calculated: 697.2700

[M+H]⁺ measured: 697.2704

### EXAMPLE 400

Methyl 2-[{[5-(5-Chloro-2-{[(3S)-3-(morpholin-4-ylmethyl)-3,4-dihydro-isoquinolin-2 (1H)-yl]carbonyl}phenyl)-1,2-dimethyl-1H-pyrrol-3-yl]carbonyl}(4-hydroxyphenyl)amino]pyridine-4-carboxylate hydrochloride

The title compound is a secondary product which forms in the course of synthesis of Example 110 (in the final Step before the step of conversion into salt form) owing to hydrolysis of the nitrile function into a methyl ester function. The compound is separated from that of Example 110 by chromatography over silica gel in a mixture of methanol and dichloromethane.

### Elemental Microanalysis: (%, Theoretical:Measured)

% C=63.90:64.43; % H=5.36:5.01; % N=9.09:9.34; % Cl⁻=4.60:4.46

### High-Resolution Mass Spectrometry (ESI+/HR and ESI−/LR):

C₄₁H₄₀Cl N₅O₆ Empirical formula:

[M+H]⁺ calculated: 734.2740

[M+H]⁺ measured: 734.2743

### EXAMPLE 401

5-(5-Chloro-2-{[(3S)-3-(morpholin-4-ylmethyl)-3,4-dihydroisoquinolin-2 (1H)-yl]carbonyl}phenyl)-N-(3-cyano-4-fluorophenyl)-N-(4-hydroxyphenyl)-1,2-dimethyl-1H-pyrrole-3-carboxamide hydrochloride

### Elemental Microanalysis: (%, Theoretical:Measured)

% C=65.25:64.23; % H=5.07:4.71; % N=9.28:9.36; % Cl=9.40:9.59; % Cl⁻=4.70:4.50

### High-Resolution Mass Spectrometry (ESI+−/FIA/HR, ESI−/FIA):

C₄₁H₃₇Cl F N₅O₄ Empirical formula:

[M+H]⁺ calculated: 718.2591

[M+H]⁺ measured: 718.2593

### EXAMPLE 402

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N—{1-[(3S OR R)-tetrahydrofuran-3-yl]-1H-pyrazol-4-yl}-1H-pyrrole-3-carboxamide hydrochloride

### Elemental Microanalysis: (%, Theoretical:Measured)

% C=65.20:65.92; % H=5.87:5.78; % N=11.13:10.36% Cl⁻=4.69:4.79)

### High-Resolution Mass Spectrometry (ESI+):

C₄₁H₄₃FN₆O₅ Empirical formula:

[M+H]⁺ calculated: 719.3359

[M+H]⁺ measured: 719.3362 and

### EXAMPLE 403

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N—{1-[(3R OR S)-tetrahydrofuran-3-yl]-1H-pyrazol-4-yl}-1H-pyrrole-3-carboxamide hydrochloride

### Elemental Microanalysis: (%, Theoretical:Measured)

% C=65.20:66.04; % H=5.87:5.87; % N=11.13:10.62; % Cl⁻=4.69:4.76

### High-Resolution Mass Spectrometry (ESI/+):

C₄₁H₄₃FN₆O₅ Empirical formula:

[M+H]⁺ calculated: 719.3359

[M+H]⁺ measured: 719.3350

The compounds of Examples 402 and 403 are obtained in accordance with the process of Example 1 using the acid of Preparation 8 and the compound of Preparation 3′ in Step A, and the compound of Preparation 22″ in Step C. The diastereoisomers obtained are separated by chiral chromatography and then converted into salt form and lyophilised as described in the general process to yield the title compounds.

### EXAMPLE 404

5-(5-Chloro-2-{[(3S)-3-(morpholin-4-ylmethyl)-3,4-dihydroisoquinolin-2 (1H)-yl]carbonyl}phenyl)-N-(2-cyanopyrimidin-4-yl)-N-(4-hydroxyphenyl)-1,2-dimethyl-1H-pyrrole-3-carboxamide hydrochloride

### EXAMPLE 405

5-(5-Chloro-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrrolo[2,3-B]pyridin-5-yl)-N-(PYRIDAZIN-4-yl)-1H-pyrrole-3-carboxamide dihydrochloride

The title compound is obtained in accordance with the process of Example 1 using the acid of Preparation 1 and the compound of Preparation 3′ in Step A, and the appropriate amine in Step C, it being understood that Step D is not carried out. The compound obtained is converted into salt form and lyophilised as described in the general process to obtain the title compound.

### High-Resolution Mass Spectrometry (ESI+−/FIA/HR, ESI−/FIA):

C₄₀H₃₉Cl N₈O₃ Empirical formula:

[M+H]⁺ calculated: 715.2906

[M+H]⁺ measured: 715.2909

### EXAMPLE 406

N-(5-cyano-1,2-dimethyl-1H-PYRROL-3-yl)-N-(4-hydroxyphenyl)-1,2-dimethyl-5-(2-{[(3S)-3-(morpholin-4-ylmethyl)-3,4-dihydroisoquinolin-2 (1H)-yl]carbonyl}phenyl)-1H-pyrrole-3-carboxamide hydrochloride

The title compound is obtained in accordance with the process of Example 1 using the acid of Preparation 25 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=68.46:68.27; % H=6.03:5.12; % N=11.68:11.75; % Cl⁻=4.93:4.73

### High-Resolution Mass Spectrometry (ESI/FIA/HR and MS/MS, ESI−/FIA):

C₄₁H₄₂N₆O₄ Empirical formula:

[M+H]⁺ calculated: 683.3340

[M+H]⁺ measured: 683.3334

### EXAMPLE 407

N-(3-fluoro-4-hydroxyphenyl)-5-(5-methoxy-2-{I[(3S)-3-(morpholin-4-ylmethyl)-3,4-dihydroisoquinolin-2 (1H)-yl]carbonyl}phenyl)-1,2-dimethyl-N-(1-methyl-1H-pyrazol-4-yl)-1H-pyrrole-3-carboxamide hydrochloride

The title compound is obtained in accordance with the process of Example 1 using the acid of Preparation 12 and the compound of Preparation 3′ in Step A, and the compound of Preparation 20″ in Step C. The compound obtained is converted into salt form and lyophilised as described in the general process to obtain the title compound.

### Elemental Microanalysis: (%, Theoretical:Measured)

% C=64.23:63.94; % H=5.80:5.00; % N=11.52:11.56; % Cl⁻=4.86:4.99

### High-Resolution Mass Spectrometry (ESI+−/FIA/HR and MS/MS, ESI−/FIA):

C₃₉H₄₁F N₆O₅ Empirical formula:

[M+H]⁺ calculated: 693.3195

[M+H]⁺ measured: 693.3191

### EXAMPLE 408

N-(5-cyano-1,2-dimethyl-1H-pyrrol-3-yl)-N-(4-hydroxyphenyl)-5-(5-methoxy-2-{[(3S)-3-(morpholin-4-ylmethyl)-3,4-dihydroisoquinolin-2 (1H)-yl]carbonyl}phenyl)-1,2-dimethyl-1H-pyrrole-3-carboxamide hydrochloride

The title compound is obtained in accordance with the process of Example 1 using the acid of Preparation 12 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=67.32:67.30; % H=6.05:5.28; % N=11.22:11.15; % Cl⁻=4.73:4.59

### High-Resolution Mass Spectrometry (ESI+−/FIA/HR and ESI−/FIA):

C₄₂H₄₄N₆O₅ Empirical formula:

[M+H]⁺ calculated: 713.3446

[M+H]⁺ measured: 713.3443

### EXAMPLE 409

N-(4-hydroxyphenyl)-1,2-dimethyl-N-(1-methyl-1H-pyrrolo[2,3-B]pyridin-5-yl)-5-(2-{[(3S)-3-(morpholin-4-ylmethyl)-3,4-dihydroisoquinolin-2 (1H)-yl]-carbonyl}phenyl)-1H-pyrrole-3-carboxamide hydrochloride

The title compound is obtained in accordance with the process of Example 1 using the acid of Preparation 25 and the compound of Preparation 3′ in Step A, and the compound of Preparation 11″ in Step C. The compound obtained is converted into salt form and lyophilised as described in the general process to obtain the title compound.

### Elemental Microanalysis: (%, Theoretical:Measured)

% C=68.98:68.95; % H=5.93:4.76; % N=11.49:11.43

### High-Resolution Mass Spectrometry (ESI/FIA/HR and MS/MS):

C₄₂H₄₂N₆O₄ Empirical formula:

[M+H]⁺ calculated: 695.3340

[M+H]⁺ measured: 695.3341

### EXAMPLE 410

N-(4-hydroxyphenyl)-1,2-dimethyl-N-(1-methyl-2,3-dihydro-1H-pyrrolo[2,3-B]pyridin-5-yl)-5-(2-{[(3S)-3-(morpholin-4-ylmethyl)-3,4-dihydroisoquinolin-2 (1H)-yl]carbonyl}phenyl)-1H-pyrrole-3-carboxamide hydrochloride

The title compound is obtained in accordance with the process described in Step B of Example 49 using Example 409 as starting material. The compound obtained is converted into salt form and lyophilised as described in the general process to obtain the title compound.

### High-Resolution Mass Spectrometry (ESI/FIA/HR and ESI−/FIA):

C₄₂H₄₄N₆O₄ Empirical formula:

[M+H]⁺ calculated: 697.3497

[M+H]⁺ measured: 697.3497

### EXAMPLE 411

5-(5-Chloro-2-{[(3R)-3-methyl-3,4-dihydroisoquinolin-2 (1H)-yl]carbonyl}phenyl)-N-(4-hydroxyphenyl)-2-methyl-N-(1-methyl-1H-pyrazol-4-yl)-1-[2-(morpholin-4-yl)ethyl]-1H-pyrrole-3-carboxamide hydrochloride

The title compound is obtained in accordance with the process of Example 96 using the acid of Preparation 30, the compound of Preparation 1′, and the compound of Preparation 1″. The compound obtained is converted into salt form and lyophilised as described in the general process to obtain the title compound.

### High-Resolution Mass Spectrometry (ESI/FIA/HR and MS/MS):

C₃₉H₄₁Cl N₆O₄ Empirical formula:

[M+H]⁺ calculated: 693.2951

[M+H]⁺ measured: 693.2947

### EXAMPLE 412

5-(5-Chloro-2-{[(3R)-3-[3-(morpholin-4-yl)propyl]-3,4-dihydroisoquinolin-2 (1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 6′ in Step A, and the compound of Preparation 18″ in Step C. The compound obtained is converted into salt form and lyophilised as described in the general process to obtain the title compound.

### Elemental Microanalysis: (%, Theoretical:Measured)

% C=66.06:65.61; % H=5.93:5.22; % N=10.75:10.69; % Cl⁻=4.53:4.68

### High-Resolution Mass Spectrometry (ESI/FIA/HR and MS/MS, ESI−/FIA):

C₄₃H₄₅Cl N₆O₄ Empirical formula:

[M+H]⁺ calculated: 745.3264

[M+H]⁺ measured: 745.3260

### EXAMPLE 413

N-(5-cyano-1,2-dimethyl-1H-pyrrol-3-yl)-N-(4-hydroxyphenyl)-1,2-dimethyl-5-(7-{[(3S)-3-(morpholin-4-ylmethyl)-3,4-dihydroisoquinolin-2 (1H)-yl]-carbonyl}-2,3-dihydro-1,4-benzodioxin-6-yl)-1H-pyrrole-3-carboxamide hydrochloride

The title compound is obtained in accordance with the process of Example 1 using the acid of Preparation 13 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=66.44:66.40; % H=5.83:4.84; % N=10.81:10.79; % Cl⁻=4.56:4.22

### High-Resolution Mass Spectrometry (ESI/FIA/HR and MS/MS, ESI−/FIA):

C₄₃H₄₄N₆O₆ Empirical formula:

[M+H]⁺ calculated: 741.3395

[M+H]⁺ measured: 741.3397

### EXAMPLE 414

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(5-methyl-5H-pyrrolo[3,2-D]pyrimidin-2-yl)-1H-pyrrole-3-carboxamide hydrochloride

The title compound is obtained in accordance with the process of Example 1 using the acid of Preparation 1 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=64.23:64.38; % H=5.39:5.25; % N=12.79:12.62; % Cl⁻=4.62:4.39

### High-Resolution Mass Spectrometry (ESI/FIA/HR and MS/MS, ESI−/FIA):

C₄₁H₄₀Cl N₇O₄ Empirical formula:

[M+H]⁺ calculated: 730.2903

[M+H]⁺ measured: 730.2904

### EXAMPLE 415

5-(5-Chloro-2-{[(3S)-3-(morpholin-4-ylmethyl)-3,4-dihydroisoquinolin-2 (1H)-yl]carbonyl}phenyl)-N-(5-cyano-1,2-dimethyl-1H-pyrrol-3-yl)-N-(4-hydroxyphenyl)-1-methyl-1H-pyrrole-3-carboxamide hydrochloride

The title compound is obtained in accordance with the process of Example 1 using the acid of Preparation 3 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=64.95:65.14; % H=5.45:5.34; % N=11.36:11.36; % Cl⁻=4.79:4.67

### High-Resolution Mass Spectrometry (ESI+−/FIA/HR, ESI−/FIA):

C₄₀H₃₉Cl N₆O₄ Empirical formula:

[M+H]⁺ calculated: 703.2794

[M+H]⁺ measured: 703.2795

### EXAMPLE 416

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[11-(trideuteriomethyl)-1H-pyrazol-4-yl]-1H-pyrrole-3-carboxamide hydrochloride

The title compound is obtained in accordance with the process of Example 1 using the acid of Preparation 1 and the compound of Preparation 3′ in Step A, and the compound of Preparation 25″ in Step C. The compound obtained is converted into salt form and lyophilised as described in the general process to obtain the title compound.

### Elemental Microanalysis: (%, Theoretical:Measured)

% C=63.51:63.41; % H=5.63:5.42; % N=11.69:11.61; % Cl⁻=4.93:4.85

### High-Resolution Mass Spectrometry (ESI+−/FIA/HR, ESI−/FIA):

C₃₈H₃₆Cl D₃N₆O₄ Empirical formula:

[M+H]⁺ calculated: 682.2982

[M+H]⁺ measured: 682.2986

### EXAMPLE 417

N-(5-cyano-1,2-dimethyl-1H-pyrrol-3-yl)-N-(4-hydroxyphenyl)-5-(4-methoxy-2-{[(3S)-3-(morpholin-4-ylmethyl)-3,4-dihydroisoquinolin-2 (1H)-yl]carbonyl}phenyl)-1,2-dimethyl-1H-pyrrole-3-carboxamide hydrochloride

The title compound is obtained in accordance with the process of Example 1 using the acid of Preparation 31 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=67.32:67.56; % H=6.05:5.84; % N=11.22:11.21; % Cl⁻=4.73:4.71

### High-Resolution Mass Spectrometry (ESI/FIA/HR and MS/MS, ESI−/FIA):

C₄₂H₄₄N₆O₅ Empirical formula:

[M+H]⁺ calculated: 713.3446

[M+H]⁺ measured: 713.3446

### EXAMPLE 418

N-(4-hydroxyphenyl)-5-(4-methoxy-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrazol-4-yl)-1H-pyrrole-3-carboxamide hydrochloride

The title compound is obtained in accordance with the process of Example 1 using the acid of Preparation 31 and the compound of Preparation 3′ in Step A, and the compound of Preparation 1″ in Step C. The compound obtained is converted into salt form and lyophilised as described in the general process to obtain the title compound.

### Elemental Microanalysis: (%, Theoretical:Measured)

% C=65.86:65.51; % H=6.09:6.09; % N=11.82:11.73; % Cl⁻=4.98:5.14

### High-Resolution Mass Spectrometry (ESI/FIA/HR, ESI−/FIA):

C₃₉H₄₂N₆O₅ Empirical formula:

[M+H]⁺ calculated: 675.3289

[M+H]⁺ measured: 675.3286

### EXAMPLE 419. N-(3-CYANOPHENYL)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the compound of Preparation 41″ in Step C. The compound obtained is converted into salt form and lyophilised as described in the general process to obtain the title compound.

### High-Resolution Mass Spectrometry (ESI+):

C₄₁H₃₈FN₅O₄ Empirical formula:

[M+H]⁺ calculated: 684.2988

[M+H]⁺ measured: 684.2975

### EXAMPLE 420

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(trideuteriomethyl)-1H-pyrazol-4-yl]-1H-pyrrole-3-carboxamide hydrochloride

The title compound is obtained in accordance with the process of Example 1 using the acid of Preparation 8 and the compound of Preparation 3′ in Step A, and the compound of Preparation 25″ in Step C. The compound obtained is converted into salt form and lyophilised as described in the general process to obtain the title compound.

### High-Resolution Mass Spectrometry (ESI/+):

C₃₈D₃H₃₆FN₆O₄ Empirical formula:

[M+H]⁺ calculated: 666.3285

[M+H]⁺ measured: 666.3265

### EXAMPLE 421

N-(5-cyano-1,2-dimethyl-1H-pyrrol-3-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the compound of Preparation 18″ in Step C. The compound obtained is converted into salt form and lyophilised as described in the general process to obtain the title compound.

### High-Resolution Mass Spectrometry (ESI/FIA/HR and MS/MS):

C₄₁H₄₁F N₆O₄ Empirical formula:

[M+H]⁺ calculated: 701.3246

[M+H]⁺ measured: 701.3282

### EXAMPLE 422

N-(4-hydroxyphenyl)-1,2-dimethyl-N-(1-methyl-1H-pyrazol-4-yl)-5-(2-{[(3S)-3-(morpholin-4-ylmethyl)-3,4-dihydroisoquinolin-2 (1H)-yl]carbonyl}phenyl)-1H-pyrrole-3-carboxamide hydrochloride

The title compound is obtained in accordance with the process of Example 1 using the acid of Preparation 25 and the compound of Preparation 3′ in Step A, and the compound of Preparation 1″ in Step C. The compound obtained is converted into salt form and lyophilised as described in the general process to obtain the title compound.

### Elemental Microanalysis: (%, Theoretical:Measured)

% C=67.00:67.47; % H=6.07:5.54; % N=12.34:12.46; % Cl⁻=5.20:4.58

### High-Resolution Mass Spectrometry (ESI+−/FIA/HR, ESI−/FIA):

C₃₈H₄₀N₆O₄ Empirical formula:

[M+H]⁺ calculated: 645.3184

[M+H]⁺ measured: 645.3182

### EXAMPLE 423

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(oxetan-3-yl)-1H-pyrazol-4-yl]-1H-pyrrole-3-carboxamide

The title compound is obtained in accordance with the process of Example 1 using the acid of Preparation 8 and the compound of Preparation 3′ in Step A, and the compound of Preparation 26″ in Step C.

### Elemental Microanalysis: (%, Theoretical:Measured)

% C=68.17:67.82; % H=5.86:5.97; % N=11.92:11.48

### High-Resolution Mass Spectrometry (ESI/FIA/HR and MS/MS):

C₄₀H₄₁FN₆O₅ Empirical formula:

[M+H]⁺ calculated: 705.3202

[M+H]⁺ measured: 705.3207

### EXAMPLE 424

N-(4-hydroxyphenyl)-N-(2-methoxypyrimidin-5-yl)-1,2-dimethyl-5-(2-{[(3S)-3-(morpholin-4-ylmethyl)-3,4-dihydroisoquinolin-2 (1H)-yl]carbonyl}phenyl)-1H-pyrrole-3-carboxamide hydrochloride

The title compound is obtained in accordance with the process of Example 1 using the acid of Preparation 25 and the compound of Preparation 3′ in Step A, and the compound of Preparation 12″ in Step C. The compound obtained is converted into salt form and lyophilised as described in the general process to obtain the title compound.

### Elemental Microanalysis: (%, Theoretical:Measured)

% C=66.05:65.63; % H=5.83:5.45; % N=11.85:11.93; % Cl⁻=5.00:4.91

### High-Resolution Mass Spectrometry (ESI+−/FIA/HR, ESI−/FIA):

C₃₉H₄₀N₆O₅ Empirical formula:

[M+H]⁺ calculated: 673.3133

[M+H]⁺ measured: 673.3129

### EXAMPLE 425

N-(3-cyano-5-methoxyphenyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=67.25:66.55; % H=5.51:5.28; % N=9.33:8.55% Cl⁻=4.73:4.67

### EXAMPLE 426

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-N-(2-methoxypyrimidin-5-yl)-1,2-dimethyl-1H-pyrrole-3-carboxamide

The title compound is obtained in accordance with the process of Example 1 using the acid of Preparation 8 and the compound of Preparation 3′ in Step A, and the compound of Preparation 12″ in Step C.

### High-Resolution Mass Spectrometry (ESI/FIA/HR and MS/MS):

C₃₉H₃₉FN₆O₅ Empirical formula:

[M+H]⁺ calculated: 691.3038

[M+H]⁺ measured: 691.3060

### EXAMPLE 427

N-(3-cyano-4-methoxyphenyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=67.24:66.41; % H=5.51:5.35; % N=9.33:8.97% Cl⁻=4.73:4.81

### EXAMPLE 428

N-(5-cyano-1,2-dimethyl-1H-pyrrol-3-yl)-5-(4-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 26 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=66.79:66.97; % H=5.74:5.36; % N=11.40:11.45; % Cl⁻=4.81:4.53

### High-Resolution Mass Spectrometry (ESI+−/FIA/HR, ESI−/FIA):

C₄₁H₄₁F N₆O₄ Empirical formula:

[M+H]⁺ calculated: 701.3246

[M+H]⁺ measured: 701.3245

### EXAMPLE 429

N-(5-cyano-1,2-dimethyl-1H-pyrrol-3-yl)-5-(4-fluoro-5-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 27 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=65.75:65.43; % H=5.78:5.57; % N=10.95:10.81; % Cl⁻=4.62:4.54

### High-Resolution Mass Spectrometry (ESI/FIA/HR and MS/MS, ESI−/FIA):

C₄₂H₄₃F N₆O₅ Empirical formula:

[M+H]⁺ calculated: 731.3352

[M+H]⁺ measured: 731.3351

### EXAMPLE 430

5-(5-Chloro-2-{[3-(trifluoromethyl)-3,4-dihydroisoquinolin-2 (1H)-yl]-carbonyl}phenyl)-N-(4-hydroxyphenyl)-1,2-dimethyl-N-(1-methyl-1H-pyrrolo[2,3-B]-pyridin-5-yl)-1H-pyrrole-3-carboxamide

The title compound is obtained in accordance with the process of Example 1 using the acid of Preparation 1 and racemic 3-(trifluoromethyl)-1,2,3,4-tetrahydroisoquinoline in Step A, and the compound of Preparation 11″ in Step C.

### High-Resolution Mass Spectrometry (ESI+−/FIA/HR and MS/MS, ESI−/FIA):

C₃₈H₃₁Cl F₃N₅O₃ Empirical formula:

[M+H]⁺ calculated: 698.2140

[M+H]⁺ measured: 698.2144

### EXAMPLE 431

5-(5-Chloro-2-{[(3S)-3-(morpholin-4-ylmethyl)-3,4-dihydroisoquinolin-2 (1H)-yl]carbonyl}phenyl)-N-(1,3-dimethyl-1H-pyrazol-4-yl)-N-(4-hydroxyphenyl)-1,2-dimethyl-1H-pyrrole-3-carboxamide hydrochloride

### Elemental Microanalysis: (%, Theoretical:Measured)

% C=64.19:64.37; % H=5.80:5.18; % N=11.52:11.55; % Cl⁻=4.86:4.68

### High-Resolution Mass Spectrometry (ESI+−/FIA/HR, ESI−/FIA):

C₃₉H₄₁Cl N₆O₄ Empirical formula:

[M+H]⁺ calculated: 693.2951

[M+H]⁺ measured: 693.2952

and

### EXAMPLE 432

5-(5-Chloro-2-{[(3S)-3-(morpholin-4-ylmethyl)-3,4-dihydroisoquinolin-2 (1H)-yl]carbonyl}phenyl)-N-(1,5-dimethyl-1H-pyrazol-4-yl)-N-(4-hydroxyphenyl)-1,2-dimethyl-1H-pyrrole-3-carboxamide hydrochloride

### Elemental Microanalysis: (%, Theoretical:Measured)

% C=64.19:64.43; % H=5.80:5.22; % N=11.52:11.60; % Cl⁻=4.86:4.66

### High-Resolution Mass Spectrometry (ESI+−/FIA/HR, ESI−/FIA):

C₃₉H₄₁Cl N₆O₄ Empirical formula:

[M+H]⁺ calculated: 693.2951

[M+H]⁺ measured: 693.2953

The compounds of Examples 431 and 432 are obtained in accordance with the process of Example 1 using the acid of Preparation 1 and the compound of Preparation 3′ in Step A, and the mixture of Preparation 27″ in Step C. At the end of Step D, the isomers are separated by preparative HPLC using acetonitrile and water-TFA as eluants. After evaporating off the solvent and neutralising with sodium bicarbonate, the products are subjected to a step of conversion into salt form in the presence of 1M HCl in ether. After filtration and lyophilisation in a mixture of acetonitrile/water, the title products are obtained.

### EXAMPLE 433

N-(5-cyano-1-methyl-1H-pyrrol-3-yl)-N-(4-hydroxyphenyl)-1,2-dimethyl-5-(2-{[(3S)-3-(morpholin-4-ylmethyl)-3,4-dihydroisoquinolin-2 (1H)-yl]carbonyl}phenyl)-1H-pyrrole-3-carboxamide hydrochloride

The title compound is obtained in accordance with the process of Example 1 using the acid of Preparation 25 and the compound of Preparation 3′ in Step A, and the compound of Preparation 19″ in Step C. The compound obtained is converted into salt form and lyophilised as described in the general process to obtain the title compound.

### Elemental Microanalysis: (%, Theoretical:Measured)

% C=68.12:68.29; % H=5.86:5.40; % N=11.92:12.05; % Cl⁻=5.03:4.92

### High-Resolution Mass Spectrometry (ESI/FIA/HR and MS/MS):

C₄₀H₄₀N₆O₄ Empirical formula:

[M+H]⁺ calculated: 669.3184

[M+H]⁺ measured: 669.3184

### EXAMPLE 434

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-2,3-dihydro-1H-pyrrolo[2,3-B]pyridin-5-yl)-1H-pyrrole-3-carboxamide hydrochloride

The title compound is obtained in accordance with the process described in Step B of Example 49 using Example 385 as starting material. The compound obtained is converted into salt form and lyophilised as described in the general process to obtain the title compound.

### Elemental Microanalysis: (%, Theoretical:Measured)

% C=67.15:68.03; % H=5.90:5.50; % N=11.19:10.59% Cl⁻=4.72:5.55

### EXAMPLE 435

N-(4-cyanopyridin-2-yl)-5-(5-fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 14 and the compound of Preparation 3′ in Step A, and the appropriate amine in Step C. The compound obtained is converted into salt form and lyophilised as described in the general process to obtain the title compound.

### High-Resolution Mass Spectrometry (ESI+−/FIA/HR, ESI−/FIA):

C₄₁H₃₉F N₆O₅ Empirical formula:

[M+H]⁺ calculated: 715.3039

[M+H]⁺ measured: 715.3040

### EXAMPLE 436

5-(5-Chloro-2-{[(3S)-3-(morpholin-4-ylmethyl)-3,4-dihydroisoquinolin-2 (1H)-yl]carbonyl}phenyl)-N-(5-cyanothiophen-2-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=63.07:63.09; % H=5.02:4.78; % N=9.43:9.35; % S=4.32:4.09; % Cl⁻=4.77:4.59

### High-Resolution Mass Spectrometry (ESI/FIA/HR and MS/MS, ESI+−/FIA):

C₃₉H₃₆Cl N₅O₄S Empirical formula:

[M+H]⁺ calculated: 706.2249:

[M+H]⁺ measured: 706.2250

### EXAMPLE 437

N-(3-cyano-4-fluorophenyl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 8 and the compound of Preparation 3′ in Step A, and the appropriate amine in Step C. The compound obtained is converted into salt form and lyophilised as described in the general process to obtain the title compound.

### High-Resolution Mass Spectrometry (ES+):

C₄₁H₃₇F₂N₅O₄ Empirical formula:

[M+H]⁺ calculated: 702.2894

[M+H]⁺ measured: 702.2886

### EXAMPLE 438

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(PYRAZIN-2-yl)-1H-pyrrole-3-carboxamide hydrochloride

The title compound is obtained in accordance with the process of Example 1 using the acid of Preparation 1 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=63.95:63.96; % H=5.37:5.17; % N=11.78:11.61; % Cl⁻=4.97:4.57

### High-Resolution Mass Spectrometry (ESI+/HR, ESI−/LR):

C₃₈H₃₇Cl N₆O₄ Empirical formula:

[M+H]⁺ calculated: 677.2638

[M+H]⁺ measured: 677.2639

### EXAMPLE 439

5-(5-fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-N-(2-methoxypyrimidin-5-yl)-1,2-dimethyl-1H-pyrrole-3-carboxamide hydrochloride

The title compound is obtained in accordance with the process of Example 1 using the acid of Preparation 14 and the compound of Preparation 3′ in Step A, and the compound of Preparation 12″ in Step C. The compound obtained is converted into salt form and lyophilised as described in the general process to obtain the title compound.

### High-Resolution Mass Spectrometry (ESI/FIA/HR and MS/MS, ESI−/FIA):

C₄₀H₄₁F N₆O₆ Empirical formula:

[M+H]⁺ calculated: 721.3144

[M+H]⁺ measured: 721.3144

### EXAMPLE 440

N-(5-cyano-1,2-dimethyl-1H-pyrrol-3-yl)-5-(5-fluoro-4-methoxy-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

The title compound is obtained in accordance with the process of Example 1 using the acid of Preparation 14 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=65.75:65.98; % H=5.78:5.50; % N=10.95:10.87; % Cl⁻=4.62:4.42

### High-Resolution Mass Spectrometry (ESI+/HR, ESI−/LR):

C₄₂H₄₃F N₆O₅ Empirical formula:

[M+H]⁺ calculated: 731.3352

[M+H]⁺ measured: 731.3353

### EXAMPLE 441

5-(5-Chloro-2-{[(3S)-3-(morpholin-4-ylmethyl)-3,4-dihydroisoquinolin-2 (1H)-yl]carbonyl}phenyl)-N-(5-cyano-1,2-dimethyl-1H-pyrrol-3-yl)-1,2-dimethyl-N-phenyl-1H-pyrrole-3-carboxamide hydrochloride

The title compound is obtained in accordance with the process of Example 1 using the acid of Preparation 1 and the compound of Preparation 3′ in Step A, and the compound of Preparation 29″ in Step C, it being understood that Step D is not carried out. The compound obtained is converted into salt form and lyophilised as described in the general process to obtain the title compound.

### Elemental Microanalysis: (%, Theoretical:Measured)

% C=66.75:66.44; % H=5.74:5.59; % N=11.39:11.45; % Cl⁻=4.81:4.43

### High-Resolution Mass Spectrometry (ESI+/HR, ESI−/LR):

C₄₁H₄₁Cl N₆O₃ Empirical formula:

[M+H]⁺ calculated: 701.3001

[M+H]⁺ measured: 701.2998

### EXAMPLE 442

5-(5-Chloro-2-{[(3S)-3-(morpholin-4-ylmethyl)-3,4-dihydroisoquinolin-2 (1H)-yl]carbonyl}phenyl)-N-(5-cyanothiophen-3-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 42″ in Step C. The compound obtained is converted into salt form and lyophilised as described in the general process to obtain the title compound.

### Elemental Microanalysis: (%, Theoretical:Measured)

% C=63.07:63.14; % H=5.02:4.87; % N=9.43:9.41; % S=4.32:4.24; % Cl⁻=4.77:4.57

### High-Resolution Mass Spectrometry (ESI+/HR, ESI−/LR):

C₃₉H₃₆Cl N₅O₄S Empirical formula:

[M+H]⁺ calculated: 706.2249

[M+H]⁺ measured: 706.2252

### EXAMPLE 443

5-(5-fluoro-4-hydroxy-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrrolo[2,3-B]pyridin-5-yl)-N-phenyl-1H-pyrrole-3-carboxamide
Step A: 5-(5-Fluoro-4-methoxy-2-{[(3S)-3-(morpholin-4-ylmethyl)-3,4-dihydroiso-quinolin-2 (1H)-yl]carbonyl}phenyl)-1,2-dimethyl-N-(1-methyl-1H-pyrrolo[2,3-b]pyridin-5-yl)-N-phenyl-1H-pyrrole-3-carboxamide

The title compound is obtained in accordance with the process of Example 1 (Steps A to C) using the acid of Preparation 28 and the compound from Preparation 3′ in Step A, and the compound of Preparation 30″ in Step C.

Step B: 5-(5-Fluoro-4-hydroxy-2-{[(3S)-3-(morpholin-4-ylmethyl)-3,4-dihydroisoquinolin-2 (1H)-yl]carbonyl}phenyl)-1,2-dimethyl-N-(1-methyl-1H-pyrrolo[2,3-b]pyridin-5-yl)-N-phenyl-1H-pyrrole-3-carboxamide

To a solution of the compound of Step A (1 g; 1.37 mmol) in anhydrous dichloromethane (10 mL) there is added, dropwise at 0° C., a 1M solution of boron tribromide in dichloromethane (1.8 mL; 1.8 mmol). After stirring for 15 hours at ambient temperature, the reaction mixture is poured dropwise into a solution of ethanol (15 mL) at −10° C. After stirring for one hour, saturated aqueous sodium bicarbonate solution is added, and the reaction mixture is extracted with dichloromethane. After drying over MgSO₄, the residue is purified on a silica gel column using a mixture of dichloromethane and methanol as eluant to yield the expected product.

### High-Resolution Mass Spectrometry (ESI+/HR, ESI−/LR):

C₄₂H₄₁F N₆O₄ Empirical formula:

[M+H]⁺ calculated: 713.3246

[M+H]⁺ measured: 713.3244

### EXAMPLE 444

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-N-(5-methoxypyrazin-2-yl)-1,2-dimethyl-1H-pyrrole-3-carboxamide hydrochloride

The title compound is obtained in accordance with the process of Example 1 using the acid of Preparation 1 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=62.99:62.72; % H=5.42:5.24; % N=11.30:11.19; % Cl⁻=4.77:4.67

### High-Resolution Mass Spectrometry (ESI+/HR, ESI−/LR):

C₃₉H₃₉Cl N₆O₅ Empirical formula:

[M+H]⁺ calculated: 707.2743

[M+H]⁺ measured: 707.2747

### EXAMPLE 445

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(trideuteriomethyl)-2,3-dihydro-1H-pyrrolo[2,3-B]pyridin-5-yl]-1H-pyrrole-3-carboxamide hydrochloride
Step A: 5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(trideuteriomethyl)-1H-pyrrolo[2,3-b]pyridin-5-yl]-1H-pyrrole-3-carboxamide

The intermediate is obtained in accordance with the process of Example 1 using the acid of Preparation 1 and the compound of Preparation 3′ in Step A, and the compound of Preparation 31″ in Step C.

Step B: 5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-[1-(trideuteriomethyl)-2,3-dihydro-1H-pyrrolo[2,3-b]pyridin-5-yl]-1H-pyrrole-3-carboxamide hydrochloride

The procedure is in accordance with the protocol described in Step B of Example 49, it being understood that the product is then subjected to a step of conversion into salt form in the presence of 1M HCl in ether. After filtration and lyophilisation in a mixture of acetonitrile/water, the title product is obtained.

### Elemental Microanalysis: (%, Theoretical:Measured)

% C=65.45:65.33; % H=5.78:5.59; % N=10.90:10.82; % Cl⁻=4.60:4.28

### High-Resolution Mass Spectrometry (ESI+/HR, ESI−/LR):

C₄₂H₄₀Cl D₃N₆O₄ Empirical formula:

[M+H]⁺ calculated: 734.3295

[M+H]⁺ measured: 734.3300

### EXAMPLE 446

5-(5-Chloro-2-{[(3S)-3-(morpholin-4-ylmethyl)-3,4-dihydroisoquinolin-2 (1H)-yl]carbonyl}phenyl)-N-(5-fluoropyrazin-2-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=62.28:62.16; % H=5.10:4.97; % N=11.04:11.35; % Cl⁻=4.85:4.48

### High-Resolution Mass Spectrometry (ESI+/HR, ESI−/LR):

C₃₈H₃₆Cl F N₆O₄ Empirical formula:

[M+H]⁺ calculated: 695.2543

[M+H]⁺ measured: 695.2545

### EXAMPLE 447

5-(4-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide hydrochloride

The title compound is obtained in accordance with the process of Example 1 using the acid of Preparation 26 and the compound of Preparation 3′ in Step A, and the compound of Preparation 1″ in Step C. The compound obtained is converted into salt form and lyophilised as described in the general process to obtain the title compound.

### Elemental Microanalysis: (%, Theoretical:Measured)

% C=65.28:65.27; % H=5.77:5.51; % N=12.02:1.90; % Cl⁻=5.07:4.74

### High-Resolution Mass Spectrometry (ESI+/HR, ESI−/LR):

C₃₈H₃₉F N₆O₄ Empirical formula:

[M+H]⁺ calculated: 663.3090

[M+H]⁺ measured: 663.3084

### EXAMPLE 448

5-(5-Chloro-2-{[(3S)-3-(morpholin-4-ylmethyl)-3,4-dihydroisoquinolin-2 (1H)-yl]carbonyl}phenyl)-N-[5-cyano-1-(trideuteriomethyl)-1H-pyrrol-3-yl]-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 32″ in Step C. The compound obtained is converted into salt form and lyophilised as described in the general process to obtain the title compound.

### Elemental Microanalysis: (%, Theoretical:Measured)

% C=64.69:64.23; % H=5.45:5.47; % N=11.32:11.16; % Cl⁻=4.77:4.56

### High-Resolution Mass Spectrometry (ESI+/HR, ESI−/LR):

C₄₀H₃₆Cl D₃N₆O₄ Empirical formula:

[M+H]⁺ calculated: 706.2982

[M+H]⁺ measured: 706.2985

### EXAMPLE 449

5-(5-Chloro-2-{[(3S)-3-(morpholin-4-ylmethyl)-3,4-dihydroisoquinolin-2 (1H)-yl]carbonyl}phenyl)-N-[5-cyano-2-methyl-1-(trideuteriomethyl)-1H-pyrrol-3-yl]-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 33″ in Step C. The compound obtained is converted into salt form and lyophilised as described in the general process to obtain the title compound.

### Elemental Microanalysis: (%, Theoretical:Measured)

% C=65.07:64.55; % H=5.62:5.51; % N=11.11:10.98; % Cl⁻=4.68:4.58

### High-Resolution Mass Spectrometry (ESI+/HR, ESI−/LR):

C₄₁H₃₈Cl D₃N₆O₄ Empirical formula:

[M+H]⁺ calculated: 720.3139

[M+H]⁺ measured: 720.3143

### EXAMPLE 450

5-(5-Chloro-2-{[(3S)-3-(morpholin-4-ylmethyl)-3,4-dihydroisoquinolin-2 (1H)-yl]carbonyl}phenyl)-N-(3-cyano-1-methyl-1H-pyrazol-5-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 34″ in Step C. The compound obtained is converted into salt form and lyophilised as described in the general process to obtain the title compound.

### Elemental Microanalysis: (%, Theoretical:Measured)

% C=63.24:62.62; % H=5.31:5.09; % N=13.24:13.04; % Cl⁻=4.79:4.37

### High-Resolution Mass Spectrometry (ESI+/HR, ESI−/LR):

C₃₉H₃₈Cl N₇O₄ Empirical formula:

[M+H]⁺ calculated: 704.2747

[M+H]⁺ measured: 704.2747

### EXAMPLE 451

5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazolo[3,4-B]pyridin-5-yl)-1H-pyrrole-3-carboxamide hydrochloride

The title compound is obtained in accordance with the process of Example 1 using the acid of Preparation 8 and the compound of Preparation 3′ in Step A, and the compound of Preparation 17″ in Step C. The compound obtained is converted into salt form and lyophilised as described in the general process to obtain the title compound.

### Elemental Microanalysis: (%, Theoretical:Measured)

% C=65.64:66.28; % H=5.51:5.45; % N=13.07:12.17% Cl⁻=4.73:5.51

### EXAMPLE 452

N-(1,3-dimethyl-1H-pyrazol-4-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

### Elemental Microanalysis: (%, Theoretical:Measured)

% C=65.68:64.77; % H=5.94:5.55; % N=11.78:10.69% Cl⁻=4.97:6.48

and

### EXAMPLE 453

N-(1,5-dimethyl-1H-pyrazol-4-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide hydrochloride

### Elemental Microanalysis: (%, Theoretical:Measured)

% C=65.68:65.43; % H=5.94:5.62; % N=11.78:10.95% Cl⁻=4.97:5.60

The compounds of Examples 452 and 453 are obtained in accordance with the process of Example 1 using the acid of Preparation 8 and the compound of Preparation 3′ in Step A, and the mixture of Preparation 27″ in Step C. At the end of Step D, the isomers are separated by preparative HPLC using acetonitrile and water-TFA as eluants. After evaporating off the solvent and neutralising with sodium bicarbonate, the products are subjected to a step of conversion into salt form in the presence of 1M HCl in ether. After filtration and lyophilisation in a mixture of acetonitrile/water, the title products are obtained.

### EXAMPLE 454

5-(5-Chloro-2-{[3-(trifluoromethyl)-3,4-dihydroisoquinolin-2 (1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide

The title compound is obtained in accordance with the process of Example 1 using the acid from Preparation 1 and racemic 3-(trifluoromethyl)-1,2,3,4-tetrahydroisoquinoline in Step A, and the compound of Preparation 18″ in Step C.

### Elemental Microanalysis: (%, Theoretical:Measured)

% C=64.77:64.81; % H=4.55:4.52; % N=10.21:10.33

### High-Resolution Mass Spectrometry (ESI+/HR):

C₃₇H₃₁Cl F₃N₅O₃ Empirical formula:

[M+H]⁺ calculated: 686.2140

[M+H]⁺ measured: 686.2145

### EXAMPLE 455

5-(5-Chloro-2-{[(3R)-3-methyl-3,4-dihydroisoquinolin-2 (1H)-yl]-carbonyl}phenyl)-N—{5-cyano-2-methyl-1-[2-(morpholin-4-yl)ethyl]-1H-pyrrol-3-yl}-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation F in Step A, and the compound of Preparation 35″ in Step C. The compound obtained is converted into salt form and lyophilised as described in the general process to obtain the title compound.

### Elemental Microanalysis: (%, Theoretical:Measured)

% C=65.71:66.07; % H=5.78:5.82; % N=10.95:10.66; % Cl⁻=4.62:4.45

### High-Resolution Mass Spectrometry (ESI+/HR, ESI−/LR):

C₄₂H₄₃Cl N₆O₄ Empirical formula:

[M+H]⁺ calculated: 731.3107

[M+H]⁺ measured: 731.3109

### EXAMPLE 456

5-(5-Chloro-2-{[(3R)-3-methyl-3,4-dihydroisoquinolin-2 (1H)-yl]-carbonyl}phenyl)-N-(4-hydroxyphenyl)-1,2-dimethyl-N—{2-[2-(morpholin-4-yl)-ethoxy]pyrimidin-5-yl}-1H-pyrrole-3-carboxamide hydrochloride

The title compound is obtained in accordance with the process of Example 1 using the acid of Preparation 1 and the compound of Preparation 1′ in Step A, and the compound of Preparation 36″ in Step C. The compound obtained is converted into salt form and lyophilised as described in the general process to obtain the title compound.

### Elemental Microanalysis: (%, Theoretical:Measured)

% C=63.41:63.51; % H=5.59:5.26; % N=11.09:11.10; % Cl⁻=4.68:4.46

### High-Resolution Mass Spectrometry (ESI+/HR, ESI−/LR):

C₄₀H₄₁Cl N₆O₅ Empirical formula:

[M+H]⁺ calculated: 721.2900

[M+H]⁺ measured: 721.2907

### EXAMPLE 457

5-(5-Chloro-2-{[(3S)-3-(morpholin-4-ylmethyl)-3,4-dihydroisoquinolin-2 (1H)-yl]carbonyl}phenyl)-N-(6-cyano-5-methoxypyridin-2-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the appropriate amine in Step C. The compound obtained is converted into salt form and lyophilised as described in the general process to obtain the title compound.

### Elemental Microanalysis: (%, Theoretical:Measured)

% C=64.15:63.95; % H=5.25:4.79; % N=10.95:10.97; % Cl⁻=4.62:4.22

### High-Resolution Mass Spectrometry (ESI+/HR):

C₄₁H₃₉Cl N₆O₅ Empirical formula:

[M+H]⁺ calculated: 731.2743

[M+H]⁺ measured: 731.2746

### EXAMPLE 458

5-(5-Chloro-2-{[(3S)-3-(morpholin-4-ylmethyl)-3,4-dihydroisoquinolin-2 (1H)-yl]carbonyl}phenyl)-N-(5-cyano-1,4-dimethyl-1H-pyrrol-3-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 37″ in Step C. The compound obtained is converted into salt form and lyophilised as described in the general process to obtain the title compound.

### Elemental Microanalysis: (%, Theoretical:Measured)

% C=65.34:65.68; % H=5.62:5.31; % N=11.15:11.15; % Cl³¹=4.70:4.33

### High-Resolution Mass Spectrometry (ESI+/HR, ESI−/LR):

C₄₁H₄₁Cl N₆O₄ Empirical formula:

[M+H]⁺ calculated: 717.2951

[M+H]⁺ measured: 717.2954

### EXAMPLE 459

5-(5-Chloro-2-{[(3S)-3-(morpholin-4-ylmethyl)-3,4-dihydroisoquinolin-2 (1H)-yl]carbonyl}phenyl)-N-(5-cyano-1-ethyl-2-methyl-1H-pyrrol-3-yl)-N-(4-hydroxyphenyl)-1,2-dimethyl-1H-pyrrole-3-carboxamide hydrochloride

The title compound is obtained in accordance with the process of Example 1 using the acid of Preparation 1 and the compound of Preparation 3′ in Step A, and the compound of Preparation 38″ in Step C. The compound obtained is converted into salt form and lyophilised as described in the general process to obtain the title compound.

### Elemental Microanalysis: (%, Theoretical:Measured)

% C=65.71; 65.29; % H=5.78:5.51; % N=10.95:10.95; % Cl⁻=4.62:4.39

### High-Resolution Mass Spectrometry (ESI+/HR, ESI−/LR):

C₄₂H₄₃Cl N₆O₄ Empirical formula:

[M+H]⁺ calculated: 731.3107

[M+H]⁺ measured: 731.3109

### EXAMPLE 460

5-(5-Chloro-2-{[(3R)-3-[3-(morpholin-4-yl)propyl]-3,4-dihydroisoquinolin-2 (1H)-yl]carbonyl}phenyl)-N-(4-hydroxyphenyl)-1,2-dimethyl-N-[1-(tetrahydrofuran-3-yl)-1H-pyrazol-4-yl]-1H-pyrrole-3-carboxamide hydrochloride

The title compound is obtained in accordance with the process of Example 1 using the acid of Preparation 1 and the compound of Preparation 6′ in Step A, and the compound of Preparation 22″ in Step C. The compound obtained is converted into salt form and lyophilised as described in the general process to obtain the title compound.

### Elemental Microanalysis: (%, Theoretical:Measured)

% C=64.58:64.24; % H=6.05:5.88; % N=10.51:10.53; % Cl⁻=4.43:4.39

### High-Resolution Mass Spectrometry (ESI+/HR, ESI−/LR):

C₄₃H₄₇Cl N₆O₅ Empirical formula:

[M+H]⁺ calculated: 763.3369

[M+H]⁺ measured: 763.3371

### EXAMPLE 461

5-(5-Chloro-2-{[(3R)-3-[3-(morpholin-4-yl)propyl]-3,4-dihydroisoquinolin-2 (1H)-yl]carbonyl}phenyl)-N-(5-cyano-1,2-dimethyl-1H-pyrrol-3-yl)-1,2-dimethyl-N-phenyl-1H-pyrrole-3-carboxamide hydrochloride

The title compound is obtained in accordance with the process of Example 1 using the acid of Preparation 1 and the compound of Preparation 6′ in Step A, and the compound of Preparation 29″ in Step C, it being understood that Step D is not carried out. The compound obtained is converted into salt form and lyophilised as described in the general process to obtain the title compound.

### Elemental Microanalysis: (%, Theoretical:Measured)

% C=67.44:66.68; % H=6.05:5.80; % N=10.97:10.95; % Cl⁻=4.63:4.57

### High-Resolution Mass Spectrometry (ESI+/HR):

C₄₃H₄₅Cl N₆O₃ Empirical formula:

[M+H]⁺ calculated: 729.3314

[M+H]⁺ measured: 729.3316

### EXAMPLE 462

5-(5-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(5-methyl-1,2-oxazol-3-yl)-1H-pyrrole-3-carboxamide

The title compound is obtained in accordance with the process of Example 1 using the acid of Preparation 1 and the compound of Preparation 3′ in Step A, and the appropriate amine in Step C.

### Elemental Microanalysis: (%, Theoretical:Measured)

% C=67.10:66.64; % H=5.63:5.40; % N=10.30:10.24

### High-Resolution Mass Spectrometry (ESI+/HR, ESI−/LR):

C₃₈H₃₈Cl N₅O₅ Empirical formula:

[M+H]⁺ calculated: 680.2634

[M+H]⁺ measured: 680.2637

### EXAMPLE 463

N-(2-ethoxypyrimidin-5-yl)-5-(5-fluoro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-1H-pyrrole-3-carboxamide

The title compound is obtained in accordance with the process of Example 1 using the acid of Preparation 8 and the compound of Preparation 3′ in Step A, and the compound of Preparation 39″ in Step C.

### Elemental Microanalysis: (%, Theoretical:Measured)

% C=68.17:67.52; % H=5.86:5.60; % N=11.92:11.43

### EXAMPLE 464

5-(5-Chloro-2-{[(3S)-3-(morpholin-4-ylmethyl)-3,4-dihydroisoquinolin-2 (1H)-yl]carbonyl}phenyl)-N-(5-hydroxypyridin-2-yl)-1,2-dimethyl-N-(1-methyl-1H-pyrrolo[2,3-B]pyridin-5-yl)-1H-pyrrole-3-carboxamide dihydrochloride

The title compound is obtained in accordance with the process of Example 1 using the acid of Preparation 1 and the compound of Preparation 3′ in Step A, and the compound of Preparation 40″ in Step C. The compound obtained is converted into salt form and lyophilised as described in the general process to obtain the title compound.

### High-Resolution Mass Spectrometry (ESI+/HR, ESI−/LR):

C₄₁H₄₀Cl N₇O₄ Empirical formula:

[M+H]⁺ calculated: 730.2903

[M+H]⁺ measured: 730.2907

### EXAMPLE 465

5-(4-Chloro-2-{[(3S)-3-(morpholin-4-ylmethyl)-3,4-dihydroisoquinolin-2 (1H)-yl]carbonyl}phenyl)-N-(5-cyano-1,2-dimethyl-1H-pyrrol-3-yl)-N-(4-hydroxyphenyl)-1,2-dimethyl-1H-pyrrole-3-carboxamide hydrochloride

The title compound is obtained in accordance with the process of Example 1 using the acid of Preparation 29 and the compound of Preparation 3′ in Step A, and the compound of Preparation 18″ in Step C. The compound obtained is converted into salt form and lyophilised as described in the general process to obtain the title compound.

### Elemental Microanalysis: (%, Theoretical:Measured)

% C=65.34:64.81; % H=5.62:5.27; % N=11.15:10.95% Cl⁻=4.70:5.09

### High-Resolution Mass Spectrometry (ESI+):

C₄₁H₄₁Cl N₆O₄ Empirical formula:

[M+H]⁺ calculated: 717.2951

[M+H]⁺ measured: 717.2952

### EXAMPLE 466

5-(4-Chloro-2-{[(3S)-3-(morpholin-4-ylmethyl)-3,4-dihydroisoquinolin-2 (1H)-yl]carbonyl}phenyl)-N-(4-hydroxyphenyl)-1,2-dimethyl-N-(1-methyl-1H-pyrazol-4-yl)-1H-pyrrole-3-carboxamide hydrochloride

The title compound is obtained in accordance with the process of Example 1 using the acid of Preparation 29 and the compound of Preparation 3′ in Step A, and the compound of Preparation 1″ in Step C. The compound obtained is converted into salt form and lyophilised as described in the general process to obtain the title compound.

### Elemental Microanalysis: (%, Theoretical:Measured)

% C=66.77:62.82; % H=5.63:5.29; % N=11.74:11.75; % Cl⁻=5.01:5.23

### High-Resolution Mass Spectrometry (ESI+/HR, ESI−/LR):

C₃₈H₃₉Cl N₆O₄ Empirical formula:

[M+H]⁺ calculated: 679.2794

[M+H]⁺ measured: 679.2796

### EXAMPLE 467

5-(5-Chloro-2-{[3,4-dihydroisoquinolin-2 (1H)-yl]carbonyl}phenyl)-N-(4-hydroxyphenyl)-1,2-dimethyl-N-phenyl-1H-pyrrole-3-carboxamide

The title compound is obtained in accordance with the process of Example 1 using the acid of Preparation 1 and 1,2,3,4-tetrahydroisoquinoline in Step A, and the compound of Preparation 2″ in Step C.

### Elemental Microanalysis: (%, Theoretical:Measured)

% C=72.97:72.93; % H=5.25:5.08; % N=7.29:7.34

### High-Resolution Mass Spectrometry (ESI/FIA/HR and MS/MS):

C₃₅H₃₀Cl N₃O₃ Empirical formula:

[M+H]⁺ calculated: 576.2048

[M+H]⁺ measured: 576.2067

### Pharmacological Study

### EXAMPLE A: INHIBITION OF BCL-2 BY THE FLUORESCENCE POLARISATION TECHNIQUE

The fluorescence polarisation tests were carried out on microplates (384 wells). The Bcl-2 protein, labelled (histag-Bcl-2 such that Bcl-2 corresponds to the UniProtKB® primary accession number: P10415), at a final concentration of 2.50×10⁻⁸ M, is mixed with a fluorescent peptide (Fluorescein-REIGAQLRRMADDLNAQY), at a final concentration of 1.00×10⁻⁸ M in a buffer solution (Hepes 10 mM, NaCl 150 mM, Tween20 0.05%, pH 7.4), in the presence or absence of increasing concentrations of test compounds. After incubation for 2 hours, the fluorescence polarisation is measured.

The results are expressed in IC₅₀ (the concentration of compound that inhibits fluorescence polarisation by 50%) and are presented in Table 1 below.

The results show that the compounds of the invention inhibit interaction between the Bcl-2 protein and the fluorescent peptide described hereinbefore.

### EXAMPLE B: IN VITRO CYTOTOXICITY

The cytotoxicity studies were carried out on the RS4;11 leukaemia tumour line.

The cells are distributed onto microplates and exposed to the test compounds for 48 hours.

The cell viability is then quantified by a colorimetric assay, the Microculture Tetrazolium Assay (Cancer Res., 1987, 47, 939-942).

The results are expressed in IC₅₀ (the concentration of compound that inhibits cell viability by 50%) and are presented in Table 1 below.

The results show that the compounds of the invention are cytotoxic.

TABLE 1
IC₅₀ of Bcl-2 inhibition (fluorescence polarisation
test) and of cytotoxicity for RS4; 11 cells

| | IC₅₀ (nM) Bcl-2 FP | IC₅₀ (nM) MTT RS4; 11 |
|---|---|---|
| Example 1 | 5.0 | 33.6 |
| Example 2 | ND | 1660 |
| Example 3 | 24.6 | 94.9 |
| Example 4 | ND | 231 |
| Example 5 | 21.2 | 44.8 |
| Example 6 | 25.2 | 69 |
| Example 7 | 25.6 | 90.6 |
| Example 8 | ND | 255 |
| Example 9 | 22.4 | 87.5 |
| Example 10 | 16.2 | 205 |
| Example 11 | 14.2 | 202 |
| Example 12 | 5.5 | 39.6 |
| Example 13 | 4.4 | 19.8 |
| Example 14 | 3.7 | 8.23 |
| Example 15 | 11.1 | 69.4 |
| Example 16 | 12.6 | 22.7 |
| Example 17 | 8.0 | 75.2 |
| Example 18 | 3.9 | 27.6 |
| Example 19 | 6.0 | 65.5 |
| Example 20 | 4.9 | 164 |
| Example 21 | 4.7 | 79.9 |
| Example 22 | 6.6 | 45.7 |
| Example 23 | 3.6 | 25.4 |
| Example 24 | 6.2 | 79.1 |
| Example 25 | 4.0 | 33.3 |
| Example 26 | 4.1 | 541 |
| Example 27 | 5.2 | 93.4 |
| Example 28 | 7.5 | 95.3 |
| Example 29 | 6.6 | 47.5 |
| Example 30 | 4.7 | 771 |
| Example 31 | 7.2 | 89.3 |
| Example 32 | 13.3 | 240 |
| Example 33 | 10.9 | 57.8 |
| Example 34 | 8.2 | 47 |
| Example 35 | 50.0 | 560 |
| Example 36 | 71.4 | >600 |
| Example 37 | 60.4 | >600 |
| Example 38 | 7.5 | 134 |
| Example 39 | 7.2 | 19.7 |
| Example 40 | 64.4 | 431 |
| Example 41 | 10.0 | 22.6 |
| Example 42 | 5.2 | 4.36 |
| Example 43 | 5.1 | 28.9 |
| Example 44 | 3.0 | 5.41 |
| Example 45 | 38.9 | 403 |
| Example 46 | 76.6 | >600 |
| Example 47 | 5.9 | 44.5 |
| Example 48 | 4.4 | 14.9 |
| Example 49 | 4.0 | 14.1 |
| Example 50 | 5.9 | 33.1 |
| Example 51 | 26.7 | 354 |
| Example 52 | 28.9 | 433 |
| Example 53 | 43.5 | 293 |
| Example 54 | 18.0 | 30.5 |
| Example 55 | 209.6 | >600 |
| Example 56 | 75.0 | >600 |
| Example 57 | 80.0 | >600 |
| Example 58 | 133.3 | >600 |
| Example 59 | 134.0 | >600 |
| Example 60 | ND | 18.3 |
| Example 61 | 4.5 | 37.8 |
| Example 62 | 14.3 | 127 |
| Example 63 | 5.1 | 11.2 |
| Example 65 | 21.9 | 113 |
| Example 66 | 16.9 | 241 |
| Example 67 | 12.5 | 98.6 |
| Example 68 | 2.1 | 20.7 |
| Example 69 | 5.3 | 3.68 |
| Example 70 | 8.5 | 63.5 |
| Example 71 | 6.1 | 12.9 |
| Example 72 | 7.7 | 43.6 |
| Example 73 | 8 | 42.6 |
| Example 74 | 4 | 45.4 |
| Example 75 | 52.9 | 367 |
| Example 81 | ND | 249 |
| Example 82 | 19.5 | 427 |
| Example 88 | 15.9 | 69.6 |
| Example 92 | 8.4 | 37.8 |
| Example 93 | 19.7 | 368 |
| Example 94 | 8.6 | 104 |
| Example 95 | 7 | 174 |
| Example 96 | 13.5 | 161 |
| Example 97 | 19 | 98 |
| Example 98 | 7.6 | 68.3 |
| Example 99 | 6.4 | 108 |
| Example 100 | 22.9 | 193 |
| Example 101 | 21.1 | 743 |
| Example 102 | 2 | 6.51 |
| Example 108 | 3.9 | 15.4 |
| Example 109 | 91.5 | 930 |
| Example 110 | 8.5 | 39.5 |
| Example 115 | 3.6 | 18.3 |
| Example 116 | 3.4 | 47.8 |
| Example 122 | 3.4 | 82.4 |
| Example 126 | 4.2 | 49.2 |
| Example 127 | 5.3 | 111 |
| Example 129 | 18.1 | 275 |
| Example 207 | 4.6 | 32.2 |
| Example 209 | 7.4 | 25.4 |
| Example 210 | 8.8 | 47.2 |
| Example 214 | 6.3 | 236 |
| Example 230 | 5.1 | 18.3 |
| Example 358 | 14.8 | 165 |
| Example 384 | 9.7 | 216 |
| Example 385 | 5.7 | 28.7 |
| Example 386 | 2.6 | 9.23 |
| Example 387 | 20.6 | 243 |
| Example 388 | 3.6 | 16.5 |
| Example 389 | 14.7 | 208 |
| Example 390 | 21.2 | 173 |
| Example 391 | 30.3 | 255 |
| Example 392 | 3.8 | 11.4 |
| Example 393 | 2.1 | 1.95 |
| Example 394 | 2.5 | 2.76 |
| Example 395 | 3.5 | 7.57 |
| Example 396 | 15.7 | 116 |
| Example 397 | 4.5 | 37.1 |
| Example 398 | 3.9 | 13.4 |
| Example 399 | 9.9 | 155 |
| Example 400 | 49 | 469 |
| Example 401 | 8 | 24.3 |
| Example 402 | 26.1 | 241 |
| Example 403 | 24.2 | 289 |
| Example 404 | 24.4 | 85.5 |
| Example 406 | 4.2 | 10.6 |
| Example 407 | 20.6 | 150 |
| Example 408 | 5.4 | 4.46 |
| Example 409 | 12.2 | 41.9 |
| Example 410 | 9.9 | 61.6 |
| Example 411 | 5.5 | 32.8 |
| Example 412 | 3.1 | 0.398 |
| Example 413 | 3.1 | 9.43 |
| Example 415 | 3.8 | 29.9 |
| Example 416 | 2.9 | 42.5 |
| Example 417 | 3 | 11 |
| Example 418 | 10.1 | 159 |
| Example 419 | 2.8 | 15.8 |
| Example 420 | 6.1 | 51.8 |
| Example 421 | 2.5 | 3.73 |
| Example 422 | 9.2 | 101 |
| Example 423 | 11.7 | 344 |
| Example 424 | 13.6 | 102 |
| Example 425 | 4.2 | 62 |
| Example 426 | 5.2 | 139 |
| Example 427 | 2.2 | 23.3 |
| Example 428 | 3.5 | 59.2 |
| Example 429 | 3.1 | 109 |
| Example 430 | 19.9 | 328 |
| Example 431 | 3 | 89 |
| Example 432 | 3.4 | 76.6 |
| Example 433 | 2.6 | 48.2 |
| Example 434 | 3.5 | 45.7 |
| Example 435 | 14.9 | 441 |
| Example 436 | 49.1 | 338 |
| Example 437 | 4.4 | 70.5 |
| Example 438 | 11.3 | 379 |
| Example 439 | 6.1 | 329 |
| Example 440 | 2.6 | 13.6 |
| Example 441 | 3.5 | 132 |
| Example 442 | 3 | 42.8 |
| Example 443 | ND | 57.9 |
| Example 444 | 5.5 | 181 |
| Example 445 | 2 | 19 |
| Example 446 | 7.8 | 249 |
| Example 447 | 20 | >600 |
| Example 448 | 2.4 | 12.1 |
| Example 449 | 1.7 | 11.9 |
| Example 450 | 2.7 | 53.2 |
| Example 451 | 2.9 | 102 |
| Example 452 | 8.3 | 112 |
| Example 453 | 6.9 | 99.9 |
| Example 454 | 6.5 | 158 |
| Example 455 | 1.9 | 11.8 |
| Example 456 | 2.7 | 80.6 |
| Example 457 | ND | 80.4 |
| Example 458 | 10.5 | 197 |
| Example 459 | 2.5 | 5.93 |
| Example 460 | 2.8 | 22.9 |
| Example 461 | 3.1 | 18.6 |
| Example 462 | 13.3 | 368 |
| Example 463 | ND | 40.8 |
| Example 464 | 36.6 | 723 |
| Example 465 | ND | 50.6 |
| Example 466 | ND | 493 |
| Example 467 | 19.2 | 188 |

ND: not determined

### EXAMPLE C: INDUCTION OF CASPASE ACTIVITY IN VIVO

The ability of the compounds of the invention to activate caspase 3 is evaluated in a xenograft model of RS4;11 leukaemia cells.

1×10⁷ RS4;11 cells are grafted sub-cutaneously into immunosuppressed mice (SCID strain). 25 to 30 days after the graft, the animals are treated orally with the various compounds. Sixteen hours after treatment, the tumour masses are recovered and lysed, and the caspase 3 activity is measured in the tumour lysates.

This enzymatic measurement is carried out by assaying the appearance of a fluorigenic cleavage product (DEVDase activity, Promega). It is expressed in the form of an activation factor corresponding to the ratio between the two caspase activities: the activity for the treated mice divided by the activity for the control mice.

The results obtained show that the compounds of the invention are capable of inducing apoptosis in RS4;11 tumour cells in vivo.

### EXAMPLE D: QUANTIFICATION OF THE CLEAVED FORM OF CASPASE 3 IN VIVO

The ability of the compounds of the invention to activate caspase 3 is evaluated in a xenograft model of RS4;11 leukaemia cells.

1×10⁷ RS4;11 cells are grafted sub-cutaneously into immunosuppressed mice (SCID strain). 25 to 30 days after the graft, the animals are treated orally with the various compounds. After treatment, the tumour masses are recovered and lysed, and the cleaved (activated) form of caspase 3 is quantified in the tumour lysates.

The quantification is carried out using the “Meso Scale Discovery (MSD) ELISA platform” test, which specifically assays the cleaved form of caspase 3. It is expressed in the form of an activation factor corresponding to the ratio between the quantity of cleaved caspase 3 in the treated mice divided by the quantity of cleaved caspase 3 in the control mice.

The results show that the compounds of the invention are capable of inducing apoptosis in RS4;11 tumour cells in vivo.

TABLE 2
Caspase activation factors (cleaved caspase 3 MSD test in
the tumours of treated mice versus control mice) in vivo,
after treatment by the oral route (exact doses in brackets)

| | Time period after | | |
|---|---|---|---|
| | which the tumour | Activation factor ± | |
| Compound tested | is removed (T) | SEM (versus control) | |
| Example 25 | 2 hours | 45.7 ± 2.0 | (25 mg/kg) |
| Example 28 | 2 hours | 72.3 ± 5.4 | (12.5 mg/kg) |
| Example 47 | 2 hours | 12.3 ± 2.4 | (25 mg/kg) |
| Example 61 | 2 hours | 76.0 ± 5.2 | (12.5 mg/kg) |
| Example 67 | 2 hours | 29.8 ± 4.0 | (25 mg/kg) |
| Example 71 | 2 hours | 46.8 ± 16.1 | (25 mg/kg) |
| Example 74 | 2 hours | 24.5 ± 7.4 | (12.5 mg/kg) |
| Example 108 | 2 hours | 22.6 ± 2.4 | (12.5 mg/kg) |
| Example 230 | 2 hours | 42.2 ± 9.3 | (25 mg/kg) |
| Example 386 | 2 hours | 52.0 ± 8.6 | (12.5 mg/kg) |
| Example 388 | 2 hours | 85.7 ± 3.7 | (25 mg/kg) |
| Example 421 | 2 hours | 38.7 ± 10.7 | (12.5 mg/kg) |
| Example 449 | 2 hours | 50.5 ± 3.4 | (12.5 mg/kg) |

### EXAMPLE E: ANTI-TUMOUR ACTIVITY IN VIVO

The anti-tumour activity of the compounds of the invention is evaluated in a xenograft model of RS4;11 leukaemia cells.

1×10⁷ RS4;11 cells are grafted sub-cutaneously into immunosuppressed mice (SCID strain). 25 to 30 days after the graft, when the tumour mass has reached about 150 mm³, the mice are treated orally with the various compounds in two different regimes (daily treatment for five days per week for two weeks, or two treatments weekly for two weeks). The tumour mass is measured twice weekly from the start of treatment.

The results obtained accordingly show that the compounds of the invention are capable of inducing significant tumour regression, which can be total, during the treatment period.

### EXAMPLE F: PHARMACEUTICAL COMPOSITION: TABLETS

| | | |
|---|---|---|
| 1000 tablets containing a dose of 5 mg of | 5 | g |
| a compound selected from Examples 1 to 467 | | |
| Wheat starch | 20 | g |
| Maize starch | 20 | g |
| Lactose | 30 | g |
| Magnesium stearate | 2 | g |
| Silica | 1 | g |
| Hydroxypropylcellulose | 2 | g |

1. A process for the preparation of a compound of formula (I): 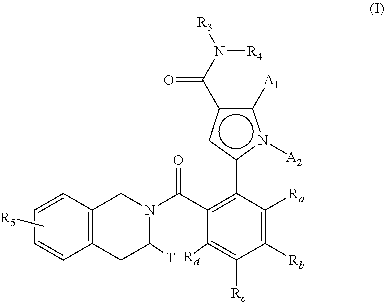
wherein:
A₁ and A₂, each independently of the other, represent a hydrogen atom, a halogen atom, a linear or branched (C₁-C₆)polyhaloalkyl group, a linear or branched (C₁-C₆)alkyl group or a cycloalkyl group;
T represents a hydrogen atom, a linear or branched (C₁-C₆)alkyl group optionally substituted by from one to three halogen atoms, a (C₁-C₄)alkyl-NR₁R₂ group, or a (C₁-C₄)alkyl-OR₆ group;
R₁ and R₂, each independently of the other, represent a hydrogen atom or a linear or branched (C₁-C₆)alkyl group,
or R₁ and R₂ form with the nitrogen atom carrying them a heterocycloalkyl;
R₃ represents a linear or branched (C₁-C₆)alkyl group or a heteroaryl group, wherein one or more of the carbon atoms of the preceding groups, or of their possible substituents, may be deuterated;
R₄ represents an aryl group, a heteroaryl group, a cycloalkyl group or a linear or branched (C₁-C₆)alkyl group, wherein one or more of the carbon atoms of the preceding groups, or of their possible substituents, may be deuterated;
R₅ represents a hydrogen or halogen atom, a linear or branched (C₁-C₆)alkyl group, or a linear or branched (C₁-C₆)alkoxy group;
R₆ represents a hydrogen atom or a linear or branched (C₁-C₆)alkyl group;
Rₐ, R_{b} and R_{d} each represent a hydrogen atom and R_{c} represents a group selected from R₇—CO—NH—(C₀-C₆)alkyl-, R₇—SO₂—NH—(C₀-C₆)alkyl-, R₇—NH—CO—NH—(C₀-C₆)alkyl- and R₇—O—CO—NH—(C₀-C₆)alkyl-;
R₇ represents a hydrogen atom, a linear or branched (C₁-C₆)alkyl group, a linear or branched (C₂-C₆)alkenyl group, a linear or branched (C₂-C₆)alkynyl group, an aryl group or a heteroaryl group;
it being understood that:
“aryl” means a phenyl, naphthyl, biphenyl or indenyl group,
“heteroaryl” means any mono- or bi-cyclic group composed of from 5 to 10 ring members, having at least one aromatic moiety and having from 1 to 4 hetero atoms selected from oxygen, sulphur and nitrogen (including quaternary nitrogens),
“cycloalkyl” means any mono- or bi-cyclic, non-aromatic, carbocyclic group having from 3 to 10 ring members,
“heterocycloalkyl” means any mono- or bi-cyclic, non-aromatic, condensed or spiro group composed of from 3 to 10 ring members and having from 1 to 3 hetero atoms selected from oxygen, sulphur, SO, SO₂ and nitrogen,
wherein the aryl, heteroaryl, cycloalkyl and heterocycloalkyl groups so defined and the alkyl, alkenyl, alkynyl and alkoxy groups may be optionally substituted by from 1 to 3 groups selected from optionally substituted, linear or branched (C₁-C₆)alkyl, (C₃-C₆)spiro, optionally substituted, linear or branched (C₁-C₆)alkoxy, (C₁-C₆)alkyl-S—, hydroxy, oxo (or N-oxide where appropriate), nitro, cyano, —COOR′, —OCOR′, NR′R″, linear or branched (C₁-C₆)polyhaloalkyl, trifluoromethoxy, (C₁-C₆)alkylsulphonyl, halogen, optionally substituted aryl, heteroaryl, aryloxy, arylthio, cycloalkyl, heterocycloalkyl optionally substituted by one or more halogen atoms or alkyl groups, wherein R′ and R″, each independently of the other, represent a hydrogen atom or an optionally substituted, linear or branched (C₁-C₆)alkyl group,
or an enantiomer, a diastereoisomer, or an addition salt thereof with a pharmaceutically acceptable acid or base,
which process comprises which process comprises subjecting a compound of formula (II) 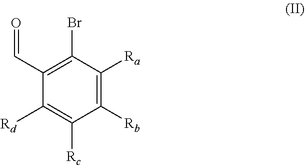
to a Heck reaction in an aqueous or organic medium, in the presence of a palladium catalyst, a base, a phosphine and a compound of formula (III): 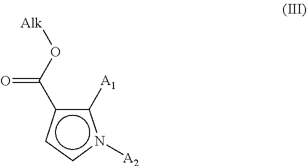
wherein Alk represents a linear or branched (C₁-C₆)alkyl, to obtain the compound of formula (IV): 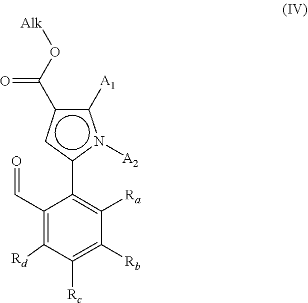
the aldehyde function of which compound of formula (IV) is oxidized to a carboxylic acid to form a compound of formula (V): 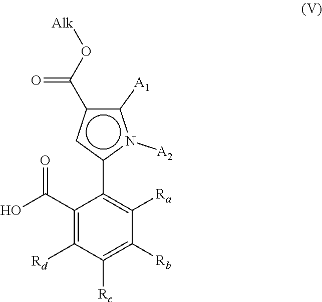
which compound of formula (V) is then subjected to peptide coupling with a compound of formula (VI): 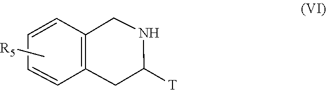
to yield a compound of formula (VII): 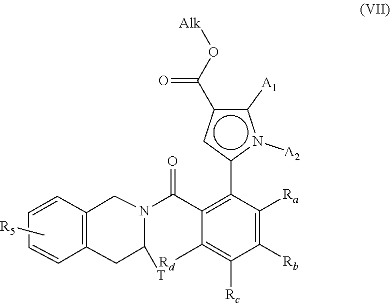
the ester function of which compound of formula (VII) is hydrolyzed to yield the corresponding carboxylic acid or carboxylate, which may be converted into an acid derivative, including an acyl chloride or anhydride, which compound is then coupled with an amine NHR₃R₄, to yield the compound of formula (I),
which compound of formula (I) may be purified according to a conventional separation technique, which is converted, if desired, into its addition salts with a pharmaceutically acceptable acid or base and which is optionally separated into its isomers according to a conventional separation technique,
wherein, at any time considered appropriate in the course of the above-described process, certain groups of the reagents or intermediates of synthesis may be protected and then deprotected according to the requirements of synthesis.

2. The process according to claim 1, wherein one of the groups R₃ or R₄ of the amine NHR₃R₄ is substituted by a hydroxy function, which hydroxyl function is subjected to a protection reaction prior to any coupling with the carboxylic acid formed from the compound of formula (VII), or with a corresponding acid derivative thereof, and wherein the resulting protected compound of formula (I) undergoes a deprotection reaction and is then optionally converted into one of its addition salts with a pharmaceutically acceptable acid or base.

3. The process according to claim 1, wherein A₁ represents a hydrogen atom or a methyl group.

4. The process according to claim 1, wherein A₂ represents a linear or branched (C₁-C₆)alkyl group optionally substituted by a group selected from halogen, hydroxy, linear or branched (C₁-C₆)alkoxy, NR′R″ and morpholine.

5. The process according to claim 1, wherein A₂ represents a linear or branched (C₁-C₆)polyhaloalkyl group or a cyclopropyl group.

6. The process according to claim 1, wherein A₁ and A₂ both represent a methyl group.

7. The process according to claim 1, wherein T represents methyl, aminomethyl, (morpholin-4-yl)methyl, (4-methylpipérazin-1-yl)methyl, 2-(morpholin-4-yl)ethyl, [2-(morpholin-4-yl)ethoxy]methyl, hydroxymethyl, [2-(dimethylamino)ethoxy]methyl, hexahydropyrazino[2,1-c][1,4]oxazin-8 (1H)-ylmethyl, 1-oxa-6-azaspiro[3.3]hept-6-ylmethyl, 3-(morpholin-4-yl)propyl or trifluoromethyl.

8. The process according to claim 1, wherein R₄ represents phenyl, 4-hydroxyphenyl, 3-fluoro-4-hydroxyphenyl, 2-hydroxypyrimidine or 3-hydroxypyridine.

9. The process according to claim 1, wherein R₃ represents a heteroaryl group.

10. The process according to claim 1, wherein R₃ represents a group selected from methyl, 1H-pyrazole, 1H-indole, 1H-indazole, pyridine, pyrimidine, 1H-pyrrolo[2,3-b]pyridine, 2,3-dihydro-1H-pyrrolo[2,3-b]pyridine, 1H-benzimidazole, 1H-pyrrole, 1H-pyrrolo[2,3-c]pyridine, 1H-pyrrolo[3,2-b]pyridine, 5H-pyrrolo[3,2-d]pyrimidine, thiophene, pyrazine, 1H-pyrazolo[3,4-b]pyridine, 1,2-oxazole, and 1H-pyrazolo[1,5-a]pyrimidine, which groups may be optionally substituted by one or more substituents selected from halogen, linear or branched (C₁-C₆)alkyl, linear or branched (C₁-C₆)alkoxy, cyano, cyclopropyl, oxetane, tetrahydrofuran, —CO—O—CH₃, trideuteriomethyl, 2-(morpholin-4-yl)ethyl and 2-(morpholin-4-yl)ethoxy.
